SEQUENCE LISTING (136 sequences)

SEQ ID NO: 1 - DNA, 4103 bp, Streptococcus pyogenese
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgac

SEQ ID NO: 2 - DNA, 4113 bp, Artificial Sequence
  Codon optimized Cas9
atggataaaaagtatagtattggtttagatattggtactaactctgtgggttgggcagttatcaccgacgaatataaagttccatcaaagaaatttaaggtgttaggtaacactgacagacactcaataaaaaagaatcttatcggtgctcttttgttcgactccggtgaaactgccgaggctacacgtttaaaaagaacagcaagaagaagatatacccgtagaaaaaatagaatatgttatttacaagaaatcttttctaatgaaatggctaaagttgatgattcctttttccatagattggaagagtcatttttggttgaagaagacaaaaagcatgagagacatccaatctttgggaatatagttgatgaagtggcttaccatgaaaaatatcctaccatttatcatttaagaaagaaattggtagattcaactgataaagctgaccttagattaatctatttagcacttgcccatatgattaaatttagaggtcattttttgattgaaggtgatttgaacccagataattctgacgtggataaattatttattcaattagtccaaacctacaaccaattatttgaggaaaatccaattaatgctagtggtgtcgatgccaaagctatattatcagccagattatcaaaatctagacgtttggaaaatttgattgcccaattgccaggagaaaaaaagaatggattatttggaaacttgatcgcattatcattgggtttgacaccaaattttaaatctaattttgatttagctgaagatgctaaattacaattatcaaaagacacctatgacgacgatttggacaatttacttgctcaaattggtgatcaatatgcagatttgttcttagctgctaaaaacttatctgatgctattttgttgtctgatattttgagagtgaacacagaaataaccaaagctccattatcagcatctatgatcaaacgttatgatgaacaccatcaggatttgactttattgaaagctttggtgagacaacaattgccagagaagtataaagaaatctttttcgatcaatctaaaaacgggtatgcaggttatattgatgggggtgcctcccaagaggaattttacaaatttataaaacctattttagaaaagatggatgggactgaggaacttttggtcaaattgaacagagaagatttgttacgtaaacagagaacttttgataatggtagtatacctcaccaaattcatttgggtgagttgcatgcaattttaagaagacaagaagatttttatccatttttaaaagataatagagaaaaaatcgagaaaattttaacctttagaattccatactatgttgggcctttggctagaggtaattcaagatttgcctggatgacacgtaaatcagaagaaactattaccccttggaattttgaagaggttgttgataaaggagcatcagcacagagttttattgaaagaatgaccaatttcgataaaaacttaccaaatgaaaaagttttaccaaaacattccttgttatacgaatattttactgtttacaatgaacttacaaaggttaaatatgttactgaaggtatgcgtaagccagcctttttatctggagaacagaaaaaggcaatagttgatttattgtttaaaacaaatagaaaagttactgttaaacaattaaaagaagattactttaagaaaattgaatgttttgattcagttgaaatcagtggtgttgaagacagatttaatgctagtttaggaacttaccatgatttacttaaaattatcaaagataaagatttcttggataacgaagaaaatgaagacattttagaagacattgttttaaccttaactttattcgaagatagagagatgattgaagaacgtttgaagacttatgcacatttgtttgacgataaagtgatgaaacagttgaaaagaagacgttatactggatggggtagattgtctcgtaaattgatcaatggaattagagataaacaaagtggtaaaactatcttggactttttgaaatctgacggatttgctaatagaaatttcatgcaattgatccacgacgatagtttgacatttaaagaagacatccaaaaggcccaagtgagtgggcaaggtgattcattacatgaacatattgcaaatttagccggatctcctgctattaagaaagggatattacaaactgttaaagttgtggatgaattagtgaaagtaatgggaagacataaacctgaaaacattgtcattgagatggcaagagaaaatcaaactacacaaaaaggacagaaaaatagtagagaacgtatgaaaagaatagaagagggtattaaagaattgggtagtcaaatattgaaagaacacccagtggaaaatacccagttgcaaaatgaaaaattatatctttactaccttcaaaatggacgtgatatgtatgttgatcaggaattagatataaatagactttcagattatgatgtagatcatatagttccacaatctttcttgaaagatgattccatagacaataaagtattaactagaagtgataaaaatagaggtaaaagtgataatgtcccaagtgaggaagtcgtcaaaaagatgaaaaattactggcgtcaacttttgaatgctaaattaattactcaaagaaaatttgataatttgactaaagcagaaagaggtgggctttctgaattagataaagccgggttcattaaaagacaattggtcgaaactagacaaattactaaacatgttgcccaaattttagattcccgtatgaacactaagtatgacgaaaatgataagttaatacgtgaggttaaagtcattactttaaaatcaaaacttgtctctgatttcagaaaggatttccaattctataaagttagagaaattaataattatcatcatgctcatgatgcatatttgaatgctgtagttggaactgctttaatcaagaaataccctaaattagaatctgaatttgtatatggtgattacaaagtctatgatgttagaaagatgattgctaaatcagaacaagaaattggtaaagctacagctaaatacttcttttactctaacattatgaatttctttaaaacagaaattactttggcaaacggtgaaattagaaaaagacctcttattgaaacaaatggtgagactggagagatagtttgggacaaagggcgtgatttcgctactgttagaaaagttttatcaatgccacaagttaacattgtaaagaaaacagaggttcaaactggtggtttctcaaaagaaagtattttgcctaaaagaaatagtgataaattgattgccagaaaaaaggattgggatccaaagaaatatggtggtttcgactcaccaaccgtagcctattctgttttggttgtggcaaaggttgaaaagggtaaaagtaaaaagcttaaatcagtaaaagaacttttgggtattacaataatggaaagaagttcctttgaaaagaaccctattgattttttggaagctaaaggttataaggaagtaaagaaggacttaataatcaaattgcctaaatattctttatttgaattagaaaatgggagaaaaagaatgttggcttctgctggagaattgcaaaagggtaatgaattagcattgccttccaaatatgttaacttcttgtatttagcttcacactatgaaaagttgaaagggtcaccagaagataacgagcaaaaacaattatttgttgaacaacacaaacactacttagatgagattatagaacaaattagtgaattcagtaaaagagtgatattagctgatgcaaatttagataaagttttgtcagcctataacaaacatagagataagccaattagagaacaagcagaaaacattattcacttatttacccttaccaatttaggagcacctgctgctttcaagtattttgatacaacaattgatcgtaaaagatatacctcaacaaaagaagtcttagacgccaccttaattcatcaatcaatcactggattgtatgagacaagaattgatttgtctcaattgggtggtgatgaaggggct

SEQ ID NO: 3 - DNA, 4113 bp, Artificial Sequence
  Nuclease-inactive codon optimized Cas9
atggataaaaagtatagtattggtttagctattggtactaactctgtgggttgggcagttatcaccgacgaatataaagttccatcaaagaaatttaaggtgttaggtaacactgacagacactcaataaaaaagaatcttatcggtgctcttttgttcgactccggtgaaactgccgaggctacacgtttaaaaagaacagcaagaagaagatatacccgtagaaaaaatagaatatgttatttacaagaaatcttttctaatgaaatggctaaagttgatgattcctttttccatagattggaagagtcatttttggttgaagaagacaaaaagcatgagagacatccaatctttgggaatatagttgatgaagtggcttaccatgaaaaatatcctaccatttatcatttaagaaagaaattggtagattcaactgataaagctgaccttagattaatctatttagcacttgcccatatgattaaatttagaggtcattttttgattgaaggtgatttgaacccagataattctgacgtggataaattatttattcaattagtccaaacctacaaccaattatttgaggaaaatccaattaatgctagtggtgtcgatgccaaagctatattatcagccagattatcaaaatctagacgtttggaaaatttgattgcccaattgccaggagaaaaaaagaatggattatttggaaacttgatcgcattatcattgggtttgacaccaaattttaaatctaattttgatttagctgaagatgctaaattacaattatcaaaagacacctatgacgacgatttggacaatttacttgctcaaattggtgatcaatatgcagatttgttcttagctgctaaaaacttatctgatgctattttgttgtctgatattttgagagtgaacacagaaataaccaaagctccattatcagcatctatgatcaaacgttatgatgaacaccatcaggatttgactttattgaaagctttggtgagacaacaattgccagagaagtataaagaaatctttttcgatcaatctaaaaacgggtatgcaggttatattgatgggggtgcctcccaagaggaattttacaaatttataaaacctattttagaaaagatggatgggactgaggaacttttggtcaaattgaacagagaagatttgttacgtaaacagagaacttttgataatggtagtatacctcaccaaattcatttgggtgagttgcatgcaattttaagaagacaagaagatttttatccatttttaaaagataatagagaaaaaatcgagaaaattttaacctttagaattccatactatgttgggcctttggctagaggtaattcaagatttgcctggatgacacgtaaatcagaagaaactattaccccttggaattttgaagaggttgttgataaaggagcatcagcacagagttttattgaaagaatgaccaatttcgataaaaacttaccaaatgaaaaagttttaccaaaacattccttgttatacgaatattttactgtttacaatgaacttacaaaggttaaatatgttactgaaggtatgcgtaagccagcctttttatctggagaacagaaaaaggcaatagttgatttattgtttaaaacaaatagaaaagttactgttaaacaattaaaagaagattactttaagaaaattgaatgttttgattcagttgaaatcagtggtgttgaagacagatttaatgctagtttaggaacttaccatgatttacttaaaattatcaaagataaagatttcttggataacgaagaaaatgaagacattttagaagacattgttttaaccttaactttattcgaagatagagagatgattgaagaacgtttgaagacttatgcacatttgtttgacgataaagtgatgaaacagttgaaaagaagacgttatactggatggggtagattgtctcgtaaattgatcaatggaattagagataaacaaagtggtaaaactatcttggactttttgaaatctgacggatttgctaatagaaatttcatgcaattgatccacgacgatagtttgacatttaaagaagacatccaaaaggcccaagtgagtgggcaaggtgattcattacatgaacatattgcaaatttagccggatctcctgctattaagaaagggatattacaaactgttaaagttgtggatgaattagtgaaagtaatgggaagacataaacctgaaaacattgtcattgagatggcaagagaaaatcaaactacacaaaaaggacagaaaaatagtagagaacgtatgaaaagaatagaagagggtattaaagaattgggtagtcaaatattgaaagaacacccagtggaaaatacccagttgcaaaatgaaaaattatatctttactaccttcaaaatggacgtgatatgtatgttgatcaggaattagatataaatagactttcagattatgatgtagatgcaatagttccacaatctttcttgaaagatgattccatagacaataaagtattaactagaagtgataaaaatagaggtaaaagtgataatgtcccaagtgaggaagtcgtcaaaaagatgaaaaattactggcgtcaacttttgaatgctaaattaattactcaaagaaaatttgataatttgactaaagcagaaagaggtgggctttctgaattagataaagccgggttcattaaaagacaattggtcgaaactagacaaattactaaacatgttgcccaaattttagattcccgtatgaacactaagtatgacgaaaatgataagttaatacgtgaggttaaagtcattactttaaaatcaaaacttgtctctgatttcagaaaggatttccaattctataaagttagagaaattaataattatcatcatgctcatgatgcatatttgaatgctgtagttggaactgctttaatcaagaaataccctaaattagaatctgaatttgtatatggtgattacaaagtctatgatgttagaaagatgattgctaaatcagaacaagaaattggtaaagctacagctaaatacttcttttactctaacattatgaatttctttaaaacagaaattactttggcaaacggtgaaattagaaaaagacctcttattgaaacaaatggtgagactggagagatagtttgggacaaagggcgtgatttcgctactgttagaaaagttttatcaatgccacaagttaacattgtaaagaaaacagaggttcaaactggtggtttctcaaaagaaagtattttgcctaaaagaaatagtgataaattgattgccagaaaaaaggattgggatccaaagaaatatggtggtttcgactcaccaaccgtagcctattctgttttggttgtggcaaaggttgaaaagggtaaaagtaaaaagcttaaatcagtaaaagaacttttgggtattacaataatggaaagaagttcctttgaaaagaaccctattgattttttggaagctaaaggttataaggaagtaaagaaggacttaataatcaaattgcctaaatattctttatttgaattagaaaatgggagaaaaagaatgttggcttctgctggagaattgcaaaagggtaatgaattagcattgccttccaaatatgttaacttcttgtatttagcttcacactatgaaaagttgaaagggtcaccagaagataacgagcaaaaacaattatttgttgaacaacacaaacactacttagatgagattatagaacaaattagtgaattcagtaaaagagtgatattagctgatgcaaatttagataaagttttgtcagcctataacaaacatagagataagccaattagagaacaagcagaaaacattattcacttatttacccttaccaatttaggagcacctgctgctttcaagtattttgatacaacaattgatcgtaaaagatatacctcaacaaaagaagtcttagacgccaccttaattcatcaatcaatcactggattgtatgagacaagaattgatttgtctcaattgggtggtgatgaaggggct

SEQ ID NO: 4 - DNA, 144 bp, Artificial Sequence
  Flag fusion sequence
gatcctaagaagaaaagaaaagttgatccaaagaaaaagcgtaaggtggatcctaagaaaaagagaaaggttgactacaaagaccatgacggtgattataaagatcatgacatcgactacaaggatgacgatgacaagtgataa

SEQ ID NO: 5 - protein, 1371 aa, Streptococcus pyogenese
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGA

SEQ ID NO: 6 - protein, 1371 aa, Artificial Sequence
  Nuclease-inactive Cas9
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGA

SEQ ID NO: 7 - protein, 46 aa, Artificial Sequence
  Flag fusion sequence
DPKKKRKVDPKKKRKVDPKKKRKVDYKDHDGDYKDHDIDYKDDDDK

SEQ ID NO: 8 - DNA, 1008 bp, Candida albicans
gcggccgcaagtgattagacttagtccgttcaaatcaagcacaactctgttcattgtttcaacaagaattaattcaaaaacaggttcggtgcataatttgcaaaaaaatattgcagcttctgtggctcgaacacagtacctccagatttcaggtttgaaatacttcagtctgacgctctcccagatgagctaaagctgcaataagaaaacccacgccgggattcgaacccggaatcctttgattagaagtcaaaagcgataaccatttcgccacgcaggcctacttgatgggtttgtaaatggtctactttttcagacctaacagaaattttaatgaaagtcatattcttatacaataaaactgtgtcataaaagcagatattcgactttcgtagattatataggacccaagaactaaaatttaatgccatattatgcatttttaatctgtaaaagtgttgtttccaacctatcacaagtacgttcttgtaacttgtgtttgtagggttgcaaatgaatcataacaacatctcaacagaacatgtatagcaaagcttagtataaaatcagtgttttgagaggcaatccaagaatgtttacatcaaagtttcaataaatatcgaccgaaactgaaaatctttttaggttattgttcacttttttgtaaatatttaaacattttttggacctaaaaaaatacaaacaccaattacgtaccaagaagcatctaatcaactcccagatcaccactatacatttaaaagtcattggtcaataactatactcgagtattgcctcatcaaagaaacaatcaaatattatagatactcactccatcacgtgataatttcactggtatggaaaagtggaaaattttataaaaaaaaatttgatgcctttggcatagctgaaacttcggcccaataggattggagaatatgttttcgcagcgttcttacaattaaattgtggtggaagttcgagacttgcgtaaactatttttaattt

SEQ ID NO: 9 - DNA, 1561 bp, Unknown
  5′ ENO1 target
ctgccactactaccactgggagtttcgttcttctcgatactattagctttacttcctgcactagcagtggttggatcaacagaatcttcataatcatcaaaatcgtcttttgaagaccccccgtttgatgtatggccctgtcttttcatcaaactttttatatagttgactgaactgaggctaaatatgtgatcatcttcactatagacaatctttctcttatttgcaccaccgccaccactagtctttgagaaattctcaaaaccttttacgatattaccaagcgggctctcttcgaaataatctatctctttttgatatatcgaatcctctagcgtggttagctttctagttagttcttgcttcttaagaatttgctggattagtttatttttcaattcaacgtatttctcagagtcatctttagattttgatgaagatgtgcgttcattcgctatatccttcttggtcgtgtcttttcgatcctccttggctggcactgaactcgtcttttttggcgttgctgttccagacagacttatctcattagatttggaacttgtgggtttaacatcatttgtatctttagtagacatgattgtgcaataccgtgattatttgttttgaaaggtctgtcatatttctatcaatttcaaaacaaaatgttcatcagaaaaaagccaaaaatgtctcttctagtttcttagtggtgtcgcataatacacaatgtcgctcaacaatccacattcccggcgcatagctcaaatcacatgactacagctaacaattacacaaaaaaaattctctttttgatgtagcaactatcttcaactaaaacattttctccttcggcccatgattgtcctccgggtcgacagcaagccgttacaattgagatggaaagcgacctaccttcactcgataaggtgcttaattgtacttcatataaatctggcccggatctaaacaaatgagttccattaagccgtgggttctcaattagggtttttgtttttgatttagaaaaaagagatcaagatttgtttacaggtgatgcctttttttagaacttatgcgttgcaaaagttgactaacgatttctataaggtgatccacactaattatacaaacgtacaaacagacatacttttcctgcgttcacctgatgttggccagatttctctcttcattgcatagaacataaccacactagggcaacagaaaaaaaaaaaaaaagtgcatcgggaagttgtgttccattcattatatgtctactactgcatatgagtagcccacccaccaccaccatagtaagtttttgtgtatgcgcgccgtcaggttatttcatttctgaatttttcaaccaccttactccctttattgttgattgacaattttgctcacagtaagatcttttagactccaattaatataaaataagtctgattttccaattcctgttttttctttttttttctgtttctatttctttccttttctcccttttttttaattcttcattcaatcatcaattgataattcaggaatattacaacaaccc

SEQ ID NO: 10 - DNA, 2007 bp, Unknown
  3′ ENO1 target
gggtttgcctctgattaaataaaaaaaagctggtgctttttttttcttttataggaacatcttgaatatatgaactaattaaatgataattttttacccatctttactcttaatcactgagctgcagtcaaagaaaaagggatacagcacctggtgaagagatgaacggagactaacttagacgcgttgattctttttaattgcacattttattaatcgatgctaacgtctatttacatatattctttagagatattatctagggcttcaaataatctctggacagcaataaaagtctcttcaaaagtattgtataacggcaatggggctaatctgattacatctggtcttctttcgtcacagattatagcatgatcatgcaagtacgcattaactcgttccatgacgttcttgtccttttcatcgaaatgcggttgaaacataatggacaattgacatcctctttcagctggattcaaaggagttaaaattttaaacccaaatttggagtttgatgtactggattgtggtatgtaatacttggaattcgtcaatagatcctgtaaaaattgagtcaaagcaacacttttttcacgaagtttagatactccacccactttagcatacacttccaatgacgacttcacagcaacaacatcaagaacagaaggatttgactgtctgtaagaaagagccgagtttattggatcaaactcttctaacattttgaatcgttcttgggagttattgccccaccaaccagctagtctaggaacgaaactgcttttcttgttctctatggtgtatttttcatgcacaaaaatcccacctatggctccaggtcccgagtttaaatatttgtaggaacaccaagcagcaaaatctactccccaatcatgtaaatttaatgggacattcccaactgcatgggcaagatcccacccaactttaatttgttggctcttttccttagcgtatttagttatttcctctatcttgaaaaattgaccagtgtagtattggataccaggaaaacacactagagccaattcatccaggttctcatctatagccttgattattctttctgttttaatataagtttcaccaggttgaacttccaattgaatcaaatgtttctcgtcgtatccgaacaatttaacaatgttcaaaaatgcatagtagtcagaaggaaatgcttgtttttcaaataaaattttggttcttttcccctcaggtttgtaaaaatggatcaacaatgcattcaagtttgctgttaaagaacccataactgcaacttcgttttcctttgcaccaacaatgggggctattaatggtaataagggtaaatcgatgtctacccacggtgttaacagtttgtcaggatgattgaaatgagactcaacccctcgttcaacccatgcatttaattcatcattgatagctttctttgtattcttaggcatcaacccaagagagtttccacataaataaatagactcagttgatgactcatatttattatttttgatacctaatgatccaaaagttggtatggcaaactcatttttaaaagttgggaactttttgtccaatttctttgcctcggctaatgacatctgataataaaatggggttggagtagttggtggtataaccggagagatagaattgaagaaaaaaatcggaaacaacaaaaaaagttgataccctgtattatgtgggagataattgcgaatggtggaaaaaaaaaagacgccattgagtctcaacaacaattctgtcagctgaagagctttacaatcgagaaactatgattcattccgttttaatatgtatgtgtttagtaaactcatgaattttatttgtggtctactttagtactaacataatcattggatagtcaataatgatggtcttccgagactaatgaaattctataccaaagtcgatattccaacacagaaattgctcttgcaacaagtgcacctgttgatatctagagct

SEQ ID NO: 11 - DNA, 556 bp, Unknown
  5′ RP10 target
tggttgttaagtcagtagatgatttgttgttgtcgtttgattttgttacagcgtaaccagtgcgttttgtttgtttccacatcatacacttcactgaaactaaataagtttgtttacattttgagacttcaggtacgacccagggttgcgacaaagtttaggtagtttgtcgtctgaatgtcgcaacaaaatagggctgtagccctagtcatgtgatgtgaattaacagaacaagaagaactgctggtgcgcaaaaagattatgtgtattttatgtgcgttgttatcctgcacactaaaattgagcagtgtacacacacacatcttgggctgtatttttattcttgtttttctggtgttctctcactgttaagctctaagtgaatttgtgtgtgctgtaatagtgtgtgtgttccaagtcccagctctcacagatactcacgcacgcccatactactgaaaatttcctgactttctgtatctaaaaattttttactaggaatttttttcttttacgtttttcacttgtttcatataatcaccaactcaagtacaac

SEQ ID NO: 12 - DNA, 1000 bp, Unknown
  3′ RP10 target
tgtttaaggataatgataactgaagagaagaattagttttttcaagtgtataatatagtttctctctattaccttttccaataatagcattttaagttttctattttattttgtataaaaaacataatgaaaaatacgtataagtaatataaatgagtgtgggattaagtgaatacgagatgttgtagtgataataggggaaactctttggcgaaactacaagagagagtgatgtgctaataatgaacgaagaaatatgtgatttttgtatgaaatttgcaattattctgattgaatttgggtacttgacattgaatccagaacgactatacaaatgtgctactttgtcaaaatatcctttttgagaatcggcatatttatggccctgaatatcgactaccacattccttttacaacactacgtaaccttttgagaaagtacaagtgaaagaagtatagaattcagtgtttagtttaacgtaagtattactgtggaatgctttcttcgcgacacaagcaacttgtacctgcacccttcacacaatttatttcctaaaactactccagtgcgaaaacaatagtgctaaatatgatgatgagagaattcttaacgaacggagtaggaatgtacatactatcactagtttccaaataacaaaaataaaaaaaaaaataacatggaacttgtattgctaaataaattactagattttataagcaataaaaagaatttgaaaaggatgcttcatcacaactaatagtttagtttctttacttctcccctgtttactgggttattttatttagattatgctaatataattttttaatacaagaatttttatttttttaatttatgttgctgattgcccctaaaatttcaaattcctgaaattccctgagtgacttgaacccagacacacattcactcactcacacaaacaaatacacaaaattagagaacctgaatttcagattctcaaattccaaaacagcaaag

SEQ ID NO: 13 - DNA, 15526 bp, Artificial Sequence
  Plasmid
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacctttatcagcaagtagaaaacaaccaaagctcttgaaattgtgcaatgaagatttcatcaaacataaaaccattatgaacgcttggaagttgttacaacgaagaagaataacccaacaatctgaaaaattgtctaagcaatataaaagcattgtcaatgccatggaagatttgaagcaaacaagtcccgaattgttcgaagctgcaaatgctaaaaaccctaaacgtttcactaccttcccaatagagatgagagtgcctaccgattatccacctaacaagccatggacttacaactttgttccttcaaaaacccatcattagactgggttcagatgtaaatagatattatattataaatgtacataatcgaatagattgttattatttgttcaactcgtcctaatcctccaatactctcgcctttctttttctactaggtgtgccactactaccactgggcgtctcgttcttctcgatactattagctttacttcctgcactagcagtggttggatcaacagaatcttcataatcatcaaaatcgtcttttgaagaccccccgtttgatgtatggccctgtcttttcatcaaactttttatatagttgactgaactgaggctaaatatgtgatcatcttcactatagacaatctttctcttatttgcaccaccgccaccactagtctttgagaaattctcaaaaccttttacgatattaccaagcgggctctcttcgaaataatctatctctttttgatatatcgaatcctctagcgtggttagctttctagttagttcttgcttcttaagaatttgctggattagtttatttttcaattcaacgtatttctcagagtcatctttagattttgatgaagatgtgcgttcattcgctatatccttcttggtcgtgtcttttcgatcctccttggctggcactgaactcgtcttttttggcgttgctgttccagacagacttatctcattagatttggaacttgtgggtttaacatcatttgtatctttagtagacatgattgtgcaataccgtgattatttgttttgaaaggtctgtcatatttctatcaatttcaaaacaaaatgttcatcagaaaaaagccaaaaatgtctcttctagtttcttagtggtgtcgcataatacacaatgtcgctcaacaatccacattcccggcgcatagctcaaatcacatgactacagctaacaattacacaaaaaaaattctctttttgatgtagcaactatcttcaactaaaacattttctccttcggcccatgattgtcctccgggtcgacagcaagccgttacaattgagatggaaagcgacctaccttcactcgataaggtgcttaattgtacttcatataaatctggcccggatctaaacaaatgagttccattaagccgtgggttctcaattagggtttttgtttttgatttagaaaaaagagatcaagatttgtttacaggtgatgcctttttttagaacttatgcgttgcaaaagttgactaacgatttctataaggtgatccacactaattatacaaacgtacaaacagacatacttttcctgcgttcacctgatgttggccagatttctctcttcattgcatagaacataaccacactagggcaacagaaaaaaaaaaaaaaagtgcatcgggaagttgtgttccattcattatatgtctactactgcatatgagtagcccacccaccaccaccatagtaagtttttgtgtatgcgcgccgtcaggttatttcatttctgaatttttcaaccaccttactccctttattgttgattgacaattttgctcacagtaagatcttttagactccaattaatataaaataagtctgattttccaattcctgttttttctttttttttctgtttctatttctttccttttctcccttttttttaattcttcattcaatcatcaattgataattcaggaatattacaacaacccgggatggataaaaagtatagtattggtttagatattggtactaactctgtgggttgggcagttatcaccgacgaatataaagttccatcaaagaaatttaaggtgttaggtaacactgacagacactcaataaaaaagaatcttatcggtgctcttttgttcgactccggtgaaactgccgaggctacacgtttaaaaagaacagcaagaagaagatatacccgtagaaaaaatagaatatgttatttacaagaaatcttttctaatgaaatggctaaagttgatgattcctttttccatagattggaagagtcatttttggttgaagaagacaaaaagcatgagagacatccaatctttgggaatatagttgatgaagtggcttaccatgaaaaatatcctaccatttatcatttaagaaagaaattggtagattcaactgataaagctgaccttagattaatctatttagcacttgcccatatgattaaatttagaggtcattttttgattgaaggtgatttgaacccagataattctgacgtggataaattatttattcaattagtccaaacctacaaccaattatttgaggaaaatccaattaatgctagtggtgtcgatgccaaagctatattatcagccagattatcaaaatctagacgtttggaaaatttgattgcccaattgccaggagaaaaaaagaatggattatttggaaacttgatcgcattatcattgggtttgacaccaaattttaaatctaattttgatttagctgaagatgctaaattacaattatcaaaagacacctatgacgacgatttggacaatttacttgctcaaattggtgatcaatatgcagatttgttcttagctgctaaaaacttatctgatgctattttgttgtctgatattttgagagtgaacacagaaataaccaaagctccattatcagcatctatgatcaaacgttatgatgaacaccatcaggatttgactttattgaaagctttggtgagacaacaattgccagagaagtataaagaaatctttttcgatcaatctaaaaacgggtatgcaggttatattgatgggggtgcctcccaagaggaattttacaaatttataaaacctattttagaaaagatggatgggactgaggaacttttggtcaaattgaacagagaagatttgttacgtaaacagagaacttttgataatggtagtatacctcaccaaattcatttgggtgagttgcatgcaattttaagaagacaagaagatttttatccatttttaaaagataatagagaaaaaatcgagaaaattttaacctttagaattccatactatgttgggcctttggctagaggtaattcaagatttgcctggatgacacgtaaatcagaagaaactattaccccttggaattttgaagaggttgttgataaaggagcatcagcacagagttttattgaaagaatgaccaatttcgataaaaacttaccaaatgaaaaagttttaccaaaacattccttgttatacgaatattttactgtttacaatgaacttacaaaggttaaatatgttactgaaggtatgcgtaagccagcctttttatctggagaacagaaaaaggcaatagttgatttattgtttaaaacaaatagaaaagttactgttaaacaattaaaagaagattactttaagaaaattgaatgttttgattcagttgaaatcagtggtgttgaagacagatttaatgctagtttaggaacttaccatgatttacttaaaattatcaaagataaagatttcttggataacgaagaaaatgaagacattttagaagacattgttttaaccttaactttattcgaagatagagagatgattgaagaacgtttgaagacttatgcacatttgtttgacgataaagtgatgaaacagttgaaaagaagacgttatactggatggggtagattgtctcgtaaattgatcaatggaattagagataaacaaagtggtaaaactatcttggactttttgaaatctgacggatttgctaatagaaatttcatgcaattgatccacgacgatagtttgacatttaaagaagacatccaaaaggcccaagtgagtgggcaaggtgattcattacatgaacatattgcaaatttagccggatctcctgctattaagaaagggatattacaaactgttaaagttgtggatgaattagtgaaagtaatgggaagacataaacctgaaaacattgtcattgagatggcaagagaaaatcaaactacacaaaaaggacagaaaaatagtagagaacgtatgaaaagaatagaagagggtattaaagaattgggtagtcaaatattgaaagaacacccagtggaaaatacccagttgcaaaatgaaaaattatatctttactaccttcaaaatggacgtgatatgtatgttgatcaggaattagatataaatagactttcagattatgatgtagatcatatagttccacaatctttcttgaaagatgattccatagacaataaagtattaactagaagtgataaaaatagaggtaaaagtgataatgtcccaagtgaggaagtcgtcaaaaagatgaaaaattactggcgtcaacttttgaatgctaaattaattactcaaagaaaatttgataatttgactaaagcagaaagaggtgggctttctgaattagataaagccgggttcattaaaagacaattggtcgaaactagacaaattactaaacatgttgcccaaattttagattcccgtatgaacactaagtatgacgaaaatgataagttaatacgtgaggttaaagtcattactttaaaatcaaaacttgtctctgatttcagaaaggatttccaattctataaagttagagaaattaataattatcatcatgctcatgatgcatatttgaatgctgtagttggaactgctttaatcaagaaataccctaaattagaatctgaatttgtatatggtgattacaaagtctatgatgttagaaagatgattgctaaatcagaacaagaaattggtaaagctacagctaaatacttcttttactctaacattatgaatttctttaaaacagaaattactttggcaaacggtgaaattagaaaaagacctcttattgaaacaaatggtgagactggagagatagtttgggacaaagggcgtgatttcgctactgttagaaaagttttatcaatgccacaagttaacattgtaaagaaaacagaggttcaaactggtggtttctcaaaagaaagtattttgcctaaaagaaatagtgataaattgattgccagaaaaaaggattgggatccaaagaaatatggtggtttcgactcaccaaccgtagcctattctgttttggttgtggcaaaggttgaaaagggtaaaagtaaaaagcttaaatcagtaaaagaacttttgggtattacaataatggaaagaagttcctttgaaaagaaccctattgattttttggaagctaaaggttataaggaagtaaagaaggacttaataatcaaattgcctaaatattctttatttgaattagaaaatgggagaaaaagaatgttggcttctgctggagaattgcaaaagggtaatgaattagcattgccttccaaatatgttaacttcttgtatttagcttcacactatgaaaagttgaaagggtcaccagaagataacgagcaaaaacaattatttgttgaacaacacaaacactacttagatgagattatagaacaaattagtgaattcagtaaaagagtgatattagctgatgcaaatttagataaagttttgtcagcctataacaaacatagagataagccaattagagaacaagcagaaaacattattcacttatttacccttaccaatttaggagcacctgctgctttcaagtattttgatacaacaattgatcgtaaaagatatacctcaacaaaagaagtcttagacgccaccttaattcatcaatcaatcactggattgtatgagacaagaattgatttgtctcaattgggtggtgatgaaggggctgatcctaagaagaaaagaaaagttgatccaaagaaaaagcgtaaggtggatcctaagaaaaagagaaaggttgactacaaagaccatgacggtgattataaagatcatgacatcgactacaaggatgacgatgacaagtgataatgactgcagagatccatcgacctgccgccaagctaattccgggcgaatttctgtcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccgggccccccctcgaggaagttcctatactttctagagaataggaacttcggatccactagttctagatttttgcaagcatttaaatattgccaagtaaaaacttcaaattttctttccccttggaactttgactttatttttttgacagattattttgacacacacacaccaaatgtgttaccccttaaaacaaaaaaacacttttttacaatttcttggtatccagaatcattctaagcatcattcaattataatttcaatccaaaaaagtagttttagtttgacttgaaacgtcaacaaacacaaatttcaaatcataacctctcctgttgcctgtcaacaacacaccataaggagaaggaataggaggaggaggagatagaaacttgcacggcaccacaaaacacaaaattgatttcaaccaatacggtgacaacaacaatagatttccgatagaaataatgattatcggaataagctagctttgctttgctttgctttgctttttgacttgctctaatttttcgaaaataataatggagaaaagttcaaggtgtttaatgcatcaactaaaacagaaaataatacattagactaaacttttaatctttctagtaccaataattcacgcgtgcgttttaatcccaatcatgaaatgaagaagttatttccctttttctttcatcaaaaaagaactaaattattttttaaattttagtaaacaaaacctggaaatcggggaaaccgggggaggggggcagaaggtgaaacgggtaatattgataaatttaatctataattgataaagttaaatttaaattgatttgaattgatttgaattgaatgaaatgcatttgaataaacggcatcaaactaaaaaaatatagatcacattcatagtaaaacgataacaaagaacaccacaatttatagcaatgataataaacatctaaaaagaaaagggtacgagaaggagaatgaaaaaaaacaataagctagttcttaatctgttcagatatctaatttcaaaaaaaagaatagtataaaaggatagttgattcctcttggttgttgaaaatttgaataatatcaatcaattaatcaatcaaataacaacaacccactagacatcaccattgtcgacatgccacaatttgatatattatgtaaaacaccacctaaggtgcttgttcgtcagtttgtggaaaggtttgaaagaccttcaggtgagaaaatagcattatgtgctgctgaactaacctatttatgttggatgattacacataacggaacagcaatcaagagagccacattcatgagctataatactatcataagcaattcgttgagtttcgatattgtcaataaatcactccagtttaaatacaagacgcaaaaagcaacaattttggaagcctcattaaagaaattgattcctgcttgggaatttacaattattccttactatggacaaaaacatcaatctgatatcactgatattgtaagtagtttgcaattacagttcgaatcatcggaagaagcagataagggaaatagccacagtaaaaaaatgcttaaagcacttctaagtgagggtgaaagcatctgggagatcactgagaaaatactaaattcgtttgagtatacttcgagatttacaaaaacaaaaactttataccaattcctcttcctagctactttcatcaattgtggaagattcagcgatattaagaacgttgatccgaaatcatttaaattagtccaaaataagtatttgggagtaataatccagtgtttagtgacagagacaaagacaagcgttagtaggcacatatacttctttagcgcaaggggtaggatcgatccacttgtatatttggatgaatttttgaggaattctgaaccagtcctaaaacgagtaaataggaccggcaattcttcaagcaataaacaggaataccaattattaaaagataacttagtcagatcgtacaataaagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagagtgaaattctggaaatctggaaatctggttttgtattcttgttattcttctttttgttattacatatataacttgttacttttttaaaaaaatctttgtttattttataaatatataaaactaaatttaagaaaaagagaaaaatgttttatttgagagattgatattttacttgaatttagcttagcttttataaagtattattatgtaaaaaaacaaaacaaatatacattaaaaagttaagactataaaatagccacccaaggcatttctatatcttgttgttgttgttttcatcttctgtatcagaggaacttattttattattttcgtcacgggtattttctcttgtttgatgattcatcccattcattccatcataaaatgtcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactgactcgagttattatggacatggcatagacatatacaaagcttgttcaccatcggaagcagtaccatcgtataaagcagtatccaaaccacacaaagtgaaacccattcttctataagcatgaatagctggagcattaacattggtaacttccaaccacaaatgaccagcacctctttctctggcgaattcagtagccaaacccatcaaagctctaccaacaccatgacctctatgttctggagcaacttcaatatcttcaacagtcaatcttctgttccaaccagaataagaaacaacaacgaaaccagccaaatcaccatcatcaccataagcaacgaaagttctagaatctggatcaccatcttcaccagcatcggattcatcatcggattcatcatctgggaaaaccttagtcaatggtggatcaactggaacttctctcaaagtgaaaccatcaccagtagcagtaactctaaaaacagtatcggtagtgaaagaaccatccaaagcttcaatagcttcagcatcacctggaacagaagttctgtatctataagcagtatcatccaaagtagtagacataattgtaggatccggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggcaagcttgatggaagttcctatactttctagagaataggaacttcagatccactagttctagagcggccgccaccgcgggtttgcctctgattaaataaaaaaaagctggtgctttttttttcttttataggaacatcttgaatatatgaactaattaaatgataattttttacccatctttactcttaatcactgagctgcagtcaaagaaaaagggatacagcacctggtgaagagatgaacggagactaacttagacgcgttgattctttttaattgcacattttattaatcgatgctaacgtctatttacatatattctttagagatattatctagggcttcaaataatctctggacagcaataaaagtctcttcaaaagtattgtataacggcaatggggctaatctgattacatctggtcttctttcgtcacagattatagcatgatcatgcaagtacgcattaactcgttccatgacgttcttgtccttttcatcgaaatgcggttgaaacataatggacaattgacatcctctttcagctggattcaaaggagttaaaattttaaacccaaatttggagtttgatgtactggattgtggtatgtaatacttggaattcgtcaatagatcctgtaaaaattgagtcaaagcaacacttttttcacgaagtttagatactccacccactttagcatacacttccaatgacgacttcacagcaacaacatcaagaacagaaggatttgactgtctgtaagaaagagccgagtttattggatcaaactcttctaacattttgaatcgttcttgggagttattgccccaccaaccagctagtctaggaacgaaactgcttttcttgttctctatggtgtatttttcatgcacaaaaatcccacctatggctccaggtcccgagtttaaatatttgtaggaacaccaagcagcaaaatctactccccaatcatgtaaatttaatgggacattcccaactgcatgggcaagatcccacccaactttaatttgttggctcttttccttagcgtatttagttatttcctctatcttgaaaaattgaccagtgtagtattggataccaggaaaacacactagagccaattcatccaggttctcatctatagccttgattattctttctgttttaatataagtttcaccaggttgaacttccaattgaatcaaatgtttctcgtcgtatccgaacaatttaacaatgttcaaaaatgcatagtagtcagaaggaaatgcttgtttttcaaataaaattttggttcttttcccctcaggtttgtaaaaatggatcaacaatgcattcaagtttgctgttaaagaacccataactgcaacttcgttttcctttgcaccaacaatgggggctattaatggtaataagggtaaatcgatgtctacccacggtgttaacagtttgtcaggatgattgaaatgagactcaacccctcgttcaacccatgcatttaattcatcattgatagctttctttgtattcttaggcatcaacccaagagagtttccacataaataaatagactcagttgatgactcatatttattatttttgatacctaatgatccaaaagttggtatggcaaactcatttttaaaagttgggaactttttgtccaatttctttgcctcggctaatgacatctgataataaaatggggttggagtagttggtggtataaccggagagatagaattgaagaaaaaaatcggaaacaacaaaaaaagttgataccctgtattatgtgggagataattgcgaatggtggaaaaaaaaaagacgccattgagtctcaacaacaattctgtcagctgaagagctttacaatcgagaaactatgattcattccgttttaatatgtatgtgtttagtaaactcatgaattttatttgtggtctactttagtactaacataatcattggatagtcaataatgatggtcttccgagactaatgaaattctataccaaagtcgatattccaacacagaaattgctcttgcaacaagtgcacctgttgatatctagagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 14 - DNA, 7418 bp, Artificial Sequence
  Plasmid
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacctggttgttaagtcagtagatgatttgttgttgtcgtttgattttgttacagcgtaaccagtgcgttttgtttgtttccacatcatacacttcactgaaactaaataagtttgtttacattttgagacttcaggtacgacccagggttgcgacaaagtttaggtagtttgtcgtctgaatgtcgcaacaaaatagggctgtagccctagtcatgtgatgtgaattaacagaacaagaagaactgctggtgcgcaaaaagattatgtgtattttatgtgcgttgttatcctgcacactaaaattgagcagtgtacacacacacatcttgggctgtatttttattcttgtttttctggtgttctctcactgttaagctctaagtgaatttgtgtgtgctgtaatagtgtgtgtgttccaagtcccagctctcacagatactcacgcacgcccatactactgaaaatttcctgactttctgtatctaaaaattttttactaggaatttttttcttttacgtttttcacttgtttcatataatcaccaactcaagtacaacagatctggaccacctttgattgtaaatagtaataattaccacccttatctaattatttatttaacttatttatttatttattatacatatatacaaatctaataaagtgaaaatctcccccttcacacttcacatatgttaggcgtcatcctgtgctcccgagaaccagtaccagtacatcgctgtttcgttcgagacttgaggtctagttttatacgtgaagaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaattttcatatgagaaccgttatcgataactaaagcagcaacttcttctataaaaatgggttagtatgacagtcatttaaataaggaatttttcagttggcttggtttcaattcaatgttcgttttttttttttcttgctgtgtttgtgtttgtgttgtttatagttgtgtgcactgatcgtcgaaaaaaaaaattcatagtgagccgggaaatctgtatagcccagataacaacacaagtccaaactagaaactcgtcaaacaccaaaagcaatgttgaatcaattgccttgcacaagtacacgtaggaaaacataaaacattgcaattttgaatattgagccttttgtcgtaacattgattgataggattactcaccgaatggttttgaaaccactgccgacagatcaatcaatcaatcaaaaaacgtgaactttgaaaaaggggaagaacagatacattgaagttagccatttccactgatcgtcacaacatatctgataaattactttcaaaattataagctgatgtgtgtgtattattaatgtgacagtaacatcccaaacgagaaatattatctcgacaacaaaaaagtttgatctgaattgaaaatgaagttttcccaccctacccatttgtcatattgaaaccaatcaactgattaatcaatcaattagaattgaagctaaactaaaacataccaccgtccattttgaatgattatatttttttaatattaatatcgagataatgtttctaagaaagaaagaaaaccaggagtgaaaattagaaaaggaaaggaaaggaaaaaaagaaaaatctgaaaatatataaaaaaaaattgtttcgttggcaataaatcttggtgagaacagcgaccgaaagcaaataagaacaaaatatgagtgtattacgttgaacaactaattaacgtgtgtgtatggatctttttttcttttttctctttaaccgactataaacaacaaacatttttgggcagtgcacacactacttaatatacacagcataaattacacgattagaaacaaattagcttattaaaataacctaatcaaaccgaatattttatggtattatgagtaaactatataatataaatagcacacacccacaacaacaacaaaggaaaactaaaaggttttttctttttgaaaagatcgttttctttattattctctagttttgacggcggccgcaagtgattagacttagtccgttcaaatcaagcacaactctgttcattgtttcaacaagaattaattcaaaaacaggttcggtgcataatttgcaaaaaaatattgcagcttctgtggctcgaacacagtacctccagatttcaggtttgaaatacttcagtctgacgctctcccagatgagctaaagctgcaataagaaaacccacgccgggattcgaacccggaatcctttgattagaagtcaaaagcgataaccatttcgccacgcaggcctacttgatgggtttgtaaatggtctactttttcagacctaacagaaattttaatgaaagtcatattcttatacaataaaactgtgtcataaaagcagatattcgactttcgtagattatataggacccaagaactaaaatttaatgccatattatgcatttttaatctgtaaaagtgttgtttccaacctatcacaagtacgttcttgtaacttgtgtttgtagggttgcaaatgaatcataacaacatctcaacagaacatgtatagcaaagcttagtataaaatcagtgttttgagaggcaatccaagaatgtttacatcaaagtttcaataaatatcgaccgaaactgaaaatctttttaggttattgttcacttttttgtaaatatttaaacattttttggacctaaaaaaatacaaacaccaattacgtaccaagaagcatctaatcaactcccagatcaccactatacatttaaaagtcattggtcaataactatactcgagtattgcctcatcaaagaaacaatcaaatattatagatactcactccatcacgtgataatttcactggtatggaaaagtggaaaattttataaaaaaaaatttgatgcctttggcatagctgaaacttcggcccaataggattggagaatatgttttcgcagcgttcttacaattaaattgtggtggaagttcgagacttgcgtaaactatttttaatttggagacggaattccgtctcgttttagagctagaaatagcaagttaaaataaggctagtccgttatcaacttgaaaaagtggcaccgagtcggtgctttttttctcgagtttttttatcgagtgtttaaggataatgataactgaagagaagaattagttttttcaagtgtataatatagtttctctctattaccttttccaataatagcattttaagttttctattttattttgtataaaaaacataatgaaaaatacgtataagtaatataaatgagtgtgggattaagtgaatacgagatgttgtagtgataataggggaaactctttggcgaaactacaagagagagtgatgtgctaataatgaacgaagaaatatgtgatttttgtatgaaatttgcaattattctgattgaatttgggtacttgacattgaatccagaacgactatacaaatgtgctactttgtcaaaatatcctttttgagaatcggcatatttatggccctgaatatcgactaccacattccttttacaacactacgtaaccttttgagaaagtacaagtgaaagaagtatagaattcagtgtttagtttaacgtaagtattactgtggaatgctttcttcgcgacacaagcaacttgtacctgcacccttcacacaatttatttcctaaaactactccagtgcgaaaacaatagtgctaaatatgatgatgagagaattcttaacgaacggagtaggaatgtacatactatcactagtttccaaataacaaaaataaaaaaaaaaataacatggaacttgtattgctaaataaattactagattttataagcaataaaaagaatttgaaaaggatgcttcatcacaactaatagtttagtttctttacttctcccctgtttactgggttattttatttagattatgctaatataattttttaatacaagaatttttatttttttaatttatgttgctgattgcccctaaaatttcaaattcctgaaattccctgagtgacttgaacccagacacacattcactcactcacacaaacaaatacacaaaattagagaacctgaatttcagattctcaaattccaaaacagcaaagccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 15 - DNA, 14062 bp, Artificial Sequence
  Plasmid
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacctgccactactaccactgggagtttcgttcttctcgatactattagctttacttcctgcactagcagtggttggatcaacagaatcttcataatcatcaaaatcgtcttttgaagaccccccgtttgatgtatggccctgtcttttcatcaaactttttatatagttgactgaactgaggctaaatatgtgatcatcttcactatagacaatctttctcttatttgcaccaccgccaccactagtctttgagaaattctcaaaaccttttacgatattaccaagcgggctctcttcgaaataatctatctctttttgatatatcgaatcctctagcgtggttagctttctagttagttcttgcttcttaagaatttgctggattagtttatttttcaattcaacgtatttctcagagtcatctttagattttgatgaagatgtgcgttcattcgctatatccttcttggtcgtgtcttttcgatcctccttggctggcactgaactcgtcttttttggcgttgctgttccagacagacttatctcattagatttggaacttgtgggtttaacatcatttgtatctttagtagacatgattgtgcaataccgtgattatttgttttgaaaggtctgtcatatttctatcaatttcaaaacaaaatgttcatcagaaaaaagccaaaaatgtctcttctagtttcttagtggtgtcgcataatacacaatgtcgctcaacaatccacattcccggcgcatagctcaaatcacatgactacagctaacaattacacaaaaaaaattctctttttgatgtagcaactatcttcaactaaaacattttctccttcggcccatgattgtcctccgggtcgacagcaagccgttacaattgagatggaaagcgacctaccttcactcgataaggtgcttaattgtacttcatataaatctggcccggatctaaacaaatgagttccattaagccgtgggttctcaattagggtttttgtttttgatttagaaaaaagagatcaagatttgtttacaggtgatgcctttttttagaacttatgcgttgcaaaagttgactaacgatttctataaggtgatccacactaattatacaaacgtacaaacagacatacttttcctgcgttcacctgatgttggccagatttctctcttcattgcatagaacataaccacactagggcaacagaaaaaaaaaaaaaaagtgcatcgggaagttgtgttccattcattatatgtctactactgcatatgagtagcccacccaccaccaccatagtaagtttttgtgtatgcgcgccgtcaggttatttcatttctgaatttttcaaccaccttactccctttattgttgattgacaattttgctcacagtaagatcttttagactccaattaatataaaataagtctgattttccaattcctgttttttctttttttttctgtttctatttctttccttttctcccttttttttaattcttcattcaatcatcaattgataattcaggaatattacaacaacccgggatggataaaaagtatagtattggtttagatattggtactaactctgtgggttgggcagttatcaccgacgaatataaagttccatcaaagaaatttaaggtgttaggtaacactgacagacactcaataaaaaagaatcttatcggtgctcttttgttcgactccggtgaaactgccgaggctacacgtttaaaaagaacagcaagaagaagatatacccgtagaaaaaatagaatatgttatttacaagaaatcttttctaatgaaatggctaaagttgatgattcctttttccatagattggaagagtcatttttggttgaagaagacaaaaagcatgagagacatccaatctttgggaatatagttgatgaagtggcttaccatgaaaaatatcctaccatttatcatttaagaaagaaattggtagattcaactgataaagctgaccttagattaatctatttagcacttgcccatatgattaaatttagaggtcattttttgattgaaggtgatttgaacccagataattctgacgtggataaattatttattcaattagtccaaacctacaaccaattatttgaggaaaatccaattaatgctagtggtgtcgatgccaaagctatattatcagccagattatcaaaatctagacgtttggaaaatttgattgcccaattgccaggagaaaaaaagaatggattatttggaaacttgatcgcattatcattgggtttgacaccaaattttaaatctaattttgatttagctgaagatgctaaattacaattatcaaaagacacctatgacgacgatttggacaatttacttgctcaaattggtgatcaatatgcagatttgttcttagctgctaaaaacttatctgatgctattttgttgtctgatattttgagagtgaacacagaaataaccaaagctccattatcagcatctatgatcaaacgttatgatgaacaccatcaggatttgactttattgaaagctttggtgagacaacaattgccagagaagtataaagaaatctttttcgatcaatctaaaaacgggtatgcaggttatattgatgggggtgcctcccaagaggaattttacaaatttataaaacctattttagaaaagatggatgggactgaggaacttttggtcaaattgaacagagaagatttgttacgtaaacagagaacttttgataatggtagtatacctcaccaaattcatttgggtgagttgcatgcaattttaagaagacaagaagatttttatccatttttaaaagataatagagaaaaaatcgagaaaattttaacctttagaattccatactatgttgggcctttggctagaggtaattcaagatttgcctggatgacacgtaaatcagaagaaactattaccccttggaattttgaagaggttgttgataaaggagcatcagcacagagttttattgaaagaatgaccaatttcgataaaaacttaccaaatgaaaaagttttaccaaaacattccttgttatacgaatattttactgtttacaatgaacttacaaaggttaaatatgttactgaaggtatgcgtaagccagcctttttatctggagaacagaaaaaggcaatagttgatttattgtttaaaacaaatagaaaagttactgttaaacaattaaaagaagattactttaagaaaattgaatgttttgattcagttgaaatcagtggtgttgaagacagatttaatgctagtttaggaacttaccatgatttacttaaaattatcaaagataaagatttcttggataacgaagaaaatgaagacattttagaagacattgttttaaccttaactttattcgaagatagagagatgattgaagaacgtttgaagacttatgcacatttgtttgacgataaagtgatgaaacagttgaaaagaagacgttatactggatggggtagattgtctcgtaaattgatcaatggaattagagataaacaaagtggtaaaactatcttggactttttgaaatctgacggatttgctaatagaaatttcatgcaattgatccacgacgatagtttgacatttaaagaagacatccaaaaggcccaagtgagtgggcaaggtgattcattacatgaacatattgcaaatttagccggatctcctgctattaagaaagggatattacaaactgttaaagttgtggatgaattagtgaaagtaatgggaagacataaacctgaaaacattgtcattgagatggcaagagaaaatcaaactacacaaaaaggacagaaaaatagtagagaacgtatgaaaagaatagaagagggtattaaagaattgggtagtcaaatattgaaagaacacccagtggaaaatacccagttgcaaaatgaaaaattatatctttactaccttcaaaatggacgtgatatgtatgttgatcaggaattagatataaatagactttcagattatgatgtagatcatatagttccacaatctttcttgaaagatgattccatagacaataaagtattaactagaagtgataaaaatagaggtaaaagtgataatgtcccaagtgaggaagtcgtcaaaaagatgaaaaattactggcgtcaacttttgaatgctaaattaattactcaaagaaaatttgataatttgactaaagcagaaagaggtgggctttctgaattagataaagccgggttcattaaaagacaattggtcgaaactagacaaattactaaacatgttgcccaaattttagattcccgtatgaacactaagtatgacgaaaatgataagttaatacgtgaggttaaagtcattactttaaaatcaaaacttgtctctgatttcagaaaggatttccaattctataaagttagagaaattaataattatcatcatgctcatgatgcatatttgaatgctgtagttggaactgctttaatcaagaaataccctaaattagaatctgaatttgtatatggtgattacaaagtctatgatgttagaaagatgattgctaaatcagaacaagaaattggtaaagctacagctaaatacttcttttactctaacattatgaatttctttaaaacagaaattactttggcaaacggtgaaattagaaaaagacctcttattgaaacaaatggtgagactggagagatagtttgggacaaagggcgtgatttcgctactgttagaaaagttttatcaatgccacaagttaacattgtaaagaaaacagaggttcaaactggtggtttctcaaaagaaagtattttgcctaaaagaaatagtgataaattgattgccagaaaaaaggattgggatccaaagaaatatggtggtttcgactcaccaaccgtagcctattctgttttggttgtggcaaaggttgaaaagggtaaaagtaaaaagcttaaatcagtaaaagaacttttgggtattacaataatggaaagaagttcctttgaaaagaaccctattgattttttggaagctaaaggttataaggaagtaaagaaggacttaataatcaaattgcctaaatattctttatttgaattagaaaatgggagaaaaagaatgttggcttctgctggagaattgcaaaagggtaatgaattagcattgccttccaaatatgttaacttcttgtatttagcttcacactatgaaaagttgaaagggtcaccagaagataacgagcaaaaacaattatttgttgaacaacacaaacactacttagatgagattatagaacaaattagtgaattcagtaaaagagtgatattagctgatgcaaatttagataaagttttgtcagcctataacaaacatagagataagccaattagagaacaagcagaaaacattattcacttatttacccttaccaatttaggagcacctgctgctttcaagtattttgatacaacaattgatcgtaaaagatatacctcaacaaaagaagtcttagacgccaccttaattcatcaatcaatcactggattgtatgagacaagaattgatttgtctcaattgggtggtgatgaaggggctgatcctaagaagaaaagaaaagttgatccaaagaaaaagcgtaaggtggatcctaagaaaaagagaaaggttgactacaaagaccatgacggtgattataaagatcatgacatcgactacaaggatgacgatgacaagtgataatgactgcagagatccatcgacctgccgccaagctaattccgggcgaatttctgtcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccgggccccccctaactcaagtacaacagatctggaccacctttgattgtaaatagtaataattaccacccttatctaattatttatttaacttatttatttatttattatacatatatacaaatctaataaagtgaaaatctcccccttcacacttcacatatgttaggcgtcatcctgtgctcccgagaaccagtaccagtacatcgctgtttcgttcgagacttgaggtctagttttatacgtgaagaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaattttcatatgagaaccgttatcgataactaaagcagcaacttcttctataaaaatgggttagtatgacagtcatttaaataaggaatttttcagttggcttggtttcaattcaatgttcgttttttttttttcttgctgtgtttgtgtttgtgttgtttatagttgtgtgcactgatcgtcgaaaaaaaaaattcatagtgagccgggaaatctgtatagcccagataacaacacaagtccaaactagaaactcgtcaaacaccaaaagcaatgttgaatcaattgccttgcacaagtacacgtaggaaaacataaaacattgcaattttgaatattgagccttttgtcgtaacattgattgataggattactcaccgaatggttttgaaaccactgccgacagatcaatcaatcaatcaaaaaacgtgaactttgaaaaaggggaagaacagatacattgaagttagccatttccactgatcgtcacaacatatctgataaattactttcaaaattataagctgatgtgtgtgtattattaatgtgacagtaacatcccaaacgagaaatattatctcgacaacaaaaaagtttgatctgaattgaaaatgaagttttcccaccctacccatttgtcatattgaaaccaatcaactgattaatcaatcaattagaattgaagctaaactaaaacataccaccgtccattttgaatgattatatttttttaatattaatatcgagataatgtttctaagaaagaaagaaaaccaggagtgaaaattagaaaaggaaaggaaaggaaaaaaagaaaaatctgaaaatatataaaaaaaaattgtttcgttggcaataaatcttggtgagaacagcgaccgaaagcaaataagaacaaaatatgagtgtattacgttgaacaactaattaacgtgtgtgtatggatctttttttcttttttctctttaaccgactataaacaacaaacatttttgggcagtgcacacactacttaatatacacagcataaattacacgattagaaacaaattagcttattaaaataacctaatcaaaccgaatattttatggtattatgagtaaactatataatataaatagcacacacccacaacaacaacaaaggaaaactaaaaggttttttctttttgaaaagatcgttttctttattattctctagttttgacggcggccgcaagtgattagacttagtccgttcaaatcaagcacaactctgttcattgtttcaacaagaattaattcaaaaacaggttcggtgcataatttgcaaaaaaatattgcagcttctgtggctcgaacacagtacctccagatttcaggtttgaaatacttcagtctgacgctctcccagatgagctaaagctgcaataagaaaacccacgccgggattcgaacccggaatcctttgattagaagtcaaaagcgataaccatttcgccacgcaggcctacttgatgggtttgtaaatggtctactttttcagacctaacagaaattttaatgaaagtcatattcttatacaataaaactgtgtcataaaagcagatattcgactttcgtagattatataggacccaagaactaaaatttaatgccatattatgcatttttaatctgtaaaagtgttgtttccaacctatcacaagtacgttcttgtaacttgtgtttgtagggttgcaaatgaatcataacaacatctcaacagaacatgtatagcaaagcttagtataaaatcagtgttttgagaggcaatccaagaatgtttacatcaaagtttcaataaatatcgaccgaaactgaaaatctttttaggttattgttcacttttttgtaaatatttaaacattttttggacctaaaaaaatacaaacaccaattacgtaccaagaagcatctaatcaactcccagatcaccactatacatttaaaagtcattggtcaataactatactcgagtattgcctcatcaaagaaacaatcaaatattatagatactcactccatcacgtgataatttcactggtatggaaaagtggaaaattttataaaaaaaaatttgatgcctttggcatagctgaaacttcggcccaataggattggagaatatgttttcgcagcgttcttacaattaaattgtggtggaagttcgagacttgcgtaaactatttttaatttggagacggaattccgtctcgttttagagctagaaatagcaagttaaaataaggctagtccgttatcaacttgaaaaagtggcaccgagtcggtgctttttttctcgagtttttttatcgagtgtttaaggataatgataactgaagagaagaattagttttgccgccaccgcgggtttgcctctgattaaataaaaaaaagctggtgctttttttttcttttataggaacatcttgaatatatgaactaattaaatgataattttttacccatctttactcttaatcactgagctgcagtcaaagaaaaagggatacagcacctggtgaagagatgaacggagactaacttagacgcgttgattctttttaattgcacattttattaatcgatgctaacgtctatttacatatattctttagagatattatctagggcttcaaataatctctggacagcaataaaagtctcttcaaaagtattgtataacggcaatggggctaatctgattacatctggtcttctttcgtcacagattatagcatgatcatgcaagtacgcattaactcgttccatgacgttcttgtccttttcatcgaaatgcggttgaaacataatggacaattgacatcctctttcagctggattcaaaggagttaaaattttaaacccaaatttggagtttgatgtactggattgtggtatgtaatacttggaattcgtcaatagatcctgtaaaaattgagtcaaagcaacacttttttcacgaagtttagatactccacccactttagcatacacttccaatgacgacttcacagcaacaacatcaagaacagaaggatttgactgtctgtaagaaagagccgagtttattggatcaaactcttctaacattttgaatcgttcttgggagttattgccccaccaaccagctagtctaggaacgaaactgcttttcttgttctctatggtgtatttttcatgcacaaaaatcccacctatggctccaggtcccgagtttaaatatttgtaggaacaccaagcagcaaaatctactccccaatcatgtaaatttaatgggacattcccaactgcatgggcaagatcccacccaactttaatttgttggctcttttccttagcgtatttagttatttcctctatcttgaaaaattgaccagtgtagtattggataccaggaaaacacactagagccaattcatccaggttctcatctatagccttgattattctttctgttttaatataagtttcaccaggttgaacttccaattgaatcaaatgtttctcgtcgtatccgaacaatttaacaatgttcaaaaatgcatagtagtcagaaggaaatgcttgtttttcaaataaaattttggttcttttcccctcaggtttgtaaaaatggatcaacaatgcattcaagtttgctgttaaagaacccataactgcaacttcgttttcctttgcaccaacaatgggggctattaatggtaataagggtaaatcgatgtctacccacggtgttaacagtttgtcaggatgattgaaatgagactcaacccctcgttcaacccatgcatttaattcatcattgatagctttctttgtattcttaggcatcaacccaagagagtttccacataaataaatagactcagttgatgactcatatttattatttttgatacctaatgatccaaaagttggtatggcaaactcatttttaaaagttgggaactttttgtccaatttctttgcctcggctaatgacatctgataataaaatggggttggagtagttggtggtataaccggagagatagaattgaagaaaaaaatcggaaacaacaaaaaaagttgataccctgtattatgtgggagataattgcgaatggtggaaaaaaaaaagacgccattgagtctcaacaacaattctgtcagctgaagagctttacaatcgagaaactatgattcattccgttttaatatgtatgtgtttagtaaactcatgaattttatttgtggtctactttagtactaacataatcattggatagtcaataatgatggtcttccgagactaatgaaattctataccaaagtcgatattccaacacagaaattgctcttgcaacaagtgcacctgttgatatctagagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 16 - DNA, 14070 bp, Artificial Sequence
  Plasmid
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacctgccactactaccactgggagtttcgttcttctcgatactattagctttacttcctgcactagcagtggttggatcaacagaatcttcataatcatcaaaatcgtcttttgaagaccccccgtttgatgtatggccctgtcttttcatcaaactttttatatagttgactgaactgaggctaaatatgtgatcatcttcactatagacaatctttctcttatttgcaccaccgccaccactagtctttgagaaattctcaaaaccttttacgatattaccaagcgggctctcttcgaaataatctatctctttttgatatatcgaatcctctagcgtggttagctttctagttagttcttgcttcttaagaatttgctggattagtttatttttcaattcaacgtatttctcagagtcatctttagattttgatgaagatgtgcgttcattcgctatatccttcttggtcgtgtcttttcgatcctccttggctggcactgaactcgtcttttttggcgttgctgttccagacagacttatctcattagatttggaacttgtgggtttaacatcatttgtatctttagtagacatgattgtgcaataccgtgattatttgttttgaaaggtctgtcatatttctatcaatttcaaaacaaaatgttcatcagaaaaaagccaaaaatgtctcttctagtttcttagtggtgtcgcataatacacaatgtcgctcaacaatccacattcccggcgcatagctcaaatcacatgactacagctaacaattacacaaaaaaaattctctttttgatgtagcaactatcttcaactaaaacattttctccttcggcccatgattgtcctccgggtcgacagcaagccgttacaattgagatggaaagcgacctaccttcactcgataaggtgcttaattgtacttcatataaatctggcccggatctaaacaaatgagttccattaagccgtgggttctcaattagggtttttgtttttgatttagaaaaaagagatcaagatttgtttacaggtgatgcctttttttagaacttatgcgttgcaaaagttgactaacgatttctataaggtgatccacactaattatacaaacgtacaaacagacatacttttcctgcgttcacctgatgttggccagatttctctcttcattgcatagaacataaccacactagggcaacagaaaaaaaaaaaaaaagtgcatcgggaagttgtgttccattcattatatgtctactactgcatatgagtagcccacccaccaccaccatagtaagtttttgtgtatgcgcgccgtcaggttatttcatttctgaatttttcaaccaccttactccctttattgttgattgacaattttgctcacagtaagatcttttagactccaattaatataaaataagtctgattttccaattcctgttttttctttttttttctgtttctatttctttccttttctcccttttttttaattcttcattcaatcatcaattgataattcaggaatattacaacaacccgggatggataaaaagtatagtattggtttagatattggtactaactctgtgggttgggcagttatcaccgacgaatataaagttccatcaaagaaatttaaggtgttaggtaacactgacagacactcaataaaaaagaatcttatcggtgctcttttgttcgactccggtgaaactgccgaggctacacgtttaaaaagaacagcaagaagaagatatacccgtagaaaaaatagaatatgttatttacaagaaatcttttctaatgaaatggctaaagttgatgattcctttttccatagattggaagagtcatttttggttgaagaagacaaaaagcatgagagacatccaatctttgggaatatagttgatgaagtggcttaccatgaaaaatatcctaccatttatcatttaagaaagaaattggtagattcaactgataaagctgaccttagattaatctatttagcacttgcccatatgattaaatttagaggtcattttttgattgaaggtgatttgaacccagataattctgacgtggataaattatttattcaattagtccaaacctacaaccaattatttgaggaaaatccaattaatgctagtggtgtcgatgccaaagctatattatcagccagattatcaaaatctagacgtttggaaaatttgattgcccaattgccaggagaaaaaaagaatggattatttggaaacttgatcgcattatcattgggtttgacaccaaattttaaatctaattttgatttagctgaagatgctaaattacaattatcaaaagacacctatgacgacgatttggacaatttacttgctcaaattggtgatcaatatgcagatttgttcttagctgctaaaaacttatctgatgctattttgttgtctgatattttgagagtgaacacagaaataaccaaagctccattatcagcatctatgatcaaacgttatgatgaacaccatcaggatttgactttattgaaagctttggtgagacaacaattgccagagaagtataaagaaatctttttcgatcaatctaaaaacgggtatgcaggttatattgatgggggtgcctcccaagaggaattttacaaatttataaaacctattttagaaaagatggatgggactgaggaacttttggtcaaattgaacagagaagatttgttacgtaaacagagaacttttgataatggtagtatacctcaccaaattcatttgggtgagttgcatgcaattttaagaagacaagaagatttttatccatttttaaaagataatagagaaaaaatcgagaaaattttaacctttagaattccatactatgttgggcctttggctagaggtaattcaagatttgcctggatgacacgtaaatcagaagaaactattaccccttggaattttgaagaggttgttgataaaggagcatcagcacagagttttattgaaagaatgaccaatttcgataaaaacttaccaaatgaaaaagttttaccaaaacattccttgttatacgaatattttactgtttacaatgaacttacaaaggttaaatatgttactgaaggtatgcgtaagccagcctttttatctggagaacagaaaaaggcaatagttgatttattgtttaaaacaaatagaaaagttactgttaaacaattaaaagaagattactttaagaaaattgaatgttttgattcagttgaaatcagtggtgttgaagacagatttaatgctagtttaggaacttaccatgatttacttaaaattatcaaagataaagatttcttggataacgaagaaaatgaagacattttagaagacattgttttaaccttaactttattcgaagatagagagatgattgaagaacgtttgaagacttatgcacatttgtttgacgataaagtgatgaaacagttgaaaagaagacgttatactggatggggtagattgtctcgtaaattgatcaatggaattagagataaacaaagtggtaaaactatcttggactttttgaaatctgacggatttgctaatagaaatttcatgcaattgatccacgacgatagtttgacatttaaagaagacatccaaaaggcccaagtgagtgggcaaggtgattcattacatgaacatattgcaaatttagccggatctcctgctattaagaaagggatattacaaactgttaaagttgtggatgaattagtgaaagtaatgggaagacataaacctgaaaacattgtcattgagatggcaagagaaaatcaaactacacaaaaaggacagaaaaatagtagagaacgtatgaaaagaatagaagagggtattaaagaattgggtagtcaaatattgaaagaacacccagtggaaaatacccagttgcaaaatgaaaaattatatctttactaccttcaaaatggacgtgatatgtatgttgatcaggaattagatataaatagactttcagattatgatgtagatcatatagttccacaatctttcttgaaagatgattccatagacaataaagtattaactagaagtgataaaaatagaggtaaaagtgataatgtcccaagtgaggaagtcgtcaaaaagatgaaaaattactggcgtcaacttttgaatgctaaattaattactcaaagaaaatttgataatttgactaaagcagaaagaggtgggctttctgaattagataaagccgggttcattaaaagacaattggtcgaaactagacaaattactaaacatgttgcccaaattttagattcccgtatgaacactaagtatgacgaaaatgataagttaatacgtgaggttaaagtcattactttaaaatcaaaacttgtctctgatttcagaaaggatttccaattctataaagttagagaaattaataattatcatcatgctcatgatgcatatttgaatgctgtagttggaactgctttaatcaagaaataccctaaattagaatctgaatttgtatatggtgattacaaagtctatgatgttagaaagatgattgctaaatcagaacaagaaattggtaaagctacagctaaatacttcttttactctaacattatgaatttctttaaaacagaaattactttggcaaacggtgaaattagaaaaagacctcttattgaaacaaatggtgagactggagagatagtttgggacaaagggcgtgatttcgctactgttagaaaagttttatcaatgccacaagttaacattgtaaagaaaacagaggttcaaactggtggtttctcaaaagaaagtattttgcctaaaagaaatagtgataaattgattgccagaaaaaaggattgggatccaaagaaatatggtggtttcgactcaccaaccgtagcctattctgttttggttgtggcaaaggttgaaaagggtaaaagtaaaaagcttaaatcagtaaaagaacttttgggtattacaataatggaaagaagttcctttgaaaagaaccctattgattttttggaagctaaaggttataaggaagtaaagaaggacttaataatcaaattgcctaaatattctttatttgaattagaaaatgggagaaaaagaatgttggcttctgctggagaattgcaaaagggtaatgaattagcattgccttccaaatatgttaacttcttgtatttagcttcacactatgaaaagttgaaagggtcaccagaagataacgagcaaaaacaattatttgttgaacaacacaaacactacttagatgagattatagaacaaattagtgaattcagtaaaagagtgatattagctgatgcaaatttagataaagttttgtcagcctataacaaacatagagataagccaattagagaacaagcagaaaacattattcacttatttacccttaccaatttaggagcacctgctgctttcaagtattttgatacaacaattgatcgtaaaagatatacctcaacaaaagaagtcttagacgccaccttaattcatcaatcaatcactggattgtatgagacaagaattgatttgtctcaattgggtggtgatgaaggggctgatcctaagaagaaaagaaaagttgatccaaagaaaaagcgtaaggtggatcctaagaaaaagagaaaggttgactacaaagaccatgacggtgattataaagatcatgacatcgactacaaggatgacgatgacaagtgataatgactgcagagatccatcgacctgccgccaagctaattccgggcgaatttctgtcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccgggccccccctaactcaagtacaacagatctggaccacctttgattgtaaatagtaataattaccacccttatctaattatttatttaacttatttatttatttattatacatatatacaaatctaataaagtgaaaatctcccccttcacacttcacatatgttaggcgtcatcctgtgctcccgagaaccagtaccagtacatcgctgtttcgttcgagacttgaggtctagttttatacgtgaagaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaattttcatatgagaaccgttatcgataactaaagcagcaacttcttctataaaaatgggttagtatgacagtcatttaaataaggaatttttcagttggcttggtttcaattcaatgttcgttttttttttttcttgctgtgtttgtgtttgtgttgtttatagttgtgtgcactgatcgtcgaaaaaaaaaattcatagtgagccgggaaatctgtatagcccagataacaacacaagtccaaactagaaactcgtcaaacaccaaaagcaatgttgaatcaattgccttgcacaagtacacgtaggaaaacataaaacattgcaattttgaatattgagccttttgtcgtaacattgattgataggattactcaccgaatggttttgaaaccactgccgacagatcaatcaatcaatcaaaaaacgtgaactttgaaaaaggggaagaacagatacattgaagttagccatttccactgatcgtcacaacatatctgataaattactttcaaaattataagctgatgtgtgtgtattattaatgtgacagtaacatcccaaacgagaaatattatctcgacaacaaaaaagtttgatctgaattgaaaatgaagttttcccaccctacccatttgtcatattgaaaccaatcaactgattaatcaatcaattagaattgaagctaaactaaaacataccaccgtccattttgaatgattatatttttttaatattaatatcgagataatgtttctaagaaagaaagaaaaccaggagtgaaaattagaaaaggaaaggaaaggaaaaaaagaaaaatctgaaaatatataaaaaaaaattgtttcgttggcaataaatcttggtgagaacagcgaccgaaagcaaataagaacaaaatatgagtgtattacgttgaacaactaattaacgtgtgtgtatggatctttttttcttttttctctttaaccgactataaacaacaaacatttttgggcagtgcacacactacttaatatacacagcataaattacacgattagaaacaaattagcttattaaaataacctaatcaaaccgaatattttatggtattatgagtaaactatataatataaatagcacacacccacaacaacaacaaaggaaaactaaaaggttttttctttttgaaaagatcgttttctttattattctctagttttgacggcggccgcaagtgattagacttagtccgttcaaatcaagcacaactctgttcattgtttcaacaagaattaattcaaaaacaggttcggtgcataatttgcaaaaaaatattgcagcttctgtggctcgaacacagtacctccagatttcaggtttgaaatacttcagtctgacgctctcccagatgagctaaagctgcaataagaaaacccacgccgggattcgaacccggaatcctttgattagaagtcaaaagcgataaccatttcgccacgcaggcctacttgatgggtttgtaaatggtctactttttcagacctaacagaaattttaatgaaagtcatattcttatacaataaaactgtgtcataaaagcagatattcgactttcgtagattatataggacccaagaactaaaatttaatgccatattatgcatttttaatctgtaaaagtgttgtttccaacctatcacaagtacgttcttgtaacttgtgtttgtagggttgcaaatgaatcataacaacatctcaacagaacatgtatagcaaagcttagtataaaatcagtgttttgagaggcaatccaagaatgtttacatcaaagtttcaataaatatcgaccgaaactgaaaatctttttaggttattgttcacttttttgtaaatatttaaacattttttggacctaaaaaaatacaaacaccaattacgtaccaagaagcatctaatcaactcccagatcaccactatacatttaaaagtcattggtcaataactatactcgagtattgcctcatcaaagaaacaatcaaatattatagatactcactccatcacgtgataatttcactggtatggaaaagtggaaaattttataaaaaaaaatttgatgcctttggcatagctgaaacttcggcccaataggattggagaatatgttttcgcagcgttcttacaattaaattgtggtggaagttcgagacttgcgtaaactatttttaatttggatccgcaacaatcatacgacctaatgttttagagctagaaatagcaagttaaaataaggctagtccgttatcaacttgaaaaagtggcaccgagtcggtgctttttttctcgagtttttttatcgagtgtttaaggataatgataactgaagagaagaattagttttgccgccaccgcgggtttgcctctgattaaataaaaaaaagctggtgctttttttttcttttataggaacatcttgaatatatgaactaattaaatgataattttttacccatctttactcttaatcactgagctgcagtcaaagaaaaagggatacagcacctggtgaagagatgaacggagactaacttagacgcgttgattctttttaattgcacattttattaatcgatgctaacgtctatttacatatattctttagagatattatctagggcttcaaataatctctggacagcaataaaagtctcttcaaaagtattgtataacggcaatggggctaatctgattacatctggtcttctttcgtcacagattatagcatgatcatgcaagtacgcattaactcgttccatgacgttcttgtccttttcatcgaaatgcggttgaaacataatggacaattgacatcctctttcagctggattcaaaggagttaaaattttaaacccaaatttggagtttgatgtactggattgtggtatgtaatacttggaattcgtcaatagatcctgtaaaaattgagtcaaagcaacacttttttcacgaagtttagatactccacccactttagcatacacttccaatgacgacttcacagcaacaacatcaagaacagaaggatttgactgtctgtaagaaagagccgagtttattggatcaaactcttctaacattttgaatcgttcttgggagttattgccccaccaaccagctagtctaggaacgaaactgcttttcttgttctctatggtgtatttttcatgcacaaaaatcccacctatggctccaggtcccgagtttaaatatttgtaggaacaccaagcagcaaaatctactccccaatcatgtaaatttaatgggacattcccaactgcatgggcaagatcccacccaactttaatttgttggctcttttccttagcgtatttagttatttcctctatcttgaaaaattgaccagtgtagtattggataccaggaaaacacactagagccaattcatccaggttctcatctatagccttgattattctttctgttttaatataagtttcaccaggttgaacttccaattgaatcaaatgtttctcgtcgtatccgaacaatttaacaatgttcaaaaatgcatagtagtcagaaggaaatgcttgtttttcaaataaaattttggttcttttcccctcaggtttgtaaaaatggatcaacaatgcattcaagtttgctgttaaagaacccataactgcaacttcgttttcctttgcaccaacaatgggggctattaatggtaataagggtaaatcgatgtctacccacggtgttaacagtttgtcaggatgattgaaatgagactcaacccctcgttcaacccatgcatttaattcatcattgatagctttctttgtattcttaggcatcaacccaagagagtttccacataaataaatagactcagttgatgactcatatttattatttttgatacctaatgatccaaaagttggtatggcaaactcatttttaaaagttgggaactttttgtccaatttctttgcctcggctaatgacatctgataataaaatggggttggagtagttggtggtataaccggagagatagaattgaagaaaaaaatcggaaacaacaaaaaaagttgataccctgtattatgtgggagataattgcgaatggtggaaaaaaaaaagacgccattgagtctcaacaacaattctgtcagctgaagagctttacaatcgagaaactatgattcattccgttttaatatgtatgtgtttagtaaactcatgaattttatttgtggtctactttagtactaacataatcattggatagtcaataatgatggtcttccgagactaatgaaattctataccaaagtcgatattccaacacagaaattgctcttgcaacaagtgcacctgttgatatctagagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 17 - DNA, 14070 bp, Artificial Sequence
  Plasmid
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacctgccactactaccactgggagtttcgttcttctcgatactattagctttacttcctgcactagcagtggttggatcaacagaatcttcataatcatcaaaatcgtcttttgaagaccccccgtttgatgtatggccctgtcttttcatcaaactttttatatagttgactgaactgaggctaaatatgtgatcatcttcactatagacaatctttctcttatttgcaccaccgccaccactagtctttgagaaattctcaaaaccttttacgatattaccaagcgggctctcttcgaaataatctatctctttttgatatatcgaatcctctagcgtggttagctttctagttagttcttgcttcttaagaatttgctggattagtttatttttcaattcaacgtatttctcagagtcatctttagattttgatgaagatgtgcgttcattcgctatatccttcttggtcgtgtcttttcgatcctccttggctggcactgaactcgtcttttttggcgttgctgttccagacagacttatctcattagatttggaacttgtgggtttaacatcatttgtatctttagtagacatgattgtgcaataccgtgattatttgttttgaaaggtctgtcatatttctatcaatttcaaaacaaaatgttcatcagaaaaaagccaaaaatgtctcttctagtttcttagtggtgtcgcataatacacaatgtcgctcaacaatccacattcccggcgcatagctcaaatcacatgactacagctaacaattacacaaaaaaaattctctttttgatgtagcaactatcttcaactaaaacattttctccttcggcccatgattgtcctccgggtcgacagcaagccgttacaattgagatggaaagcgacctaccttcactcgataaggtgcttaattgtacttcatataaatctggcccggatctaaacaaatgagttccattaagccgtgggttctcaattagggtttttgtttttgatttagaaaaaagagatcaagatttgtttacaggtgatgcctttttttagaacttatgcgttgcaaaagttgactaacgatttctataaggtgatccacactaattatacaaacgtacaaacagacatacttttcctgcgttcacctgatgttggccagatttctctcttcattgcatagaacataaccacactagggcaacagaaaaaaaaaaaaaaagtgcatcgggaagttgtgttccattcattatatgtctactactgcatatgagtagcccacccaccaccaccatagtaagtttttgtgtatgcgcgccgtcaggttatttcatttctgaatttttcaaccaccttactccctttattgttgattgacaattttgctcacagtaagatcttttagactccaattaatataaaataagtctgattttccaattcctgttttttctttttttttctgtttctatttctttccttttctcccttttttttaattcttcattcaatcatcaattgataattcaggaatattacaacaacccgggatggataaaaagtatagtattggtttagatattggtactaactctgtgggttgggcagttatcaccgacgaatataaagttccatcaaagaaatttaaggtgttaggtaacactgacagacactcaataaaaaagaatcttatcggtgctcttttgttcgactccggtgaaactgccgaggctacacgtttaaaaagaacagcaagaagaagatatacccgtagaaaaaatagaatatgttatttacaagaaatcttttctaatgaaatggctaaagttgatgattcctttttccatagattggaagagtcatttttggttgaagaagacaaaaagcatgagagacatccaatctttgggaatatagttgatgaagtggcttaccatgaaaaatatcctaccatttatcatttaagaaagaaattggtagattcaactgataaagctgaccttagattaatctatttagcacttgcccatatgattaaatttagaggtcattttttgattgaaggtgatttgaacccagataattctgacgtggataaattatttattcaattagtccaaacctacaaccaattatttgaggaaaatccaattaatgctagtggtgtcgatgccaaagctatattatcagccagattatcaaaatctagacgtttggaaaatttgattgcccaattgccaggagaaaaaaagaatggattatttggaaacttgatcgcattatcattgggtttgacaccaaattttaaatctaattttgatttagctgaagatgctaaattacaattatcaaaagacacctatgacgacgatttggacaatttacttgctcaaattggtgatcaatatgcagatttgttcttagctgctaaaaacttatctgatgctattttgttgtctgatattttgagagtgaacacagaaataaccaaagctccattatcagcatctatgatcaaacgttatgatgaacaccatcaggatttgactttattgaaagctttggtgagacaacaattgccagagaagtataaagaaatctttttcgatcaatctaaaaacgggtatgcaggttatattgatgggggtgcctcccaagaggaattttacaaatttataaaacctattttagaaaagatggatgggactgaggaacttttggtcaaattgaacagagaagatttgttacgtaaacagagaacttttgataatggtagtatacctcaccaaattcatttgggtgagttgcatgcaattttaagaagacaagaagatttttatccatttttaaaagataatagagaaaaaatcgagaaaattttaacctttagaattccatactatgttgggcctttggctagaggtaattcaagatttgcctggatgacacgtaaatcagaagaaactattaccccttggaattttgaagaggttgttgataaaggagcatcagcacagagttttattgaaagaatgaccaatttcgataaaaacttaccaaatgaaaaagttttaccaaaacattccttgttatacgaatattttactgtttacaatgaacttacaaaggttaaatatgttactgaaggtatgcgtaagccagcctttttatctggagaacagaaaaaggcaatagttgatttattgtttaaaacaaatagaaaagttactgttaaacaattaaaagaagattactttaagaaaattgaatgttttgattcagttgaaatcagtggtgttgaagacagatttaatgctagtttaggaacttaccatgatttacttaaaattatcaaagataaagatttcttggataacgaagaaaatgaagacattttagaagacattgttttaaccttaactttattcgaagatagagagatgattgaagaacgtttgaagacttatgcacatttgtttgacgataaagtgatgaaacagttgaaaagaagacgttatactggatggggtagattgtctcgtaaattgatcaatggaattagagataaacaaagtggtaaaactatcttggactttttgaaatctgacggatttgctaatagaaatttcatgcaattgatccacgacgatagtttgacatttaaagaagacatccaaaaggcccaagtgagtgggcaaggtgattcattacatgaacatattgcaaatttagccggatctcctgctattaagaaagggatattacaaactgttaaagttgtggatgaattagtgaaagtaatgggaagacataaacctgaaaacattgtcattgagatggcaagagaaaatcaaactacacaaaaaggacagaaaaatagtagagaacgtatgaaaagaatagaagagggtattaaagaattgggtagtcaaatattgaaagaacacccagtggaaaatacccagttgcaaaatgaaaaattatatctttactaccttcaaaatggacgtgatatgtatgttgatcaggaattagatataaatagactttcagattatgatgtagatcatatagttccacaatctttcttgaaagatgattccatagacaataaagtattaactagaagtgataaaaatagaggtaaaagtgataatgtcccaagtgaggaagtcgtcaaaaagatgaaaaattactggcgtcaacttttgaatgctaaattaattactcaaagaaaatttgataatttgactaaagcagaaagaggtgggctttctgaattagataaagccgggttcattaaaagacaattggtcgaaactagacaaattactaaacatgttgcccaaattttagattcccgtatgaacactaagtatgacgaaaatgataagttaatacgtgaggttaaagtcattactttaaaatcaaaacttgtctctgatttcagaaaggatttccaattctataaagttagagaaattaataattatcatcatgctcatgatgcatatttgaatgctgtagttggaactgctttaatcaagaaataccctaaattagaatctgaatttgtatatggtgattacaaagtctatgatgttagaaagatgattgctaaatcagaacaagaaattggtaaagctacagctaaatacttcttttactctaacattatgaatttctttaaaacagaaattactttggcaaacggtgaaattagaaaaagacctcttattgaaacaaatggtgagactggagagatagtttgggacaaagggcgtgatttcgctactgttagaaaagttttatcaatgccacaagttaacattgtaaagaaaacagaggttcaaactggtggtttctcaaaagaaagtattttgcctaaaagaaatagtgataaattgattgccagaaaaaaggattgggatccaaagaaatatggtggtttcgactcaccaaccgtagcctattctgttttggttgtggcaaaggttgaaaagggtaaaagtaaaaagcttaaatcagtaaaagaacttttgggtattacaataatggaaagaagttcctttgaaaagaaccctattgattttttggaagctaaaggttataaggaagtaaagaaggacttaataatcaaattgcctaaatattctttatttgaattagaaaatgggagaaaaagaatgttggcttctgctggagaattgcaaaagggtaatgaattagcattgccttccaaatatgttaacttcttgtatttagcttcacactatgaaaagttgaaagggtcaccagaagataacgagcaaaaacaattatttgttgaacaacacaaacactacttagatgagattatagaacaaattagtgaattcagtaaaagagtgatattagctgatgcaaatttagataaagttttgtcagcctataacaaacatagagataagccaattagagaacaagcagaaaacattattcacttatttacccttaccaatttaggagcacctgctgctttcaagtattttgatacaacaattgatcgtaaaagatatacctcaacaaaagaagtcttagacgccaccttaattcatcaatcaatcactggattgtatgagacaagaattgatttgtctcaattgggtggtgatgaaggggctgatcctaagaagaaaagaaaagttgatccaaagaaaaagcgtaaggtggatcctaagaaaaagagaaaggttgactacaaagaccatgacggtgattataaagatcatgacatcgactacaaggatgacgatgacaagtgataatgactgcagagatccatcgacctgccgccaagctaattccgggcgaatttctgtcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccgggccccccctaactcaagtacaacagatctggaccacctttgattgtaaatagtaataattaccacccttatctaattatttatttaacttatttatttatttattatacatatatacaaatctaataaagtgaaaatctcccccttcacacttcacatatgttaggcgtcatcctgtgctcccgagaaccagtaccagtacatcgctgtttcgttcgagacttgaggtctagttttatacgtgaagaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaattttcatatgagaaccgttatcgataactaaagcagcaacttcttctataaaaatgggttagtatgacagtcatttaaataaggaatttttcagttggcttggtttcaattcaatgttcgttttttttttttcttgctgtgtttgtgtttgtgttgtttatagttgtgtgcactgatcgtcgaaaaaaaaaattcatagtgagccgggaaatctgtatagcccagataacaacacaagtccaaactagaaactcgtcaaacaccaaaagcaatgttgaatcaattgccttgcacaagtacacgtaggaaaacataaaacattgcaattttgaatattgagccttttgtcgtaacattgattgataggattactcaccgaatggttttgaaaccactgccgacagatcaatcaatcaatcaaaaaacgtgaactttgaaaaaggggaagaacagatacattgaagttagccatttccactgatcgtcacaacatatctgataaattactttcaaaattataagctgatgtgtgtgtattattaatgtgacagtaacatcccaaacgagaaatattatctcgacaacaaaaaagtttgatctgaattgaaaatgaagttttcccaccctacccatttgtcatattgaaaccaatcaactgattaatcaatcaattagaattgaagctaaactaaaacataccaccgtccattttgaatgattatatttttttaatattaatatcgagataatgtttctaagaaagaaagaaaaccaggagtgaaaattagaaaaggaaaggaaaggaaaaaaagaaaaatctgaaaatatataaaaaaaaattgtttcgttggcaataaatcttggtgagaacagcgaccgaaagcaaataagaacaaaatatgagtgtattacgttgaacaactaattaacgtgtgtgtatggatctttttttcttttttctctttaaccgactataaacaacaaacatttttgggcagtgcacacactacttaatatacacagcataaattacacgattagaaacaaattagcttattaaaataacctaatcaaaccgaatattttatggtattatgagtaaactatataatataaatagcacacacccacaacaacaacaaaggaaaactaaaaggttttttctttttgaaaagatcgttttctttattattctctagttttgacggcggccgcaagtgattagacttagtccgttcaaatcaagcacaactctgttcattgtttcaacaagaattaattcaaaaacaggttcggtgcataatttgcaaaaaaatattgcagcttctgtggctcgaacacagtacctccagatttcaggtttgaaatacttcagtctgacgctctcccagatgagctaaagctgcaataagaaaacccacgccgggattcgaacccggaatcctttgattagaagtcaaaagcgataaccatttcgccacgcaggcctacttgatgggtttgtaaatggtctactttttcagacctaacagaaattttaatgaaagtcatattcttatacaataaaactgtgtcataaaagcagatattcgactttcgtagattatataggacccaagaactaaaatttaatgccatattatgcatttttaatctgtaaaagtgttgtttccaacctatcacaagtacgttcttgtaacttgtgtttgtagggttgcaaatgaatcataacaacatctcaacagaacatgtatagcaaagcttagtataaaatcagtgttttgagaggcaatccaagaatgtttacatcaaagtttcaataaatatcgaccgaaactgaaaatctttttaggttattgttcacttttttgtaaatatttaaacattttttggacctaaaaaaatacaaacaccaattacgtaccaagaagcatctaatcaactcccagatcaccactatacatttaaaagtcattggtcaataactatactcgagtattgcctcatcaaagaaacaatcaaatattatagatactcactccatcacgtgataatttcactggtatggaaaagtggaaaattttataaaaaaaaatttgatgcctttggcatagctgaaacttcggcccaataggattggagaatatgttttcgcagcgttcttacaattaaattgtggtggaagttcgagacttgcgtaaactatttttaatttggatccggttttggggagacccggtgcgttttagagctagaaatagcaagttaaaataaggctagtccgttatcaacttgaaaaagtggcaccgagtcggtgctttttttctcgagtttttttatcgagtgtttaaggataatgataactgaagagaagaattagttttgccgccaccgcgggtttgcctctgattaaataaaaaaaagctggtgctttttttttcttttataggaacatcttgaatatatgaactaattaaatgataattttttacccatctttactcttaatcactgagctgcagtcaaagaaaaagggatacagcacctggtgaagagatgaacggagactaacttagacgcgttgattctttttaattgcacattttattaatcgatgctaacgtctatttacatatattctttagagatattatctagggcttcaaataatctctggacagcaataaaagtctcttcaaaagtattgtataacggcaatggggctaatctgattacatctggtcttctttcgtcacagattatagcatgatcatgcaagtacgcattaactcgttccatgacgttcttgtccttttcatcgaaatgcggttgaaacataatggacaattgacatcctctttcagctggattcaaaggagttaaaattttaaacccaaatttggagtttgatgtactggattgtggtatgtaatacttggaattcgtcaatagatcctgtaaaaattgagtcaaagcaacacttttttcacgaagtttagatactccacccactttagcatacacttccaatgacgacttcacagcaacaacatcaagaacagaaggatttgactgtctgtaagaaagagccgagtttattggatcaaactcttctaacattttgaatcgttcttgggagttattgccccaccaaccagctagtctaggaacgaaactgcttttcttgttctctatggtgtatttttcatgcacaaaaatcccacctatggctccaggtcccgagtttaaatatttgtaggaacaccaagcagcaaaatctactccccaatcatgtaaatttaatgggacattcccaactgcatgggcaagatcccacccaactttaatttgttggctcttttccttagcgtatttagttatttcctctatcttgaaaaattgaccagtgtagtattggataccaggaaaacacactagagccaattcatccaggttctcatctatagccttgattattctttctgttttaatataagtttcaccaggttgaacttccaattgaatcaaatgtttctcgtcgtatccgaacaatttaacaatgttcaaaaatgcatagtagtcagaaggaaatgcttgtttttcaaataaaattttggttcttttcccctcaggtttgtaaaaatggatcaacaatgcattcaagtttgctgttaaagaacccataactgcaacttcgttttcctttgcaccaacaatgggggctattaatggtaataagggtaaatcgatgtctacccacggtgttaacagtttgtcaggatgattgaaatgagactcaacccctcgttcaacccatgcatttaattcatcattgatagctttctttgtattcttaggcatcaacccaagagagtttccacataaataaatagactcagttgatgactcatatttattatttttgatacctaatgatccaaaagttggtatggcaaactcatttttaaaagttgggaactttttgtccaatttctttgcctcggctaatgacatctgataataaaatggggttggagtagttggtggtataaccggagagatagaattgaagaaaaaaatcggaaacaacaaaaaaagttgataccctgtattatgtgggagataattgcgaatggtggaaaaaaaaaagacgccattgagtctcaacaacaattctgtcagctgaagagctttacaatcgagaaactatgattcattccgttttaatatgtatgtgtttagtaaactcatgaattttatttgtggtctactttagtactaacataatcattggatagtcaataatgatggtcttccgagactaatgaaattctataccaaagtcgatattccaacacagaaattgctcttgcaacaagtgcacctgttgatatctagagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 18 - DNA, 14064 bp, Artificial Sequence
  Plasmid
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacctgccactactaccactgggagtttcgttcttctcgatactattagctttacttcctgcactagcagtggttggatcaacagaatcttcataatcatcaaaatcgtcttttgaagaccccccgtttgatgtatggccctgtcttttcatcaaactttttatatagttgactgaactgaggctaaatatgtgatcatcttcactatagacaatctttctcttatttgcaccaccgccaccactagtctttgagaaattctcaaaaccttttacgatattaccaagcgggctctcttcgaaataatctatctctttttgatatatcgaatcctctagcgtggttagctttctagttagttcttgcttcttaagaatttgctggattagtttatttttcaattcaacgtatttctcagagtcatctttagattttgatgaagatgtgcgttcattcgctatatccttcttggtcgtgtcttttcgatcctccttggctggcactgaactcgtcttttttggcgttgctgttccagacagacttatctcattagatttggaacttgtgggtttaacatcatttgtatctttagtagacatgattgtgcaataccgtgattatttgttttgaaaggtctgtcatatttctatcaatttcaaaacaaaatgttcatcagaaaaaagccaaaaatgtctcttctagtttcttagtggtgtcgcataatacacaatgtcgctcaacaatccacattcccggcgcatagctcaaatcacatgactacagctaacaattacacaaaaaaaattctctttttgatgtagcaactatcttcaactaaaacattttctccttcggcccatgattgtcctccgggtcgacagcaagccgttacaattgagatggaaagcgacctaccttcactcgataaggtgcttaattgtacttcatataaatctggcccggatctaaacaaatgagttccattaagccgtgggttctcaattagggtttttgtttttgatttagaaaaaagagatcaagatttgtttacaggtgatgcctttttttagaacttatgcgttgcaaaagttgactaacgatttctataaggtgatccacactaattatacaaacgtacaaacagacatacttttcctgcgttcacctgatgttggccagatttctctcttcattgcatagaacataaccacactagggcaacagaaaaaaaaaaaaaaagtgcatcgggaagttgtgttccattcattatatgtctactactgcatatgagtagcccacccaccaccaccatagtaagtttttgtgtatgcgcgccgtcaggttatttcatttctgaatttttcaaccaccttactccctttattgttgattgacaattttgctcacagtaagatcttttagactccaattaatataaaataagtctgattttccaattcctgttttttctttttttttctgtttctatttctttccttttctcccttttttttaattcttcattcaatcatcaattgataattcaggaatattacaacaacccgggatggataaaaagtatagtattggtttagatattggtactaactctgtgggttgggcagttatcaccgacgaatataaagttccatcaaagaaatttaaggtgttaggtaacactgacagacactcaataaaaaagaatcttatcggtgctcttttgttcgactccggtgaaactgccgaggctacacgtttaaaaagaacagcaagaagaagatatacccgtagaaaaaatagaatatgttatttacaagaaatcttttctaatgaaatggctaaagttgatgattcctttttccatagattggaagagtcatttttggttgaagaagacaaaaagcatgagagacatccaatctttgggaatatagttgatgaagtggcttaccatgaaaaatatcctaccatttatcatttaagaaagaaattggtagattcaactgataaagctgaccttagattaatctatttagcacttgcccatatgattaaatttagaggtcattttttgattgaaggtgatttgaacccagataattctgacgtggataaattatttattcaattagtccaaacctacaaccaattatttgaggaaaatccaattaatgctagtggtgtcgatgccaaagctatattatcagccagattatcaaaatctagacgtttggaaaatttgattgcccaattgccaggagaaaaaaagaatggattatttggaaacttgatcgcattatcattgggtttgacaccaaattttaaatctaattttgatttagctgaagatgctaaattacaattatcaaaagacacctatgacgacgatttggacaatttacttgctcaaattggtgatcaatatgcagatttgttcttagctgctaaaaacttatctgatgctattttgttgtctgatattttgagagtgaacacagaaataaccaaagctccattatcagcatctatgatcaaacgttatgatgaacaccatcaggatttgactttattgaaagctttggtgagacaacaattgccagagaagtataaagaaatctttttcgatcaatctaaaaacgggtatgcaggttatattgatgggggtgcctcccaagaggaattttacaaatttataaaacctattttagaaaagatggatgggactgaggaacttttggtcaaattgaacagagaagatttgttacgtaaacagagaacttttgataatggtagtatacctcaccaaattcatttgggtgagttgcatgcaattttaagaagacaagaagatttttatccatttttaaaagataatagagaaaaaatcgagaaaattttaacctttagaattccatactatgttgggcctttggctagaggtaattcaagatttgcctggatgacacgtaaatcagaagaaactattaccccttggaattttgaagaggttgttgataaaggagcatcagcacagagttttattgaaagaatgaccaatttcgataaaaacttaccaaatgaaaaagttttaccaaaacattccttgttatacgaatattttactgtttacaatgaacttacaaaggttaaatatgttactgaaggtatgcgtaagccagcctttttatctggagaacagaaaaaggcaatagttgatttattgtttaaaacaaatagaaaagttactgttaaacaattaaaagaagattactttaagaaaattgaatgttttgattcagttgaaatcagtggtgttgaagacagatttaatgctagtttaggaacttaccatgatttacttaaaattatcaaagataaagatttcttggataacgaagaaaatgaagacattttagaagacattgttttaaccttaactttattcgaagatagagagatgattgaagaacgtttgaagacttatgcacatttgtttgacgataaagtgatgaaacagttgaaaagaagacgttatactggatggggtagattgtctcgtaaattgatcaatggaattagagataaacaaagtggtaaaactatcttggactttttgaaatctgacggatttgctaatagaaatttcatgcaattgatccacgacgatagtttgacatttaaagaagacatccaaaaggcccaagtgagtgggcaaggtgattcattacatgaacatattgcaaatttagccggatctcctgctattaagaaagggatattacaaactgttaaagttgtggatgaattagtgaaagtaatgggaagacataaacctgaaaacattgtcattgagatggcaagagaaaatcaaactacacaaaaaggacagaaaaatagtagagaacgtatgaaaagaatagaagagggtattaaagaattgggtagtcaaatattgaaagaacacccagtggaaaatacccagttgcaaaatgaaaaattatatctttactaccttcaaaatggacgtgatatgtatgttgatcaggaattagatataaatagactttcagattatgatgtagatcatatagttccacaatctttcttgaaagatgattccatagacaataaagtattaactagaagtgataaaaatagaggtaaaagtgataatgtcccaagtgaggaagtcgtcaaaaagatgaaaaattactggcgtcaacttttgaatgctaaattaattactcaaagaaaatttgataatttgactaaagcagaaagaggtgggctttctgaattagataaagccgggttcattaaaagacaattggtcgaaactagacaaattactaaacatgttgcccaaattttagattcccgtatgaacactaagtatgacgaaaatgataagttaatacgtgaggttaaagtcattactttaaaatcaaaacttgtctctgatttcagaaaggatttccaattctataaagttagagaaattaataattatcatcatgctcatgatgcatatttgaatgctgtagttggaactgctttaatcaagaaataccctaaattagaatctgaatttgtatatggtgattacaaagtctatgatgttagaaagatgattgctaaatcagaacaagaaattggtaaagctacagctaaatacttcttttactctaacattatgaatttctttaaaacagaaattactttggcaaacggtgaaattagaaaaagacctcttattgaaacaaatggtgagactggagagatagtttgggacaaagggcgtgatttcgctactgttagaaaagttttatcaatgccacaagttaacattgtaaagaaaacagaggttcaaactggtggtttctcaaaagaaagtattttgcctaaaagaaatagtgataaattgattgccagaaaaaaggattgggatccaaagaaatatggtggtttcgactcaccaaccgtagcctattctgttttggttgtggcaaaggttgaaaagggtaaaagtaaaaagcttaaatcagtaaaagaacttttgggtattacaataatggaaagaagttcctttgaaaagaaccctattgattttttggaagctaaaggttataaggaagtaaagaaggacttaataatcaaattgcctaaatattctttatttgaattagaaaatgggagaaaaagaatgttggcttctgctggagaattgcaaaagggtaatgaattagcattgccttccaaatatgttaacttcttgtatttagcttcacactatgaaaagttgaaagggtcaccagaagataacgagcaaaaacaattatttgttgaacaacacaaacactacttagatgagattatagaacaaattagtgaattcagtaaaagagtgatattagctgatgcaaatttagataaagttttgtcagcctataacaaacatagagataagccaattagagaacaagcagaaaacattattcacttatttacccttaccaatttaggagcacctgctgctttcaagtattttgatacaacaattgatcgtaaaagatatacctcaacaaaagaagtcttagacgccaccttaattcatcaatcaatcactggattgtatgagacaagaattgatttgtctcaattgggtggtgatgaaggggctgatcctaagaagaaaagaaaagttgatccaaagaaaaagcgtaaggtggatcctaagaaaaagagaaaggttgactacaaagaccatgacggtgattataaagatcatgacatcgactacaaggatgacgatgacaagtgataatgactgcagagatccatcgacctgccgccaagctaattccgggcgaatttctgtcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccgggccccccctaactcaagtacaacagatctggaccacctttgattgtaaatagtaataattaccacccttatctaattatttatttaacttatttatttatttattatacatatatacaaatctaataaagtgaaaatctcccccttcacacttcacatatgttaggcgtcatcctgtgctcccgagaaccagtaccagtacatcgctgtttcgttcgagacttgaggtctagttttatacgtgaagaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaattttcatatgagaaccgttatcgataactaaagcagcaacttcttctataaaaatgggttagtatgacagtcatttaaataaggaatttttcagttggcttggtttcaattcaatgttcgttttttttttttcttgctgtgtttgtgtttgtgttgtttatagttgtgtgcactgatcgtcgaaaaaaaaaattcatagtgagccgggaaatctgtatagcccagataacaacacaagtccaaactagaaactcgtcaaacaccaaaagcaatgttgaatcaattgccttgcacaagtacacgtaggaaaacataaaacattgcaattttgaatattgagccttttgtcgtaacattgattgataggattactcaccgaatggttttgaaaccactgccgacagatcaatcaatcaatcaaaaaacgtgaactttgaaaaaggggaagaacagatacattgaagttagccatttccactgatcgtcacaacatatctgataaattactttcaaaattataagctgatgtgtgtgtattattaatgtgacagtaacatcccaaacgagaaatattatctcgacaacaaaaaagtttgatctgaattgaaaatgaagttttcccaccctacccatttgtcatattgaaaccaatcaactgattaatcaatcaattagaattgaagctaaactaaaacataccaccgtccattttgaatgattatatttttttaatattaatatcgagataatgtttctaagaaagaaagaaaaccaggagtgaaaattagaaaaggaaaggaaaggaaaaaaagaaaaatctgaaaatatataaaaaaaaattgtttcgttggcaataaatcttggtgagaacagcgaccgaaagcaaataagaacaaaatatgagtgtattacgttgaacaactaattaacgtgtgtgtatggatctttttttcttttttctctttaaccgactataaacaacaaacatttttgggcagtgcacacactacttaatatacacagcataaattacacgattagaaacaaattagcttattaaaataacctaatcaaaccgaatattttatggtattatgagtaaactatataatataaatagcacacacccacaacaacaacaaaggaaaactaaaaggttttttctttttgaaaagatcgttttctttattattctctagttttgacggcggccgcaagtgattagacttagtccgttcaaatcaagcacaactctgttcattgtttcaacaagaattaattcaaaaacaggttcggtgcataatttgcaaaaaaatattgcagcttctgtggctcgaacacagtacctccagatttcaggtttgaaatacttcagtctgacgctctcccagatgagctaaagctgcaataagaaaacccacgccgggattcgaacccggaatcctttgattagaagtcaaaagcgataaccatttcgccacgcaggcctacttgatgggtttgtaaatggtctactttttcagacctaacagaaattttaatgaaagtcatattcttatacaataaaactgtgtcataaaagcagatattcgactttcgtagattatataggacccaagaactaaaatttaatgccatattatgcatttttaatctgtaaaagtgttgtttccaacctatcacaagtacgttcttgtaacttgtgtttgtagggttgcaaatgaatcataacaacatctcaacagaacatgtatagcaaagcttagtataaaatcagtgttttgagaggcaatccaagaatgtttacatcaaagtttcaataaatatcgaccgaaactgaaaatctttttaggttattgttcacttttttgtaaatatttaaacattttttggacctaaaaaaatacaaacaccaattacgtaccaagaagcatctaatcaactcccagatcaccactatacatttaaaagtcattggtcaataactatactcgagtattgcctcatcaaagaaacaatcaaatattatagatactcactccatcacgtgataatttcactggtatggaaaagtggaaaattttataaaaaaaaatttgatgcctttggcatagctgaaacttcggcccaataggattggagaatatgttttcgcagcgttcttacaattaaattgtggtggaagttcgagacttgcgtaaactatttttaatttgagtttctgctctctcactatgttttagagctagaaatagcaagttaaaataaggctagtccgttatcaacttgaaaaagtggcaccgagtcggtgctttttttctcgagtttttttatcgagtgtttaaggataatgataactgaagagaagaattagttttgccgccaccgcgggtttgcctctgattaaataaaaaaaagctggtgctttttttttcttttataggaacatcttgaatatatgaactaattaaatgataattttttacccatctttactcttaatcactgagctgcagtcaaagaaaaagggatacagcacctggtgaagagatgaacggagactaacttagacgcgttgattctttttaattgcacattttattaatcgatgctaacgtctatttacatatattctttagagatattatctagggcttcaaataatctctggacagcaataaaagtctcttcaaaagtattgtataacggcaatggggctaatctgattacatctggtcttctttcgtcacagattatagcatgatcatgcaagtacgcattaactcgttccatgacgttcttgtccttttcatcgaaatgcggttgaaacataatggacaattgacatcctctttcagctggattcaaaggagttaaaattttaaacccaaatttggagtttgatgtactggattgtggtatgtaatacttggaattcgtcaatagatcctgtaaaaattgagtcaaagcaacacttttttcacgaagtttagatactccacccactttagcatacacttccaatgacgacttcacagcaacaacatcaagaacagaaggatttgactgtctgtaagaaagagccgagtttattggatcaaactcttctaacattttgaatcgttcttgggagttattgccccaccaaccagctagtctaggaacgaaactgcttttcttgttctctatggtgtatttttcatgcacaaaaatcccacctatggctccaggtcccgagtttaaatatttgtaggaacaccaagcagcaaaatctactccccaatcatgtaaatttaatgggacattcccaactgcatgggcaagatcccacccaactttaatttgttggctcttttccttagcgtatttagttatttcctctatcttgaaaaattgaccagtgtagtattggataccaggaaaacacactagagccaattcatccaggttctcatctatagccttgattattctttctgttttaatataagtttcaccaggttgaacttccaattgaatcaaatgtttctcgtcgtatccgaacaatttaacaatgttcaaaaatgcatagtagtcagaaggaaatgcttgtttttcaaataaaattttggttcttttcccctcaggtttgtaaaaatggatcaacaatgcattcaagtttgctgttaaagaacccataactgcaacttcgttttcctttgcaccaacaatgggggctattaatggtaataagggtaaatcgatgtctacccacggtgttaacagtttgtcaggatgattgaaatgagactcaacccctcgttcaacccatgcatttaattcatcattgatagctttctttgtattcttaggcatcaacccaagagagtttccacataaataaatagactcagttgatgactcatatttattatttttgatacctaatgatccaaaagttggtatggcaaactcatttttaaaagttgggaactttttgtccaatttctttgcctcggctaatgacatctgataataaaatggggttggagtagttggtggtataaccggagagatagaattgaagaaaaaaatcggaaacaacaaaaaaagttgataccctgtattatgtgggagataattgcgaatggtggaaaaaaaaaagacgccattgagtctcaacaacaattctgtcagctgaagagctttacaatcgagaaactatgattcattccgttttaatatgtatgtgtttagtaaactcatgaattttatttgtggtctactttagtactaacataatcattggatagtcaataatgatggtcttccgagactaatgaaattctataccaaagtcgatattccaacacagaaattgctcttgcaacaagtgcacctgttgatatctagagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 19 - DNA, 14064 bp, Artificial Sequence
  Plasmid
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacctgccactactaccactgggagtttcgttcttctcgatactattagctttacttcctgcactagcagtggttggatcaacagaatcttcataatcatcaaaatcgtcttttgaagaccccccgtttgatgtatggccctgtcttttcatcaaactttttatatagttgactgaactgaggctaaatatgtgatcatcttcactatagacaatctttctcttatttgcaccaccgccaccactagtctttgagaaattctcaaaaccttttacgatattaccaagcgggctctcttcgaaataatctatctctttttgatatatcgaatcctctagcgtggttagctttctagttagttcttgcttcttaagaatttgctggattagtttatttttcaattcaacgtatttctcagagtcatctttagattttgatgaagatgtgcgttcattcgctatatccttcttggtcgtgtcttttcgatcctccttggctggcactgaactcgtcttttttggcgttgctgttccagacagacttatctcattagatttggaacttgtgggtttaacatcatttgtatctttagtagacatgattgtgcaataccgtgattatttgttttgaaaggtctgtcatatttctatcaatttcaaaacaaaatgttcatcagaaaaaagccaaaaatgtctcttctagtttcttagtggtgtcgcataatacacaatgtcgctcaacaatccacattcccggcgcatagctcaaatcacatgactacagctaacaattacacaaaaaaaattctctttttgatgtagcaactatcttcaactaaaacattttctccttcggcccatgattgtcctccgggtcgacagcaagccgttacaattgagatggaaagcgacctaccttcactcgataaggtgcttaattgtacttcatataaatctggcccggatctaaacaaatgagttccattaagccgtgggttctcaattagggtttttgtttttgatttagaaaaaagagatcaagatttgtttacaggtgatgcctttttttagaacttatgcgttgcaaaagttgactaacgatttctataaggtgatccacactaattatacaaacgtacaaacagacatacttttcctgcgttcacctgatgttggccagatttctctcttcattgcatagaacataaccacactagggcaacagaaaaaaaaaaaaaaagtgcatcgggaagttgtgttccattcattatatgtctactactgcatatgagtagcccacccaccaccaccatagtaagtttttgtgtatgcgcgccgtcaggttatttcatttctgaatttttcaaccaccttactccctttattgttgattgacaattttgctcacagtaagatcttttagactccaattaatataaaataagtctgattttccaattcctgttttttctttttttttctgtttctatttctttccttttctcccttttttttaattcttcattcaatcatcaattgataattcaggaatattacaacaacccgggatggataaaaagtatagtattggtttagatattggtactaactctgtgggttgggcagttatcaccgacgaatataaagttccatcaaagaaatttaaggtgttaggtaacactgacagacactcaataaaaaagaatcttatcggtgctcttttgttcgactccggtgaaactgccgaggctacacgtttaaaaagaacagcaagaagaagatatacccgtagaaaaaatagaatatgttatttacaagaaatcttttctaatgaaatggctaaagttgatgattcctttttccatagattggaagagtcatttttggttgaagaagacaaaaagcatgagagacatccaatctttgggaatatagttgatgaagtggcttaccatgaaaaatatcctaccatttatcatttaagaaagaaattggtagattcaactgataaagctgaccttagattaatctatttagcacttgcccatatgattaaatttagaggtcattttttgattgaaggtgatttgaacccagataattctgacgtggataaattatttattcaattagtccaaacctacaaccaattatttgaggaaaatccaattaatgctagtggtgtcgatgccaaagctatattatcagccagattatcaaaatctagacgtttggaaaatttgattgcccaattgccaggagaaaaaaagaatggattatttggaaacttgatcgcattatcattgggtttgacaccaaattttaaatctaattttgatttagctgaagatgctaaattacaattatcaaaagacacctatgacgacgatttggacaatttacttgctcaaattggtgatcaatatgcagatttgttcttagctgctaaaaacttatctgatgctattttgttgtctgatattttgagagtgaacacagaaataaccaaagctccattatcagcatctatgatcaaacgttatgatgaacaccatcaggatttgactttattgaaagctttggtgagacaacaattgccagagaagtataaagaaatctttttcgatcaatctaaaaacgggtatgcaggttatattgatgggggtgcctcccaagaggaattttacaaatttataaaacctattttagaaaagatggatgggactgaggaacttttggtcaaattgaacagagaagatttgttacgtaaacagagaacttttgataatggtagtatacctcaccaaattcatttgggtgagttgcatgcaattttaagaagacaagaagatttttatccatttttaaaagataatagagaaaaaatcgagaaaattttaacctttagaattccatactatgttgggcctttggctagaggtaattcaagatttgcctggatgacacgtaaatcagaagaaactattaccccttggaattttgaagaggttgttgataaaggagcatcagcacagagttttattgaaagaatgaccaatttcgataaaaacttaccaaatgaaaaagttttaccaaaacattccttgttatacgaatattttactgtttacaatgaacttacaaaggttaaatatgttactgaaggtatgcgtaagccagcctttttatctggagaacagaaaaaggcaatagttgatttattgtttaaaacaaatagaaaagttactgttaaacaattaaaagaagattactttaagaaaattgaatgttttgattcagttgaaatcagtggtgttgaagacagatttaatgctagtttaggaacttaccatgatttacttaaaattatcaaagataaagatttcttggataacgaagaaaatgaagacattttagaagacattgttttaaccttaactttattcgaagatagagagatgattgaagaacgtttgaagacttatgcacatttgtttgacgataaagtgatgaaacagttgaaaagaagacgttatactggatggggtagattgtctcgtaaattgatcaatggaattagagataaacaaagtggtaaaactatcttggactttttgaaatctgacggatttgctaatagaaatttcatgcaattgatccacgacgatagtttgacatttaaagaagacatccaaaaggcccaagtgagtgggcaaggtgattcattacatgaacatattgcaaatttagccggatctcctgctattaagaaagggatattacaaactgttaaagttgtggatgaattagtgaaagtaatgggaagacataaacctgaaaacattgtcattgagatggcaagagaaaatcaaactacacaaaaaggacagaaaaatagtagagaacgtatgaaaagaatagaagagggtattaaagaattgggtagtcaaatattgaaagaacacccagtggaaaatacccagttgcaaaatgaaaaattatatctttactaccttcaaaatggacgtgatatgtatgttgatcaggaattagatataaatagactttcagattatgatgtagatcatatagttccacaatctttcttgaaagatgattccatagacaataaagtattaactagaagtgataaaaatagaggtaaaagtgataatgtcccaagtgaggaagtcgtcaaaaagatgaaaaattactggcgtcaacttttgaatgctaaattaattactcaaagaaaatttgataatttgactaaagcagaaagaggtgggctttctgaattagataaagccgggttcattaaaagacaattggtcgaaactagacaaattactaaacatgttgcccaaattttagattcccgtatgaacactaagtatgacgaaaatgataagttaatacgtgaggttaaagtcattactttaaaatcaaaacttgtctctgatttcagaaaggatttccaattctataaagttagagaaattaataattatcatcatgctcatgatgcatatttgaatgctgtagttggaactgctttaatcaagaaataccctaaattagaatctgaatttgtatatggtgattacaaagtctatgatgttagaaagatgattgctaaatcagaacaagaaattggtaaagctacagctaaatacttcttttactctaacattatgaatttctttaaaacagaaattactttggcaaacggtgaaattagaaaaagacctcttattgaaacaaatggtgagactggagagatagtttgggacaaagggcgtgatttcgctactgttagaaaagttttatcaatgccacaagttaacattgtaaagaaaacagaggttcaaactggtggtttctcaaaagaaagtattttgcctaaaagaaatagtgataaattgattgccagaaaaaaggattgggatccaaagaaatatggtggtttcgactcaccaaccgtagcctattctgttttggttgtggcaaaggttgaaaagggtaaaagtaaaaagcttaaatcagtaaaagaacttttgggtattacaataatggaaagaagttcctttgaaaagaaccctattgattttttggaagctaaaggttataaggaagtaaagaaggacttaataatcaaattgcctaaatattctttatttgaattagaaaatgggagaaaaagaatgttggcttctgctggagaattgcaaaagggtaatgaattagcattgccttccaaatatgttaacttcttgtatttagcttcacactatgaaaagttgaaagggtcaccagaagataacgagcaaaaacaattatttgttgaacaacacaaacactacttagatgagattatagaacaaattagtgaattcagtaaaagagtgatattagctgatgcaaatttagataaagttttgtcagcctataacaaacatagagataagccaattagagaacaagcagaaaacattattcacttatttacccttaccaatttaggagcacctgctgctttcaagtattttgatacaacaattgatcgtaaaagatatacctcaacaaaagaagtcttagacgccaccttaattcatcaatcaatcactggattgtatgagacaagaattgatttgtctcaattgggtggtgatgaaggggctgatcctaagaagaaaagaaaagttgatccaaagaaaaagcgtaaggtggatcctaagaaaaagagaaaggttgactacaaagaccatgacggtgattataaagatcatgacatcgactacaaggatgacgatgacaagtgataatgactgcagagatccatcgacctgccgccaagctaattccgggcgaatttctgtcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccgggccccccctaactcaagtacaacagatctggaccacctttgattgtaaatagtaataattaccacccttatctaattatttatttaacttatttatttatttattatacatatatacaaatctaataaagtgaaaatctcccccttcacacttcacatatgttaggcgtcatcctgtgctcccgagaaccagtaccagtacatcgctgtttcgttcgagacttgaggtctagttttatacgtgaagaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaattttcatatgagaaccgttatcgataactaaagcagcaacttcttctataaaaatgggttagtatgacagtcatttaaataaggaatttttcagttggcttggtttcaattcaatgttcgttttttttttttcttgctgtgtttgtgtttgtgttgtttatagttgtgtgcactgatcgtcgaaaaaaaaaattcatagtgagccgggaaatctgtatagcccagataacaacacaagtccaaactagaaactcgtcaaacaccaaaagcaatgttgaatcaattgccttgcacaagtacacgtaggaaaacataaaacattgcaattttgaatattgagccttttgtcgtaacattgattgataggattactcaccgaatggttttgaaaccactgccgacagatcaatcaatcaatcaaaaaacgtgaactttgaaaaaggggaagaacagatacattgaagttagccatttccactgatcgtcacaacatatctgataaattactttcaaaattataagctgatgtgtgtgtattattaatgtgacagtaacatcccaaacgagaaatattatctcgacaacaaaaaagtttgatctgaattgaaaatgaagttttcccaccctacccatttgtcatattgaaaccaatcaactgattaatcaatcaattagaattgaagctaaactaaaacataccaccgtccattttgaatgattatatttttttaatattaatatcgagataatgtttctaagaaagaaagaaaaccaggagtgaaaattagaaaaggaaaggaaaggaaaaaaagaaaaatctgaaaatatataaaaaaaaattgtttcgttggcaataaatcttggtgagaacagcgaccgaaagcaaataagaacaaaatatgagtgtattacgttgaacaactaattaacgtgtgtgtatggatctttttttcttttttctctttaaccgactataaacaacaaacatttttgggcagtgcacacactacttaatatacacagcataaattacacgattagaaacaaattagcttattaaaataacctaatcaaaccgaatattttatggtattatgagtaaactatataatataaatagcacacacccacaacaacaacaaaggaaaactaaaaggttttttctttttgaaaagatcgttttctttattattctctagttttgacggcggccgcaagtgattagacttagtccgttcaaatcaagcacaactctgttcattgtttcaacaagaattaattcaaaaacaggttcggtgcataatttgcaaaaaaatattgcagcttctgtggctcgaacacagtacctccagatttcaggtttgaaatacttcagtctgacgctctcccagatgagctaaagctgcaataagaaaacccacgccgggattcgaacccggaatcctttgattagaagtcaaaagcgataaccatttcgccacgcaggcctacttgatgggtttgtaaatggtctactttttcagacctaacagaaattttaatgaaagtcatattcttatacaataaaactgtgtcataaaagcagatattcgactttcgtagattatataggacccaagaactaaaatttaatgccatattatgcatttttaatctgtaaaagtgttgtttccaacctatcacaagtacgttcttgtaacttgtgtttgtagggttgcaaatgaatcataacaacatctcaacagaacatgtatagcaaagcttagtataaaatcagtgttttgagaggcaatccaagaatgtttacatcaaagtttcaataaatatcgaccgaaactgaaaatctttttaggttattgttcacttttttgtaaatatttaaacattttttggacctaaaaaaatacaaacaccaattacgtaccaagaagcatctaatcaactcccagatcaccactatacatttaaaagtcattggtcaataactatactcgagtattgcctcatcaaagaaacaatcaaatattatagatactcactccatcacgtgataatttcactggtatggaaaagtggaaaattttataaaaaaaaatttgatgcctttggcatagctgaaacttcggcccaataggattggagaatatgttttcgcagcgttcttacaattaaattgtggtggaagttcgagacttgcgtaaactatttttaatttgaaattagttgttgttggagggttttagagctagaaatagcaagttaaaataaggctagtccgttatcaacttgaaaaagtggcaccgagtcggtgctttttttctcgagtttttttatcgagtgtttaaggataatgataactgaagagaagaattagttttgccgccaccgcgggtttgcctctgattaaataaaaaaaagctggtgctttttttttcttttataggaacatcttgaatatatgaactaattaaatgataattttttacccatctttactcttaatcactgagctgcagtcaaagaaaaagggatacagcacctggtgaagagatgaacggagactaacttagacgcgttgattctttttaattgcacattttattaatcgatgctaacgtctatttacatatattctttagagatattatctagggcttcaaataatctctggacagcaataaaagtctcttcaaaagtattgtataacggcaatggggctaatctgattacatctggtcttctttcgtcacagattatagcatgatcatgcaagtacgcattaactcgttccatgacgttcttgtccttttcatcgaaatgcggttgaaacataatggacaattgacatcctctttcagctggattcaaaggagttaaaattttaaacccaaatttggagtttgatgtactggattgtggtatgtaatacttggaattcgtcaatagatcctgtaaaaattgagtcaaagcaacacttttttcacgaagtttagatactccacccactttagcatacacttccaatgacgacttcacagcaacaacatcaagaacagaaggatttgactgtctgtaagaaagagccgagtttattggatcaaactcttctaacattttgaatcgttcttgggagttattgccccaccaaccagctagtctaggaacgaaactgcttttcttgttctctatggtgtatttttcatgcacaaaaatcccacctatggctccaggtcccgagtttaaatatttgtaggaacaccaagcagcaaaatctactccccaatcatgtaaatttaatgggacattcccaactgcatgggcaagatcccacccaactttaatttgttggctcttttccttagcgtatttagttatttcctctatcttgaaaaattgaccagtgtagtattggataccaggaaaacacactagagccaattcatccaggttctcatctatagccttgattattctttctgttttaatataagtttcaccaggttgaacttccaattgaatcaaatgtttctcgtcgtatccgaacaatttaacaatgttcaaaaatgcatagtagtcagaaggaaatgcttgtttttcaaataaaattttggttcttttcccctcaggtttgtaaaaatggatcaacaatgcattcaagtttgctgttaaagaacccataactgcaacttcgttttcctttgcaccaacaatgggggctattaatggtaataagggtaaatcgatgtctacccacggtgttaacagtttgtcaggatgattgaaatgagactcaacccctcgttcaacccatgcatttaattcatcattgatagctttctttgtattcttaggcatcaacccaagagagtttccacataaataaatagactcagttgatgactcatatttattatttttgatacctaatgatccaaaagttggtatggcaaactcatttttaaaagttgggaactttttgtccaatttctttgcctcggctaatgacatctgataataaaatggggttggagtagttggtggtataaccggagagatagaattgaagaaaaaaatcggaaacaacaaaaaaagttgataccctgtattatgtgggagataattgcgaatggtggaaaaaaaaaagacgccattgagtctcaacaacaattctgtcagctgaagagctttacaatcgagaaactatgattcattccgttttaatatgtatgtgtttagtaaactcatgaattttatttgtggtctactttagtactaacataatcattggatagtcaataatgatggtcttccgagactaatgaaattctataccaaagtcgatattccaacacagaaattgctcttgcaacaagtgcacctgttgatatctagagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 20 - DNA, 14064 bp, Artificial Sequence
  Plasmid
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacctgccactactaccactgggagtttcgttcttctcgatactattagctttacttcctgcactagcagtggttggatcaacagaatcttcataatcatcaaaatcgtcttttgaagaccccccgtttgatgtatggccctgtcttttcatcaaactttttatatagttgactgaactgaggctaaatatgtgatcatcttcactatagacaatctttctcttatttgcaccaccgccaccactagtctttgagaaattctcaaaaccttttacgatattaccaagcgggctctcttcgaaataatctatctctttttgatatatcgaatcctctagcgtggttagctttctagttagttcttgcttcttaagaatttgctggattagtttatttttcaattcaacgtatttctcagagtcatctttagattttgatgaagatgtgcgttcattcgctatatccttcttggtcgtgtcttttcgatcctccttggctggcactgaactcgtcttttttggcgttgctgttccagacagacttatctcattagatttggaacttgtgggtttaacatcatttgtatctttagtagacatgattgtgcaataccgtgattatttgttttgaaaggtctgtcatatttctatcaatttcaaaacaaaatgttcatcagaaaaaagccaaaaatgtctcttctagtttcttagtggtgtcgcataatacacaatgtcgctcaacaatccacattcccggcgcatagctcaaatcacatgactacagctaacaattacacaaaaaaaattctctttttgatgtagcaactatcttcaactaaaacattttctccttcggcccatgattgtcctccgggtcgacagcaagccgttacaattgagatggaaagcgacctaccttcactcgataaggtgcttaattgtacttcatataaatctggcccggatctaaacaaatgagttccattaagccgtgggttctcaattagggtttttgtttttgatttagaaaaaagagatcaagatttgtttacaggtgatgcctttttttagaacttatgcgttgcaaaagttgactaacgatttctataaggtgatccacactaattatacaaacgtacaaacagacatacttttcctgcgttcacctgatgttggccagatttctctcttcattgcatagaacataaccacactagggcaacagaaaaaaaaaaaaaaagtgcatcgggaagttgtgttccattcattatatgtctactactgcatatgagtagcccacccaccaccaccatagtaagtttttgtgtatgcgcgccgtcaggttatttcatttctgaatttttcaaccaccttactccctttattgttgattgacaattttgctcacagtaagatcttttagactccaattaatataaaataagtctgattttccaattcctgttttttctttttttttctgtttctatttctttccttttctcccttttttttaattcttcattcaatcatcaattgataattcaggaatattacaacaacccgggatggataaaaagtatagtattggtttagatattggtactaactctgtgggttgggcagttatcaccgacgaatataaagttccatcaaagaaatttaaggtgttaggtaacactgacagacactcaataaaaaagaatcttatcggtgctcttttgttcgactccggtgaaactgccgaggctacacgtttaaaaagaacagcaagaagaagatatacccgtagaaaaaatagaatatgttatttacaagaaatcttttctaatgaaatggctaaagttgatgattcctttttccatagattggaagagtcatttttggttgaagaagacaaaaagcatgagagacatccaatctttgggaatatagttgatgaagtggcttaccatgaaaaatatcctaccatttatcatttaagaaagaaattggtagattcaactgataaagctgaccttagattaatctatttagcacttgcccatatgattaaatttagaggtcattttttgattgaaggtgatttgaacccagataattctgacgtggataaattatttattcaattagtccaaacctacaaccaattatttgaggaaaatccaattaatgctagtggtgtcgatgccaaagctatattatcagccagattatcaaaatctagacgtttggaaaatttgattgcccaattgccaggagaaaaaaagaatggattatttggaaacttgatcgcattatcattgggtttgacaccaaattttaaatctaattttgatttagctgaagatgctaaattacaattatcaaaagacacctatgacgacgatttggacaatttacttgctcaaattggtgatcaatatgcagatttgttcttagctgctaaaaacttatctgatgctattttgttgtctgatattttgagagtgaacacagaaataaccaaagctccattatcagcatctatgatcaaacgttatgatgaacaccatcaggatttgactttattgaaagctttggtgagacaacaattgccagagaagtataaagaaatctttttcgatcaatctaaaaacgggtatgcaggttatattgatgggggtgcctcccaagaggaattttacaaatttataaaacctattttagaaaagatggatgggactgaggaacttttggtcaaattgaacagagaagatttgttacgtaaacagagaacttttgataatggtagtatacctcaccaaattcatttgggtgagttgcatgcaattttaagaagacaagaagatttttatccatttttaaaagataatagagaaaaaatcgagaaaattttaacctttagaattccatactatgttgggcctttggctagaggtaattcaagatttgcctggatgacacgtaaatcagaagaaactattaccccttggaattttgaagaggttgttgataaaggagcatcagcacagagttttattgaaagaatgaccaatttcgataaaaacttaccaaatgaaaaagttttaccaaaacattccttgttatacgaatattttactgtttacaatgaacttacaaaggttaaatatgttactgaaggtatgcgtaagccagcctttttatctggagaacagaaaaaggcaatagttgatttattgtttaaaacaaatagaaaagttactgttaaacaattaaaagaagattactttaagaaaattgaatgttttgattcagttgaaatcagtggtgttgaagacagatttaatgctagtttaggaacttaccatgatttacttaaaattatcaaagataaagatttcttggataacgaagaaaatgaagacattttagaagacattgttttaaccttaactttattcgaagatagagagatgattgaagaacgtttgaagacttatgcacatttgtttgacgataaagtgatgaaacagttgaaaagaagacgttatactggatggggtagattgtctcgtaaattgatcaatggaattagagataaacaaagtggtaaaactatcttggactttttgaaatctgacggatttgctaatagaaatttcatgcaattgatccacgacgatagtttgacatttaaagaagacatccaaaaggcccaagtgagtgggcaaggtgattcattacatgaacatattgcaaatttagccggatctcctgctattaagaaagggatattacaaactgttaaagttgtggatgaattagtgaaagtaatgggaagacataaacctgaaaacattgtcattgagatggcaagagaaaatcaaactacacaaaaaggacagaaaaatagtagagaacgtatgaaaagaatagaagagggtattaaagaattgggtagtcaaatattgaaagaacacccagtggaaaatacccagttgcaaaatgaaaaattatatctttactaccttcaaaatggacgtgatatgtatgttgatcaggaattagatataaatagactttcagattatgatgtagatcatatagttccacaatctttcttgaaagatgattccatagacaataaagtattaactagaagtgataaaaatagaggtaaaagtgataatgtcccaagtgaggaagtcgtcaaaaagatgaaaaattactggcgtcaacttttgaatgctaaattaattactcaaagaaaatttgataatttgactaaagcagaaagaggtgggctttctgaattagataaagccgggttcattaaaagacaattggtcgaaactagacaaattactaaacatgttgcccaaattttagattcccgtatgaacactaagtatgacgaaaatgataagttaatacgtgaggttaaagtcattactttaaaatcaaaacttgtctctgatttcagaaaggatttccaattctataaagttagagaaattaataattatcatcatgctcatgatgcatatttgaatgctgtagttggaactgctttaatcaagaaataccctaaattagaatctgaatttgtatatggtgattacaaagtctatgatgttagaaagatgattgctaaatcagaacaagaaattggtaaagctacagctaaatacttcttttactctaacattatgaatttctttaaaacagaaattactttggcaaacggtgaaattagaaaaagacctcttattgaaacaaatggtgagactggagagatagtttgggacaaagggcgtgatttcgctactgttagaaaagttttatcaatgccacaagttaacattgtaaagaaaacagaggttcaaactggtggtttctcaaaagaaagtattttgcctaaaagaaatagtgataaattgattgccagaaaaaaggattgggatccaaagaaatatggtggtttcgactcaccaaccgtagcctattctgttttggttgtggcaaaggttgaaaagggtaaaagtaaaaagcttaaatcagtaaaagaacttttgggtattacaataatggaaagaagttcctttgaaaagaaccctattgattttttggaagctaaaggttataaggaagtaaagaaggacttaataatcaaattgcctaaatattctttatttgaattagaaaatgggagaaaaagaatgttggcttctgctggagaattgcaaaagggtaatgaattagcattgccttccaaatatgttaacttcttgtatttagcttcacactatgaaaagttgaaagggtcaccagaagataacgagcaaaaacaattatttgttgaacaacacaaacactacttagatgagattatagaacaaattagtgaattcagtaaaagagtgatattagctgatgcaaatttagataaagttttgtcagcctataacaaacatagagataagccaattagagaacaagcagaaaacattattcacttatttacccttaccaatttaggagcacctgctgctttcaagtattttgatacaacaattgatcgtaaaagatatacctcaacaaaagaagtcttagacgccaccttaattcatcaatcaatcactggattgtatgagacaagaattgatttgtctcaattgggtggtgatgaaggggctgatcctaagaagaaaagaaaagttgatccaaagaaaaagcgtaaggtggatcctaagaaaaagagaaaggttgactacaaagaccatgacggtgattataaagatcatgacatcgactacaaggatgacgatgacaagtgataatgactgcagagatccatcgacctgccgccaagctaattccgggcgaatttctgtcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccgggccccccctaactcaagtacaacagatctggaccacctttgattgtaaatagtaataattaccacccttatctaattatttatttaacttatttatttatttattatacatatatacaaatctaataaagtgaaaatctcccccttcacacttcacatatgttaggcgtcatcctgtgctcccgagaaccagtaccagtacatcgctgtttcgttcgagacttgaggtctagttttatacgtgaagaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaattttcatatgagaaccgttatcgataactaaagcagcaacttcttctataaaaatgggttagtatgacagtcatttaaataaggaatttttcagttggcttggtttcaattcaatgttcgttttttttttttcttgctgtgtttgtgtttgtgttgtttatagttgtgtgcactgatcgtcgaaaaaaaaaattcatagtgagccgggaaatctgtatagcccagataacaacacaagtccaaactagaaactcgtcaaacaccaaaagcaatgttgaatcaattgccttgcacaagtacacgtaggaaaacataaaacattgcaattttgaatattgagccttttgtcgtaacattgattgataggattactcaccgaatggttttgaaaccactgccgacagatcaatcaatcaatcaaaaaacgtgaactttgaaaaaggggaagaacagatacattgaagttagccatttccactgatcgtcacaacatatctgataaattactttcaaaattataagctgatgtgtgtgtattattaatgtgacagtaacatcccaaacgagaaatattatctcgacaacaaaaaagtttgatctgaattgaaaatgaagttttcccaccctacccatttgtcatattgaaaccaatcaactgattaatcaatcaattagaattgaagctaaactaaaacataccaccgtccattttgaatgattatatttttttaatattaatatcgagataatgtttctaagaaagaaagaaaaccaggagtgaaaattagaaaaggaaaggaaaggaaaaaaagaaaaatctgaaaatatataaaaaaaaattgtttcgttggcaataaatcttggtgagaacagcgaccgaaagcaaataagaacaaaatatgagtgtattacgttgaacaactaattaacgtgtgtgtatggatctttttttcttttttctctttaaccgactataaacaacaaacatttttgggcagtgcacacactacttaatatacacagcataaattacacgattagaaacaaattagcttattaaaataacctaatcaaaccgaatattttatggtattatgagtaaactatataatataaatagcacacacccacaacaacaacaaaggaaaactaaaaggttttttctttttgaaaagatcgttttctttattattctctagttttgacggcggccgcaagtgattagacttagtccgttcaaatcaagcacaactctgttcattgtttcaacaagaattaattcaaaaacaggttcggtgcataatttgcaaaaaaatattgcagcttctgtggctcgaacacagtacctccagatttcaggtttgaaatacttcagtctgacgctctcccagatgagctaaagctgcaataagaaaacccacgccgggattcgaacccggaatcctttgattagaagtcaaaagcgataaccatttcgccacgcaggcctacttgatgggtttgtaaatggtctactttttcagacctaacagaaattttaatgaaagtcatattcttatacaataaaactgtgtcataaaagcagatattcgactttcgtagattatataggacccaagaactaaaatttaatgccatattatgcatttttaatctgtaaaagtgttgtttccaacctatcacaagtacgttcttgtaacttgtgtttgtagggttgcaaatgaatcataacaacatctcaacagaacatgtatagcaaagcttagtataaaatcagtgttttgagaggcaatccaagaatgtttacatcaaagtttcaataaatatcgaccgaaactgaaaatctttttaggttattgttcacttttttgtaaatatttaaacattttttggacctaaaaaaatacaaacaccaattacgtaccaagaagcatctaatcaactcccagatcaccactatacatttaaaagtcattggtcaataactatactcgagtattgcctcatcaaagaaacaatcaaatattatagatactcactccatcacgtgataatttcactggtatggaaaagtggaaaattttataaaaaaaaatttgatgcctttggcatagctgaaacttcggcccaataggattggagaatatgttttcgcagcgttcttacaattaaattgtggtggaagttcgagacttgcgtaaactatttttaatttgatataagaatgaagacaacggttttagagctagaaatagcaagttaaaataaggctagtccgttatcaacttgaaaaagtggcaccgagtcggtgctttttttctcgagtttttttatcgagtgtttaaggataatgataactgaagagaagaattagttttgccgccaccgcgggtttgcctctgattaaataaaaaaaagctggtgctttttttttcttttataggaacatcttgaatatatgaactaattaaatgataattttttacccatctttactcttaatcactgagctgcagtcaaagaaaaagggatacagcacctggtgaagagatgaacggagactaacttagacgcgttgattctttttaattgcacattttattaatcgatgctaacgtctatttacatatattctttagagatattatctagggcttcaaataatctctggacagcaataaaagtctcttcaaaagtattgtataacggcaatggggctaatctgattacatctggtcttctttcgtcacagattatagcatgatcatgcaagtacgcattaactcgttccatgacgttcttgtccttttcatcgaaatgcggttgaaacataatggacaattgacatcctctttcagctggattcaaaggagttaaaattttaaacccaaatttggagtttgatgtactggattgtggtatgtaatacttggaattcgtcaatagatcctgtaaaaattgagtcaaagcaacacttttttcacgaagtttagatactccacccactttagcatacacttccaatgacgacttcacagcaacaacatcaagaacagaaggatttgactgtctgtaagaaagagccgagtttattggatcaaactcttctaacattttgaatcgttcttgggagttattgccccaccaaccagctagtctaggaacgaaactgcttttcttgttctctatggtgtatttttcatgcacaaaaatcccacctatggctccaggtcccgagtttaaatatttgtaggaacaccaagcagcaaaatctactccccaatcatgtaaatttaatgggacattcccaactgcatgggcaagatcccacccaactttaatttgttggctcttttccttagcgtatttagttatttcctctatcttgaaaaattgaccagtgtagtattggataccaggaaaacacactagagccaattcatccaggttctcatctatagccttgattattctttctgttttaatataagtttcaccaggttgaacttccaattgaatcaaatgtttctcgtcgtatccgaacaatttaacaatgttcaaaaatgcatagtagtcagaaggaaatgcttgtttttcaaataaaattttggttcttttcccctcaggtttgtaaaaatggatcaacaatgcattcaagtttgctgttaaagaacccataactgcaacttcgttttcctttgcaccaacaatgggggctattaatggtaataagggtaaatcgatgtctacccacggtgttaacagtttgtcaggatgattgaaatgagactcaacccctcgttcaacccatgcatttaattcatcattgatagctttctttgtattcttaggcatcaacccaagagagtttccacataaataaatagactcagttgatgactcatatttattatttttgatacctaatgatccaaaagttggtatggcaaactcatttttaaaagttgggaactttttgtccaatttctttgcctcggctaatgacatctgataataaaatggggttggagtagttggtggtataaccggagagatagaattgaagaaaaaaatcggaaacaacaaaaaaagttgataccctgtattatgtgggagataattgcgaatggtggaaaaaaaaaagacgccattgagtctcaacaacaattctgtcagctgaagagctttacaatcgagaaactatgattcattccgttttaatatgtatgtgtttagtaaactcatgaattttatttgtggtctactttagtactaacataatcattggatagtcaataatgatggtcttccgagactaatgaaattctataccaaagtcgatattccaacacagaaattgctcttgcaacaagtgcacctgttgatatctagagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 21 - DNA, 14064 bp, Artificial Sequence
  Plasmid
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacctgccactactaccactgggagtttcgttcttctcgatactattagctttacttcctgcactagcagtggttggatcaacagaatcttcataatcatcaaaatcgtcttttgaagaccccccgtttgatgtatggccctgtcttttcatcaaactttttatatagttgactgaactgaggctaaatatgtgatcatcttcactatagacaatctttctcttatttgcaccaccgccaccactagtctttgagaaattctcaaaaccttttacgatattaccaagcgggctctcttcgaaataatctatctctttttgatatatcgaatcctctagcgtggttagctttctagttagttcttgcttcttaagaatttgctggattagtttatttttcaattcaacgtatttctcagagtcatctttagattttgatgaagatgtgcgttcattcgctatatccttcttggtcgtgtcttttcgatcctccttggctggcactgaactcgtcttttttggcgttgctgttccagacagacttatctcattagatttggaacttgtgggtttaacatcatttgtatctttagtagacatgattgtgcaataccgtgattatttgttttgaaaggtctgtcatatttctatcaatttcaaaacaaaatgttcatcagaaaaaagccaaaaatgtctcttctagtttcttagtggtgtcgcataatacacaatgtcgctcaacaatccacattcccggcgcatagctcaaatcacatgactacagctaacaattacacaaaaaaaattctctttttgatgtagcaactatcttcaactaaaacattttctccttcggcccatgattgtcctccgggtcgacagcaagccgttacaattgagatggaaagcgacctaccttcactcgataaggtgcttaattgtacttcatataaatctggcccggatctaaacaaatgagttccattaagccgtgggttctcaattagggtttttgtttttgatttagaaaaaagagatcaagatttgtttacaggtgatgcctttttttagaacttatgcgttgcaaaagttgactaacgatttctataaggtgatccacactaattatacaaacgtacaaacagacatacttttcctgcgttcacctgatgttggccagatttctctcttcattgcatagaacataaccacactagggcaacagaaaaaaaaaaaaaaagtgcatcgggaagttgtgttccattcattatatgtctactactgcatatgagtagcccacccaccaccaccatagtaagtttttgtgtatgcgcgccgtcaggttatttcatttctgaatttttcaaccaccttactccctttattgttgattgacaattttgctcacagtaagatcttttagactccaattaatataaaataagtctgattttccaattcctgttttttctttttttttctgtttctatttctttccttttctcccttttttttaattcttcattcaatcatcaattgataattcaggaatattacaacaacccgggatggataaaaagtatagtattggtttagatattggtactaactctgtgggttgggcagttatcaccgacgaatataaagttccatcaaagaaatttaaggtgttaggtaacactgacagacactcaataaaaaagaatcttatcggtgctcttttgttcgactccggtgaaactgccgaggctacacgtttaaaaagaacagcaagaagaagatatacccgtagaaaaaatagaatatgttatttacaagaaatcttttctaatgaaatggctaaagttgatgattcctttttccatagattggaagagtcatttttggttgaagaagacaaaaagcatgagagacatccaatctttgggaatatagttgatgaagtggcttaccatgaaaaatatcctaccatttatcatttaagaaagaaattggtagattcaactgataaagctgaccttagattaatctatttagcacttgcccatatgattaaatttagaggtcattttttgattgaaggtgatttgaacccagataattctgacgtggataaattatttattcaattagtccaaacctacaaccaattatttgaggaaaatccaattaatgctagtggtgtcgatgccaaagctatattatcagccagattatcaaaatctagacgtttggaaaatttgattgcccaattgccaggagaaaaaaagaatggattatttggaaacttgatcgcattatcattgggtttgacaccaaattttaaatctaattttgatttagctgaagatgctaaattacaattatcaaaagacacctatgacgacgatttggacaatttacttgctcaaattggtgatcaatatgcagatttgttcttagctgctaaaaacttatctgatgctattttgttgtctgatattttgagagtgaacacagaaataaccaaagctccattatcagcatctatgatcaaacgttatgatgaacaccatcaggatttgactttattgaaagctttggtgagacaacaattgccagagaagtataaagaaatctttttcgatcaatctaaaaacgggtatgcaggttatattgatgggggtgcctcccaagaggaattttacaaatttataaaacctattttagaaaagatggatgggactgaggaacttttggtcaaattgaacagagaagatttgttacgtaaacagagaacttttgataatggtagtatacctcaccaaattcatttgggtgagttgcatgcaattttaagaagacaagaagatttttatccatttttaaaagataatagagaaaaaatcgagaaaattttaacctttagaattccatactatgttgggcctttggctagaggtaattcaagatttgcctggatgacacgtaaatcagaagaaactattaccccttggaattttgaagaggttgttgataaaggagcatcagcacagagttttattgaaagaatgaccaatttcgataaaaacttaccaaatgaaaaagttttaccaaaacattccttgttatacgaatattttactgtttacaatgaacttacaaaggttaaatatgttactgaaggtatgcgtaagccagcctttttatctggagaacagaaaaaggcaatagttgatttattgtttaaaacaaatagaaaagttactgttaaacaattaaaagaagattactttaagaaaattgaatgttttgattcagttgaaatcagtggtgttgaagacagatttaatgctagtttaggaacttaccatgatttacttaaaattatcaaagataaagatttcttggataacgaagaaaatgaagacattttagaagacattgttttaaccttaactttattcgaagatagagagatgattgaagaacgtttgaagacttatgcacatttgtttgacgataaagtgatgaaacagttgaaaagaagacgttatactggatggggtagattgtctcgtaaattgatcaatggaattagagataaacaaagtggtaaaactatcttggactttttgaaatctgacggatttgctaatagaaatttcatgcaattgatccacgacgatagtttgacatttaaagaagacatccaaaaggcccaagtgagtgggcaaggtgattcattacatgaacatattgcaaatttagccggatctcctgctattaagaaagggatattacaaactgttaaagttgtggatgaattagtgaaagtaatgggaagacataaacctgaaaacattgtcattgagatggcaagagaaaatcaaactacacaaaaaggacagaaaaatagtagagaacgtatgaaaagaatagaagagggtattaaagaattgggtagtcaaatattgaaagaacacccagtggaaaatacccagttgcaaaatgaaaaattatatctttactaccttcaaaatggacgtgatatgtatgttgatcaggaattagatataaatagactttcagattatgatgtagatcatatagttccacaatctttcttgaaagatgattccatagacaataaagtattaactagaagtgataaaaatagaggtaaaagtgataatgtcccaagtgaggaagtcgtcaaaaagatgaaaaattactggcgtcaacttttgaatgctaaattaattactcaaagaaaatttgataatttgactaaagcagaaagaggtgggctttctgaattagataaagccgggttcattaaaagacaattggtcgaaactagacaaattactaaacatgttgcccaaattttagattcccgtatgaacactaagtatgacgaaaatgataagttaatacgtgaggttaaagtcattactttaaaatcaaaacttgtctctgatttcagaaaggatttccaattctataaagttagagaaattaataattatcatcatgctcatgatgcatatttgaatgctgtagttggaactgctttaatcaagaaataccctaaattagaatctgaatttgtatatggtgattacaaagtctatgatgttagaaagatgattgctaaatcagaacaagaaattggtaaagctacagctaaatacttcttttactctaacattatgaatttctttaaaacagaaattactttggcaaacggtgaaattagaaaaagacctcttattgaaacaaatggtgagactggagagatagtttgggacaaagggcgtgatttcgctactgttagaaaagttttatcaatgccacaagttaacattgtaaagaaaacagaggttcaaactggtggtttctcaaaagaaagtattttgcctaaaagaaatagtgataaattgattgccagaaaaaaggattgggatccaaagaaatatggtggtttcgactcaccaaccgtagcctattctgttttggttgtggcaaaggttgaaaagggtaaaagtaaaaagcttaaatcagtaaaagaacttttgggtattacaataatggaaagaagttcctttgaaaagaaccctattgattttttggaagctaaaggttataaggaagtaaagaaggacttaataatcaaattgcctaaatattctttatttgaattagaaaatgggagaaaaagaatgttggcttctgctggagaattgcaaaagggtaatgaattagcattgccttccaaatatgttaacttcttgtatttagcttcacactatgaaaagttgaaagggtcaccagaagataacgagcaaaaacaattatttgttgaacaacacaaacactacttagatgagattatagaacaaattagtgaattcagtaaaagagtgatattagctgatgcaaatttagataaagttttgtcagcctataacaaacatagagataagccaattagagaacaagcagaaaacattattcacttatttacccttaccaatttaggagcacctgctgctttcaagtattttgatacaacaattgatcgtaaaagatatacctcaacaaaagaagtcttagacgccaccttaattcatcaatcaatcactggattgtatgagacaagaattgatttgtctcaattgggtggtgatgaaggggctgatcctaagaagaaaagaaaagttgatccaaagaaaaagcgtaaggtggatcctaagaaaaagagaaaggttgactacaaagaccatgacggtgattataaagatcatgacatcgactacaaggatgacgatgacaagtgataatgactgcagagatccatcgacctgccgccaagctaattccgggcgaatttctgtcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccgggccccccctaactcaagtacaacagatctggaccacctttgattgtaaatagtaataattaccacccttatctaattatttatttaacttatttatttatttattatacatatatacaaatctaataaagtgaaaatctcccccttcacacttcacatatgttaggcgtcatcctgtgctcccgagaaccagtaccagtacatcgctgtttcgttcgagacttgaggtctagttttatacgtgaagaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaattttcatatgagaaccgttatcgataactaaagcagcaacttcttctataaaaatgggttagtatgacagtcatttaaataaggaatttttcagttggcttggtttcaattcaatgttcgttttttttttttcttgctgtgtttgtgtttgtgttgtttatagttgtgtgcactgatcgtcgaaaaaaaaaattcatagtgagccgggaaatctgtatagcccagataacaacacaagtccaaactagaaactcgtcaaacaccaaaagcaatgttgaatcaattgccttgcacaagtacacgtaggaaaacataaaacattgcaattttgaatattgagccttttgtcgtaacattgattgataggattactcaccgaatggttttgaaaccactgccgacagatcaatcaatcaatcaaaaaacgtgaactttgaaaaaggggaagaacagatacattgaagttagccatttccactgatcgtcacaacatatctgataaattactttcaaaattataagctgatgtgtgtgtattattaatgtgacagtaacatcccaaacgagaaatattatctcgacaacaaaaaagtttgatctgaattgaaaatgaagttttcccaccctacccatttgtcatattgaaaccaatcaactgattaatcaatcaattagaattgaagctaaactaaaacataccaccgtccattttgaatgattatatttttttaatattaatatcgagataatgtttctaagaaagaaagaaaaccaggagtgaaaattagaaaaggaaaggaaaggaaaaaaagaaaaatctgaaaatatataaaaaaaaattgtttcgttggcaataaatcttggtgagaacagcgaccgaaagcaaataagaacaaaatatgagtgtattacgttgaacaactaattaacgtgtgtgtatggatctttttttcttttttctctttaaccgactataaacaacaaacatttttgggcagtgcacacactacttaatatacacagcataaattacacgattagaaacaaattagcttattaaaataacctaatcaaaccgaatattttatggtattatgagtaaactatataatataaatagcacacacccacaacaacaacaaaggaaaactaaaaggttttttctttttgaaaagatcgttttctttattattctctagttttgacggcggccgcaagtgattagacttagtccgttcaaatcaagcacaactctgttcattgtttcaacaagaattaattcaaaaacaggttcggtgcataatttgcaaaaaaatattgcagcttctgtggctcgaacacagtacctccagatttcaggtttgaaatacttcagtctgacgctctcccagatgagctaaagctgcaataagaaaacccacgccgggattcgaacccggaatcctttgattagaagtcaaaagcgataaccatttcgccacgcaggcctacttgatgggtttgtaaatggtctactttttcagacctaacagaaattttaatgaaagtcatattcttatacaataaaactgtgtcataaaagcagatattcgactttcgtagattatataggacccaagaactaaaatttaatgccatattatgcatttttaatctgtaaaagtgttgtttccaacctatcacaagtacgttcttgtaacttgtgtttgtagggttgcaaatgaatcataacaacatctcaacagaacatgtatagcaaagcttagtataaaatcagtgttttgagaggcaatccaagaatgtttacatcaaagtttcaataaatatcgaccgaaactgaaaatctttttaggttattgttcacttttttgtaaatatttaaacattttttggacctaaaaaaatacaaacaccaattacgtaccaagaagcatctaatcaactcccagatcaccactatacatttaaaagtcattggtcaataactatactcgagtattgcctcatcaaagaaacaatcaaatattatagatactcactccatcacgtgataatttcactggtatggaaaagtggaaaattttataaaaaaaaatttgatgcctttggcatagctgaaacttcggcccaataggattggagaatatgttttcgcagcgttcttacaattaaattgtggtggaagttcgagacttgcgtaaactatttttaatttgacaagacatgaattcacatcgttttagagctagaaatagcaagttaaaataaggctagtccgttatcaacttgaaaaagtggcaccgagtcggtgctttttttctcgagtttttttatcgagtgtttaaggataatgataactgaagagaagaattagttttgccgccaccgcgggtttgcctctgattaaataaaaaaaagctggtgctttttttttcttttataggaacatcttgaatatatgaactaattaaatgataattttttacccatctttactcttaatcactgagctgcagtcaaagaaaaagggatacagcacctggtgaagagatgaacggagactaacttagacgcgttgattctttttaattgcacattttattaatcgatgctaacgtctatttacatatattctttagagatattatctagggcttcaaataatctctggacagcaataaaagtctcttcaaaagtattgtataacggcaatggggctaatctgattacatctggtcttctttcgtcacagattatagcatgatcatgcaagtacgcattaactcgttccatgacgttcttgtccttttcatcgaaatgcggttgaaacataatggacaattgacatcctctttcagctggattcaaaggagttaaaattttaaacccaaatttggagtttgatgtactggattgtggtatgtaatacttggaattcgtcaatagatcctgtaaaaattgagtcaaagcaacacttttttcacgaagtttagatactccacccactttagcatacacttccaatgacgacttcacagcaacaacatcaagaacagaaggatttgactgtctgtaagaaagagccgagtttattggatcaaactcttctaacattttgaatcgttcttgggagttattgccccaccaaccagctagtctaggaacgaaactgcttttcttgttctctatggtgtatttttcatgcacaaaaatcccacctatggctccaggtcccgagtttaaatatttgtaggaacaccaagcagcaaaatctactccccaatcatgtaaatttaatgggacattcccaactgcatgggcaagatcccacccaactttaatttgttggctcttttccttagcgtatttagttatttcctctatcttgaaaaattgaccagtgtagtattggataccaggaaaacacactagagccaattcatccaggttctcatctatagccttgattattctttctgttttaatataagtttcaccaggttgaacttccaattgaatcaaatgtttctcgtcgtatccgaacaatttaacaatgttcaaaaatgcatagtagtcagaaggaaatgcttgtttttcaaataaaattttggttcttttcccctcaggtttgtaaaaatggatcaacaatgcattcaagtttgctgttaaagaacccataactgcaacttcgttttcctttgcaccaacaatgggggctattaatggtaataagggtaaatcgatgtctacccacggtgttaacagtttgtcaggatgattgaaatgagactcaacccctcgttcaacccatgcatttaattcatcattgatagctttctttgtattcttaggcatcaacccaagagagtttccacataaataaatagactcagttgatgactcatatttattatttttgatacctaatgatccaaaagttggtatggcaaactcatttttaaaagttgggaactttttgtccaatttctttgcctcggctaatgacatctgataataaaatggggttggagtagttggtggtataaccggagagatagaattgaagaaaaaaatcggaaacaacaaaaaaagttgataccctgtattatgtgggagataattgcgaatggtggaaaaaaaaaagacgccattgagtctcaacaacaattctgtcagctgaagagctttacaatcgagaaactatgattcattccgttttaatatgtatgtgtttagtaaactcatgaattttatttgtggtctactttagtactaacataatcattggatagtcaataatgatggtcttccgagactaatgaaattctataccaaagtcgatattccaacacagaaattgctcttgcaacaagtgcacctgttgatatctagagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 22 - DNA, 14064 bp, Artificial Sequence
  Plasmid
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacctgccactactaccactgggagtttcgttcttctcgatactattagctttacttcctgcactagcagtggttggatcaacagaatcttcataatcatcaaaatcgtcttttgaagaccccccgtttgatgtatggccctgtcttttcatcaaactttttatatagttgactgaactgaggctaaatatgtgatcatcttcactatagacaatctttctcttatttgcaccaccgccaccactagtctttgagaaattctcaaaaccttttacgatattaccaagcgggctctcttcgaaataatctatctctttttgatatatcgaatcctctagcgtggttagctttctagttagttcttgcttcttaagaatttgctggattagtttatttttcaattcaacgtatttctcagagtcatctttagattttgatgaagatgtgcgttcattcgctatatccttcttggtcgtgtcttttcgatcctccttggctggcactgaactcgtcttttttggcgttgctgttccagacagacttatctcattagatttggaacttgtgggtttaacatcatttgtatctttagtagacatgattgtgcaataccgtgattatttgttttgaaaggtctgtcatatttctatcaatttcaaaacaaaatgttcatcagaaaaaagccaaaaatgtctcttctagtttcttagtggtgtcgcataatacacaatgtcgctcaacaatccacattcccggcgcatagctcaaatcacatgactacagctaacaattacacaaaaaaaattctctttttgatgtagcaactatcttcaactaaaacattttctccttcggcccatgattgtcctccgggtcgacagcaagccgttacaattgagatggaaagcgacctaccttcactcgataaggtgcttaattgtacttcatataaatctggcccggatctaaacaaatgagttccattaagccgtgggttctcaattagggtttttgtttttgatttagaaaaaagagatcaagatttgtttacaggtgatgcctttttttagaacttatgcgttgcaaaagttgactaacgatttctataaggtgatccacactaattatacaaacgtacaaacagacatacttttcctgcgttcacctgatgttggccagatttctctcttcattgcatagaacataaccacactagggcaacagaaaaaaaaaaaaaaagtgcatcgggaagttgtgttccattcattatatgtctactactgcatatgagtagcccacccaccaccaccatagtaagtttttgtgtatgcgcgccgtcaggttatttcatttctgaatttttcaaccaccttactccctttattgttgattgacaattttgctcacagtaagatcttttagactccaattaatataaaataagtctgattttccaattcctgttttttctttttttttctgtttctatttctttccttttctcccttttttttaattcttcattcaatcatcaattgataattcaggaatattacaacaacccgggatggataaaaagtatagtattggtttagatattggtactaactctgtgggttgggcagttatcaccgacgaatataaagttccatcaaagaaatttaaggtgttaggtaacactgacagacactcaataaaaaagaatcttatcggtgctcttttgttcgactccggtgaaactgccgaggctacacgtttaaaaagaacagcaagaagaagatatacccgtagaaaaaatagaatatgttatttacaagaaatcttttctaatgaaatggctaaagttgatgattcctttttccatagattggaagagtcatttttggttgaagaagacaaaaagcatgagagacatccaatctttgggaatatagttgatgaagtggcttaccatgaaaaatatcctaccatttatcatttaagaaagaaattggtagattcaactgataaagctgaccttagattaatctatttagcacttgcccatatgattaaatttagaggtcattttttgattgaaggtgatttgaacccagataattctgacgtggataaattatttattcaattagtccaaacctacaaccaattatttgaggaaaatccaattaatgctagtggtgtcgatgccaaagctatattatcagccagattatcaaaatctagacgtttggaaaatttgattgcccaattgccaggagaaaaaaagaatggattatttggaaacttgatcgcattatcattgggtttgacaccaaattttaaatctaattttgatttagctgaagatgctaaattacaattatcaaaagacacctatgacgacgatttggacaatttacttgctcaaattggtgatcaatatgcagatttgttcttagctgctaaaaacttatctgatgctattttgttgtctgatattttgagagtgaacacagaaataaccaaagctccattatcagcatctatgatcaaacgttatgatgaacaccatcaggatttgactttattgaaagctttggtgagacaacaattgccagagaagtataaagaaatctttttcgatcaatctaaaaacgggtatgcaggttatattgatgggggtgcctcccaagaggaattttacaaatttataaaacctattttagaaaagatggatgggactgaggaacttttggtcaaattgaacagagaagatttgttacgtaaacagagaacttttgataatggtagtatacctcaccaaattcatttgggtgagttgcatgcaattttaagaagacaagaagatttttatccatttttaaaagataatagagaaaaaatcgagaaaattttaacctttagaattccatactatgttgggcctttggctagaggtaattcaagatttgcctggatgacacgtaaatcagaagaaactattaccccttggaattttgaagaggttgttgataaaggagcatcagcacagagttttattgaaagaatgaccaatttcgataaaaacttaccaaatgaaaaagttttaccaaaacattccttgttatacgaatattttactgtttacaatgaacttacaaaggttaaatatgttactgaaggtatgcgtaagccagcctttttatctggagaacagaaaaaggcaatagttgatttattgtttaaaacaaatagaaaagttactgttaaacaattaaaagaagattactttaagaaaattgaatgttttgattcagttgaaatcagtggtgttgaagacagatttaatgctagtttaggaacttaccatgatttacttaaaattatcaaagataaagatttcttggataacgaagaaaatgaagacattttagaagacattgttttaaccttaactttattcgaagatagagagatgattgaagaacgtttgaagacttatgcacatttgtttgacgataaagtgatgaaacagttgaaaagaagacgttatactggatggggtagattgtctcgtaaattgatcaatggaattagagataaacaaagtggtaaaactatcttggactttttgaaatctgacggatttgctaatagaaatttcatgcaattgatccacgacgatagtttgacatttaaagaagacatccaaaaggcccaagtgagtgggcaaggtgattcattacatgaacatattgcaaatttagccggatctcctgctattaagaaagggatattacaaactgttaaagttgtggatgaattagtgaaagtaatgggaagacataaacctgaaaacattgtcattgagatggcaagagaaaatcaaactacacaaaaaggacagaaaaatagtagagaacgtatgaaaagaatagaagagggtattaaagaattgggtagtcaaatattgaaagaacacccagtggaaaatacccagttgcaaaatgaaaaattatatctttactaccttcaaaatggacgtgatatgtatgttgatcaggaattagatataaatagactttcagattatgatgtagatcatatagttccacaatctttcttgaaagatgattccatagacaataaagtattaactagaagtgataaaaatagaggtaaaagtgataatgtcccaagtgaggaagtcgtcaaaaagatgaaaaattactggcgtcaacttttgaatgctaaattaattactcaaagaaaatttgataatttgactaaagcagaaagaggtgggctttctgaattagataaagccgggttcattaaaagacaattggtcgaaactagacaaattactaaacatgttgcccaaattttagattcccgtatgaacactaagtatgacgaaaatgataagttaatacgtgaggttaaagtcattactttaaaatcaaaacttgtctctgatttcagaaaggatttccaattctataaagttagagaaattaataattatcatcatgctcatgatgcatatttgaatgctgtagttggaactgctttaatcaagaaataccctaaattagaatctgaatttgtatatggtgattacaaagtctatgatgttagaaagatgattgctaaatcagaacaagaaattggtaaagctacagctaaatacttcttttactctaacattatgaatttctttaaaacagaaattactttggcaaacggtgaaattagaaaaagacctcttattgaaacaaatggtgagactggagagatagtttgggacaaagggcgtgatttcgctactgttagaaaagttttatcaatgccacaagttaacattgtaaagaaaacagaggttcaaactggtggtttctcaaaagaaagtattttgcctaaaagaaatagtgataaattgattgccagaaaaaaggattgggatccaaagaaatatggtggtttcgactcaccaaccgtagcctattctgttttggttgtggcaaaggttgaaaagggtaaaagtaaaaagcttaaatcagtaaaagaacttttgggtattacaataatggaaagaagttcctttgaaaagaaccctattgattttttggaagctaaaggttataaggaagtaaagaaggacttaataatcaaattgcctaaatattctttatttgaattagaaaatgggagaaaaagaatgttggcttctgctggagaattgcaaaagggtaatgaattagcattgccttccaaatatgttaacttcttgtatttagcttcacactatgaaaagttgaaagggtcaccagaagataacgagcaaaaacaattatttgttgaacaacacaaacactacttagatgagattatagaacaaattagtgaattcagtaaaagagtgatattagctgatgcaaatttagataaagttttgtcagcctataacaaacatagagataagccaattagagaacaagcagaaaacattattcacttatttacccttaccaatttaggagcacctgctgctttcaagtattttgatacaacaattgatcgtaaaagatatacctcaacaaaagaagtcttagacgccaccttaattcatcaatcaatcactggattgtatgagacaagaattgatttgtctcaattgggtggtgatgaaggggctgatcctaagaagaaaagaaaagttgatccaaagaaaaagcgtaaggtggatcctaagaaaaagagaaaggttgactacaaagaccatgacggtgattataaagatcatgacatcgactacaaggatgacgatgacaagtgataatgactgcagagatccatcgacctgccgccaagctaattccgggcgaatttctgtcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccgggccccccctaactcaagtacaacagatctggaccacctttgattgtaaatagtaataattaccacccttatctaattatttatttaacttatttatttatttattatacatatatacaaatctaataaagtgaaaatctcccccttcacacttcacatatgttaggcgtcatcctgtgctcccgagaaccagtaccagtacatcgctgtttcgttcgagacttgaggtctagttttatacgtgaagaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaattttcatatgagaaccgttatcgataactaaagcagcaacttcttctataaaaatgggttagtatgacagtcatttaaataaggaatttttcagttggcttggtttcaattcaatgttcgttttttttttttcttgctgtgtttgtgtttgtgttgtttatagttgtgtgcactgatcgtcgaaaaaaaaaattcatagtgagccgggaaatctgtatagcccagataacaacacaagtccaaactagaaactcgtcaaacaccaaaagcaatgttgaatcaattgccttgcacaagtacacgtaggaaaacataaaacattgcaattttgaatattgagccttttgtcgtaacattgattgataggattactcaccgaatggttttgaaaccactgccgacagatcaatcaatcaatcaaaaaacgtgaactttgaaaaaggggaagaacagatacattgaagttagccatttccactgatcgtcacaacatatctgataaattactttcaaaattataagctgatgtgtgtgtattattaatgtgacagtaacatcccaaacgagaaatattatctcgacaacaaaaaagtttgatctgaattgaaaatgaagttttcccaccctacccatttgtcatattgaaaccaatcaactgattaatcaatcaattagaattgaagctaaactaaaacataccaccgtccattttgaatgattatatttttttaatattaatatcgagataatgtttctaagaaagaaagaaaaccaggagtgaaaattagaaaaggaaaggaaaggaaaaaaagaaaaatctgaaaatatataaaaaaaaattgtttcgttggcaataaatcttggtgagaacagcgaccgaaagcaaataagaacaaaatatgagtgtattacgttgaacaactaattaacgtgtgtgtatggatctttttttcttttttctctttaaccgactataaacaacaaacatttttgggcagtgcacacactacttaatatacacagcataaattacacgattagaaacaaattagcttattaaaataacctaatcaaaccgaatattttatggtattatgagtaaactatataatataaatagcacacacccacaacaacaacaaaggaaaactaaaaggttttttctttttgaaaagatcgttttctttattattctctagttttgacggcggccgcaagtgattagacttagtccgttcaaatcaagcacaactctgttcattgtttcaacaagaattaattcaaaaacaggttcggtgcataatttgcaaaaaaatattgcagcttctgtggctcgaacacagtacctccagatttcaggtttgaaatacttcagtctgacgctctcccagatgagctaaagctgcaataagaaaacccacgccgggattcgaacccggaatcctttgattagaagtcaaaagcgataaccatttcgccacgcaggcctacttgatgggtttgtaaatggtctactttttcagacctaacagaaattttaatgaaagtcatattcttatacaataaaactgtgtcataaaagcagatattcgactttcgtagattatataggacccaagaactaaaatttaatgccatattatgcatttttaatctgtaaaagtgttgtttccaacctatcacaagtacgttcttgtaacttgtgtttgtagggttgcaaatgaatcataacaacatctcaacagaacatgtatagcaaagcttagtataaaatcagtgttttgagaggcaatccaagaatgtttacatcaaagtttcaataaatatcgaccgaaactgaaaatctttttaggttattgttcacttttttgtaaatatttaaacattttttggacctaaaaaaatacaaacaccaattacgtaccaagaagcatctaatcaactcccagatcaccactatacatttaaaagtcattggtcaataactatactcgagtattgcctcatcaaagaaacaatcaaatattatagatactcactccatcacgtgataatttcactggtatggaaaagtggaaaattttataaaaaaaaatttgatgcctttggcatagctgaaacttcggcccaataggattggagaatatgttttcgcagcgttcttacaattaaattgtggtggaagttcgagacttgcgtaaactatttttaatttggggtgaactatttgttcgccgttttagagctagaaatagcaagttaaaataaggctagtccgttatcaacttgaaaaagtggcaccgagtcggtgctttttttctcgagtttttttatcgagtgtttaaggataatgataactgaagagaagaattagttttgccgccaccgcgggtttgcctctgattaaataaaaaaaagctggtgctttttttttcttttataggaacatcttgaatatatgaactaattaaatgataattttttacccatctttactcttaatcactgagctgcagtcaaagaaaaagggatacagcacctggtgaagagatgaacggagactaacttagacgcgttgattctttttaattgcacattttattaatcgatgctaacgtctatttacatatattctttagagatattatctagggcttcaaataatctctggacagcaataaaagtctcttcaaaagtattgtataacggcaatggggctaatctgattacatctggtcttctttcgtcacagattatagcatgatcatgcaagtacgcattaactcgttccatgacgttcttgtccttttcatcgaaatgcggttgaaacataatggacaattgacatcctctttcagctggattcaaaggagttaaaattttaaacccaaatttggagtttgatgtactggattgtggtatgtaatacttggaattcgtcaatagatcctgtaaaaattgagtcaaagcaacacttttttcacgaagtttagatactccacccactttagcatacacttccaatgacgacttcacagcaacaacatcaagaacagaaggatttgactgtctgtaagaaagagccgagtttattggatcaaactcttctaacattttgaatcgttcttgggagttattgccccaccaaccagctagtctaggaacgaaactgcttttcttgttctctatggtgtatttttcatgcacaaaaatcccacctatggctccaggtcccgagtttaaatatttgtaggaacaccaagcagcaaaatctactccccaatcatgtaaatttaatgggacattcccaactgcatgggcaagatcccacccaactttaatttgttggctcttttccttagcgtatttagttatttcctctatcttgaaaaattgaccagtgtagtattggataccaggaaaacacactagagccaattcatccaggttctcatctatagccttgattattctttctgttttaatataagtttcaccaggttgaacttccaattgaatcaaatgtttctcgtcgtatccgaacaatttaacaatgttcaaaaatgcatagtagtcagaaggaaatgcttgtttttcaaataaaattttggttcttttcccctcaggtttgtaaaaatggatcaacaatgcattcaagtttgctgttaaagaacccataactgcaacttcgttttcctttgcaccaacaatgggggctattaatggtaataagggtaaatcgatgtctacccacggtgttaacagtttgtcaggatgattgaaatgagactcaacccctcgttcaacccatgcatttaattcatcattgatagctttctttgtattcttaggcatcaacccaagagagtttccacataaataaatagactcagttgatgactcatatttattatttttgatacctaatgatccaaaagttggtatggcaaactcatttttaaaagttgggaactttttgtccaatttctttgcctcggctaatgacatctgataataaaatggggttggagtagttggtggtataaccggagagatagaattgaagaaaaaaatcggaaacaacaaaaaaagttgataccctgtattatgtgggagataattgcgaatggtggaaaaaaaaaagacgccattgagtctcaacaacaattctgtcagctgaagagctttacaatcgagaaactatgattcattccgttttaatatgtatgtgtttagtaaactcatgaattttatttgtggtctactttagtactaacataatcattggatagtcaataatgatggtcttccgagactaatgaaattctataccaaagtcgatattccaacacagaaattgctcttgcaacaagtgcacctgttgatatctagagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 23 - DNA, 14064 bp, Artificial Sequence
  Plasmid
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacctgccactactaccactgggagtttcgttcttctcgatactattagctttacttcctgcactagcagtggttggatcaacagaatcttcataatcatcaaaatcgtcttttgaagaccccccgtttgatgtatggccctgtcttttcatcaaactttttatatagttgactgaactgaggctaaatatgtgatcatcttcactatagacaatctttctcttatttgcaccaccgccaccactagtctttgagaaattctcaaaaccttttacgatattaccaagcgggctctcttcgaaataatctatctctttttgatatatcgaatcctctagcgtggttagctttctagttagttcttgcttcttaagaatttgctggattagtttatttttcaattcaacgtatttctcagagtcatctttagattttgatgaagatgtgcgttcattcgctatatccttcttggtcgtgtcttttcgatcctccttggctggcactgaactcgtcttttttggcgttgctgttccagacagacttatctcattagatttggaacttgtgggtttaacatcatttgtatctttagtagacatgattgtgcaataccgtgattatttgttttgaaaggtctgtcatatttctatcaatttcaaaacaaaatgttcatcagaaaaaagccaaaaatgtctcttctagtttcttagtggtgtcgcataatacacaatgtcgctcaacaatccacattcccggcgcatagctcaaatcacatgactacagctaacaattacacaaaaaaaattctctttttgatgtagcaactatcttcaactaaaacattttctccttcggcccatgattgtcctccgggtcgacagcaagccgttacaattgagatggaaagcgacctaccttcactcgataaggtgcttaattgtacttcatataaatctggcccggatctaaacaaatgagttccattaagccgtgggttctcaattagggtttttgtttttgatttagaaaaaagagatcaagatttgtttacaggtgatgcctttttttagaacttatgcgttgcaaaagttgactaacgatttctataaggtgatccacactaattatacaaacgtacaaacagacatacttttcctgcgttcacctgatgttggccagatttctctcttcattgcatagaacataaccacactagggcaacagaaaaaaaaaaaaaaagtgcatcgggaagttgtgttccattcattatatgtctactactgcatatgagtagcccacccaccaccaccatagtaagtttttgtgtatgcgcgccgtcaggttatttcatttctgaatttttcaaccaccttactccctttattgttgattgacaattttgctcacagtaagatcttttagactccaattaatataaaataagtctgattttccaattcctgttttttctttttttttctgtttctatttctttccttttctcccttttttttaattcttcattcaatcatcaattgataattcaggaatattacaacaacccgggatggataaaaagtatagtattggtttagatattggtactaactctgtgggttgggcagttatcaccgacgaatataaagttccatcaaagaaatttaaggtgttaggtaacactgacagacactcaataaaaaagaatcttatcggtgctcttttgttcgactccggtgaaactgccgaggctacacgtttaaaaagaacagcaagaagaagatatacccgtagaaaaaatagaatatgttatttacaagaaatcttttctaatgaaatggctaaagttgatgattcctttttccatagattggaagagtcatttttggttgaagaagacaaaaagcatgagagacatccaatctttgggaatatagttgatgaagtggcttaccatgaaaaatatcctaccatttatcatttaagaaagaaattggtagattcaactgataaagctgaccttagattaatctatttagcacttgcccatatgattaaatttagaggtcattttttgattgaaggtgatttgaacccagataattctgacgtggataaattatttattcaattagtccaaacctacaaccaattatttgaggaaaatccaattaatgctagtggtgtcgatgccaaagctatattatcagccagattatcaaaatctagacgtttggaaaatttgattgcccaattgccaggagaaaaaaagaatggattatttggaaacttgatcgcattatcattgggtttgacaccaaattttaaatctaattttgatttagctgaagatgctaaattacaattatcaaaagacacctatgacgacgatttggacaatttacttgctcaaattggtgatcaatatgcagatttgttcttagctgctaaaaacttatctgatgctattttgttgtctgatattttgagagtgaacacagaaataaccaaagctccattatcagcatctatgatcaaacgttatgatgaacaccatcaggatttgactttattgaaagctttggtgagacaacaattgccagagaagtataaagaaatctttttcgatcaatctaaaaacgggtatgcaggttatattgatgggggtgcctcccaagaggaattttacaaatttataaaacctattttagaaaagatggatgggactgaggaacttttggtcaaattgaacagagaagatttgttacgtaaacagagaacttttgataatggtagtatacctcaccaaattcatttgggtgagttgcatgcaattttaagaagacaagaagatttttatccatttttaaaagataatagagaaaaaatcgagaaaattttaacctttagaattccatactatgttgggcctttggctagaggtaattcaagatttgcctggatgacacgtaaatcagaagaaactattaccccttggaattttgaagaggttgttgataaaggagcatcagcacagagttttattgaaagaatgaccaatttcgataaaaacttaccaaatgaaaaagttttaccaaaacattccttgttatacgaatattttactgtttacaatgaacttacaaaggttaaatatgttactgaaggtatgcgtaagccagcctttttatctggagaacagaaaaaggcaatagttgatttattgtttaaaacaaatagaaaagttactgttaaacaattaaaagaagattactttaagaaaattgaatgttttgattcagttgaaatcagtggtgttgaagacagatttaatgctagtttaggaacttaccatgatttacttaaaattatcaaagataaagatttcttggataacgaagaaaatgaagacattttagaagacattgttttaaccttaactttattcgaagatagagagatgattgaagaacgtttgaagacttatgcacatttgtttgacgataaagtgatgaaacagttgaaaagaagacgttatactggatggggtagattgtctcgtaaattgatcaatggaattagagataaacaaagtggtaaaactatcttggactttttgaaatctgacggatttgctaatagaaatttcatgcaattgatccacgacgatagtttgacatttaaagaagacatccaaaaggcccaagtgagtgggcaaggtgattcattacatgaacatattgcaaatttagccggatctcctgctattaagaaagggatattacaaactgttaaagttgtggatgaattagtgaaagtaatgggaagacataaacctgaaaacattgtcattgagatggcaagagaaaatcaaactacacaaaaaggacagaaaaatagtagagaacgtatgaaaagaatagaagagggtattaaagaattgggtagtcaaatattgaaagaacacccagtggaaaatacccagttgcaaaatgaaaaattatatctttactaccttcaaaatggacgtgatatgtatgttgatcaggaattagatataaatagactttcagattatgatgtagatcatatagttccacaatctttcttgaaagatgattccatagacaataaagtattaactagaagtgataaaaatagaggtaaaagtgataatgtcccaagtgaggaagtcgtcaaaaagatgaaaaattactggcgtcaacttttgaatgctaaattaattactcaaagaaaatttgataatttgactaaagcagaaagaggtgggctttctgaattagataaagccgggttcattaaaagacaattggtcgaaactagacaaattactaaacatgttgcccaaattttagattcccgtatgaacactaagtatgacgaaaatgataagttaatacgtgaggttaaagtcattactttaaaatcaaaacttgtctctgatttcagaaaggatttccaattctataaagttagagaaattaataattatcatcatgctcatgatgcatatttgaatgctgtagttggaactgctttaatcaagaaataccctaaattagaatctgaatttgtatatggtgattacaaagtctatgatgttagaaagatgattgctaaatcagaacaagaaattggtaaagctacagctaaatacttcttttactctaacattatgaatttctttaaaacagaaattactttggcaaacggtgaaattagaaaaagacctcttattgaaacaaatggtgagactggagagatagtttgggacaaagggcgtgatttcgctactgttagaaaagttttatcaatgccacaagttaacattgtaaagaaaacagaggttcaaactggtggtttctcaaaagaaagtattttgcctaaaagaaatagtgataaattgattgccagaaaaaaggattgggatccaaagaaatatggtggtttcgactcaccaaccgtagcctattctgttttggttgtggcaaaggttgaaaagggtaaaagtaaaaagcttaaatcagtaaaagaacttttgggtattacaataatggaaagaagttcctttgaaaagaaccctattgattttttggaagctaaaggttataaggaagtaaagaaggacttaataatcaaattgcctaaatattctttatttgaattagaaaatgggagaaaaagaatgttggcttctgctggagaattgcaaaagggtaatgaattagcattgccttccaaatatgttaacttcttgtatttagcttcacactatgaaaagttgaaagggtcaccagaagataacgagcaaaaacaattatttgttgaacaacacaaacactacttagatgagattatagaacaaattagtgaattcagtaaaagagtgatattagctgatgcaaatttagataaagttttgtcagcctataacaaacatagagataagccaattagagaacaagcagaaaacattattcacttatttacccttaccaatttaggagcacctgctgctttcaagtattttgatacaacaattgatcgtaaaagatatacctcaacaaaagaagtcttagacgccaccttaattcatcaatcaatcactggattgtatgagacaagaattgatttgtctcaattgggtggtgatgaaggggctgatcctaagaagaaaagaaaagttgatccaaagaaaaagcgtaaggtggatcctaagaaaaagagaaaggttgactacaaagaccatgacggtgattataaagatcatgacatcgactacaaggatgacgatgacaagtgataatgactgcagagatccatcgacctgccgccaagctaattccgggcgaatttctgtcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccgggccccccctaactcaagtacaacagatctggaccacctttgattgtaaatagtaataattaccacccttatctaattatttatttaacttatttatttatttattatacatatatacaaatctaataaagtgaaaatctcccccttcacacttcacatatgttaggcgtcatcctgtgctcccgagaaccagtaccagtacatcgctgtttcgttcgagacttgaggtctagttttatacgtgaagaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaattttcatatgagaaccgttatcgataactaaagcagcaacttcttctataaaaatgggttagtatgacagtcatttaaataaggaatttttcagttggcttggtttcaattcaatgttcgttttttttttttcttgctgtgtttgtgtttgtgttgtttatagttgtgtgcactgatcgtcgaaaaaaaaaattcatagtgagccgggaaatctgtatagcccagataacaacacaagtccaaactagaaactcgtcaaacaccaaaagcaatgttgaatcaattgccttgcacaagtacacgtaggaaaacataaaacattgcaattttgaatattgagccttttgtcgtaacattgattgataggattactcaccgaatggttttgaaaccactgccgacagatcaatcaatcaatcaaaaaacgtgaactttgaaaaaggggaagaacagatacattgaagttagccatttccactgatcgtcacaacatatctgataaattactttcaaaattataagctgatgtgtgtgtattattaatgtgacagtaacatcccaaacgagaaatattatctcgacaacaaaaaagtttgatctgaattgaaaatgaagttttcccaccctacccatttgtcatattgaaaccaatcaactgattaatcaatcaattagaattgaagctaaactaaaacataccaccgtccattttgaatgattatatttttttaatattaatatcgagataatgtttctaagaaagaaagaaaaccaggagtgaaaattagaaaaggaaaggaaaggaaaaaaagaaaaatctgaaaatatataaaaaaaaattgtttcgttggcaataaatcttggtgagaacagcgaccgaaagcaaataagaacaaaatatgagtgtattacgttgaacaactaattaacgtgtgtgtatggatctttttttcttttttctctttaaccgactataaacaacaaacatttttgggcagtgcacacactacttaatatacacagcataaattacacgattagaaacaaattagcttattaaaataacctaatcaaaccgaatattttatggtattatgagtaaactatataatataaatagcacacacccacaacaacaacaaaggaaaactaaaaggttttttctttttgaaaagatcgttttctttattattctctagttttgacggcggccgcaagtgattagacttagtccgttcaaatcaagcacaactctgttcattgtttcaacaagaattaattcaaaaacaggttcggtgcataatttgcaaaaaaatattgcagcttctgtggctcgaacacagtacctccagatttcaggtttgaaatacttcagtctgacgctctcccagatgagctaaagctgcaataagaaaacccacgccgggattcgaacccggaatcctttgattagaagtcaaaagcgataaccatttcgccacgcaggcctacttgatgggtttgtaaatggtctactttttcagacctaacagaaattttaatgaaagtcatattcttatacaataaaactgtgtcataaaagcagatattcgactttcgtagattatataggacccaagaactaaaatttaatgccatattatgcatttttaatctgtaaaagtgttgtttccaacctatcacaagtacgttcttgtaacttgtgtttgtagggttgcaaatgaatcataacaacatctcaacagaacatgtatagcaaagcttagtataaaatcagtgttttgagaggcaatccaagaatgtttacatcaaagtttcaataaatatcgaccgaaactgaaaatctttttaggttattgttcacttttttgtaaatatttaaacattttttggacctaaaaaaatacaaacaccaattacgtaccaagaagcatctaatcaactcccagatcaccactatacatttaaaagtcattggtcaataactatactcgagtattgcctcatcaaagaaacaatcaaatattatagatactcactccatcacgtgataatttcactggtatggaaaagtggaaaattttataaaaaaaaatttgatgcctttggcatagctgaaacttcggcccaataggattggagaatatgttttcgcagcgttcttacaattaaattgtggtggaagttcgagacttgcgtaaactatttttaatttgatagcagaaactgccaacaagttttagagctagaaatagcaagttaaaataaggctagtccgttatcaacttgaaaaagtggcaccgagtcggtgctttttttctcgagtttttttatcgagtgtttaaggataatgataactgaagagaagaattagttttgccgccaccgcgggtttgcctctgattaaataaaaaaaagctggtgctttttttttcttttataggaacatcttgaatatatgaactaattaaatgataattttttacccatctttactcttaatcactgagctgcagtcaaagaaaaagggatacagcacctggtgaagagatgaacggagactaacttagacgcgttgattctttttaattgcacattttattaatcgatgctaacgtctatttacatatattctttagagatattatctagggcttcaaataatctctggacagcaataaaagtctcttcaaaagtattgtataacggcaatggggctaatctgattacatctggtcttctttcgtcacagattatagcatgatcatgcaagtacgcattaactcgttccatgacgttcttgtccttttcatcgaaatgcggttgaaacataatggacaattgacatcctctttcagctggattcaaaggagttaaaattttaaacccaaatttggagtttgatgtactggattgtggtatgtaatacttggaattcgtcaatagatcctgtaaaaattgagtcaaagcaacacttttttcacgaagtttagatactccacccactttagcatacacttccaatgacgacttcacagcaacaacatcaagaacagaaggatttgactgtctgtaagaaagagccgagtttattggatcaaactcttctaacattttgaatcgttcttgggagttattgccccaccaaccagctagtctaggaacgaaactgcttttcttgttctctatggtgtatttttcatgcacaaaaatcccacctatggctccaggtcccgagtttaaatatttgtaggaacaccaagcagcaaaatctactccccaatcatgtaaatttaatgggacattcccaactgcatgggcaagatcccacccaactttaatttgttggctcttttccttagcgtatttagttatttcctctatcttgaaaaattgaccagtgtagtattggataccaggaaaacacactagagccaattcatccaggttctcatctatagccttgattattctttctgttttaatataagtttcaccaggttgaacttccaattgaatcaaatgtttctcgtcgtatccgaacaatttaacaatgttcaaaaatgcatagtagtcagaaggaaatgcttgtttttcaaataaaattttggttcttttcccctcaggtttgtaaaaatggatcaacaatgcattcaagtttgctgttaaagaacccataactgcaacttcgttttcctttgcaccaacaatgggggctattaatggtaataagggtaaatcgatgtctacccacggtgttaacagtttgtcaggatgattgaaatgagactcaacccctcgttcaacccatgcatttaattcatcattgatagctttctttgtattcttaggcatcaacccaagagagtttccacataaataaatagactcagttgatgactcatatttattatttttgatacctaatgatccaaaagttggtatggcaaactcatttttaaaagttgggaactttttgtccaatttctttgcctcggctaatgacatctgataataaaatggggttggagtagttggtggtataaccggagagatagaattgaagaaaaaaatcggaaacaacaaaaaaagttgataccctgtattatgtgggagataattgcgaatggtggaaaaaaaaaagacgccattgagtctcaacaacaattctgtcagctgaagagctttacaatcgagaaactatgattcattccgttttaatatgtatgtgtttagtaaactcatgaattttatttgtggtctactttagtactaacataatcattggatagtcaataatgatggtcttccgagactaatgaaattctataccaaagtcgatattccaacacagaaattgctcttgcaacaagtgcacctgttgatatctagagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 24 - DNA, 14064 bp, Artificial Sequence
  Plasmid
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacctgccactactaccactgggagtttcgttcttctcgatactattagctttacttcctgcactagcagtggttggatcaacagaatcttcataatcatcaaaatcgtcttttgaagaccccccgtttgatgtatggccctgtcttttcatcaaactttttatatagttgactgaactgaggctaaatatgtgatcatcttcactatagacaatctttctcttatttgcaccaccgccaccactagtctttgagaaattctcaaaaccttttacgatattaccaagcgggctctcttcgaaataatctatctctttttgatatatcgaatcctctagcgtggttagctttctagttagttcttgcttcttaagaatttgctggattagtttatttttcaattcaacgtatttctcagagtcatctttagattttgatgaagatgtgcgttcattcgctatatccttcttggtcgtgtcttttcgatcctccttggctggcactgaactcgtcttttttggcgttgctgttccagacagacttatctcattagatttggaacttgtgggtttaacatcatttgtatctttagtagacatgattgtgcaataccgtgattatttgttttgaaaggtctgtcatatttctatcaatttcaaaacaaaatgttcatcagaaaaaagccaaaaatgtctcttctagtttcttagtggtgtcgcataatacacaatgtcgctcaacaatccacattcccggcgcatagctcaaatcacatgactacagctaacaattacacaaaaaaaattctctttttgatgtagcaactatcttcaactaaaacattttctccttcggcccatgattgtcctccgggtcgacagcaagccgttacaattgagatggaaagcgacctaccttcactcgataaggtgcttaattgtacttcatataaatctggcccggatctaaacaaatgagttccattaagccgtgggttctcaattagggtttttgtttttgatttagaaaaaagagatcaagatttgtttacaggtgatgcctttttttagaacttatgcgttgcaaaagttgactaacgatttctataaggtgatccacactaattatacaaacgtacaaacagacatacttttcctgcgttcacctgatgttggccagatttctctcttcattgcatagaacataaccacactagggcaacagaaaaaaaaaaaaaaagtgcatcgggaagttgtgttccattcattatatgtctactactgcatatgagtagcccacccaccaccaccatagtaagtttttgtgtatgcgcgccgtcaggttatttcatttctgaatttttcaaccaccttactccctttattgttgattgacaattttgctcacagtaagatcttttagactccaattaatataaaataagtctgattttccaattcctgttttttctttttttttctgtttctatttctttccttttctcccttttttttaattcttcattcaatcatcaattgataattcaggaatattacaacaacccgggatggataaaaagtatagtattggtttagatattggtactaactctgtgggttgggcagttatcaccgacgaatataaagttccatcaaagaaatttaaggtgttaggtaacactgacagacactcaataaaaaagaatcttatcggtgctcttttgttcgactccggtgaaactgccgaggctacacgtttaaaaagaacagcaagaagaagatatacccgtagaaaaaatagaatatgttatttacaagaaatcttttctaatgaaatggctaaagttgatgattcctttttccatagattggaagagtcatttttggttgaagaagacaaaaagcatgagagacatccaatctttgggaatatagttgatgaagtggcttaccatgaaaaatatcctaccatttatcatttaagaaagaaattggtagattcaactgataaagctgaccttagattaatctatttagcacttgcccatatgattaaatttagaggtcattttttgattgaaggtgatttgaacccagataattctgacgtggataaattatttattcaattagtccaaacctacaaccaattatttgaggaaaatccaattaatgctagtggtgtcgatgccaaagctatattatcagccagattatcaaaatctagacgtttggaaaatttgattgcccaattgccaggagaaaaaaagaatggattatttggaaacttgatcgcattatcattgggtttgacaccaaattttaaatctaattttgatttagctgaagatgctaaattacaattatcaaaagacacctatgacgacgatttggacaatttacttgctcaaattggtgatcaatatgcagatttgttcttagctgctaaaaacttatctgatgctattttgttgtctgatattttgagagtgaacacagaaataaccaaagctccattatcagcatctatgatcaaacgttatgatgaacaccatcaggatttgactttattgaaagctttggtgagacaacaattgccagagaagtataaagaaatctttttcgatcaatctaaaaacgggtatgcaggttatattgatgggggtgcctcccaagaggaattttacaaatttataaaacctattttagaaaagatggatgggactgaggaacttttggtcaaattgaacagagaagatttgttacgtaaacagagaacttttgataatggtagtatacctcaccaaattcatttgggtgagttgcatgcaattttaagaagacaagaagatttttatccatttttaaaagataatagagaaaaaatcgagaaaattttaacctttagaattccatactatgttgggcctttggctagaggtaattcaagatttgcctggatgacacgtaaatcagaagaaactattaccccttggaattttgaagaggttgttgataaaggagcatcagcacagagttttattgaaagaatgaccaatttcgataaaaacttaccaaatgaaaaagttttaccaaaacattccttgttatacgaatattttactgtttacaatgaacttacaaaggttaaatatgttactgaaggtatgcgtaagccagcctttttatctggagaacagaaaaaggcaatagttgatttattgtttaaaacaaatagaaaagttactgttaaacaattaaaagaagattactttaagaaaattgaatgttttgattcagttgaaatcagtggtgttgaagacagatttaatgctagtttaggaacttaccatgatttacttaaaattatcaaagataaagatttcttggataacgaagaaaatgaagacattttagaagacattgttttaaccttaactttattcgaagatagagagatgattgaagaacgtttgaagacttatgcacatttgtttgacgataaagtgatgaaacagttgaaaagaagacgttatactggatggggtagattgtctcgtaaattgatcaatggaattagagataaacaaagtggtaaaactatcttggactttttgaaatctgacggatttgctaatagaaatttcatgcaattgatccacgacgatagtttgacatttaaagaagacatccaaaaggcccaagtgagtgggcaaggtgattcattacatgaacatattgcaaatttagccggatctcctgctattaagaaagggatattacaaactgttaaagttgtggatgaattagtgaaagtaatgggaagacataaacctgaaaacattgtcattgagatggcaagagaaaatcaaactacacaaaaaggacagaaaaatagtagagaacgtatgaaaagaatagaagagggtattaaagaattgggtagtcaaatattgaaagaacacccagtggaaaatacccagttgcaaaatgaaaaattatatctttactaccttcaaaatggacgtgatatgtatgttgatcaggaattagatataaatagactttcagattatgatgtagatcatatagttccacaatctttcttgaaagatgattccatagacaataaagtattaactagaagtgataaaaatagaggtaaaagtgataatgtcccaagtgaggaagtcgtcaaaaagatgaaaaattactggcgtcaacttttgaatgctaaattaattactcaaagaaaatttgataatttgactaaagcagaaagaggtgggctttctgaattagataaagccgggttcattaaaagacaattggtcgaaactagacaaattactaaacatgttgcccaaattttagattcccgtatgaacactaagtatgacgaaaatgataagttaatacgtgaggttaaagtcattactttaaaatcaaaacttgtctctgatttcagaaaggatttccaattctataaagttagagaaattaataattatcatcatgctcatgatgcatatttgaatgctgtagttggaactgctttaatcaagaaataccctaaattagaatctgaatttgtatatggtgattacaaagtctatgatgttagaaagatgattgctaaatcagaacaagaaattggtaaagctacagctaaatacttcttttactctaacattatgaatttctttaaaacagaaattactttggcaaacggtgaaattagaaaaagacctcttattgaaacaaatggtgagactggagagatagtttgggacaaagggcgtgatttcgctactgttagaaaagttttatcaatgccacaagttaacattgtaaagaaaacagaggttcaaactggtggtttctcaaaagaaagtattttgcctaaaagaaatagtgataaattgattgccagaaaaaaggattgggatccaaagaaatatggtggtttcgactcaccaaccgtagcctattctgttttggttgtggcaaaggttgaaaagggtaaaagtaaaaagcttaaatcagtaaaagaacttttgggtattacaataatggaaagaagttcctttgaaaagaaccctattgattttttggaagctaaaggttataaggaagtaaagaaggacttaataatcaaattgcctaaatattctttatttgaattagaaaatgggagaaaaagaatgttggcttctgctggagaattgcaaaagggtaatgaattagcattgccttccaaatatgttaacttcttgtatttagcttcacactatgaaaagttgaaagggtcaccagaagataacgagcaaaaacaattatttgttgaacaacacaaacactacttagatgagattatagaacaaattagtgaattcagtaaaagagtgatattagctgatgcaaatttagataaagttttgtcagcctataacaaacatagagataagccaattagagaacaagcagaaaacattattcacttatttacccttaccaatttaggagcacctgctgctttcaagtattttgatacaacaattgatcgtaaaagatatacctcaacaaaagaagtcttagacgccaccttaattcatcaatcaatcactggattgtatgagacaagaattgatttgtctcaattgggtggtgatgaaggggctgatcctaagaagaaaagaaaagttgatccaaagaaaaagcgtaaggtggatcctaagaaaaagagaaaggttgactacaaagaccatgacggtgattataaagatcatgacatcgactacaaggatgacgatgacaagtgataatgactgcagagatccatcgacctgccgccaagctaattccgggcgaatttctgtcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccgggccccccctaactcaagtacaacagatctggaccacctttgattgtaaatagtaataattaccacccttatctaattatttatttaacttatttatttatttattatacatatatacaaatctaataaagtgaaaatctcccccttcacacttcacatatgttaggcgtcatcctgtgctcccgagaaccagtaccagtacatcgctgtttcgttcgagacttgaggtctagttttatacgtgaagaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaattttcatatgagaaccgttatcgataactaaagcagcaacttcttctataaaaatgggttagtatgacagtcatttaaataaggaatttttcagttggcttggtttcaattcaatgttcgttttttttttttcttgctgtgtttgtgtttgtgttgtttatagttgtgtgcactgatcgtcgaaaaaaaaaattcatagtgagccgggaaatctgtatagcccagataacaacacaagtccaaactagaaactcgtcaaacaccaaaagcaatgttgaatcaattgccttgcacaagtacacgtaggaaaacataaaacattgcaattttgaatattgagccttttgtcgtaacattgattgataggattactcaccgaatggttttgaaaccactgccgacagatcaatcaatcaatcaaaaaacgtgaactttgaaaaaggggaagaacagatacattgaagttagccatttccactgatcgtcacaacatatctgataaattactttcaaaattataagctgatgtgtgtgtattattaatgtgacagtaacatcccaaacgagaaatattatctcgacaacaaaaaagtttgatctgaattgaaaatgaagttttcccaccctacccatttgtcatattgaaaccaatcaactgattaatcaatcaattagaattgaagctaaactaaaacataccaccgtccattttgaatgattatatttttttaatattaatatcgagataatgtttctaagaaagaaagaaaaccaggagtgaaaattagaaaaggaaaggaaaggaaaaaaagaaaaatctgaaaatatataaaaaaaaattgtttcgttggcaataaatcttggtgagaacagcgaccgaaagcaaataagaacaaaatatgagtgtattacgttgaacaactaattaacgtgtgtgtatggatctttttttcttttttctctttaaccgactataaacaacaaacatttttgggcagtgcacacactacttaatatacacagcataaattacacgattagaaacaaattagcttattaaaataacctaatcaaaccgaatattttatggtattatgagtaaactatataatataaatagcacacacccacaacaacaacaaaggaaaactaaaaggttttttctttttgaaaagatcgttttctttattattctctagttttgacggcggccgcaagtgattagacttagtccgttcaaatcaagcacaactctgttcattgtttcaacaagaattaattcaaaaacaggttcggtgcataatttgcaaaaaaatattgcagcttctgtggctcgaacacagtacctccagatttcaggtttgaaatacttcagtctgacgctctcccagatgagctaaagctgcaataagaaaacccacgccgggattcgaacccggaatcctttgattagaagtcaaaagcgataaccatttcgccacgcaggcctacttgatgggtttgtaaatggtctactttttcagacctaacagaaattttaatgaaagtcatattcttatacaataaaactgtgtcataaaagcagatattcgactttcgtagattatataggacccaagaactaaaatttaatgccatattatgcatttttaatctgtaaaagtgttgtttccaacctatcacaagtacgttcttgtaacttgtgtttgtagggttgcaaatgaatcataacaacatctcaacagaacatgtatagcaaagcttagtataaaatcagtgttttgagaggcaatccaagaatgtttacatcaaagtttcaataaatatcgaccgaaactgaaaatctttttaggttattgttcacttttttgtaaatatttaaacattttttggacctaaaaaaatacaaacaccaattacgtaccaagaagcatctaatcaactcccagatcaccactatacatttaaaagtcattggtcaataactatactcgagtattgcctcatcaaagaaacaatcaaatattatagatactcactccatcacgtgataatttcactggtatggaaaagtggaaaattttataaaaaaaaatttgatgcctttggcatagctgaaacttcggcccaataggattggagaatatgttttcgcagcgttcttacaattaaattgtggtggaagttcgagacttgcgtaaactatttttaatttgttatgagttacatcaacaacgttttagagctagaaatagcaagttaaaataaggctagtccgttatcaacttgaaaaagtggcaccgagtcggtgctttttttctcgagtttttttatcgagtgtttaaggataatgataactgaagagaagaattagttttgccgccaccgcgggtttgcctctgattaaataaaaaaaagctggtgctttttttttcttttataggaacatcttgaatatatgaactaattaaatgataattttttacccatctttactcttaatcactgagctgcagtcaaagaaaaagggatacagcacctggtgaagagatgaacggagactaacttagacgcgttgattctttttaattgcacattttattaatcgatgctaacgtctatttacatatattctttagagatattatctagggcttcaaataatctctggacagcaataaaagtctcttcaaaagtattgtataacggcaatggggctaatctgattacatctggtcttctttcgtcacagattatagcatgatcatgcaagtacgcattaactcgttccatgacgttcttgtccttttcatcgaaatgcggttgaaacataatggacaattgacatcctctttcagctggattcaaaggagttaaaattttaaacccaaatttggagtttgatgtactggattgtggtatgtaatacttggaattcgtcaatagatcctgtaaaaattgagtcaaagcaacacttttttcacgaagtttagatactccacccactttagcatacacttccaatgacgacttcacagcaacaacatcaagaacagaaggatttgactgtctgtaagaaagagccgagtttattggatcaaactcttctaacattttgaatcgttcttgggagttattgccccaccaaccagctagtctaggaacgaaactgcttttcttgttctctatggtgtatttttcatgcacaaaaatcccacctatggctccaggtcccgagtttaaatatttgtaggaacaccaagcagcaaaatctactccccaatcatgtaaatttaatgggacattcccaactgcatgggcaagatcccacccaactttaatttgttggctcttttccttagcgtatttagttatttcctctatcttgaaaaattgaccagtgtagtattggataccaggaaaacacactagagccaattcatccaggttctcatctatagccttgattattctttctgttttaatataagtttcaccaggttgaacttccaattgaatcaaatgtttctcgtcgtatccgaacaatttaacaatgttcaaaaatgcatagtagtcagaaggaaatgcttgtttttcaaataaaattttggttcttttcccctcaggtttgtaaaaatggatcaacaatgcattcaagtttgctgttaaagaacccataactgcaacttcgttttcctttgcaccaacaatgggggctattaatggtaataagggtaaatcgatgtctacccacggtgttaacagtttgtcaggatgattgaaatgagactcaacccctcgttcaacccatgcatttaattcatcattgatagctttctttgtattcttaggcatcaacccaagagagtttccacataaataaatagactcagttgatgactcatatttattatttttgatacctaatgatccaaaagttggtatggcaaactcatttttaaaagttgggaactttttgtccaatttctttgcctcggctaatgacatctgataataaaatggggttggagtagttggtggtataaccggagagatagaattgaagaaaaaaatcggaaacaacaaaaaaagttgataccctgtattatgtgggagataattgcgaatggtggaaaaaaaaaagacgccattgagtctcaacaacaattctgtcagctgaagagctttacaatcgagaaactatgattcattccgttttaatatgtatgtgtttagtaaactcatgaattttatttgtggtctactttagtactaacataatcattggatagtcaataatgatggtcttccgagactaatgaaattctataccaaagtcgatattccaacacagaaattgctcttgcaacaagtgcacctgttgatatctagagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 25 - DNA, 14064 bp, Artificial Sequence
  Plasmid
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacctgccactactaccactgggagtttcgttcttctcgatactattagctttacttcctgcactagcagtggttggatcaacagaatcttcataatcatcaaaatcgtcttttgaagaccccccgtttgatgtatggccctgtcttttcatcaaactttttatatagttgactgaactgaggctaaatatgtgatcatcttcactatagacaatctttctcttatttgcaccaccgccaccactagtctttgagaaattctcaaaaccttttacgatattaccaagcgggctctcttcgaaataatctatctctttttgatatatcgaatcctctagcgtggttagctttctagttagttcttgcttcttaagaatttgctggattagtttatttttcaattcaacgtatttctcagagtcatctttagattttgatgaagatgtgcgttcattcgctatatccttcttggtcgtgtcttttcgatcctccttggctggcactgaactcgtcttttttggcgttgctgttccagacagacttatctcattagatttggaacttgtgggtttaacatcatttgtatctttagtagacatgattgtgcaataccgtgattatttgttttgaaaggtctgtcatatttctatcaatttcaaaacaaaatgttcatcagaaaaaagccaaaaatgtctcttctagtttcttagtggtgtcgcataatacacaatgtcgctcaacaatccacattcccggcgcatagctcaaatcacatgactacagctaacaattacacaaaaaaaattctctttttgatgtagcaactatcttcaactaaaacattttctccttcggcccatgattgtcctccgggtcgacagcaagccgttacaattgagatggaaagcgacctaccttcactcgataaggtgcttaattgtacttcatataaatctggcccggatctaaacaaatgagttccattaagccgtgggttctcaattagggtttttgtttttgatttagaaaaaagagatcaagatttgtttacaggtgatgcctttttttagaacttatgcgttgcaaaagttgactaacgatttctataaggtgatccacactaattatacaaacgtacaaacagacatacttttcctgcgttcacctgatgttggccagatttctctcttcattgcatagaacataaccacactagggcaacagaaaaaaaaaaaaaaagtgcatcgggaagttgtgttccattcattatatgtctactactgcatatgagtagcccacccaccaccaccatagtaagtttttgtgtatgcgcgccgtcaggttatttcatttctgaatttttcaaccaccttactccctttattgttgattgacaattttgctcacagtaagatcttttagactccaattaatataaaataagtctgattttccaattcctgttttttctttttttttctgtttctatttctttccttttctcccttttttttaattcttcattcaatcatcaattgataattcaggaatattacaacaacccgggatggataaaaagtatagtattggtttagatattggtactaactctgtgggttgggcagttatcaccgacgaatataaagttccatcaaagaaatttaaggtgttaggtaacactgacagacactcaataaaaaagaatcttatcggtgctcttttgttcgactccggtgaaactgccgaggctacacgtttaaaaagaacagcaagaagaagatatacccgtagaaaaaatagaatatgttatttacaagaaatcttttctaatgaaatggctaaagttgatgattcctttttccatagattggaagagtcatttttggttgaagaagacaaaaagcatgagagacatccaatctttgggaatatagttgatgaagtggcttaccatgaaaaatatcctaccatttatcatttaagaaagaaattggtagattcaactgataaagctgaccttagattaatctatttagcacttgcccatatgattaaatttagaggtcattttttgattgaaggtgatttgaacccagataattctgacgtggataaattatttattcaattagtccaaacctacaaccaattatttgaggaaaatccaattaatgctagtggtgtcgatgccaaagctatattatcagccagattatcaaaatctagacgtttggaaaatttgattgcccaattgccaggagaaaaaaagaatggattatttggaaacttgatcgcattatcattgggtttgacaccaaattttaaatctaattttgatttagctgaagatgctaaattacaattatcaaaagacacctatgacgacgatttggacaatttacttgctcaaattggtgatcaatatgcagatttgttcttagctgctaaaaacttatctgatgctattttgttgtctgatattttgagagtgaacacagaaataaccaaagctccattatcagcatctatgatcaaacgttatgatgaacaccatcaggatttgactttattgaaagctttggtgagacaacaattgccagagaagtataaagaaatctttttcgatcaatctaaaaacgggtatgcaggttatattgatgggggtgcctcccaagaggaattttacaaatttataaaacctattttagaaaagatggatgggactgaggaacttttggtcaaattgaacagagaagatttgttacgtaaacagagaacttttgataatggtagtatacctcaccaaattcatttgggtgagttgcatgcaattttaagaagacaagaagatttttatccatttttaaaagataatagagaaaaaatcgagaaaattttaacctttagaattccatactatgttgggcctttggctagaggtaattcaagatttgcctggatgacacgtaaatcagaagaaactattaccccttggaattttgaagaggttgttgataaaggagcatcagcacagagttttattgaaagaatgaccaatttcgataaaaacttaccaaatgaaaaagttttaccaaaacattccttgttatacgaatattttactgtttacaatgaacttacaaaggttaaatatgttactgaaggtatgcgtaagccagcctttttatctggagaacagaaaaaggcaatagttgatttattgtttaaaacaaatagaaaagttactgttaaacaattaaaagaagattactttaagaaaattgaatgttttgattcagttgaaatcagtggtgttgaagacagatttaatgctagtttaggaacttaccatgatttacttaaaattatcaaagataaagatttcttggataacgaagaaaatgaagacattttagaagacattgttttaaccttaactttattcgaagatagagagatgattgaagaacgtttgaagacttatgcacatttgtttgacgataaagtgatgaaacagttgaaaagaagacgttatactggatggggtagattgtctcgtaaattgatcaatggaattagagataaacaaagtggtaaaactatcttggactttttgaaatctgacggatttgctaatagaaatttcatgcaattgatccacgacgatagtttgacatttaaagaagacatccaaaaggcccaagtgagtgggcaaggtgattcattacatgaacatattgcaaatttagccggatctcctgctattaagaaagggatattacaaactgttaaagttgtggatgaattagtgaaagtaatgggaagacataaacctgaaaacattgtcattgagatggcaagagaaaatcaaactacacaaaaaggacagaaaaatagtagagaacgtatgaaaagaatagaagagggtattaaagaattgggtagtcaaatattgaaagaacacccagtggaaaatacccagttgcaaaatgaaaaattatatctttactaccttcaaaatggacgtgatatgtatgttgatcaggaattagatataaatagactttcagattatgatgtagatcatatagttccacaatctttcttgaaagatgattccatagacaataaagtattaactagaagtgataaaaatagaggtaaaagtgataatgtcccaagtgaggaagtcgtcaaaaagatgaaaaattactggcgtcaacttttgaatgctaaattaattactcaaagaaaatttgataatttgactaaagcagaaagaggtgggctttctgaattagataaagccgggttcattaaaagacaattggtcgaaactagacaaattactaaacatgttgcccaaattttagattcccgtatgaacactaagtatgacgaaaatgataagttaatacgtgaggttaaagtcattactttaaaatcaaaacttgtctctgatttcagaaaggatttccaattctataaagttagagaaattaataattatcatcatgctcatgatgcatatttgaatgctgtagttggaactgctttaatcaagaaataccctaaattagaatctgaatttgtatatggtgattacaaagtctatgatgttagaaagatgattgctaaatcagaacaagaaattggtaaagctacagctaaatacttcttttactctaacattatgaatttctttaaaacagaaattactttggcaaacggtgaaattagaaaaagacctcttattgaaacaaatggtgagactggagagatagtttgggacaaagggcgtgatttcgctactgttagaaaagttttatcaatgccacaagttaacattgtaaagaaaacagaggttcaaactggtggtttctcaaaagaaagtattttgcctaaaagaaatagtgataaattgattgccagaaaaaaggattgggatccaaagaaatatggtggtttcgactcaccaaccgtagcctattctgttttggttgtggcaaaggttgaaaagggtaaaagtaaaaagcttaaatcagtaaaagaacttttgggtattacaataatggaaagaagttcctttgaaaagaaccctattgattttttggaagctaaaggttataaggaagtaaagaaggacttaataatcaaattgcctaaatattctttatttgaattagaaaatgggagaaaaagaatgttggcttctgctggagaattgcaaaagggtaatgaattagcattgccttccaaatatgttaacttcttgtatttagcttcacactatgaaaagttgaaagggtcaccagaagataacgagcaaaaacaattatttgttgaacaacacaaacactacttagatgagattatagaacaaattagtgaattcagtaaaagagtgatattagctgatgcaaatttagataaagttttgtcagcctataacaaacatagagataagccaattagagaacaagcagaaaacattattcacttatttacccttaccaatttaggagcacctgctgctttcaagtattttgatacaacaattgatcgtaaaagatatacctcaacaaaagaagtcttagacgccaccttaattcatcaatcaatcactggattgtatgagacaagaattgatttgtctcaattgggtggtgatgaaggggctgatcctaagaagaaaagaaaagttgatccaaagaaaaagcgtaaggtggatcctaagaaaaagagaaaggttgactacaaagaccatgacggtgattataaagatcatgacatcgactacaaggatgacgatgacaagtgataatgactgcagagatccatcgacctgccgccaagctaattccgggcgaatttctgtcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccgggccccccctaactcaagtacaacagatctggaccacctttgattgtaaatagtaataattaccacccttatctaattatttatttaacttatttatttatttattatacatatatacaaatctaataaagtgaaaatctcccccttcacacttcacatatgttaggcgtcatcctgtgctcccgagaaccagtaccagtacatcgctgtttcgttcgagacttgaggtctagttttatacgtgaagaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaattttcatatgagaaccgttatcgataactaaagcagcaacttcttctataaaaatgggttagtatgacagtcatttaaataaggaatttttcagttggcttggtttcaattcaatgttcgttttttttttttcttgctgtgtttgtgtttgtgttgtttatagttgtgtgcactgatcgtcgaaaaaaaaaattcatagtgagccgggaaatctgtatagcccagataacaacacaagtccaaactagaaactcgtcaaacaccaaaagcaatgttgaatcaattgccttgcacaagtacacgtaggaaaacataaaacattgcaattttgaatattgagccttttgtcgtaacattgattgataggattactcaccgaatggttttgaaaccactgccgacagatcaatcaatcaatcaaaaaacgtgaactttgaaaaaggggaagaacagatacattgaagttagccatttccactgatcgtcacaacatatctgataaattactttcaaaattataagctgatgtgtgtgtattattaatgtgacagtaacatcccaaacgagaaatattatctcgacaacaaaaaagtttgatctgaattgaaaatgaagttttcccaccctacccatttgtcatattgaaaccaatcaactgattaatcaatcaattagaattgaagctaaactaaaacataccaccgtccattttgaatgattatatttttttaatattaatatcgagataatgtttctaagaaagaaagaaaaccaggagtgaaaattagaaaaggaaaggaaaggaaaaaaagaaaaatctgaaaatatataaaaaaaaattgtttcgttggcaataaatcttggtgagaacagcgaccgaaagcaaataagaacaaaatatgagtgtattacgttgaacaactaattaacgtgtgtgtatggatctttttttcttttttctctttaaccgactataaacaacaaacatttttgggcagtgcacacactacttaatatacacagcataaattacacgattagaaacaaattagcttattaaaataacctaatcaaaccgaatattttatggtattatgagtaaactatataatataaatagcacacacccacaacaacaacaaaggaaaactaaaaggttttttctttttgaaaagatcgttttctttattattctctagttttgacggcggccgcaagtgattagacttagtccgttcaaatcaagcacaactctgttcattgtttcaacaagaattaattcaaaaacaggttcggtgcataatttgcaaaaaaatattgcagcttctgtggctcgaacacagtacctccagatttcaggtttgaaatacttcagtctgacgctctcccagatgagctaaagctgcaataagaaaacccacgccgggattcgaacccggaatcctttgattagaagtcaaaagcgataaccatttcgccacgcaggcctacttgatgggtttgtaaatggtctactttttcagacctaacagaaattttaatgaaagtcatattcttatacaataaaactgtgtcataaaagcagatattcgactttcgtagattatataggacccaagaactaaaatttaatgccatattatgcatttttaatctgtaaaagtgttgtttccaacctatcacaagtacgttcttgtaacttgtgtttgtagggttgcaaatgaatcataacaacatctcaacagaacatgtatagcaaagcttagtataaaatcagtgttttgagaggcaatccaagaatgtttacatcaaagtttcaataaatatcgaccgaaactgaaaatctttttaggttattgttcacttttttgtaaatatttaaacattttttggacctaaaaaaatacaaacaccaattacgtaccaagaagcatctaatcaactcccagatcaccactatacatttaaaagtcattggtcaataactatactcgagtattgcctcatcaaagaaacaatcaaatattatagatactcactccatcacgtgataatttcactggtatggaaaagtggaaaattttataaaaaaaaatttgatgcctttggcatagctgaaacttcggcccaataggattggagaatatgttttcgcagcgttcttacaattaaattgtggtggaagttcgagacttgcgtaaactatttttaatttgttggctcaacacttgggcacgttttagagctagaaatagcaagttaaaataaggctagtccgttatcaacttgaaaaagtggcaccgagtcggtgctttttttctcgagtttttttatcgagtgtttaaggataatgataactgaagagaagaattagttttgccgccaccgcgggtttgcctctgattaaataaaaaaaagctggtgctttttttttcttttataggaacatcttgaatatatgaactaattaaatgataattttttacccatctttactcttaatcactgagctgcagtcaaagaaaaagggatacagcacctggtgaagagatgaacggagactaacttagacgcgttgattctttttaattgcacattttattaatcgatgctaacgtctatttacatatattctttagagatattatctagggcttcaaataatctctggacagcaataaaagtctcttcaaaagtattgtataacggcaatggggctaatctgattacatctggtcttctttcgtcacagattatagcatgatcatgcaagtacgcattaactcgttccatgacgttcttgtccttttcatcgaaatgcggttgaaacataatggacaattgacatcctctttcagctggattcaaaggagttaaaattttaaacccaaatttggagtttgatgtactggattgtggtatgtaatacttggaattcgtcaatagatcctgtaaaaattgagtcaaagcaacacttttttcacgaagtttagatactccacccactttagcatacacttccaatgacgacttcacagcaacaacatcaagaacagaaggatttgactgtctgtaagaaagagccgagtttattggatcaaactcttctaacattttgaatcgttcttgggagttattgccccaccaaccagctagtctaggaacgaaactgcttttcttgttctctatggtgtatttttcatgcacaaaaatcccacctatggctccaggtcccgagtttaaatatttgtaggaacaccaagcagcaaaatctactccccaatcatgtaaatttaatgggacattcccaactgcatgggcaagatcccacccaactttaatttgttggctcttttccttagcgtatttagttatttcctctatcttgaaaaattgaccagtgtagtattggataccaggaaaacacactagagccaattcatccaggttctcatctatagccttgattattctttctgttttaatataagtttcaccaggttgaacttccaattgaatcaaatgtttctcgtcgtatccgaacaatttaacaatgttcaaaaatgcatagtagtcagaaggaaatgcttgtttttcaaataaaattttggttcttttcccctcaggtttgtaaaaatggatcaacaatgcattcaagtttgctgttaaagaacccataactgcaacttcgttttcctttgcaccaacaatgggggctattaatggtaataagggtaaatcgatgtctacccacggtgttaacagtttgtcaggatgattgaaatgagactcaacccctcgttcaacccatgcatttaattcatcattgatagctttctttgtattcttaggcatcaacccaagagagtttccacataaataaatagactcagttgatgactcatatttattatttttgatacctaatgatccaaaagttggtatggcaaactcatttttaaaagttgggaactttttgtccaatttctttgcctcggctaatgacatctgataataaaatggggttggagtagttggtggtataaccggagagatagaattgaagaaaaaaatcggaaacaacaaaaaaagttgataccctgtattatgtgggagataattgcgaatggtggaaaaaaaaaagacgccattgagtctcaacaacaattctgtcagctgaagagctttacaatcgagaaactatgattcattccgttttaatatgtatgtgtttagtaaactcatgaattttatttgtggtctactttagtactaacataatcattggatagtcaataatgatggtcttccgagactaatgaaattctataccaaagtcgatattccaacacagaaattgctcttgcaacaagtgcacctgttgatatctagagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 26 - DNA, 14064 bp, Artificial Sequence
  Plasmid
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacctgccactactaccactgggagtttcgttcttctcgatactattagctttacttcctgcactagcagtggttggatcaacagaatcttcataatcatcaaaatcgtcttttgaagaccccccgtttgatgtatggccctgtcttttcatcaaactttttatatagttgactgaactgaggctaaatatgtgatcatcttcactatagacaatctttctcttatttgcaccaccgccaccactagtctttgagaaattctcaaaaccttttacgatattaccaagcgggctctcttcgaaataatctatctctttttgatatatcgaatcctctagcgtggttagctttctagttagttcttgcttcttaagaatttgctggattagtttatttttcaattcaacgtatttctcagagtcatctttagattttgatgaagatgtgcgttcattcgctatatccttcttggtcgtgtcttttcgatcctccttggctggcactgaactcgtcttttttggcgttgctgttccagacagacttatctcattagatttggaacttgtgggtttaacatcatttgtatctttagtagacatgattgtgcaataccgtgattatttgttttgaaaggtctgtcatatttctatcaatttcaaaacaaaatgttcatcagaaaaaagccaaaaatgtctcttctagtttcttagtggtgtcgcataatacacaatgtcgctcaacaatccacattcccggcgcatagctcaaatcacatgactacagctaacaattacacaaaaaaaattctctttttgatgtagcaactatcttcaactaaaacattttctccttcggcccatgattgtcctccgggtcgacagcaagccgttacaattgagatggaaagcgacctaccttcactcgataaggtgcttaattgtacttcatataaatctggcccggatctaaacaaatgagttccattaagccgtgggttctcaattagggtttttgtttttgatttagaaaaaagagatcaagatttgtttacaggtgatgcctttttttagaacttatgcgttgcaaaagttgactaacgatttctataaggtgatccacactaattatacaaacgtacaaacagacatacttttcctgcgttcacctgatgttggccagatttctctcttcattgcatagaacataaccacactagggcaacagaaaaaaaaaaaaaaagtgcatcgggaagttgtgttccattcattatatgtctactactgcatatgagtagcccacccaccaccaccatagtaagtttttgtgtatgcgcgccgtcaggttatttcatttctgaatttttcaaccaccttactccctttattgttgattgacaattttgctcacagtaagatcttttagactccaattaatataaaataagtctgattttccaattcctgttttttctttttttttctgtttctatttctttccttttctcccttttttttaattcttcattcaatcatcaattgataattcaggaatattacaacaacccgggatggataaaaagtatagtattggtttagatattggtactaactctgtgggttgggcagttatcaccgacgaatataaagttccatcaaagaaatttaaggtgttaggtaacactgacagacactcaataaaaaagaatcttatcggtgctcttttgttcgactccggtgaaactgccgaggctacacgtttaaaaagaacagcaagaagaagatatacccgtagaaaaaatagaatatgttatttacaagaaatcttttctaatgaaatggctaaagttgatgattcctttttccatagattggaagagtcatttttggttgaagaagacaaaaagcatgagagacatccaatctttgggaatatagttgatgaagtggcttaccatgaaaaatatcctaccatttatcatttaagaaagaaattggtagattcaactgataaagctgaccttagattaatctatttagcacttgcccatatgattaaatttagaggtcattttttgattgaaggtgatttgaacccagataattctgacgtggataaattatttattcaattagtccaaacctacaaccaattatttgaggaaaatccaattaatgctagtggtgtcgatgccaaagctatattatcagccagattatcaaaatctagacgtttggaaaatttgattgcccaattgccaggagaaaaaaagaatggattatttggaaacttgatcgcattatcattgggtttgacaccaaattttaaatctaattttgatttagctgaagatgctaaattacaattatcaaaagacacctatgacgacgatttggacaatttacttgctcaaattggtgatcaatatgcagatttgttcttagctgctaaaaacttatctgatgctattttgttgtctgatattttgagagtgaacacagaaataaccaaagctccattatcagcatctatgatcaaacgttatgatgaacaccatcaggatttgactttattgaaagctttggtgagacaacaattgccagagaagtataaagaaatctttttcgatcaatctaaaaacgggtatgcaggttatattgatgggggtgcctcccaagaggaattttacaaatttataaaacctattttagaaaagatggatgggactgaggaacttttggtcaaattgaacagagaagatttgttacgtaaacagagaacttttgataatggtagtatacctcaccaaattcatttgggtgagttgcatgcaattttaagaagacaagaagatttttatccatttttaaaagataatagagaaaaaatcgagaaaattttaacctttagaattccatactatgttgggcctttggctagaggtaattcaagatttgcctggatgacacgtaaatcagaagaaactattaccccttggaattttgaagaggttgttgataaaggagcatcagcacagagttttattgaaagaatgaccaatttcgataaaaacttaccaaatgaaaaagttttaccaaaacattccttgttatacgaatattttactgtttacaatgaacttacaaaggttaaatatgttactgaaggtatgcgtaagccagcctttttatctggagaacagaaaaaggcaatagttgatttattgtttaaaacaaatagaaaagttactgttaaacaattaaaagaagattactttaagaaaattgaatgttttgattcagttgaaatcagtggtgttgaagacagatttaatgctagtttaggaacttaccatgatttacttaaaattatcaaagataaagatttcttggataacgaagaaaatgaagacattttagaagacattgttttaaccttaactttattcgaagatagagagatgattgaagaacgtttgaagacttatgcacatttgtttgacgataaagtgatgaaacagttgaaaagaagacgttatactggatggggtagattgtctcgtaaattgatcaatggaattagagataaacaaagtggtaaaactatcttggactttttgaaatctgacggatttgctaatagaaatttcatgcaattgatccacgacgatagtttgacatttaaagaagacatccaaaaggcccaagtgagtgggcaaggtgattcattacatgaacatattgcaaatttagccggatctcctgctattaagaaagggatattacaaactgttaaagttgtggatgaattagtgaaagtaatgggaagacataaacctgaaaacattgtcattgagatggcaagagaaaatcaaactacacaaaaaggacagaaaaatagtagagaacgtatgaaaagaatagaagagggtattaaagaattgggtagtcaaatattgaaagaacacccagtggaaaatacccagttgcaaaatgaaaaattatatctttactaccttcaaaatggacgtgatatgtatgttgatcaggaattagatataaatagactttcagattatgatgtagatcatatagttccacaatctttcttgaaagatgattccatagacaataaagtattaactagaagtgataaaaatagaggtaaaagtgataatgtcccaagtgaggaagtcgtcaaaaagatgaaaaattactggcgtcaacttttgaatgctaaattaattactcaaagaaaatttgataatttgactaaagcagaaagaggtgggctttctgaattagataaagccgggttcattaaaagacaattggtcgaaactagacaaattactaaacatgttgcccaaattttagattcccgtatgaacactaagtatgacgaaaatgataagttaatacgtgaggttaaagtcattactttaaaatcaaaacttgtctctgatttcagaaaggatttccaattctataaagttagagaaattaataattatcatcatgctcatgatgcatatttgaatgctgtagttggaactgctttaatcaagaaataccctaaattagaatctgaatttgtatatggtgattacaaagtctatgatgttagaaagatgattgctaaatcagaacaagaaattggtaaagctacagctaaatacttcttttactctaacattatgaatttctttaaaacagaaattactttggcaaacggtgaaattagaaaaagacctcttattgaaacaaatggtgagactggagagatagtttgggacaaagggcgtgatttcgctactgttagaaaagttttatcaatgccacaagttaacattgtaaagaaaacagaggttcaaactggtggtttctcaaaagaaagtattttgcctaaaagaaatagtgataaattgattgccagaaaaaaggattgggatccaaagaaatatggtggtttcgactcaccaaccgtagcctattctgttttggttgtggcaaaggttgaaaagggtaaaagtaaaaagcttaaatcagtaaaagaacttttgggtattacaataatggaaagaagttcctttgaaaagaaccctattgattttttggaagctaaaggttataaggaagtaaagaaggacttaataatcaaattgcctaaatattctttatttgaattagaaaatgggagaaaaagaatgttggcttctgctggagaattgcaaaagggtaatgaattagcattgccttccaaatatgttaacttcttgtatttagcttcacactatgaaaagttgaaagggtcaccagaagataacgagcaaaaacaattatttgttgaacaacacaaacactacttagatgagattatagaacaaattagtgaattcagtaaaagagtgatattagctgatgcaaatttagataaagttttgtcagcctataacaaacatagagataagccaattagagaacaagcagaaaacattattcacttatttacccttaccaatttaggagcacctgctgctttcaagtattttgatacaacaattgatcgtaaaagatatacctcaacaaaagaagtcttagacgccaccttaattcatcaatcaatcactggattgtatgagacaagaattgatttgtctcaattgggtggtgatgaaggggctgatcctaagaagaaaagaaaagttgatccaaagaaaaagcgtaaggtggatcctaagaaaaagagaaaggttgactacaaagaccatgacggtgattataaagatcatgacatcgactacaaggatgacgatgacaagtgataatgactgcagagatccatcgacctgccgccaagctaattccgggcgaatttctgtcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccgggccccccctaactcaagtacaacagatctggaccacctttgattgtaaatagtaataattaccacccttatctaattatttatttaacttatttatttatttattatacatatatacaaatctaataaagtgaaaatctcccccttcacacttcacatatgttaggcgtcatcctgtgctcccgagaaccagtaccagtacatcgctgtttcgttcgagacttgaggtctagttttatacgtgaagaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaattttcatatgagaaccgttatcgataactaaagcagcaacttcttctataaaaatgggttagtatgacagtcatttaaataaggaatttttcagttggcttggtttcaattcaatgttcgttttttttttttcttgctgtgtttgtgtttgtgttgtttatagttgtgtgcactgatcgtcgaaaaaaaaaattcatagtgagccgggaaatctgtatagcccagataacaacacaagtccaaactagaaactcgtcaaacaccaaaagcaatgttgaatcaattgccttgcacaagtacacgtaggaaaacataaaacattgcaattttgaatattgagccttttgtcgtaacattgattgataggattactcaccgaatggttttgaaaccactgccgacagatcaatcaatcaatcaaaaaacgtgaactttgaaaaaggggaagaacagatacattgaagttagccatttccactgatcgtcacaacatatctgataaattactttcaaaattataagctgatgtgtgtgtattattaatgtgacagtaacatcccaaacgagaaatattatctcgacaacaaaaaagtttgatctgaattgaaaatgaagttttcccaccctacccatttgtcatattgaaaccaatcaactgattaatcaatcaattagaattgaagctaaactaaaacataccaccgtccattttgaatgattatatttttttaatattaatatcgagataatgtttctaagaaagaaagaaaaccaggagtgaaaattagaaaaggaaaggaaaggaaaaaaagaaaaatctgaaaatatataaaaaaaaattgtttcgttggcaataaatcttggtgagaacagcgaccgaaagcaaataagaacaaaatatgagtgtattacgttgaacaactaattaacgtgtgtgtatggatctttttttcttttttctctttaaccgactataaacaacaaacatttttgggcagtgcacacactacttaatatacacagcataaattacacgattagaaacaaattagcttattaaaataacctaatcaaaccgaatattttatggtattatgagtaaactatataatataaatagcacacacccacaacaacaacaaaggaaaactaaaaggttttttctttttgaaaagatcgttttctttattattctctagttttgacggcggccgcaagtgattagacttagtccgttcaaatcaagcacaactctgttcattgtttcaacaagaattaattcaaaaacaggttcggtgcataatttgcaaaaaaatattgcagcttctgtggctcgaacacagtacctccagatttcaggtttgaaatacttcagtctgacgctctcccagatgagctaaagctgcaataagaaaacccacgccgggattcgaacccggaatcctttgattagaagtcaaaagcgataaccatttcgccacgcaggcctacttgatgggtttgtaaatggtctactttttcagacctaacagaaattttaatgaaagtcatattcttatacaataaaactgtgtcataaaagcagatattcgactttcgtagattatataggacccaagaactaaaatttaatgccatattatgcatttttaatctgtaaaagtgttgtttccaacctatcacaagtacgttcttgtaacttgtgtttgtagggttgcaaatgaatcataacaacatctcaacagaacatgtatagcaaagcttagtataaaatcagtgttttgagaggcaatccaagaatgtttacatcaaagtttcaataaatatcgaccgaaactgaaaatctttttaggttattgttcacttttttgtaaatatttaaacattttttggacctaaaaaaatacaaacaccaattacgtaccaagaagcatctaatcaactcccagatcaccactatacatttaaaagtcattggtcaataactatactcgagtattgcctcatcaaagaaacaatcaaatattatagatactcactccatcacgtgataatttcactggtatggaaaagtggaaaattttataaaaaaaaatttgatgcctttggcatagctgaaacttcggcccaataggattggagaatatgttttcgcagcgttcttacaattaaattgtggtggaagttcgagacttgcgtaaactatttttaatttgatataatgtgtattacttctgttttagagctagaaatagcaagttaaaataaggctagtccgttatcaacttgaaaaagtggcaccgagtcggtgctttttttctcgagtttttttatcgagtgtttaaggataatgataactgaagagaagaattagttttgccgccaccgcgggtttgcctctgattaaataaaaaaaagctggtgctttttttttcttttataggaacatcttgaatatatgaactaattaaatgataattttttacccatctttactcttaatcactgagctgcagtcaaagaaaaagggatacagcacctggtgaagagatgaacggagactaacttagacgcgttgattctttttaattgcacattttattaatcgatgctaacgtctatttacatatattctttagagatattatctagggcttcaaataatctctggacagcaataaaagtctcttcaaaagtattgtataacggcaatggggctaatctgattacatctggtcttctttcgtcacagattatagcatgatcatgcaagtacgcattaactcgttccatgacgttcttgtccttttcatcgaaatgcggttgaaacataatggacaattgacatcctctttcagctggattcaaaggagttaaaattttaaacccaaatttggagtttgatgtactggattgtggtatgtaatacttggaattcgtcaatagatcctgtaaaaattgagtcaaagcaacacttttttcacgaagtttagatactccacccactttagcatacacttccaatgacgacttcacagcaacaacatcaagaacagaaggatttgactgtctgtaagaaagagccgagtttattggatcaaactcttctaacattttgaatcgttcttgggagttattgccccaccaaccagctagtctaggaacgaaactgcttttcttgttctctatggtgtatttttcatgcacaaaaatcccacctatggctccaggtcccgagtttaaatatttgtaggaacaccaagcagcaaaatctactccccaatcatgtaaatttaatgggacattcccaactgcatgggcaagatcccacccaactttaatttgttggctcttttccttagcgtatttagttatttcctctatcttgaaaaattgaccagtgtagtattggataccaggaaaacacactagagccaattcatccaggttctcatctatagccttgattattctttctgttttaatataagtttcaccaggttgaacttccaattgaatcaaatgtttctcgtcgtatccgaacaatttaacaatgttcaaaaatgcatagtagtcagaaggaaatgcttgtttttcaaataaaattttggttcttttcccctcaggtttgtaaaaatggatcaacaatgcattcaagtttgctgttaaagaacccataactgcaacttcgttttcctttgcaccaacaatgggggctattaatggtaataagggtaaatcgatgtctacccacggtgttaacagtttgtcaggatgattgaaatgagactcaacccctcgttcaacccatgcatttaattcatcattgatagctttctttgtattcttaggcatcaacccaagagagtttccacataaataaatagactcagttgatgactcatatttattatttttgatacctaatgatccaaaagttggtatggcaaactcatttttaaaagttgggaactttttgtccaatttctttgcctcggctaatgacatctgataataaaatggggttggagtagttggtggtataaccggagagatagaattgaagaaaaaaatcggaaacaacaaaaaaagttgataccctgtattatgtgggagataattgcgaatggtggaaaaaaaaaagacgccattgagtctcaacaacaattctgtcagctgaagagctttacaatcgagaaactatgattcattccgttttaatatgtatgtgtttagtaaactcatgaattttatttgtggtctactttagtactaacataatcattggatagtcaataatgatggtcttccgagactaatgaaattctataccaaagtcgatattccaacacagaaattgctcttgcaacaagtgcacctgttgatatctagagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 27 - DNA, 16241 bp, Artificial Sequence
  Plasmid
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacctgccactactaccactgggagtttcgttcttctcgatactattagctttacttcctgcactagcagtggttggatcaacagaatcttcataatcatcaaaatcgtcttttgaagaccccccgtttgatgtatggccctgtcttttcatcaaactttttatatagttgactgaactgaggctaaatatgtgatcatcttcactatagacaatctttctcttatttgcaccaccgccaccactagtctttgagaaattctcaaaaccttttacgatattaccaagcgggctctcttcgaaataatctatctctttttgatatatcgaatcctctagcgtggttagctttctagttagttcttgcttcttaagaatttgctggattagtttatttttcaattcaacgtatttctcagagtcatctttagattttgatgaagatgtgcgttcattcgctatatccttcttggtcgtgtcttttcgatcctccttggctggcactgaactcgtcttttttggcgttgctgttccagacagacttatctcattagatttggaacttgtgggtttaacatcatttgtatctttagtagacatgattgtgcaataccgtgattatttgttttgaaaggtctgtcatatttctatcaatttcaaaacaaaatgttcatcagaaaaaagccaaaaatgtctcttctagtttcttagtggtgtcgcataatacacaatgtcgctcaacaatccacattcccggcgcatagctcaaatcacatgactacagctaacaattacacaaaaaaaattctctttttgatgtagcaactatcttcaactaaaacattttctccttcggcccatgattgtcctccgggtcgacagcaagccgttacaattgagatggaaagcgacctaccttcactcgataaggtgcttaattgtacttcatataaatctggcccggatctaaacaaatgagttccattaagccgtgggttctcaattagggtttttgtttttgatttagaaaaaagagatcaagatttgtttacaggtgatgcctttttttagaacttatgcgttgcaaaagttgactaacgatttctataaggtgatccacactaattatacaaacgtacaaacagacatacttttcctgcgttcacctgatgttggccagatttctctcttcattgcatagaacataaccacactagggcaacagaaaaaaaaaaaaaaagtgcatcgggaagttgtgttccattcattatatgtctactactgcatatgagtagcccacccaccaccaccatagtaagtttttgtgtatgcgcgccgtcaggttatttcatttctgaatttttcaaccaccttactccctttattgttgattgacaattttgctcacagtaagatcttttagactccaattaatataaaataagtctgattttccaattcctgttttttctttttttttctgtttctatttctttccttttctcccttttttttaattcttcattcaatcatcaattgataattcaggaatattacaacaacccgggatggataaaaagtatagtattggtttagatattggtactaactctgtgggttgggcagttatcaccgacgaatataaagttccatcaaagaaatttaaggtgttaggtaacactgacagacactcaataaaaaagaatcttatcggtgctcttttgttcgactccggtgaaactgccgaggctacacgtttaaaaagaacagcaagaagaagatatacccgtagaaaaaatagaatatgttatttacaagaaatcttttctaatgaaatggctaaagttgatgattcctttttccatagattggaagagtcatttttggttgaagaagacaaaaagcatgagagacatccaatctttgggaatatagttgatgaagtggcttaccatgaaaaatatcctaccatttatcatttaagaaagaaattggtagattcaactgataaagctgaccttagattaatctatttagcacttgcccatatgattaaatttagaggtcattttttgattgaaggtgatttgaacccagataattctgacgtggataaattatttattcaattagtccaaacctacaaccaattatttgaggaaaatccaattaatgctagtggtgtcgatgccaaagctatattatcagccagattatcaaaatctagacgtttggaaaatttgattgcccaattgccaggagaaaaaaagaatggattatttggaaacttgatcgcattatcattgggtttgacaccaaattttaaatctaattttgatttagctgaagatgctaaattacaattatcaaaagacacctatgacgacgatttggacaatttacttgctcaaattggtgatcaatatgcagatttgttcttagctgctaaaaacttatctgatgctattttgttgtctgatattttgagagtgaacacagaaataaccaaagctccattatcagcatctatgatcaaacgttatgatgaacaccatcaggatttgactttattgaaagctttggtgagacaacaattgccagagaagtataaagaaatctttttcgatcaatctaaaaacgggtatgcaggttatattgatgggggtgcctcccaagaggaattttacaaatttataaaacctattttagaaaagatggatgggactgaggaacttttggtcaaattgaacagagaagatttgttacgtaaacagagaacttttgataatggtagtatacctcaccaaattcatttgggtgagttgcatgcaattttaagaagacaagaagatttttatccatttttaaaagataatagagaaaaaatcgagaaaattttaacctttagaattccatactatgttgggcctttggctagaggtaattcaagatttgcctggatgacacgtaaatcagaagaaactattaccccttggaattttgaagaggttgttgataaaggagcatcagcacagagttttattgaaagaatgaccaatttcgataaaaacttaccaaatgaaaaagttttaccaaaacattccttgttatacgaatattttactgtttacaatgaacttacaaaggttaaatatgttactgaaggtatgcgtaagccagcctttttatctggagaacagaaaaaggcaatagttgatttattgtttaaaacaaatagaaaagttactgttaaacaattaaaagaagattactttaagaaaattgaatgttttgattcagttgaaatcagtggtgttgaagacagatttaatgctagtttaggaacttaccatgatttacttaaaattatcaaagataaagatttcttggataacgaagaaaatgaagacattttagaagacattgttttaaccttaactttattcgaagatagagagatgattgaagaacgtttgaagacttatgcacatttgtttgacgataaagtgatgaaacagttgaaaagaagacgttatactggatggggtagattgtctcgtaaattgatcaatggaattagagataaacaaagtggtaaaactatcttggactttttgaaatctgacggatttgctaatagaaatttcatgcaattgatccacgacgatagtttgacatttaaagaagacatccaaaaggcccaagtgagtgggcaaggtgattcattacatgaacatattgcaaatttagccggatctcctgctattaagaaagggatattacaaactgttaaagttgtggatgaattagtgaaagtaatgggaagacataaacctgaaaacattgtcattgagatggcaagagaaaatcaaactacacaaaaaggacagaaaaatagtagagaacgtatgaaaagaatagaagagggtattaaagaattgggtagtcaaatattgaaagaacacccagtggaaaatacccagttgcaaaatgaaaaattatatctttactaccttcaaaatggacgtgatatgtatgttgatcaggaattagatataaatagactttcagattatgatgtagatcatatagttccacaatctttcttgaaagatgattccatagacaataaagtattaactagaagtgataaaaatagaggtaaaagtgataatgtcccaagtgaggaagtcgtcaaaaagatgaaaaattactggcgtcaacttttgaatgctaaattaattactcaaagaaaatttgataatttgactaaagcagaaagaggtgggctttctgaattagataaagccgggttcattaaaagacaattggtcgaaactagacaaattactaaacatgttgcccaaattttagattcccgtatgaacactaagtatgacgaaaatgataagttaatacgtgaggttaaagtcattactttaaaatcaaaacttgtctctgatttcagaaaggatttccaattctataaagttagagaaattaataattatcatcatgctcatgatgcatatttgaatgctgtagttggaactgctttaatcaagaaataccctaaattagaatctgaatttgtatatggtgattacaaagtctatgatgttagaaagatgattgctaaatcagaacaagaaattggtaaagctacagctaaatacttcttttactctaacattatgaatttctttaaaacagaaattactttggcaaacggtgaaattagaaaaagacctcttattgaaacaaatggtgagactggagagatagtttgggacaaagggcgtgatttcgctactgttagaaaagttttatcaatgccacaagttaacattgtaaagaaaacagaggttcaaactggtggtttctcaaaagaaagtattttgcctaaaagaaatagtgataaattgattgccagaaaaaaggattgggatccaaagaaatatggtggtttcgactcaccaaccgtagcctattctgttttggttgtggcaaaggttgaaaagggtaaaagtaaaaagcttaaatcagtaaaagaacttttgggtattacaataatggaaagaagttcctttgaaaagaaccctattgattttttggaagctaaaggttataaggaagtaaagaaggacttaataatcaaattgcctaaatattctttatttgaattagaaaatgggagaaaaagaatgttggcttctgctggagaattgcaaaagggtaatgaattagcattgccttccaaatatgttaacttcttgtatttagcttcacactatgaaaagttgaaagggtcaccagaagataacgagcaaaaacaattatttgttgaacaacacaaacactacttagatgagattatagaacaaattagtgaattcagtaaaagagtgatattagctgatgcaaatttagataaagttttgtcagcctataacaaacatagagataagccaattagagaacaagcagaaaacattattcacttatttacccttaccaatttaggagcacctgctgctttcaagtattttgatacaacaattgatcgtaaaagatatacctcaacaaaagaagtcttagacgccaccttaattcatcaatcaatcactggattgtatgagacaagaattgatttgtctcaattgggtggtgatgaaggggctgatcctaagaagaaaagaaaagttgatccaaagaaaaagcgtaaggtggatcctaagaaaaagagaaaggttgactacaaagaccatgacggtgattataaagatcatgacatcgactacaaggatgacgatgacaagtgataatgactgcagagatccatcgacctgccgccaagctaattccgggcgaatttctgtcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccgggccccccctcgaggaagttcctatactttctagagaataggaacttcggatccactagttctagatttttgcaagcatttaaatattgccaagtaaaaacttcaaattttctttccccttggaactttgactttatttttttgacagattattttgacacacacacaccaaatgtgttaccccttaaaacaaaaaaacacttttttacaatttcttggtatccagaatcattctaagcatcattcaattataatttcaatccaaaaaagtagttttagtttgacttgaaacgtcaacaaacacaaatttcaaatcataacctctcctgttgcctgtcaacaacacaccataaggagaaggaataggaggaggaggagatagaaacttgcacggcaccacaaaacacaaaattgatttcaaccaatacggtgacaacaacaatagatttccgatagaaataatgattatcggaataagctagctttgctttgctttgctttgctttttgacttgctctaatttttcgaaaataataatggagaaaagttcaaggtgtttaatgcatcaactaaaacagaaaataatacattagactaaacttttaatctttctagtaccaataattcacgcgtgcgttttaatcccaatcatgaaatgaagaagttatttccctttttctttcatcaaaaaagaactaaattattttttaaattttagtaaacaaaacctggaaatcggggaaaccgggggaggggggcagaaggtgaaacgggtaatattgataaatttaatctataattgataaagttaaatttaaattgatttgaattgatttgaattgaatgaaatgcatttgaataaacggcatcaaactaaaaaaatatagatcacattcatagtaaaacgataacaaagaacaccacaatttatagcaatgataataaacatctaaaaagaaaagggtacgagaaggagaatgaaaaaaaacaataagctagttcttaatctgttcagatatctaatttcaaaaaaaagaatagtataaaaggatagttgattcctcttggttgttgaaaatttgaataatatcaatcaattaatcaatcaaataacaacaacccactagacatcaccattgtcgacatgccacaatttgatatattatgtaaaacaccacctaaggtgcttgttcgtcagtttgtggaaaggtttgaaagaccttcaggtgagaaaatagcattatgtgctgctgaactaacctatttatgttggatgattacacataacggaacagcaatcaagagagccacattcatgagctataatactatcataagcaattcgttgagtttcgatattgtcaataaatcactccagtttaaatacaagacgcaaaaagcaacaattttggaagcctcattaaagaaattgattcctgcttgggaatttacaattattccttactatggacaaaaacatcaatctgatatcactgatattgtaagtagtttgcaattacagttcgaatcatcggaagaagcagataagggaaatagccacagtaaaaaaatgcttaaagcacttctaagtgagggtgaaagcatctgggagatcactgagaaaatactaaattcgtttgagtatacttcgagatttacaaaaacaaaaactttataccaattcctcttcctagctactttcatcaattgtggaagattcagcgatattaagaacgttgatccgaaatcatttaaattagtccaaaataagtatttgggagtaataatccagtgtttagtgacagagacaaagacaagcgttagtaggcacatatacttctttagcgcaaggggtaggatcgatccacttgtatatttggatgaatttttgaggaattctgaaccagtcctaaaacgagtaaataggaccggcaattcttcaagcaataaacaggaataccaattattaaaagataacttagtcagatcgtacaataaagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagagtgaaattctggaaatctggaaatctggttttgtattcttgttattcttctttttgttattacatatataacttgttacttttttaaaaaaatctttgtttattttataaatatataaaactaaatttaagaaaaagagaaaaatgttttatttgagagattgatattttacttgaatttagcttagcttttataaagtattattatgtaaaaaaacaaaacaaatatacattaaaaagttaagactataaaatagccacccaaggcatttctatatcttgttgttgttgttttcatcttctgtatcagaggaacttattttattattttcgtcacgggtattttctcttgtttgatgattcatcccattcattccatcataaaatgtcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactgactcgagttattatggacatggcatagacatatacaaagcttgttcaccatcggaagcagtaccatcgtataaagcagtatccaaaccacacaaagtgaaacccattcttctataagcatgaatagctggagcattaacattggtaacttccaaccacaaatgaccagcacctctttctctggcgaattcagtagccaaacccatcaaagctctaccaacaccatgacctctatgttctggagcaacttcaatatcttcaacagtcaatcttctgttccaaccagaataagaaacaacaacgaaaccagccaaatcaccatcatcaccataagcaacgaaagttctagaatctggatcaccatcttcaccagcatcggattcatcatcggattcatcatctgggaaaaccttagtcaatggtggatcaactggaacttctctcaaagtgaaaccatcaccagtagcagtaactctaaaaacagtatcggtagtgaaagaaccatccaaagcttcaatagcttcagcatcacctggaacagaagttctgtatctataagcagtatcatccaaagtagtagacataattgtaggatccggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggcaagcttgatgtgcggccgcaagtgattagacttagtccgttcaaatcaagcacaactctgttcattgtttcaacaagaattaattcaaaaacaggttcggtgcataatttgcaaaaaaatattgcagcttctgtggctcgaacacagtacctccagatttcaggtttgaaatacttcagtctgacgctctcccagatgagctaaagctgcaataagaaaacccacgccgggattcgaacccggaatcctttgattagaagtcaaaagcgataaccatttcgccacgcaggcctacttgatgggtttgtaaatggtctactttttcagacctaacagaaattttaatgaaagtcatattcttatacaataaaactgtgtcataaaagcagatattcgactttcgtagattatataggacccaagaactaaaatttaatgccatattatgcatttttaatctgtaaaagtgttgtttccaacctatcacaagtacgttcttgtaacttgtgtttgtagggttgcaaatgaatcataacaacatctcaacagaacatgtatagcaaagcttagtataaaatcagtgttttgagaggcaatccaagaatgtttacatcaaagtttcaataaatatcgaccgaaactgaaaatctttttaggttattgttcacttttttgtaaatatttaaacattttttggacctaaaaaaatacaaacaccaattacgtaccaagaagcatctaatcaactcccagatcaccactatacatttaaaagtcattggtcaataactatactcgagtattgcctcatcaaagaaacaatcaaatattatagatactcactccatcacgtgataatttcactggtatggaaaagtggaaaattttataaaaaaaaatttgatgcctttggcatagctgaaacttcggcccaataggattggagaatatgttttcgcagcgttcttacaattaaattgtggtggaagttcgagacttgcgtaaactatttttaatttggagacggaattccgtctcgttttagagctagaaatagcaagttaaaataaggctagtccgttatcaacttgaaaaagtggcaccgagtcggtgctttttttctcgagtttttttatcgagtgtttaaggataatgataactgaagagaagaattagttttgccgccaccgcgggaagttcctatactttctagagaataggaacttcacgccgggtttgcctctgattaaataaaaaaaagctggtgctttttttttcttttataggaacatcttgaatatatgaactaattaaatgataattttttacccatctttactcttaatcactgagctgcagtcaaagaaaaagggatacagcacctggtgaagagatgaacggagactaacttagacgcgttgattctttttaattgcacattttattaatcgatgctaacgtctatttacatatattctttagagatattatctagggcttcaaataatctctggacagcaataaaagtctcttcaaaagtattgtataacggcaatggggctaatctgattacatctggtcttctttcgtcacagattatagcatgatcatgcaagtacgcattaactcgttccatgacgttcttgtccttttcatcgaaatgcggttgaaacataatggacaattgacatcctctttcagctggattcaaaggagttaaaattttaaacccaaatttggagtttgatgtactggattgtggtatgtaatacttggaattcgtcaatagatcctgtaaaaattgagtcaaagcaacacttttttcacgaagtttagatactccacccactttagcatacacttccaatgacgacttcacagcaacaacatcaagaacagaaggatttgactgtctgtaagaaagagccgagtttattggatcaaactcttctaacattttgaatcgttcttgggagttattgccccaccaaccagctagtctaggaacgaaactgcttttcttgttctctatggtgtatttttcatgcacaaaaatcccacctatggctccaggtcccgagtttaaatatttgtaggaacaccaagcagcaaaatctactccccaatcatgtaaatttaatgggacattcccaactgcatgggcaagatcccacccaactttaatttgttggctcttttccttagcgtatttagttatttcctctatcttgaaaaattgaccagtgtagtattggataccaggaaaacacactagagccaattcatccaggttctcatctatagccttgattattctttctgttttaatataagtttcaccaggttgaacttccaattgaatcaaatgtttctcgtcgtatccgaacaatttaacaatgttcaaaaatgcatagtagtcagaaggaaatgcttgtttttcaaataaaattttggttcttttcccctcaggtttgtaaaaatggatcaacaatgcattcaagtttgctgttaaagaacccataactgcaacttcgttttcctttgcaccaacaatgggggctattaatggtaataagggtaaatcgatgtctacccacggtgttaacagtttgtcaggatgattgaaatgagactcaacccctcgttcaacccatgcatttaattcatcattgatagctttctttgtattcttaggcatcaacccaagagagtttccacataaataaatagactcagttgatgactcatatttattatttttgatacctaatgatccaaaagttggtatggcaaactcatttttaaaagttgggaactttttgtccaatttctttgcctcggctaatgacatctgataataaaatggggttggagtagttggtggtataaccggagagatagaattgaagaaaaaaatcggaaacaacaaaaaaagttgataccctgtattatgtgggagataattgcgaatggtggaaaaaaaaaagacgccattgagtctcaacaacaattctgtcagctgaagagctttacaatcgagaaactatgattcattccgttttaatatgtatgtgtttagtaaactcatgaattttatttgtggtctactttagtactaacataatcattggatagtcaataatgatggtcttccgagactaatgaaattctataccaaagtcgatattccaacacagaaattgctcttgcaacaagtgcacctgttgatatctagagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 28 - DNA, 18691 bp, Artificial Sequence
  Plasmid
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacctttatcagcaagtagaaaacaaccaaagctcttgaaattgtgcaatgaagatttcatcaaacataaaaccattatgaacgcttggaagttgttacaacgaagaagaataacccaacaatctgaaaaattgtctaagcaatataaaagcattgtcaatgccatggaagatttgaagcaaacaagtcccgaattgttcgaagctgcaaatgctaaaaaccctaaacgtttcactaccttcccaatagagatgagagtgcctaccgattatccacctaacaagccatggacttacaactttgttccttcaaaaacccatcattagactgggttcagatgtaaatagatattatattataaatgtacataatcgaatagattgttattatttgttcaactcgtcctaatcctccaatactctcgcctttctttttctactaggtgtgccactactaccactgggcgtctcgttcttctcgatactattagctttacttcctgcactagcagtggttggatcaacagaatcttcataatcatcaaaatcgtcttttgaagaccccccgtttgatgtatggccctgtcttttcatcaaactttttatatagttgactgaactgaggctaaatatgtgatcatcttcactatagacaatctttctcttatttgcaccaccgccaccactagtctttgagaaattctcaaaaccttttacgatattaccaagcgggctctcttcgaaataatctatctctttttgatatatcgaatcctctagcgtggttagctttctagttagttcttgcttcttaagaatttgctggattagtttatttttcaattcaacgtatttctcagagtcatctttagattttgatgaagatgtgcgttcattcgctatatccttcttggtcgtgtcttttcgatcctccttggctggcactgaactcgtcttttttggcgttgctgttccagacagacttatctcattagatttggaacttgtgggtttaacatcatttgtatctttagtagacatgattgtgcaataccgtgattatttgttttgaaaggtctgtcatatttctatcaatttcaaaacaaaatgttcatcagaaaaaagccaaaaatgtctcttctagtttcttagtggtgtcgcataatacacaatgtcgctcaacaatccacattcccggcgcatagctcaaatcacatgactacagctaacaattacacaaaaaaaattctctttttgatgtagcaactatcttcaactaaaacattttctccttcggcccatgattgtcctccgggtcgacagcaagccgttacaattgagatggaaagcgacctaccttcactcgataaggtgcttaattgtacttcatataaatctggcccggatctaaacaaatgagttccattaagccgtgggttctcaattagggtttttgtttttgatttagaaaaaagagatcaagatttgtttacaggtgatgcctttttttagaacttatgcgttgcaaaagttgactaacgatttctataaggtgatccacactaattatacaaacgtacaaacagacatacttttcctgcgttcacctgatgttggccagatttctctcttcattgcatagaacataaccacactagggcaacagaaaaaaaaaaaaaaagtgcatcgggaagttgtgttccattcattatatgtctactactgcatatgagtagcccacccaccaccaccatagtaagtttttgtgtatgcgcgccgtcaggttatttcatttctgaatttttcaaccaccttactccctttattgttgattgacaattttgctcacagtaagatcttttagactccaattaatataaaataagtctgattttccaattcctgttttttctttttttttctgtttctatttctttccttttctcccttttttttaattcttcattcaatcatcaattgataattcaggaatattacaacccgggatggataaaaagtatagtattggtttagctattggtactaactctgtgggttgggcagttatcaccgacgaatataaagttccatcaaagaaatttaaggtgttaggtaacactgacagacactcaataaaaaagaatcttatcggtgctcttttgttcgactccggtgaaactgccgaggctacacgtttaaaaagaacagcaagaagaagatatacccgtagaaaaaatagaatatgttatttacaagaaatcttttctaatgaaatggctaaagttgatgattcctttttccatagattggaagagtcatttttggttgaagaagacaaaaagcatgagagacatccaatctttgggaatatagttgatgaagtggcttaccatgaaaaatatcctaccatttatcatttaagaaagaaattggtagattcaactgataaagctgaccttagattaatctatttagcacttgcccatatgattaaatttagaggtcattttttgattgaaggtgatttgaacccagataattctgacgtggataaattatttattcaattagtccaaacctacaaccaattatttgaggaaaatccaattaatgctagtggtgtcgatgccaaagctatattatcagccagattatcaaaatctagacgtttggaaaatttgattgcccaattgccaggagaaaaaaagaatggattatttggaaacttgatcgcattatcattgggtttgacaccaaattttaaatctaattttgatttagctgaagatgctaaattacaattatcaaaagacacctatgacgacgatttggacaatttacttgctcaaattggtgatcaatatgcagatttgttcttagctgctaaaaacttatctgatgctattttgttgtctgatattttgagagtgaacacagaaataaccaaagctccattatcagcatctatgatcaaacgttatgatgaacaccatcaggatttgactttattgaaagctttggtgagacaacaattgccagagaagtataaagaaatctttttcgatcaatctaaaaacgggtatgcaggttatattgatgggggtgcctcccaagaggaattttacaaatttataaaacctattttagaaaagatggatgggactgaggaacttttggtcaaattgaacagagaagatttgttacgtaaacagagaacttttgataatggtagtatacctcaccaaattcatttgggtgagttgcatgcaattttaagaagacaagaagatttttatccatttttaaaagataatagagaaaaaatcgagaaaattttaacctttagaattccatactatgttgggcctttggctagaggtaattcaagatttgcctggatgacacgtaaatcagaagaaactattaccccttggaattttgaagaggttgttgataaaggagcatcagcacagagttttattgaaagaatgaccaatttcgataaaaacttaccaaatgaaaaagttttaccaaaacattccttgttatacgaatattttactgtttacaatgaacttacaaaggttaaatatgttactgaaggtatgcgtaagccagcctttttatctggagaacagaaaaaggcaatagttgatttattgtttaaaacaaatagaaaagttactgttaaacaattaaaagaagattactttaagaaaattgaatgttttgattcagttgaaatcagtggtgttgaagacagatttaatgctagtttaggaacttaccatgatttacttaaaattatcaaagataaagatttcttggataacgaagaaaatgaagacattttagaagacattgttttaaccttaactttattcgaagatagagagatgattgaagaacgtttgaagacttatgcacatttgtttgacgataaagtgatgaaacagttgaaaagaagacgttatactggatggggtagattgtctcgtaaattgatcaatggaattagagataaacaaagtggtaaaactatcttggactttttgaaatctgacggatttgctaatagaaatttcatgcaattgatccacgacgatagtttgacatttaaagaagacatccaaaaggcccaagtgagtgggcaaggtgattcattacatgaacatattgcaaatttagccggatctcctgctattaagaaagggatattacaaactgttaaagttgtggatgaattagtgaaagtaatgggaagacataaacctgaaaacattgtcattgagatggcaagagaaaatcaaactacacaaaaaggacagaaaaatagtagagaacgtatgaaaagaatagaagagggtattaaagaattgggtagtcaaatattgaaagaacacccagtggaaaatacccagttgcaaaatgaaaaattatatctttactaccttcaaaatggacgtgatatgtatgttgatcaggaattagatataaatagactttcagattatgatgtagatgcaatagttccacaatctttcttgaaagatgattccatagacaataaagtattaactagaagtgataaaaatagaggtaaaagtgataatgtcccaagtgaggaagtcgtcaaaaagatgaaaaattactggcgtcaacttttgaatgctaaattaattactcaaagaaaatttgataatttgactaaagcagaaagaggtgggctttctgaattagataaagccgggttcattaaaagacaattggtcgaaactagacaaattactaaacatgttgcccaaattttagattcccgtatgaacactaagtatgacgaaaatgataagttaatacgtgaggttaaagtcattactttaaaatcaaaacttgtctctgatttcagaaaggatttccaattctataaagttagagaaattaataattatcatcatgctcatgatgcatatttgaatgctgtagttggaactgctttaatcaagaaataccctaaattagaatctgaatttgtatatggtgattacaaagtctatgatgttagaaagatgattgctaaatcagaacaagaaattggtaaagctacagctaaatacttcttttactctaacattatgaatttctttaaaacagaaattactttggcaaacggtgaaattagaaaaagacctcttattgaaacaaatggtgagactggagagatagtttgggacaaagggcgtgatttcgctactgttagaaaagttttatcaatgccacaagttaacattgtaaagaaaacagaggttcaaactggtggtttctcaaaagaaagtattttgcctaaaagaaatagtgataaattgattgccagaaaaaaggattgggatccaaagaaatatggtggtttcgactcaccaaccgtagcctattctgttttggttgtggcaaaggttgaaaagggtaaaagtaaaaagcttaaatcagtaaaagaacttttgggtattacaataatggaaagaagttcctttgaaaagaaccctattgattttttggaagctaaaggttataaggaagtaaagaaggacttaataatcaaattgcctaaatattctttatttgaattagaaaatgggagaaaaagaatgttggcttctgctggagaattgcaaaagggtaatgaattagcattgccttccaaatatgttaacttcttgtatttagcttcacactatgaaaagttgaaagggtcaccagaagataacgagcaaaaacaattatttgttgaacaacacaaacactacttagatgagattatagaacaaattagtgaattcagtaaaagagtgatattagctgatgcaaatttagataaagttttgtcagcctataacaaacatagagataagccaattagagaacaagcagaaaacattattcacttatttacccttaccaatttaggagcacctgctgctttcaagtattttgatacaacaattgatcgtaaaagatatacctcaacaaaagaagtcttagacgccaccttaattcatcaatcaatcactggattgtatgagacaagaattgatttgtctcaattgggtggtgatgaaggggctgatcctaagaagaaaagaaaagttgatccaaagaaaaagcgtaaggtggatcctaagaaaaagagaaaggttatgtatgcgacagcccatacaattaaacaacaacaacaacaacaacaacaacatccaccaccacctttaaacggtggactacatgcaagtggggctcctccaaattcccatgaagcagcagctattgctcagcaacaacaacaacagcagcaacaccacaatggtcctggtatgattgttgccgcagctgcagcttctgctaaccaacaagctgtccaagccagagcccaacaacaacaacagcagcaacaacagcgattacctagttcagctgctcttaatgaaactacagtatcaacttggttagccattggttcattagccgagagtttaggtgacattgaacgtgcgacagcttcttacaattccgctttgagacattcaccaaataacccagatattttagtcaaaatagcaaatacataccgttcaaaagatcagtttcttaaggctgctgaattgtatgaacaagctcttaatttccatgttgagaatggtgaaacttggggattattgggtcattgttacttgatgttggataatttgcaaagagcttatgctgcttatcaacgtgcattgttttacttggaaaaccctaacgttccaaaattgtggcacggaattggtattttatatgacagatatggctcattagaatatgctgaagaagcctttgtgagagttttggatttggatccaaatttcgacaaggctaatgaaatttatttccgtttagggatcatttataagcatcaaggtaaactacaaccagcattagaatgtttccaatacattttgaataatccaccacacccattaactcaaccagatgtttggtttcaaattggttcagtgtatgaacaacaaaaggattggaatggtgctaaggatgcttatgaaaaagtgttacagattaatcctcatcacgctaaagttttgcaacaattgggatgtctttattcccaagcagaatcaaatccatcaacaccagctaatggtgctgcaccaccacataagccattccaacaagatttgaccattgctttaaaatatttgaaacaatctttggaagttgatcaaagtgatgctcattcatggtactatttgggtagagtagaaatgattagaggtgatttcactgctgcttatgaagctttccaacaagctgtcaatcgagatgcaagaaacccaactttctggtgttcaattggtgttttgtactatcaaataagccaatatcgtgatgcattggatgcttataccagagccattagattaaatccttatatcagtgaagtatggtatgatttggggactttgtatgagacttgtaataatcaaattagtgatgcattggatgcatatagacaagcagaaagattggatccaaataatcctcatataaaggcaagattagaacaattgacaaagtatcaacaagaaggtaatactcacccacctcaaccaccgccaagttctcaacaacctagattacctcaaggaatggttttggaaagtactcaacaacaacagcaacaacaaccaccaccacctccacaacaacaacaacaacaacttcaacaccaactgcaactgcaacctcaaccacagcaaccacctcaaacccaatcacaaccactgttacttcaacaccaatcttcattgcctcctcaacaaatccaaccattacatcaacaagctgcaaagcctttagtgaatcaacaacaaagtccaccaccacctcacttgatgaacttgggacaaccggggcaacaaccacaacaattgccaccacatcttccaccacatacccagcaaccttctcaaattcaagaaaagcctccaactcaagaacaaccacattatcaaccacctccacctccacaacatcaacagcaatcgcaatcgcaaccgcaacctccacaccaacctcaacacactcaaaatcaactgcctcaattagctcaattgccaccacaccattctaatcctccagctaagccacatggtgcacctcaacaaagaactggtttaccggatttattacacaactctgctaatatcatatcagctccatcacaagtacctcaaccacaacaacaatatcaacaaccacatattgcacctgttagacaagaacaagttaaccatgttccttcaatttatctggctcctagaccaactgagacaacacttcctcaaatcaacaacccaaatgagtcaaccacaacacaagttccacaactcaaaaaggaggaacctaaaccagaggctactgtttctgctccagttcctgaggctattaaagttcaagatcaagtgacaatccaggagtcagcaccagcagcagcagcagcagtgtcagcaccagcttctgctccagttggtgatataaaaacagatactgtatctactactacacctgctacttcaaccactgcagatgctgtgccagtatctgtgtctcaagttggtgaagcaccaaatgttgttcaagagaagaaagttccggacaccgagcagatcgtttcacaagttgaaaaacccgtggagtcacaaccagaagttacaccagctccaacaccagctccagctcttgcaacagcaccaactgaacctgcacctactgataaggacgttgtaatggctccaagtaaaagtgcaacacctgttcctcaaagtattgtggaacagaacaccagagtatctgaagctacaaaggcaccagaatccaatggtaaacatgatttagaagacaagaatgatgaagaaaaaattttaaagaggccaactgttgaaacgactactgaatctgtaccagttaaccaacctgttgagaaagaaaatgaaaaagttgaggttccaccgccactggaacaaccaagttcagaaaagagagaaaaagaagtcaacggatcaattaagaaaccattggaaaatgaaagtaaggttgatattcctcaattctcatcaaatatcacagctcaaaatgaagaagcaaaatctggagaagaaactaaaaaagatacaaccaagacaagtccagcaaaacaaggggaagttaaggaagtaataccatcatctacagaaactgtatcaaaaccagatgttgaaaaagacaataaagagaaagacaaagatgaagatgaagtgatggctgatgaagatgacgtcaaaaaagatgaaaatccagaacctccaatgagaaagattgaagaagatgaaaattatgatgatgaatagtaatgaagatccatcgacctgccgccaagctaattccgggcgaatttctgtcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccgggccccccctcgaggaagttcctatactttctagagaataggaacttcggatccactagttctagatttttgcaagcatttaaatattgccaagtaaaaacttcaaattttctttccccttggaactttgactttatttttttgacagattattttgacacacacacaccaaatgtgttaccccttaaaacaaaaaaacacttttttacaatttcttggtatccagaatcattctaagcatcattcaattataatttcaatccaaaaaagtagttttagtttgacttgaaacgtcaacaaacacaaatttcaaatcataacctctcctgttgcctgtcaacaacacaccataaggagaaggaataggaggaggaggagatagaaacttgcacggcaccacaaaacacaaaattgatttcaaccaatacggtgacaacaacaatagatttccgatagaaataatgattatcggaataagctagctttgctttgctttgctttgctttttgacttgctctaatttttcgaaaataataatggagaaaagttcaaggtgtttaatgcatcaactaaaacagaaaataatacattagactaaacttttaatctttctagtaccaataattcacgcgtgcgttttaatcccaatcatgaaatgaagaagttatttccctttttctttcatcaaaaaagaactaaattattttttaaattttagtaaacaaaacctggaaatcggggaaaccgggggaggggggcagaaggtgaaacgggtaatattgataaatttaatctataattgataaagttaaatttaaattgatttgaattgatttgaattgaatgaaatgcatttgaataaacggcatcaaactaaaaaaatatagatcacattcatagtaaaacgataacaaagaacaccacaatttatagcaatgataataaacatctaaaaagaaaagggtacgagaaggagaatgaaaaaaaacaataagctagttcttaatctgttcagatatctaatttcaaaaaaaagaatagtataaaaggatagttgattcctcttggttgttgaaaatttgaataatatcaatcaattaatcaatcaaataacaacaacccactagacatcaccattgtcgacatgccacaatttgatatattatgtaaaacaccacctaaggtgcttgttcgtcagtttgtggaaaggtttgaaagaccttcaggtgagaaaatagcattatgtgctgctgaactaacctatttatgttggatgattacacataacggaacagcaatcaagagagccacattcatgagctataatactatcataagcaattcgttgagtttcgatattgtcaataaatcactccagtttaaatacaagacgcaaaaagcaacaattttggaagcctcattaaagaaattgattcctgcttgggaatttacaattattccttactatggacaaaaacatcaatctgatatcactgatattgtaagtagtttgcaattacagttcgaatcatcggaagaagcagataagggaaatagccacagtaaaaaaatgcttaaagcacttctaagtgagggtgaaagcatctgggagatcactgagaaaatactaaattcgtttgagtatacttcgagatttacaaaaacaaaaactttataccaattcctcttcctagctactttcatcaattgtggaagattcagcgatattaagaacgttgatccgaaatcatttaaattagtccaaaataagtatttgggagtaataatccagtgtttagtgacagagacaaagacaagcgttagtaggcacatatacttctttagcgcaaggggtaggatcgatccacttgtatatttggatgaatttttgaggaattctgaaccagtcctaaaacgagtaaataggaccggcaattcttcaagcaataaacaggaataccaattattaaaagataacttagtcagatcgtacaataaagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagagtgaaattctggaaatctggaaatctggttttgtattcttgttattcttctttttgttattacatatataacttgttacttttttaaaaaaatctttgtttattttataaatatataaaactaaatttaagaaaaagagaaaaatgttttatttgagagattgatattttacttgaatttagcttagcttttataaagtattattatgtaaaaaaacaaaacaaatatacattaaaaagttaagactataaaatagccacccaaggcatttctatatcttgttgttgttgttttcatcttctgtatcagaggaacttattttattattttcgtcacgggtattttctcttgtttgatgattcatcccattcattccatcataaaatgtcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactgactcgagttattatggacatggcatagacatatacaaagcttgttcaccatcggaagcagtaccatcgtataaagcagtatccaaaccacacaaagtgaaacccattcttctataagcatgaatagctggagcattaacattggtaacttccaaccacaaatgaccagcacctctttctctggcgaattcagtagccaaacccatcaaagctctaccaacaccatgacctctatgttctggagcaacttcaatatcttcaacagtcaatcttctgttccaaccagaataagaaacaacaacgaaaccagccaaatcaccatcatcaccataagcaacgaaagttctagaatctggatcaccatcttcaccagcatcggattcatcatcggattcatcatctgggaaaaccttagtcaatggtggatcaactggaacttctctcaaagtgaaaccatcaccagtagcagtaactctaaaaacagtatcggtagtgaaagaaccatccaaagcttcaatagcttcagcatcacctggaacagaagttctgtatctataagcagtatcatccaaagtagtagacataattgtaggatccggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggcaagcttgatggaagttcctatactttctagagaataggaacttcagatccactagttctagagcggccgccaccgcgggtttgcctctgattaaataaaaaaaagctggtgctttttttttcttttataggaacatcttgaatatatgaactaattaaatgataattttttacccatctttactcttaatcactgagctgcagtcaaagaaaaagggatacagcacctggtgaagagatgaacggagactaacttagacgcgttgattctttttaattgcacattttattaatcgatgctaacgtctatttacatatattctttagagatattatctagggcttcaaataatctctggacagcaataaaagtctcttcaaaagtattgtataacggcaatggggctaatctgattacatctggtcttctttcgtcacagattatagcatgatcatgcaagtacgcattaactcgttccatgacgttcttgtccttttcatcgaaatgcggttgaaacataatggacaattgacatcctctttcagctggattcaaaggagttaaaattttaaacccaaatttggagtttgatgtactggattgtggtatgtaatacttggaattcgtcaatagatcctgtaaaaattgagtcaaagcaacacttttttcacgaagtttagatactccacccactttagcatacacttccaatgacgacttcacagcaacaacatcaagaacagaaggatttgactgtctgtaagaaagagccgagtttattggatcaaactcttctaacattttgaatcgttcttgggagttattgccccaccaaccagctagtctaggaacgaaactgcttttcttgttctctatggtgtatttttcatgcacaaaaatcccacctatggctccaggtcccgagtttaaatatttgtaggaacaccaagcagcaaaatctactccccaatcatgtaaatttaatgggacattcccaactgcatgggcaagatcccacccaactttaatttgttggctcttttccttagcgtatttagttatttcctctatcttgaaaaattgaccagtgtagtattggataccaggaaaacacactagagccaattcatccaggttctcatctatagccttgattattctttctgttttaatataagtttcaccaggttgaacttccaattgaatcaaatgtttctcgtcgtatccgaacaatttaacaatgttcaaaaatgcatagtagtcagaaggaaatgcttgtttttcaaataaaattttggttcttttcccctcaggtttgtaaaaatggatcaacaatgcattcaagtttgctgttaaagaacccataactgcaacttcgttttcctttgcaccaacaatgggggctattaatggtaataagggtaaatcgatgtctacccacggtgttaacagtttgtcaggatgattgaaatgagactcaacccctcgttcaacccatgcatttaattcatcattgatagctttctttgtattcttaggcatcaacccaagagagtttccacataaataaatagactcagttgatgactcatatttattatttttgatacctaatgatccaaaagttggtatggcaaactcatttttaaaagttgggaactttttgtccaatttctttgcctcggctaatgacatctgataataaaatggggttggagtagttggtggtataaccggagagatagaattgaagaaaaaaatcggaaacaacaaaaaaagttgataccctgtattatgtgggagataattgcgaatggtggaaaaaaaaaagacgccattgagtctcaacaacaattctgtcagctgaagagctttacaatcgagaaactatgattcattccgttttaatatgtatgtgtttagtaaactcatgaattttatttgtggtctactttagtactaacataatcattggatagtcaataatgatggtcttccgagactaatgaaattctataccaaagtcgatattccaacacagaaattgctcttgcaacaagtgcacctgttgatatctagagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 29 - DNA, 17793 bp, Artificial Sequence
  Plasmid
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacctgccactactaccactgggagtttcgttcttctcgatactattagctttacttcctgcactagcagtggttggatcaacagaatcttcataatcatcaaaatcgtcttttgaagaccccccgtttgatgtatggccctgtcttttcatcaaactttttatatagttgactgaactgaggctaaatatgtgatcatcttcactatagacaatctttctcttatttgcaccaccgccaccactagtctttgagaaattctcaaaaccttttacgatattaccaagcgggctctcttcgaaataatctatctctttttgatatatcgaatcctctagcgtggttagctttctagttagttcttgcttcttaagaatttgctggattagtttatttttcaattcaacgtatttctcagagtcatctttagattttgatgaagatgtgcgttcattcgctatatccttcttggtcgtgtcttttcgatcctccttggctggcactgaactcgtcttttttggcgttgctgttccagacagacttatctcattagatttggaacttgtgggtttaacatcatttgtatctttagtagacatgattgtgcaataccgtgattatttgttttgaaaggtctgtcatatttctatcaatttcaaaacaaaatgttcatcagaaaaaagccaaaaatgtctcttctagtttcttagtggtgtcgcataatacacaatgtcgctcaacaatccacattcccggcgcatagctcaaatcacatgactacagctaacaattacacaaaaaaaattctctttttgatgtagcaactatcttcaactaaaacattttctccttcggcccatgattgtcctccgggtcgacagcaagccgttacaattgagatggaaagcgacctaccttcactcgataaggtgcttaattgtacttcatataaatctggcccggatctaaacaaatgagttccattaagccgtgggttctcaattagggtttttgtttttgatttagaaaaaagagatcaagatttgtttacaggtgatgcctttttttagaacttatgcgttgcaaaagttgactaacgatttctataaggtgatccacactaattatacaaacgtacaaacagacatacttttcctgcgttcacctgatgttggccagatttctctcttcattgcatagaacataaccacactagggcaacagaaaaaaaaaaaaaaagtgcatcgggaagttgtgttccattcattatatgtctactactgcatatgagtagcccacccaccaccaccatagtaagtttttgtgtatgcgcgccgtcaggttatttcatttctgaatttttcaaccaccttactccctttattgttgattgacaattttgctcacagtaagatcttttagactccaattaatataaaataagtctgattttccaattcctgttttttctttttttttctgtttctatttctttccttttctcccttttttttaattcttcattcaatcatcaattgataattcaggaatattacaacaacccggggagcatgatatcgtttgatatttttgtctagtaccatctgtaccattacacttaaattatctttatatctgtctaactcgactgtctggatttcattgatgtagtcgtatgcatcgttagttccaaaaaatattgtcatcaatttgatattggtttccgactctaaaatttttggaagaatttgtctagcgtgctctgagttgtagccactgaaaccacggttaataacatccaattttcggatatacacattctgtaatgctggatgaaagccatactgggtacaactaaactgggtgatggagtcaccgaacaacacaaatttaccgtattccatgattgctatggttgagaattttttttttttcttgtcccacgccatttttcaaattatgcagttgagaatgttagtttttgtgtacaccccgttcgctgaatatttcggaataattcaaagattggggagtgggggaggcgatagacgaagacacggtataaaaatgggcaaaattttccccaactttttgcagtggtttaactaataatcaactacaatgcccgggatggataaaaagtatagtattggtttagctattggtactaactctgtgggttgggcagttatcaccgacgaatataaagttccatcaaagaaatttaaggtgttaggtaacactgacagacactcaataaaaaagaatcttatcggtgctcttttgttcgactccggtgaaactgccgaggctacacgtttaaaaagaacagcaagaagaagatatacccgtagaaaaaatagaatatgttatttacaagaaatcttttctaatgaaatggctaaagttgatgattcctttttccatagattggaagagtcatttttggttgaagaagacaaaaagcatgagagacatccaatctttgggaatatagttgatgaagtggcttaccatgaaaaatatcctaccatttatcatttaagaaagaaattggtagattcaactgataaagctgaccttagattaatctatttagcacttgcccatatgattaaatttagaggtcattttttgattgaaggtgatttgaacccagataattctgacgtggataaattatttattcaattagtccaaacctacaaccaattatttgaggaaaatccaattaatgctagtggtgtcgatgccaaagctatattatcagccagattatcaaaatctagacgtttggaaaatttgattgcccaattgccaggagaaaaaaagaatggattatttggaaacttgatcgcattatcattgggtttgacaccaaattttaaatctaattttgatttagctgaagatgctaaattacaattatcaaaagacacctatgacgacgatttggacaatttacttgctcaaattggtgatcaatatgcagatttgttcttagctgctaaaaacttatctgatgctattttgttgtctgatattttgagagtgaacacagaaataaccaaagctccattatcagcatctatgatcaaacgttatgatgaacaccatcaggatttgactttattgaaagctttggtgagacaacaattgccagagaagtataaagaaatctttttcgatcaatctaaaaacgggtatgcaggttatattgatgggggtgcctcccaagaggaattttacaaatttataaaacctattttagaaaagatggatgggactgaggaacttttggtcaaattgaacagagaagatttgttacgtaaacagagaacttttgataatggtagtatacctcaccaaattcatttgggtgagttgcatgcaattttaagaagacaagaagatttttatccatttttaaaagataatagagaaaaaatcgagaaaattttaacctttagaattccatactatgttgggcctttggctagaggtaattcaagatttgcctggatgacacgtaaatcagaagaaactattaccccttggaattttgaagaggttgttgataaaggagcatcagcacagagttttattgaaagaatgaccaatttcgataaaaacttaccaaatgaaaaagttttaccaaaacattccttgttatacgaatattttactgtttacaatgaacttacaaaggttaaatatgttactgaaggtatgcgtaagccagcctttttatctggagaacagaaaaaggcaatagttgatttattgtttaaaacaaatagaaaagttactgttaaacaattaaaagaagattactttaagaaaattgaatgttttgattcagttgaaatcagtggtgttgaagacagatttaatgctagtttaggaacttaccatgatttacttaaaattatcaaagataaagatttcttggataacgaagaaaatgaagacattttagaagacattgttttaaccttaactttattcgaagatagagagatgattgaagaacgtttgaagacttatgcacatttgtttgacgataaagtgatgaaacagttgaaaagaagacgttatactggatggggtagattgtctcgtaaattgatcaatggaattagagataaacaaagtggtaaaactatcttggactttttgaaatctgacggatttgctaatagaaatttcatgcaattgatccacgacgatagtttgacatttaaagaagacatccaaaaggcccaagtgagtgggcaaggtgattcattacatgaacatattgcaaatttagccggatctcctgctattaagaaagggatattacaaactgttaaagttgtggatgaattagtgaaagtaatgggaagacataaacctgaaaacattgtcattgagatggcaagagaaaatcaaactacacaaaaaggacagaaaaatagtagagaacgtatgaaaagaatagaagagggtattaaagaattgggtagtcaaatattgaaagaacacccagtggaaaatacccagttgcaaaatgaaaaattatatctttactaccttcaaaatggacgtgatatgtatgttgatcaggaattagatataaatagactttcagattatgatgtagatgcaatagttccacaatctttcttgaaagatgattccatagacaataaagtattaactagaagtgataaaaatagaggtaaaagtgataatgtcccaagtgaggaagtcgtcaaaaagatgaaaaattactggcgtcaacttttgaatgctaaattaattactcaaagaaaatttgataatttgactaaagcagaaagaggtgggctttctgaattagataaagccgggttcattaaaagacaattggtcgaaactagacaaattactaaacatgttgcccaaattttagattcccgtatgaacactaagtatgacgaaaatgataagttaatacgtgaggttaaagtcattactttaaaatcaaaacttgtctctgatttcagaaaggatttccaattctataaagttagagaaattaataattatcatcatgctcatgatgcatatttgaatgctgtagttggaactgctttaatcaagaaataccctaaattagaatctgaatttgtatatggtgattacaaagtctatgatgttagaaagatgattgctaaatcagaacaagaaattggtaaagctacagctaaatacttcttttactctaacattatgaatttctttaaaacagaaattactttggcaaacggtgaaattagaaaaagacctcttattgaaacaaatggtgagactggagagatagtttgggacaaagggcgtgatttcgctactgttagaaaagttttatcaatgccacaagttaacattgtaaagaaaacagaggttcaaactggtggtttctcaaaagaaagtattttgcctaaaagaaatagtgataaattgattgccagaaaaaaggattgggatccaaagaaatatggtggtttcgactcaccaaccgtagcctattctgttttggttgtggcaaaggttgaaaagggtaaaagtaaaaagcttaaatcagtaaaagaacttttgggtattacaataatggaaagaagttcctttgaaaagaaccctattgattttttggaagctaaaggttataaggaagtaaagaaggacttaataatcaaattgcctaaatattctttatttgaattagaaaatgggagaaaaagaatgttggcttctgctggagaattgcaaaagggtaatgaattagcattgccttccaaatatgttaacttcttgtatttagcttcacactatgaaaagttgaaagggtcaccagaagataacgagcaaaaacaattatttgttgaacaacacaaacactacttagatgagattatagaacaaattagtgaattcagtaaaagagtgatattagctgatgcaaatttagataaagttttgtcagcctataacaaacatagagataagccaattagagaacaagcagaaaacattattcacttatttacccttaccaatttaggagcacctgctgctttcaagtattttgatacaacaattgatcgtaaaagatatacctcaacaaaagaagtcttagacgccaccttaattcatcaatcaatcactggattgtatgagacaagaattgatttgtctcaattgggtggtgatgaaggggctgatcctaagaagaaaagaaaagttgatccaaagaaaaagcgtaaggtggatcctaagaaaaagagaaaggttatgtatgcgacagcccatacaattaaacaacaacaacaacaacaacaacaacatccaccaccacctttaaacggtggactacatgcaagtggggctcctccaaattcccatgaagcagcagctattgctcagcaacaacaacaacagcagcaacaccacaatggtcctggtatgattgttgccgcagctgcagcttctgctaaccaacaagctgtccaagccagagcccaacaacaacaacagcagcaacaacagcgattacctagttcagctgctcttaatgaaactacagtatcaacttggttagccattggttcattagccgagagtttaggtgacattgaacgtgcgacagcttcttacaattccgctttgagacattcaccaaataacccagatattttagtcaaaatagcaaatacataccgttcaaaagatcagtttcttaaggctgctgaattgtatgaacaagctcttaatttccatgttgagaatggtgaaacttggggattattgggtcattgttacttgatgttggataatttgcaaagagcttatgctgcttatcaacgtgcattgttttacttggaaaaccctaacgttccaaaattgtggcacggaattggtattttatatgacagatatggctcattagaatatgctgaagaagcctttgtgagagttttggatttggatccaaatttcgacaaggctaatgaaatttatttccgtttagggatcatttataagcatcaaggtaaactacaaccagcattagaatgtttccaatacattttgaataatccaccacacccattaactcaaccagatgtttggtttcaaattggttcagtgtatgaacaacaaaaggattggaatggtgctaaggatgcttatgaaaaagtgttacagattaatcctcatcacgctaaagttttgcaacaattgggatgtctttattcccaagcagaatcaaatccatcaacaccagctaatggtgctgcaccaccacataagccattccaacaagatttgaccattgctttaaaatatttgaaacaatctttggaagttgatcaaagtgatgctcattcatggtactatttgggtagagtagaaatgattagaggtgatttcactgctgcttatgaagctttccaacaagctgtcaatcgagatgcaagaaacccaactttctggtgttcaattggtgttttgtactatcaaataagccaatatcgtgatgcattggatgcttataccagagccattagattaaatccttatatcagtgaagtatggtatgatttggggactttgtatgagacttgtaataatcaaattagtgatgcattggatgcatatagacaagcagaaagattggatccaaataatcctcatataaaggcaagattagaacaattgacaaagtatcaacaagaaggtaatactcacccacctcaaccaccgccaagttctcaacaacctagattacctcaaggaatggttttggaaagtactcaacaacaacagcaacaacaaccaccaccacctccacaacaacaacaacaacaacttcaacaccaactgcaactgcaacctcaaccacagcaaccacctcaaacccaatcacaaccactgttacttcaacaccaatcttcattgcctcctcaacaaatccaaccattacatcaacaagctgcaaagcctttagtgaatcaacaacaaagtccaccaccacctcacttgatgaacttgggacaaccggggcaacaaccacaacaattgccaccacatcttccaccacatacccagcaaccttctcaaattcaagaaaagcctccaactcaagaacaaccacattatcaaccacctccacctccacaacatcaacagcaatcgcaatcgcaaccgcaacctccacaccaacctcaacacactcaaaatcaactgcctcaattagctcaattgccaccacaccattctaatcctccagctaagccacatggtgcacctcaacaaagaactggtttaccggatttattacacaactctgctaatatcatatcagctccatcacaagtacctcaaccacaacaacaatatcaacaaccacatattgcacctgttagacaagaacaagttaaccatgttccttcaatttatctggctcctagaccaactgagacaacacttcctcaaatcaacaacccaaatgagtcaaccacaacacaagttccacaactcaaaaaggaggaacctaaaccagaggctactgtttctgctccagttcctgaggctattaaagttcaagatcaagtgacaatccaggagtcagcaccagcagcagcagcagcagtgtcagcaccagcttctgctccagttggtgatataaaaacagatactgtatctactactacacctgctacttcaaccactgcagatgctgtgccagtatctgtgtctcaagttggtgaagcaccaaatgttgttcaagagaagaaagttccggacaccgagcagatcgtttcacaagttgaaaaacccgtggagtcacaaccagaagttacaccagctccaacaccagctccagctcttgcaacagcaccaactgaacctgcacctactgataaggacgttgtaatggctccaagtaaaagtgcaacacctgttcctcaaagtattgtggaacagaacaccagagtatctgaagctacaaaggcaccagaatccaatggtaaacatgatttagaagacaagaatgatgaagaaaaaattttaaagaggccaactgttgaaacgactactgaatctgtaccagttaaccaacctgttgagaaagaaaatgaaaaagttgaggttccaccgccactggaacaaccaagttcagaaaagagagaaaaagaagtcaacggatcaattaagaaaccattggaaaatgaaagtaaggttgatattcctcaattctcatcaaatatcacagctcaaaatgaagaagcaaaatctggagaagaaactaaaaaagatacaaccaagacaagtccagcaaaacaaggggaagttaaggaagtaataccatcatctacagaaactgtatcaaaaccagatgttgaaaaagacaataaagagaaagacaaagatgaagatgaagtgatggctgatgaagatgacgtcaaaaaagatgaaaatccagaacctccaatgagaaagattgaagaagatgaaaattatgatgatgaatagtaatgaagatccatcgacctgccgccaagctaattccgggcgaatttctgtcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccgggccccccctaactcaagtacaacagatctggaccacctttgattgtaaatagtaataattaccacccttatctaattatttatttaacttatttatttatttattatacatatatacaaatctaataaagtgaaaatctcccccttcacacttcacatatgttaggcgtcatcctgtgctcccgagaaccagtaccagtacatcgctgtttcgttcgagacttgaggtctagttttatacgtgaagaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaattttcatatgagaaccgttatcgataactaaagcagcaacttcttctataaaaatgggttagtatgacagtcatttaaataaggaatttttcagttggcttggtttcaattcaatgttcgttttttttttttcttgctgtgtttgtgtttgtgttgtttatagttgtgtgcactgatcgtcgaaaaaaaaaattcatagtgagccgggaaatctgtatagcccagataacaacacaagtccaaactagaaactcgtcaaacaccaaaagcaatgttgaatcaattgccttgcacaagtacacgtaggaaaacataaaacattgcaattttgaatattgagccttttgtcgtaacattgattgataggattactcaccgaatggttttgaaaccactgccgacagatcaatcaatcaatcaaaaaacgtgaactttgaaaaaggggaagaacagatacattgaagttagccatttccactgatcgtcacaacatatctgataaattactttcaaaattataagctgatgtgtgtgtattattaatgtgacagtaacatcccaaacgagaaatattatctcgacaacaaaaaagtttgatctgaattgaaaatgaagttttcccaccctacccatttgtcatattgaaaccaatcaactgattaatcaatcaattagaattgaagctaaactaaaacataccaccgtccattttgaatgattatatttttttaatattaatatcgagataatgtttctaagaaagaaagaaaaccaggagtgaaaattagaaaaggaaaggaaaggaaaaaaagaaaaatctgaaaatatataaaaaaaaattgtttcgttggcaataaatcttggtgagaacagcgaccgaaagcaaataagaacaaaatatgagtgtattacgttgaacaactaattaacgtgtgtgtatggatctttttttcttttttctctttaaccgactataaacaacaaacatttttgggcagtgcacacactacttaatatacacagcataaattacacgattagaaacaaattagcttattaaaataacctaatcaaaccgaatattttatggtattatgagtaaactatataatataaatagcacacacccacaacaacaacaaaggaaaactaaaaggttttttctttttgaaaagatcgttttctttattattctctagttttgacggcggccgcaagtgattagacttagtccgttcaaatcaagcacaactctgttcattgtttcaacaagaattaattcaaaaacaggttcggtgcataatttgcaaaaaaatattgcagcttctgtggctcgaacacagtacctccagatttcaggtttgaaatacttcagtctgacgctctcccagatgagctaaagctgcaataagaaaacccacgccgggattcgaacccggaatcctttgattagaagtcaaaagcgataaccatttcgccacgcaggcctacttgatgggtttgtaaatggtctactttttcagacctaacagaaattttaatgaaagtcatattcttatacaataaaactgtgtcataaaagcagatattcgactttcgtagattatataggacccaagaactaaaatttaatgccatattatgcatttttaatctgtaaaagtgttgtttccaacctatcacaagtacgttcttgtaacttgtgtttgtagggttgcaaatgaatcataacaacatctcaacagaacatgtatagcaaagcttagtataaaatcagtgttttgagaggcaatccaagaatgtttacatcaaagtttcaataaatatcgaccgaaactgaaaatctttttaggttattgttcacttttttgtaaatatttaaacattttttggacctaaaaaaatacaaacaccaattacgtaccaagaagcatctaatcaactcccagatcaccactatacatttaaaagtcattggtcaataactatactcgagtattgcctcatcaaagaaacaatcaaatattatagatactcactccatcacgtgataatttcactggtatggaaaagtggaaaattttataaaaaaaaatttgatgcctttggcatagctgaaacttcggcccaataggattggagaatatgttttcgcagcgttcttacaattaaattgtggtggaagttcgagacttgcgtaaactatttttaatttggagacggaattccgtctcgttttagagctagaaatagcaagttaaaataaggctagtccgttatcaacttgaaaaagtggcaccgagtcggtgctttttttctcgagtttttttatcgagtgtttaaggataatgataactgaagagaagaattagttttgccgccaccgcgggtttgcctctgattaaataaaaaaaagctggtgctttttttttcttttataggaacatcttgaatatatgaactaattaaatgataattttttacccatctttactcttaatcactgagctgcagtcaaagaaaaagggatacagcacctggtgaagagatgaacggagactaacttagacgcgttgattctttttaattgcacattttattaatcgatgctaacgtctatttacatatattctttagagatattatctagggcttcaaataatctctggacagcaataaaagtctcttcaaaagtattgtataacggcaatggggctaatctgattacatctggtcttctttcgtcacagattatagcatgatcatgcaagtacgcattaactcgttccatgacgttcttgtccttttcatcgaaatgcggttgaaacataatggacaattgacatcctctttcagctggattcaaaggagttaaaattttaaacccaaatttggagtttgatgtactggattgtggtatgtaatacttggaattcgtcaatagatcctgtaaaaattgagtcaaagcaacacttttttcacgaagtttagatactccacccactttagcatacacttccaatgacgacttcacagcaacaacatcaagaacagaaggatttgactgtctgtaagaaagagccgagtttattggatcaaactcttctaacattttgaatcgttcttgggagttattgccccaccaaccagctagtctaggaacgaaactgcttttcttgttctctatggtgtatttttcatgcacaaaaatcccacctatggctccaggtcccgagtttaaatatttgtaggaacaccaagcagcaaaatctactccccaatcatgtaaatttaatgggacattcccaactgcatgggcaagatcccacccaactttaatttgttggctcttttccttagcgtatttagttatttcctctatcttgaaaaattgaccagtgtagtattggataccaggaaaacacactagagccaattcatccaggttctcatctatagccttgattattctttctgttttaatataagtttcaccaggttgaacttccaattgaatcaaatgtttctcgtcgtatccgaacaatttaacaatgttcaaaaatgcatagtagtcagaaggaaatgcttgtttttcaaataaaattttggttcttttcccctcaggtttgtaaaaatggatcaacaatgcattcaagtttgctgttaaagaacccataactgcaacttcgttttcctttgcaccaacaatgggggctattaatggtaataagggtaaatcgatgtctacccacggtgttaacagtttgtcaggatgattgaaatgagactcaacccctcgttcaacccatgcatttaattcatcattgatagctttctttgtattcttaggcatcaacccaagagagtttccacataaataaatagactcagttgatgactcatatttattatttttgatacctaatgatccaaaagttggtatggcaaactcatttttaaaagttgggaactttttgtccaatttctttgcctcggctaatgacatctgataataaaatggggttggagtagttggtggtataaccggagagatagaattgaagaaaaaaatcggaaacaacaaaaaaagttgataccctgtattatgtgggagataattgcgaatggtggaaaaaaaaaagacgccattgagtctcaacaacaattctgtcagctgaagagctttacaatcgagaaactatgattcattccgttttaatatgtatgtgtttagtaaactcatgaattttatttgtggtctactttagtactaacataatcattggatagtcaataatgatggtcttccgagactaatgaaattctataccaaagtcgatattccaacacagaaattgctcttgcaacaagtgcacctgttgatatctagagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 30 - DNA, 26 bp, Artificial Sequence
  sgRNA cloning primer
atttgcaacaatcatacgacctaatg

SEQ ID NO: 31 - DNA, 26 bp, Artificial Sequence
  sgRNA cloning primer
aaaacattaggtcgtatgattgttgc

SEQ ID NO: 32 - DNA, 26 bp, Artificial Sequence
  sgRNA cloning primer
atttgagtttctgctctctcactatg

SEQ ID NO: 33 - DNA, 26 bp, Artificial Sequence
  sgRNA cloning primer
aaaacatagtgagagagcagaaactc

SEQ ID NO: 34 - DNA, 26 bp, Artificial Sequence
  sgRNA cloning primer
atttgaaattagttgttgttggaggg

SEQ ID NO: 35 - DNA, 26 bp, Artificial Sequence
  sgRNA cloning primer
aaaaccctccaacaacaactaatttc

SEQ ID NO: 36 - DNA, 26 bp, Artificial Sequence
  sgRNA cloning primer
atttgatataagaatgaagacaacgg

SEQ ID NO: 37 - DNA, 26 bp, Artificial Sequence
  sgRNA cloning primer
aaaaccgttgtcttcattcttatatc

SEQ ID NO: 38 - DNA, 26 bp, Artificial Sequence
  sgRNA cloning primer
atttgacaagacatgaattcacatcg

SEQ ID NO: 39 - DNA, 26 bp, Artificial Sequence
  sgRNA cloning primer
aaaacgatgtgaattcatgtcttgtc

SEQ ID NO: 40 - DNA, 26 bp, Artificial Sequence
  sgRNA cloning primer
atttgatataatgtgtattacttctg

SEQ ID NO: 41 - DNA, 26 bp, Artificial Sequence
  sgRNA cloning primer
aaaacagaagtaatacacattatatc

SEQ ID NO: 42 - DNA, 26 bp, Artificial Sequence
  sgRNA cloning primer
atttgttggctcaacacttgggcacg

SEQ ID NO: 43 - DNA, 26 bp, Artificial Sequence
  sgRNA cloning primer
aaaacgtgcccaagtgttgagccaac

SEQ ID NO: 44 - DNA, 26 bp, Artificial Sequence
  sgRNA cloning primer
atttgatagcagaaactgccaacaag

SEQ ID NO: 45 - DNA, 26 bp, Artificial Sequence
  sgRNA cloning primer
aaaacttgttggcagtttctgctatc

SEQ ID NO: 46 - DNA, 26 bp, Artificial Sequence
  sgRNA cloning primer
atttgttatgagttacatcaacaacg

SEQ ID NO: 47 - DNA, 26 bp, Artificial Sequence
  sgRNA cloning primer
aaaacgttgttgatgtaactcataac

SEQ ID NO: 48 - DNA, 26 bp, Artificial Sequence
  sgRNA cloning primer
atttggggtgaactatttgttcgccg

SEQ ID NO: 49 - DNA, 26 bp, Artificial Sequence
  sgRNA cloning primer
aaaacggcgaacaaatagttcacccc

SEQ ID NO: 50 - DNA, 23 bp, Artificial Sequence
  PCR sequencing primer
aacaccccccaccaaaaagaatc

SEQ ID NO: 51 - DNA, 21 bp, Artificial Sequence
  PCR sequencing primer
acaagtcatcgactgtgttgg

SEQ ID NO: 52 - DNA, 22 bp, Artificial Sequence
  PCR sequencing primer
aaaacattcagaatttagccag

SEQ ID NO: 53 - DNA, 22 bp, Artificial Sequence
  PCR sequencing primer
atagaaatttaagagcttacgg

SEQ ID NO: 54 - DNA, 23 bp, Artificial Sequence
  PCR sequencing primer
aggttgccatataaacactagcc

SEQ ID NO: 55 - DNA, 26 bp, Artificial Sequence
  PCR sequencing primer
tttgttcttcaatgatgatttcaacc

SEQ ID NO: 56 - DNA, 27 bp, Artificial Sequence
  PCR sequencing primer
cataaattgatgtttacgtgaaagttc

SEQ ID NO: 57 - DNA, 25 bp, Artificial Sequence
  PCR sequencing primer
tcaattgactagatataaactcttc

SEQ ID NO: 58 - DNA, 25 bp, Artificial Sequence
  PCR sequencing primer
tccatcttcataactaacttgtctt

SEQ ID NO: 59 - DNA, 27 bp, Artificial Sequence
  PCR sequencing primer
ttcaatagtttttttctgcgtattgtg

SEQ ID NO: 60 - DNA, 25 bp, Artificial Sequence
  PCR sequencing primer
tcgatccagcaatggaagatagctt

SEQ ID NO: 61 - DNA, 23 bp, Artificial Sequence
  PCR sequencing primer
cttagtctaactttatagttgtc

SEQ ID NO: 62 - DNA, 26 bp, Artificial Sequence
  PCR sequencing primer
attctttctaataacatttcatgcaa

SEQ ID NO: 63 - DNA, 22 bp, Artificial Sequence
  PCR sequencing primer
tgtcattccgtttctccttcta

SEQ ID NO: 64 - DNA, 21 bp, Artificial Sequence
  PCR sequencing primer
gcaaattcaataaccataatg

SEQ ID NO: 65 - DNA, 28 bp, Artificial Sequence
  PCR sequencing primer
ggtatattgcacacgaccatagtgcgaa

SEQ ID NO: 66 - DNA, 20 bp, Artificial Sequence
  PCR sequencing primer
tcacttattttgacttcatc

SEQ ID NO: 67 - DNA, 23 bp, Artificial Sequence
  PCR sequencing primer
ttaaagaaacttcacatcaccaa

SEQ ID NO: 68 - DNA, 22 bp, Artificial Sequence
  PCR sequencing primer
actttgatagcataatatctac

SEQ ID NO: 69 - DNA, 66 bp, Artificial Sequence
  Repair template for mutagenesis
taatggatagcaaaactgttggtattttaggaggttaatgattaggtcgtatgattgttgaagcag

SEQ ID NO: 70 - DNA, 50 bp, Artificial Sequence
  Repair template for mutagenesis
cggtcttgatattcaatctatgtgctgcttcaacaatcatacgacctaat

SEQ ID NO: 71 - DNA, 73 bp, Artificial Sequence
  Repair template for mutagenesis
ttgatgttgatgctttaatcaaagttcaagagaaattaactaaagttgaaatatatccattactacctgaaac

SEQ ID NO: 72 - DNA, 50 bp, Artificial Sequence
  Repair template for mutagenesis
tatcttgaatcaatcttatggtttcaggtaatggatatatttcaacttta

SEQ ID NO: 73 - DNA, 60 bp, Artificial Sequence
  Repair template for mutagenesis
ccaggtgaacttactgtkgttttggggagacccggtgcttaagaattcttgttccacatt

SEQ ID NO: 74 - DNA, 60 bp, Artificial Sequence
  Repair template for mutagenesis
tgtggaaaccataagtgttaacagcaatggtctttaacaatgtggaacaagaattcttaa

SEQ ID NO: 75 - DNA, 60 bp, Artificial Sequence
  Repair template for mutagenesis
aaatagcaaacaaaagatatgacagtcaacacttaataatatagtgagagagcagaaact

SEQ ID NO: 76 - DNA, 50 bp, Artificial Sequence
  Repair template for mutagenesis
aaataatcgttgtgctactggtgaggcatgagtttctgctctctcactat

SEQ ID NO: 77 - DNA, 60 bp, Artificial Sequence
  Repair template for mutagenesis
atatccacacatatacataccatgttgagagaatataaattagttgttgttggaggtgtt

SEQ ID NO: 78 - DNA, 60 bp, Artificial Sequence
  Repair template for mutagenesis
aatcaattgaatggttaaagcggatttaccaacaccaacacctccaacaacaactaattt

SEQ ID NO: 79 - DNA, 60 bp, Artificial Sequence
  Repair template for mutagenesis
atatccacacatatacataccatgttgagagaatataaattagttgttgttggaggttaa

SEQ ID NO: 80 - DNA, 60 bp, Artificial Sequence
  Repair template for mutagenesis
aatcaattgaatggttaaagcggatttaccaacaccgaattcttaacctccaacaacaac

SEQ ID NO: 81 - DNA, 60 bp, Artificial Sequence
  Repair template for mutagenesis
tttaaaaagtgtagagaaactagttcaagcaacatcagtatataagaatgaagacaacga

SEQ ID NO: 82 - DNA, 60 bp, Artificial Sequence
  Repair template for mutagenesis
tgcctctcacgcttcaattgtaagaatatttgaattcattcgttgtcttcattcttatat

SEQ ID NO: 83 - DNA, 60 bp, Artificial Sequence
  Repair template for mutagenesis
acaacactaactcggtactcaagttatactcacatcaataacaagacatgaattcacatc

SEQ ID NO: 84 - DNA, 60 bp, Artificial Sequence
  Repair template for mutagenesis
gcaagcgttgatttatttcaaagagtgcctcggatccttaaagatgtgaattcatgtctt

SEQ ID NO: 85 - DNA, 60 bp, Artificial Sequence
  Repair template for mutagenesis
ttcacagagtgattatctgagtcgttcatacacccaagaagtttgatatttttgtctagt

SEQ ID NO: 86 - DNA, 60 bp, Artificial Sequence
  Repair template for mutagenesis
tgacatctttaactctatgttattatataatgtgtattaccattgtagttgattattagt

SEQ ID NO: 87 - DNA, 83 bp, Artificial Sequence
  Repair template for mutagenesis
ctcaagacattaggtgaagggtcatttggtaaagtgaaattggctcaacacctcggtacaggtcaaaaagttgctttgagaat

SEQ ID NO: 88 - DNA, 84 bp, Artificial Sequence
  Repair template for mutagenesis
taaatatgaaatctctctttcaacacgaccctgcatgtcgctttttgctaatgttttacgattaataattctcaaagcaacttt

SEQ ID NO: 89 - DNA, 84 bp, Artificial Sequence
  Repair template for mutagenesis
taaatatgaaatctctctttcaacacgaccctgcatgtcgctttttgctaatgttttacgattaagaattctcaaagcaacttt

SEQ ID NO: 90 - DNA, 60 bp, Artificial Sequence
  Repair template for mutagenesis
ttttctcaaaaaaatctagcagcacaaaatatagcagaaactgccaacaaataagaattc

SEQ ID NO: 91 - DNA, 59 bp, Artificial Sequence
  Repair template for mutagenesis
gttgactggtagatgtccagttgttgatgtaactcataaagaattcttatttgttggca

SEQ ID NO: 92 - DNA, 60 bp, Artificial Sequence
  Repair template for mutagenesis
tagcagcacaaaatatagcagaaactgccaacaaagggtttatgagttacatcaacaact

SEQ ID NO: 93 - DNA, 60 bp, Artificial Sequence
  Repair template for mutagenesis
actttattatcttcttgttgactggtagatgtgaattcttagttgttgatgtaactcata

SEQ ID NO: 94 - DNA, 60 bp, Artificial Sequence
  Repair template for mutagenesis
acaatttcaacaaccgcagcaacaactttattaagaattcggcgaacaaatagttcaccc

SEQ ID NO: 95 - DNA, 60 bp, Artificial Sequence
  Repair template for mutagenesis
tgttacatttgtagtattttgtccagtttgggctgcagcagggtgaactatttgttcgcc

SEQ ID NO: 96 - DNA, 20 bp, Artificial Sequence
  CDR1/2 guide sequence
gttttggggagacccggtgc

SEQ ID NO: 97 - DNA, 9 bp, Artificial Sequence
  Hypothetical target sequence
gagcatatc

SEQ ID NO: 98 - RNA, 9 nt, Artificial Sequence
  Hypothetical sgRNA
gauaugcuc

SEQ ID NO: 99 - DNA, 3240 bp, Candida albicans
atgtatgcgacagcccatacaattaaacaacaacaacaacaacaacaacaacatccaccaccacctttaaacggtggactacatgcaagtggggctcctccaaattcccatgaagcagcagctattgctcagcaacaacaacaacagcagcaacaccacaatggtcctggtatgattgttgccgcagctgcagcttctgctaaccaacaagctgtccaagccagagcccaacaacaacaacagcagcaacaacagcgattacctagttcagctgctcttaatgaaactacagtatcaacttggttagccattggttcattagccgagagtttaggtgacattgaacgtgcgacagcttcttacaattccgctttgagacattcaccaaataacccagatattttagtcaaaatagcaaatacataccgttcaaaagatcagtttcttaaggctgctgaattgtatgaacaagctcttaatttccatgttgagaatggtgaaacttggggattattgggtcattgttacttgatgttggataatttgcaaagagcttatgctgcttatcaacgtgcattgttttacttggaaaaccctaacgttccaaaattgtggcacggaattggtattttatatgacagatatggctcattagaatatgctgaagaagcctttgtgagagttttggatttggatccaaatttcgacaaggctaatgaaatttatttccgtttagggatcatttataagcatcaaggtaaactacaaccagcattagaatgtttccaatacattttgaataatccaccacacccattaactcaaccagatgtttggtttcaaattggttcagtgtatgaacaacaaaaggattggaatggtgctaaggatgcttatgaaaaagtgttacagattaatcctcatcacgctaaagttttgcaacaattgggatgtctttattcccaagcagaatcaaatccatcaacaccagctaatggtgctgcaccaccacataagccattccaacaagatttgaccattgctttaaaatatttgaaacaatctttggaagttgatcaaagtgatgctcattcatggtactatttgggtagagtagaaatgattagaggtgatttcactgctgcttatgaagctttccaacaagctgtcaatcgagatgcaagaaacccaactttctggtgttcaattggtgttttgtactatcaaataagccaatatcgtgatgcattggatgcttataccagagccattagattaaatccttatatcagtgaagtatggtatgatttggggactttgtatgagacttgtaataatcaaattagtgatgcattggatgcatatagacaagcagaaagattggatccaaataatcctcatataaaggcaagattagaacaattgacaaagtatcaacaagaaggtaatactcacccacctcaaccaccgccaagttctcaacaacctagattacctcaaggaatggttttggaaagtactcaacaacaacagcaacaacaaccaccaccacctccacaacaacaacaacaacaacttcaacaccaactgcaactgcaacctcaaccacagcaaccacctcaaacccaatcacaaccactgttacttcaacaccaatcttcattgcctcctcaacaaatccaaccattacatcaacaagctgcaaagcctttagtgaatcaacaacaaagtccaccaccacctcacttgatgaacttgggacaaccggggcaacaaccacaacaattgccaccacatcttccaccacatacccagcaaccttctcaaattcaagaaaagcctccaactcaagaacaaccacattatcaaccacctccacctccacaacatcaacagcaatcgcaatcgcaaccgcaacctccacaccaacctcaacacactcaaaatcaactgcctcaattagctcaattgccaccacaccattctaatcctccagctaagccacatggtgcacctcaacaaagaactggtttaccggatttattacacaactctgctaatatcatatcagctccatcacaagtacctcaaccacaacaacaatatcaacaaccacatattgcacctgttagacaagaacaagttaaccatgttccttcaatttatctggctcctagaccaactgagacaacacttcctcaaatcaacaacccaaatgagtcaaccacaacacaagttccacaactcaaaaaggaggaacctaaaccagaggctactgtttctgctccagttcctgaggctattaaagttcaagatcaagtgacaatccaggagtcagcaccagcagcagcagcagcagtgtcagcaccagcttctgctccagttggtgatataaaaacagatactgtatctactactacacctgctacttcaaccactgcagatgctgtgccagtatctgtgtctcaagttggtgaagcaccaaatgttgttcaagagaagaaagttccggacaccgagcagatcgtttcacaagttgaaaaacccgtggagtcacaaccagaagttacaccagctccaacaccagctccagctcttgcaacagcaccaactgaacctgcacctactgataaggacgttgtaatggctccaagtaaaagtgcaacacctgttcctcaaagtattgtggaacagaacaccagagtatctgaagctacaaaggcaccagaatccaatggtaaacatgatttagaagacaagaatgatgaagaaaaaattttaaagaggccaactgttgaaacgactactgaatctgtaccagttaaccaacctgttgagaaagaaaatgaaaaagttgaggttccaccgccactggaacaaccaagttcagaaaagagagaaaaagaagtcaacggatcaattaagaaaccattggaaaatgaaagtaaggttgatattcctcaattctcatcaaatatcacagctcaaaatgaagaagcaaaatctggagaagaaactaaaaaagatacaaccaagacaagtccagcaaaacaaggggaagttaaggaagtaataccatcatctacagaaactgtatcaaaaccagatgttgaaaaagacaataaagagaaagacaaagatgaagatgaagtgatggctgatgaagatgacgtcaaaaaagatgaaaatccagaacctccaatgagaaagattgaagaagatgaaaattatgatgatgaa

SEQ ID NO: 100 - protein, 1080 aa, Candida albicans
MYATAHTIKQQQQQQQQHPPPPLNGGLHASGAPPNSHEAAAIAQQQQQQQQHHNGPGMIVAAAAASANQQAVQARAQQQQQQQQQRLPSSAALNETTVSTWLAIGSLAESLGDIERATASYNSALRHSPNNPDILVKIANTYRSKDQFLKAAELYEQALNFHVENGETWGLLGHCYLMLDNLQRAYAAYQRALFYLENPNVPKLWHGIGILYDRYGSLEYAEEAFVRVLDLDPNFDKANEIYFRLGIIYKHQGKLQPALECFQYILNNPPHPLTQPDVWFQIGSVYEQQKDWNGAKDAYEKVLQINPHHAKVLQQLGCLYSQAESNPSTPANGAAPPHKPFQQDLTIALKYLKQSLEVDQSDAHSWYYLGRVEMIRGDFTAAYEAFQQAVNRDARNPTFWCSIGVLYYQISQYRDALDAYTRAIRLNPYISEVWYDLGTLYETCNNQISDALDAYRQAERLDPNNPHIKARLEQLTKYQQEGNTHPPQPPPSSQQPRLPQGMVLESTQQQQQQQPPPPPQQQQQQLQHQSQSQPQPQQPPQTQSQPSLLQHQSSLPPQQIQPLHQQAAKPLVNQQQSPPPPHLMNLGQPGQQPQQLPPHLPPHTQQPSQIQEKPPTQEQPHYQPPPPPQHQQQSQSQPQPPHQPQHTQNQSPQLAQLPPHHSNPPAKPHGAPQQRTGLPDLLHNSANIISAPSQVPQPQQQYQQPHIAPVRQEQVNHVPSIYSAPRPTETTLPQINNPNESTTTQVPQLKKEEPKPEATVSAPVPEAIKVQDQVTIQESAPAAAAAVSAPASAPVGDIKTDTVSTTTPATSTTADAVPVSVSQVGEAPNVVQEKKVPDTEQIVSQVEKPVESQPEVTPAPTPAPALATAPTEPAPTDKDVVMAPSKSATPVPQSIVEQNTRVSEATKAPESNGKHDLEDKNDEEKILKRPTVETTTESVPVNQPVEKENEKVEVPPPSEQPSSEKREKEVNGSIKKPLENESKVDIPQFSSNITAQNEEAKSGEETKKDTTKTSPAKQGEVKEVIPSSTETVSKPDVEKDNKEKDKDEDEVMADEDDVKKDENPEPPMRKIEEDENYDDE

SEQ ID NO: 101 - DNA, 23 bp, Candida albicans
aaaaaaaaggttggggcaaacgg

SEQ ID NO: 102 - DNA, 23 bp, Candida albicans
aaaccgatactgtccttattagg

SEQ ID NO: 103 - DNA, 23 bp, Candida albicans
accatcactaacccacctgatgg

SEQ ID NO: 104 - DNA, 23 bp, Candida albicans
agaagttcaacgtgaagaagtgg

SEQ ID NO: 105 - DNA, 23 bp, Candida albicans
tctggacgaggaggttttggtgg

SEQ ID NO: 106 - DNA, 67 bp, Artificial Sequence
  Plasmid fragment
cgtaaactatttttaatttggagacggaattccgtctcgttttagagctagaaatagcaagttaaaa

SEQ ID NO: 107 - DNA, 69 bp, Artificial Sequence
  Plasmid fragment
cgtaaactatttttaatttgcaacaatcatacgacctaatgttttagagctagaaatagcaagttaaaa

SEQ ID NO: 108 - DNA, 48 bp, Unknown
  ADE wildtype locus
ttaggaggtggccaattaggtcgtatgattgttgaagcagcacataga

SEQ ID NO: 109 - DNA, 48 bp, Artificial Sequence
  ADE mutant locus
ttaggaggttaatgattaggtcgtatgattgttgaagcagcacataga

SEQ ID NO: 110 - DNA, 38 bp, Unknown
  CDR1 and CDR2 locus fragment
ggtgaacttactgtkgttttggggagacccggtgctgg

SEQ ID NO: 111 - DNA, 10 bp, Unknown
  CDR1 and CDR2 locus fragment
ttgttccaca

SEQ ID NO: 112 - DNA, 55 bp, Artificial Sequence
  CDR1 and CDR2 locus fragment with EcoRI site
ggtgaacttactgtkgttttggggagacccggtgcttaagaattcttgttccaca

SEQ ID NO: 113 - DNA, 23 bp, Unknown
  Mtla1 locus fragment
atataagaatgaagacaacgagg

SEQ ID NO: 114 - DNA, 37 bp, Artificial Sequence
  Mtla1 locus fragment with EcoRI
atataagaatgaagacaacgaatgaattcaaatattc

SEQ ID NO: 115 - DNA, 23 bp, Unknown
  Mtla2 locus fragment
acaagacatgaattcacatctgg

SEQ ID NO: 116 - DNA, 36 bp, Artificial Sequence
  Mtla2 locus fragment with BamH1 site
acaagacatgaattcacatctttaaggatccgaggc

SEQ ID NO: 117 - DNA, 18 bp, Unknown
  TPK2 locus fragment
ccgcagcaacaactttat

SEQ ID NO: 118 - DNA, 23 bp, Unknown
  TPK2 locus fragment
ccaggcgaacaaatagttcaccc

SEQ ID NO: 119 - DNA, 47 bp, Artificial Sequence
  TPK2 locus fragment with EcoRI site
ccgcagcaacaactttattaagaattcggcgaacaaatagttcaccc

SEQ ID NO: 120 - DNA, 24 bp, Unknown
  DCR1 locus fragment
atagcagaaactgccaacaaaggg

SEQ ID NO: 121 - DNA, 39 bp, Artificial Sequence
  DCR1 locus fragment with EcoRI site
atagcagaaactgccaacaaataagaattctttatgagt

SEQ ID NO: 122 - DNA, 68 bp, Unknown
  Wild-type snf1 fragment
aaattggctcaacacttgggcacaggtcaaaagttgctttgaaaatcattaatcgtaaaacattagcc

SEQ ID NO: 123 - DNA, 69 bp, Artificial Sequence
  Mutant snf1 fragment
aaattggctcaacacctcggtacaggtcaaaaagttgctttgagaattcttaatcgtaaaacattagcc

SEQ ID NO: 124 - protein, 16 aa, Unknown
  Wild-type ADE fragment
LGGGQLGRMIVEAAHR

SEQ ID NO: 125 - protein, 11 aa, Artificial Sequence
  Mutant ADE fragment
LGRMIVEAAHR

SEQ ID NO: 126 - protein, 13 aa, Unknown
  CDR1 and CDR2 fragment
GELTVVLGRPGAG

SEQ ID NO: 127 - protein, 12 aa, Unknown
  CDR1 and CDR2 fragment
GELTVVLGRPGA

SEQ ID NO: 128 - protein, 5 aa, Artificial Sequence
  CDR1 and CDR2 fragment
DFLFH

SEQ ID NO: 129 - protein, 7 aa, Unknown
  Mtla1 locus fragment
YKNEDNE

SEQ ID NO: 130 - protein, 5 aa, Unknown
  Mtla2 locus fragment
MNSHL

SEQ ID NO: 131 - protein, 6 aa, Unknown
  TPK2 locus fragment
PQQQLY

SEQ ID NO: 132 - protein, 8 aa, Unknown
  TPK2 locus fragment
PGEQIVHP

SEQ ID NO: 133 - protein, 8 aa, Unknown
  DCR1 locus fragment
IAETANKG

SEQ ID NO: 134 - protein, 7 aa, Unknown
  DCR1 locus fragment
IAETANK

SEQ ID NO: 135 - protein, 23 aa, Unknown
  Wild-type snf1 fragment
KLAQHLGTGQKVALKIINRKTLA

SEQ ID NO: 136 - protein, 23 aa, Artificial Sequence
  Mutant snf1 fragment
KLAQHLGTGQKVALRILNRKTLA

(19) United States
(12) Patent Application Publication
(10) Pub. No.: US 2017/0166928 A1
(43) Pub. Date: Jun. 15, 2017

(54) Compositions And Methods For Genetically Modifying Yeast

(71) Applicant: Whitehead Institute for Biomedical Research, Cambridge, MA (US)

(72) Inventors: Valmik K. Vyas, Medford, MA (US); Gerald R. Fink, Chestnut Hill, MA (US)

(21) Appl. No.: 15/089,472

(22) Filed: Apr. 2, 2016

Related U.S. Application Data
(60) Provisional application No. 62/143,004, filed on Apr. 3, 2015.

(51) Int. Cl. C12N 15/90; C12N 15/81; C12N 9/16

(52) U.S. Cl. CPC C12N 15/905; C12N 9/16; C12Y 301/00; C12N 15/815

(57) ABSTRACT

The present invention provides compositions and methods for genetically modifying yeast cells using a Candida-compatible CRISPR/Cas9 nuclease system. Also provided are yeast cells that have been genetically modified using such compositions and methods.

### GOVERNMENT SUPPORT

This invention was made with government support under NIH GM035010 from the National Institutes of Health. The government has certain rights in the invention.

### BACKGROUND OF THE INVENTION

Candida albicans, the major fungal pathogen of humans, causes infections that can be fatal in immunocompromised individuals (Pfaller and Diekema, Clin Microbiol Rev 20:133-163 (2007); Wisplinghoff, et al., Clin Infect Dis 39:309-317 (2004); Wisplinghoff, et al., Int J Antimicrob Agents 43:78-81 (2014)). The study of Candida pathogenesis has been hindered by the absence of facile molecular genetics for this organism, as Candida possesses a number of characteristics that render it relatively unamenable to genetic manipulation. For example, Candida is diploid, lacks any known meiotic phase, and has no plasmid system. In addition, the Candida genome is populated by many gene families, including over 120 drug efflux pumps (Braun, et al., PLoS Genet 1:36-57 (2005); Gaur, et al., BMC Genomics 9:579 (2008); Prasad and Goffeau, Annu Rev Microbiol 66:39-63 (2012)). This redundancy impedes analysis of the resistance to antifungal agents as the construction of multiple mutations in the members of these families is beyond current technology. These pumps also give Candida a high inherent drug resistance, rendering all but one drug resistance marker useless. An added complexity to genetics in Candida is that the chromosome number is not rigidly controlled, so that many strains contain one or more additional copies of a chromosome (2n+1) (Selmecki, et al., PLoS Genet 5:e1000705 (2009); Selmecki, et al., Eukaryot Cell 9:991-1008 (2010); Selmecki, et al., Science 313:367-370 (2006); Selmecki, et al., Mol Microbiol 55:1553-1565 (2005)).

Accordingly, there is a significant unmet need for a system for manipulating the Candida genome to produce genetically-modified Candida cells that can be used, inter alia, to identify effective therapeutic agents for treating Candida infections.

### SUMMARY OF THE INVENTION

Described herein is a system for genetically modifying yeast that overcomes many of the obstacles that Candida and other CTG clade yeasts present to researchers seeking to genetically engineer these organisms. The compositions and methods described herein facilitate, e.g., the isolation of homozygous gene knockouts in Candida species, even without selection, and permit the creation of yeast strains having mutations in multiple genes, gene families, and genes that encode essential functions.

In one aspect, the present invention provides a nucleic acid comprising a Candida-compatible clustered regularly interspaced short palindromic repeat (CRISPR)-associated nuclease 9 (CaCas9) nucleotide sequence that encodes a protein having at least 90% sequence identity to SEQ ID NO: 5, or a fragment thereof, wherein each leucine in the protein is encoded by a codon other than CTG or CUG.

In a further aspect, the invention provides a nucleic acid comprising an RNA polymerase III promoter, a cloning site for introducing an sgRNA coding sequence, and a locus targeting sequence to direct integration of all or a portion of the nucleic acid into a yeast genome.

In another aspect, the invention also provides kits comprising one or more of the nucleic acids described herein.

In an additional aspect, the invention provides genetically-modified yeast cells comprising one or more of the nucleic acids described herein.

The invention also provides a method for modifying a genome of a yeast cell, comprising: a) introducing into the yeast cell a first nucleic acid comprising a Candida-compatible clustered regularly interspaced short palindromic repeat (CRISPR)-associated nuclease 9 (CaCas9) nucleotide sequence that encodes a protein sequence having at least 90% sequence identity to SEQ ID NO: 5, or a fragment thereof, wherein each leucine in the protein is encoded by a codon other than CTG or CUG; b) introducing into the yeast cell a second nucleic acid comprising an sgRNA coding sequence; and c) expressing the CaCas9 and sgRNA coding sequences in the yeast cell, thereby modifying the genome of the yeast cell.

The compositions and methods provided herein can be used to modify the yeast genome (e.g., to increase or decrease activity of a gene) and allow for the manipulation of the genome of a variety of species of yeast, including Candida. The present invention provides new opportunities to explore the biology and pathogenesis of these organisms, e.g., to generate improved strains for industrial applications, to identify potential antifungal drug targets, and to identify and/or characterize genes that contribute to antifungal drug resistance.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D illustrate CRISPR expression constructs and schematic of CaCas9-mediated mutagenesis. FIG. 1A depicts the duet system consisting of 2 plasmids: pV1025, shown before (top) and after flipout (bottom), which targets ENO1; and pV1090, which targets RP10. FIG. 1B shows the solo system consisting of 1 plasmid, pV1093, which targets ENO1. FIG. 1C illustrates how both Solo and Duet guide expression systems permit rapid cloning by digestion with BsmBI followed by ligation of annealed oligos (shaded sequences) with desired guide sequence (ADE2 guide sequence in red box). FIG. 1D is a schematic of the Cas9 mutagenesis method, which can create homozygous mutations in the gene (*) and simultaneously mutate sequences (e.g., the PAM) to prevent repeated cleavage subsequent to integration.

FIGS. 2A-2E show that Candida albicans CRISPR is an efficient mutagenesis system. FIG. 2A shows that Candida CRISPR efficiently mutagenized both ADE2 loci in SC5314, which was transformed with pV1081 and a mutagenic repair template; omission of Cas9, sgRNA, or a repair template with homology to the guide resulted in failure to obtain ade2 mutants. FIG. 2B is the sequence of the ADE2 locus in WT and mutant isolates. FIG. 2C shows the result of an assay for ura3/ura3 transformant on 5-fluoroorotic acid (FOA) plates, wherein FOA permits growth of ura3/ura3 but not URA3+ strains. FIG. 2D depicts wrinkled colony morphology of RAS1V13 on transformation plates (top) and glycogen accumulation defect/wrinkled colony morphology of RAS1V13 (bottom). Glycogen accumulation is visualized by exposing yeast to iodine vapors, which stains glycogen red. WT (left) has a smooth morphology and stains red due to accumulated glycogen (left), while RAS1V13 (right) has a wrinkled morphology and fails to stain. FIG. 2E illustrates that truncation of RAS1 at position 13 (ras1 (TAA) 13) reduced growth rate.

FIGS. 3A-3C show that CRISPR permits simultaneous targeting of CDR1 and CDR2, which mediate resistance to fluconazole and cycloheximide. FIG. 3A shows the sequence of CDR1 and CDR2 loci and verification by digestion. FIG. 3B illustrates that mutation of CDR1 and CDR2 sensitizes SC5314 (left) and fluconazole-resistant clinical isolate Can90 (right) to fluconazole (0.41 μg/mL for SC5314, 200 μg/mL for Can90). Different fluconazole concentrations were used for each strain background, because the Can90 isolate had much greater resistance. Solid lines indicate medium without fluconazole; dotted lines indicate medium with fluconazole. FIG. 3C shows simultaneous mutation of three genes (6 sites) in a single transformation, and the resulting phenotypes. Left panel is YPD, and right panel is YPD plus cycloheximide at 400 μg/ml. The poorer growth on petri plates of the ade2 cdr1 cdr2 triple is reflected in liquid growth on fluconazole. The ade2 CDR1 CDR2 has a doubling time of 6 hours, while the ade2 cdr1 cdr2 mutant has a doubling time of 12 hours when grown in 1.2 μg/ml fluconazole.

FIGS. 4A-4D illustrate that the Candida CRISPR system allows efficient isolation of mutations in essential functions. FIG. 4A shows the growth of SC5314 of the indicated genotype at 37° C. or 16° C. FIG. 4B shows the growth of indicated strains on YP with the indicated carbon source at 37° C. for 3 days. FIG. 4C shows the growth of indicated strains on YPD at the indicated temperatures. FIG. 4D shows the growth of indicated strains resulting from overnight YPD cultures which were diluted into RPMI+10% fetal bovine serum and grown for 2 hours at 37° C. Scale bar is 5 μm.

FIG. 5 illustrates a recyclable Solo system vector pV1200 which permits serial mutagenesis. The pV1200 Solo system vector is identical to the Solo system vector pV1093, except that it contains the Nat^{R}-FLP and SNR52p-sgRNA cassette flanked by FRT sites, and an inducible Flippase under the control of the SAP2 promoter. Induction of Flippase causes excision of the Nat^{R}-FLP-SNR52-sgRNA cassette (bottom), leaving a Nat sensitive strain that can be mutagenized with another sgRNA expression cassette.

FIGS. 6A-6D show components of Candida CRISPR Duet system (Cas9, sgRNA, and repair template). Strain VY959 (FIGS. 6A and 6B), which contains the integrated Cas9 from the Duet system, was transformed with pV1010 (Duet sgADE2 expression plasmid), with (FIG. 6A) or without (FIG. 6B) a mutagenic repair template, and plated on YPD+Nat. Strain SC5314 (FIG. 6C and FIG. 6D) was transformed with pV1010 with a repair template without (FIG. 6C) or with (FIG. 6D) Cas9 expression plasmid pV1025.

FIGS. 7A-7D show that Candida CRISPR Solo system requires a mutagenic repair template, but does not require selection for system components. Strain SC5314 was transformed with pV1081 (Solo system for ADE2) without (FIG. 7A) or with a mutagenic template containing the guide sequence (FIG. 7B) or 250-bp downstream (FIG. 7C), and plated on YPD+Nat. Dilution of yeast grown in FIG. 7B was plated to non-selective YPD plates (FIG. 7D).

FIGS. 8A-8D show use of Candida CRISPR to enable isolation of homozygous mutants at multiple loci, including MtlA1 (FIG. 8A), Mtlα2 (FIG. 8B), TPK2 (FIG. 8C), and DCR1 (FIG. 8D). PCR genotyping of indicated genes is shown, and numbers listed are base pair positions with respect to the ATG codon.

FIGS. 9A and 9B show results from a study demonstrating that mutation of CDR1 and CDR2 creates pleotropic drug sensitivity. Three microliters of the indicated drugs were spotted atop YPD plates containing the indicated strain (SC5314 in FIG. 9A, CDR1+/+CDR2+/+left panel and cdr1−/−cdr2−/−right panel; Can90 in FIG. 9B, CDR1+/+CDR2+/+left panel and cdr1−/−cdr2−/−right panel). Plates were allowed to grow overnight and photographed.

FIGS. 10A-10D show results from studies to assess a mutation of SNF1 in Candida. FIG. 10A shows unusual colony morphology of snf1-K81R transformants. Wrinkly colonies (two examples are marked with arrows) contain the K81R mutation, while smooth colonies are WT. FIG. 10B shows PCR confirmation of homozygous SNF1 mutation. Mutation at position K81R introduces an EcoRI site not found in the WT locus (left) and insertion of MAL2p at SNF1 increases size of PCR amplification with SNF1 primers (right). FIG. 10C depicts the sequence of WT and snf1-K81R alleles. Silent mutations were introduced into targeting region to prevent further cleavage. FIG. 10D shows growth of strains of the indicated genotype in YPD alone, with cycloheximide (400 μg/ml), or fluconazole (1 μs/ml).

FIGS. 11A-11C are schematic diagrams illustrating the CaCas9 solo construct pV1063 (FIG. 11A), and the nuclease-inactive CaCas9 solo construct pV1062 (FIG. 11B). FIG. 11C depicts the target to be modified, indicated by the arrow.

FIG. 12 shows a functional comparison of using pV1063 to silence expression, as compared to using nuclease-inactive pV1062 to repress expression, which demonstrates comparable GFP silencing.

FIG. 13A-13C illustrate additional CRISPR expression constructs for serial CRISPR mutagenesis in various yeast systems. FIG. 13A depicts pV1393, which targets the CRISPR system for insertion into the Neut5L locus; pV1393 allows complete removal of CaCas9 and the guide expression module upon induction of flippase, leaving only an FRT insertion at Neut5L. FIG. 13B depicts pV1326 and pV1382 in pRS416 vector; promoter regions are specified in the diagrams. pV1326 and pV1382 are entry plasmids for mutagenesis in S. cerevisiae and C. glabrata (after appropriate guide is cloned in). FIG. 13C depicts pV1464 for use in Naumovozyma castellii.

FIG. 14 shows results from serial mutagenesis studies in S. cerevisiae and C. glabrata using pRS416-based vectors, as indicated. pV1386 is based on the pV1382 plasmid, into which a guide directed against Saccharomyces cerevisiae ADE2 is inserted; pV1435 is based on pV1382 plasmid into which a guide directed against Candida glabrata ADE2 is inserted.

FIG. 15 shows CRISPR-derived mutations in the absence of a repair template in S. cerevisiae strains having mutations in the homologous repair machinery (e.g., Rad51, Rad52, and Rad59). pV1338 is based on the pV1326 plasmid, into which a guide directed against Saccharomyces cerevisiae ADE2 is inserted.

FIG. 16 depicts repair template requirements in C. albicans. Allele-specific guides can be used to generate loss of heterozygosity events at the locus and/or chromosome level.

### DETAILED DESCRIPTION OF THE INVENTION

A description of example embodiments of the invention follows.

The CRISPR/Cas9 system described herein circumvents many of the challenges unique to the genetic manipulation of Candida albicans. Clustered Regularly Interspaced Short Palindromic Repeats (CRISPR) together with cas (CRISPR-associated) genes was first identified as an adaptive immune system that provides acquired resistance against invading foreign nucleic acids in bacteria and archaea (Barrangou et al., 2007. Science 315:1709-12). CRISPR consists of arrays of short conserved repeat sequences interspaced by unique variable DNA sequences of similar size called spacers, which often originate from phage or plasmid DNA (Barrangou et al., 2007. Science 315:1709-12; Bolotin et al., 2005. Microbiology 151:2551-61; Mojica et al., 2005. J Mol Evol 60:174-82). In its native environment, the CRISPR/Cas system functions by acquiring short pieces of foreign DNA (spacers) which are inserted into the CRISPR region and provide immunity against subsequent exposures to phages and plasmids that carry matching sequences (Barrangou et al., 2007. Science 315:1709-12). The CRISPR/Cas9 system from Streptococcus pyogenes was first characterized as involving only a single gene encoding the Cas9 protein and two RNAs—a mature CRISPR RNA (crRNA) and a partially complementary trans-acting RNA (tracrRNA)—which were identified as necessary and sufficient for RNA-guided silencing of foreign DNAs. Since its discovery, the CRISPR/Cas system has been developed to modify or silence various genes of interest (see, e.g., WO 2014/018423; WO 2014/011237; WO 2013/176772; and WO 2013/169398).

The successful implementation of CRISPR in Candida required the solution of several technical constraints. For example, as described herein, the Cas9 gene was recoded to be consonant with the CUG codon divergence characteristic of the Candida clade (Papon, et al., Trends in Biotechnology 32(4):167-68, 2014; Wang, et al., BMC Evolutionary Biology, 9:195, 2009). In addition, suitable RNA Polymerase III promoters were identified for expression of the guide RNA in vectors. Further, guide sequences that can differentially target genes in diploid Candida were identified. These include guides that are allele specific, gene specific, and ones that could target multiple genes or gene families. Gene families, which have been historically difficult to study, can be modified in a single experiment using the present system.

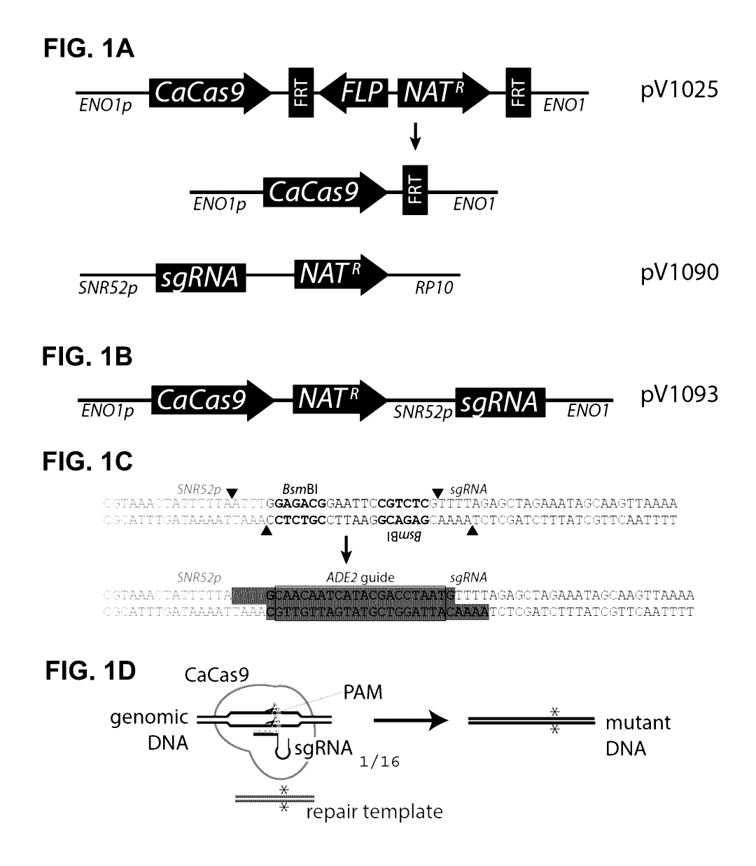

The present system, as generically depicted in FIG. 1D, comprises a Candida-compatible Cas9 nuclease and a synthetic guide RNA (sgRNA) that directs Cas9 to cleave regions in the genome that hybridize to the 20 bp guide (or protospacer) from the sgRNA when it is followed by the sequence NGG (the protospacer-adjacent motif, or “PAM”). This system has been successfully imported to diverse kingdoms ranging from fungi to plants and animals (reviewed in Doudna and Charpentier, Science 346:1258096 (2014); Terns and Terns, Trends Genet 30:111-118 (2014)). However, most of these systems do not pose the unique set of constraints found in Candida.

The present invention is based, in part, on the identification of a codon-optimized sequence for expressing Cas9 protein in various species of Candida and other species of yeast (e.g., CTG clade species of yeast). Thus, the present invention provides a CRISPR/Cas9 system compatible for use in various yeasts, including Candida.

### Candida-Compatible Nucleic Acids Encoding CRISPR/Cas9 System Components

The nucleic acids described herein relate, in part, to a “Duet” system, and a “Solo” system for performing CRISPR in yeast (e.g., Candida). The Duet system, an example of which is depicted in FIG. 1A, uses the sequential integration of two plasmids: the first comprising CaCas9 nucleotide sequence (the “Duet CaCas9 system plasmid” e.g., pV1025) and the second comprising a coding sequence for a synthetic guide RNA (sgRNA) that targets a gene of interest (the “Duet sgRNA system plasmid”, e.g., pV1090). The Duet sgRNA system plasmid allows a user to insert any suitable sgRNA coding sequence designed for a target sequence of interest. In general, the second plasmid for expression of the sgRNA against a target gene is cotransformed with a mutagenic double-stranded oligonucleotide (a “repair template”, as described herein), which is complementary to a target gene and may contain a desired modification, e.g., a mutation to the PAM sequence and a premature UAA stop codon.

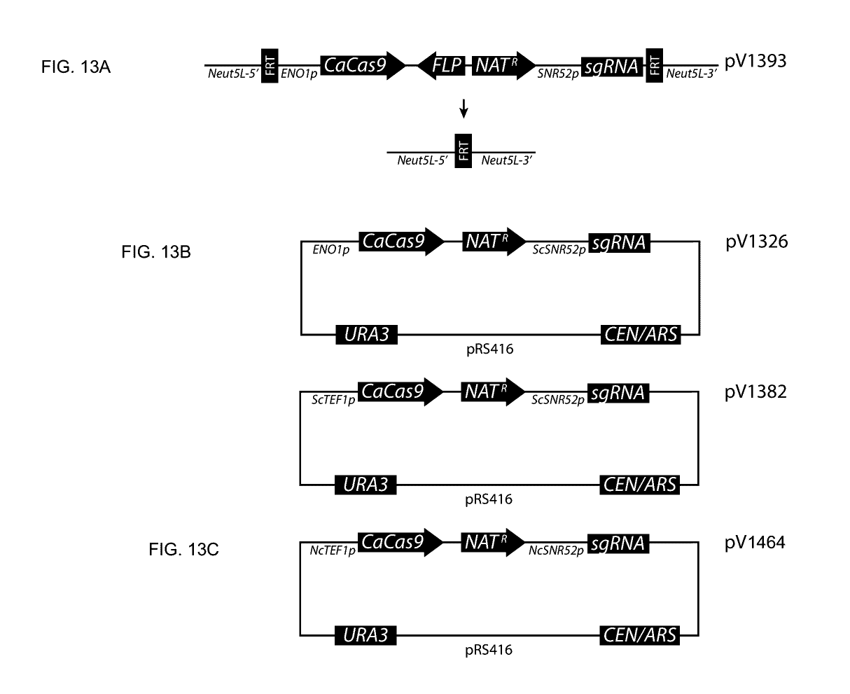

The “Solo” system, examples of which are depicted in, e.g., FIG. 1B and FIG. 13A, consolidates the CaCas9 nucleotide sequence and the sgRNA coding sequence into a single plasmid construct (the “Solo CaCas9/sgRNA system plasmid”) that can be integrated at a desired locus. Like the Duet system, a mutagenic double-stranded oligonucleotide can be cotransformed with the Solo system. Similar to the Duet sgRNA system plasmid, the Solo system allows the insertion of any suitable sgRNA coding sequence designed for a target sequence of interest.

Accordingly, in certain aspects, the invention relates to a nucleic acid comprising a Candida-compatible clustered regularly interspaced short palindromic repeat (CRISPR)-associated nuclease 9 (Cas9) (CaCas9) nucleotide sequence. As used herein, a “Candida-compatible Cas9 nucleotide sequence” or “CaCas9 nucleotide sequence” refers to a nucleotide sequence encoding a bacterial Cas9 protein (e.g., a Cas9 nuclease from any of a variety of prokaryotes, such as, for example, Streptococcus pyogenes, Staphylococcus aureus, Neisseria meningitides, Streptococcus thermophilus, and Treponema denticola), wherein the bacterial Cas9 nucleotide sequence has been optimized (e.g., codon optimized) for expression of the bacterial Cas9 protein in Candida. As those of skill in the art would appreciate in light of the present disclosure, other endonucleases known in the art can also be used in the present invention. See, e.g., Zetsche et al., Cell 163(3):759-71, 2015; Kleinstiver et al., Nature 523(7561):481-85, 2015—each incorporated herein by reference in its entirety).

Many species of Candida belong to the fungal CTG clade corresponding to a group of ascomycetous yeasts displaying a particular genetic code, such that the universal CUG codon for leucine is predominantly translated as serine and rarely as leucine (Papon, et al., Trends in Biotechnology 32(4):167-68, 2014). Thus, a CaCas9 nucleotide sequence can be prepared, for example, by encoding one or more (e.g., all), of the leucine residues in a Cas9 protein sequence (e.g., SEQ ID NO:5) with a codon other than CTG or CUG, e.g., CTC, TTG, CTT, CTA, and TTA. However, serine residues in a Cas9 protein sequence can be encoded by a CTG or CUG codon, as well as any other serine codon. In further aspects, a leucine residue in Cas9 can be encoded by CTG or CUG if a substitution of that leucine residue for serine does not substantially alter the function of Cas9. In various aspects, while “Candida-compatible” refers to a coding sequence optimized for expression in Candida, those of skill in the art will appreciate, in light of the present disclosure, that the nucleotide sequences of the present invention may be used and expressed in a variety of yeast species, as described herein. Codon optimization in yeast is described, for example, in U.S. Patent Application Publication No. 20120309073, the contents of which are incorporated herein by reference.

In one aspect, the nucleic acid is a DNA molecule. In another aspect, the nucleic acid is an RNA molecule.

In certain aspects, the present invention provides a nucleic acid comprising a Candida-compatible clustered regularly interspaced short palindromic repeat (CRISPR)-associated nuclease 9 (CaCas9) nucleotide sequence. In one aspect, the CaCas9 nucleotide sequence is a codon-optimized sequence of SEQ ID NO: 1.

In some aspects, the invention relates to a nucleic acid comprising a Candida-compatible clustered regularly interspaced short palindromic repeat (CRISPR)-associated nuclease 9 (Cas9) nucleotide sequence (CaCas9) that encodes a protein having at least about 40%, 50%, 60%, 70%, 80%, 85%, 90%, 93%, 94%, 95%, 96%, 97%, 98%, or 99% sequence identity to SEQ ID NO: 5, or a fragment thereof, wherein each leucine in the protein is encoded by a codon other than CTG, e.g., CTC, TTG, CTT, CTA, and TTA. In certain aspects, the nucleic acid comprises a CaCas9 nucleotide sequence that encodes SEQ ID NO: 5. In other aspects, the nucleic acid comprises a CaCas9 nucleotide sequence that encodes SEQ ID NO: 6.

As used herein, a “fragment” of a Cas9 protein includes any nuclease-active or nuclease-inactive portion of a Cas9 protein. For example, the nucleic acid may encode one or more fragments of Cas9 that retains nuclease activity. In a particular example, Cas9 may be expressed as two separate fragments (e.g., a nuclease lobe and an alpha-helical lobe) which form a functional, active complex in the presence of an sgRNA (see, e.g., Wright, et al., PNAS, 112 (10:2984-89), 2015). In other aspects, the nucleic acid may encode a nuclease-inactive fragment of Cas9 which may, for example, be fused to one or more other genes (e.g., a transcriptional repressor or activator).

In certain aspects, the CaCas9 nucleotide sequence has at least about 50%, 60%, 70%, 75%, 80%, 85%, 90%, 92%, 93%, 94%, 95%, 96%, 97%, 98%, or 99% sequence identity to SEQ ID NO:2. In a particular aspect, the CaCas9 nucleotide sequence comprises SEQ ID NO: 2.

The term “sequence identity” means that two nucleotide or amino acid sequences, when optimally aligned, such as by the programs GAP or BESTFIT using default gap weights, share at least, e.g., 70% sequence identity, or at least 80% sequence identity, or at least 85% sequence identity, or at least 90% sequence identity, or at least 95% sequence identity or more. For sequence comparison, typically one sequence acts as a reference sequence (e.g., parent sequence), to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols in Molecular Biology). One example of algorithm that is suitable for determining percent sequence identity and sequence similarity is the BLAST algorithm, which is described in Altschul et al., J. Mol. Biol. 215:403 (1990). Software for performing BLAST analyses is publicly available through the National Center for Biotechnology Information (publicly accessible through the National Institutes of Health NCBI internet server). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

As used herein, “wild-type” in the context of a Cas9 coding sequence or protein refers to the canonical bacterial nucleotide or amino acid sequence as found in nature (e.g., as occurs in the bacterium Streptococcus pyogenes). A particular example of a wild-type Cas9 coding sequence is SEQ ID NO:1. A particular example of a wild-type Cas9 amino acid sequence is SEQ ID NO:5.

As used herein, the term “nucleic acid” refers to a polymer comprising multiple nucleotide monomers (e.g., ribonucleotide monomers or deoxyribonucleotide monomers). “Nucleic acid” includes, for example, genomic DNA, cDNA, RNA, and DNA-RNA hybrid molecules. Nucleic acid molecules can be naturally occurring, recombinant, or synthetic. In addition, nucleic acid molecules can be single-stranded, double-stranded or triple-stranded. In some embodiments, nucleic acid molecules can be modified. Nucleic acid modifications include, for example, methylation, substitution of one or more of the naturally occurring nucleotides with a nucleotide analog, internucleotide modifications such as uncharged linkages (e.g., methyl phosphonates, phosphotriesters, phosphoamidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like). “Nucleic acid” does not refer to any particular length of polymer and therefore, can be of substantially any length, typically from about six (6) nucleotides to about 10⁹ nucleotides or larger. In the case of a double-stranded polymer, “nucleic acid” can refer to either or both strands of the molecule.

The term “nucleotide sequence,” in reference to a nucleic acid, refers to a contiguous series of nucleotides that are joined by covalent linkages, such as phosphorus linkages (e.g., phosphodiester, alkyl and aryl-phosphonate, phosphorothioate, phosphotriester bonds), and/or non-phosphorus linkages (e.g., peptide and/or sulfamate bonds).

The terms “nucleotide” and “nucleotide monomer” refer to naturally occurring ribonucleotide or deoxyribonucleotide monomers, as well as non-naturally occurring derivatives and analogs thereof. Accordingly, nucleotides can include, for example, nucleotides comprising naturally occurring bases (e.g., adenosine, thymidine, guanosine, cytidine, uridine, inosine, deoxyadenosine, deoxythymidine, deoxyguanosine, or deoxycytidine) and nucleotides comprising modified bases (e.g., 2-aminoadenosine, 2-thiothymidine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, 2-thiocytidine).

In some aspects, the CaCas9 nucleotide sequence encodes a Cas9 protein having nuclease activity. In one aspect, a Cas9 protein having nuclease activity comprises SEQ ID NO:5.

In other aspects, the CaCas9 nucleotide sequence encodes a Cas9 protein that is lacking nuclease activity, also referred to herein as a “nuclease-inactive Cas9 protein”. A nuclease-inactive Cas9 protein can be prepared, for example, by substituting amino acid residues that are required for catalytic activity in a wild type Cas9 protein with a different amino acid(s). For example, the aspartate at position 10 and the histidine at position 840 in the Cas9 protein represented by SEQ ID NO:5 can be substituted with a different amino acid (e.g., alanine) to yield a nuclease-inactive Cas9. Preferably, the substitutions are non-conservative substitutions. In a particular aspect, a nuclease-inactive Cas9 protein comprises SEQ ID NO:6. In a particular aspect, the CaCas9 nucleotide sequence encoding the nuclease-inactive Cas9 comprises SEQ ID NO:3. Methods for performing site-directed mutagenesis to produce proteins having amino acid substitutions are well known and routine to one of ordinary skill in the art. In certain aspects, the CaCas9 nucleotide sequence encodes a Cas9 protein fragment that lacks nuclease activity.

In certain aspects, the nuclease-inactive Cas9 protein is expressed as a fusion protein with all or a portion of a heterologous protein that represses gene transcription, also referred to herein as a “repressor” protein. Numerous repressor proteins that can be readily adapted for the present invention are known in the art. In one aspect, the nuclease-inactive Cas9 is fused to a Candida albicans suppressor of Snf1 6 (SSN6) protein (SEQ ID NO: 100).

In other aspects, the nuclease-inactive Cas9 protein is expressed as a fusion protein with all or a portion of a heterologous protein that activates gene transcription, also referred to herein as an “activator” protein. Numerous activator proteins that can be readily adapted for the present invention are known in the art. For example, at least two tandem copies (e.g., 4 or more copies) of a fragment (DALDDFDLDML (SEQ ID NO: 106)) derived from transcription activator VP16 can be adapted for use in the present invention (Seipel et al., Biol. Chem, Hoppe-Seyler, 375(7):463-70, 1994). Other examples of transcription activators include GAL4 and GCN4.

In some aspects, the CaCas9 nucleotide sequence encodes a Cas9 protein having a nickase activity, also referred to herein as a “Cas9 nickase”. A Cas9 nickase, which can nick one strand of a double-stranded nucleic acid, facilitates homology-directed repair in eukaryotic cells (Cong, et al., Science, 339, 819-23, 2013). A Cas9 nickase can be prepared, for example, by substituting amino acid residues that are required for catalytic activity in a wild-type Cas9 protein with a different amino acid(s). For example, a single substitution of the aspartate at position 10, the glutamic acid at position 762, the histidine at position 840, the asparagine at position 863, the histidine at position 983, or the aspartic acid at position 986 in the Cas9 protein represented by SEQ ID NO:5 can be substituted with a different amino acid (e.g., alanine) to yield a Cas9 nickase (see, e.g., Nishimasu, et al., Cell, 156:935-49, 2014). Preferably, the substitutions are non-conservative substitutions. Methods for producing proteins having amino acid substitutions (e.g., site-directed mutagenesis) are well known and routine to one of ordinary skill in the art.

In other aspects, the CaCas9 nucleotide sequence encodes a Cas9 protein having a relaxed requirement for the NGG sequence, referred to herein as “CaCas9-PAM”. Cas9 directs cleavage at sites in the genome which match the appropriate region specified by the sgRNA when they are followed by the sequence NGG. Substituting two amino acids—arginine at position 1333 and arginine at position 1335 of SEQ ID NO: 5—relaxes the requirement for the NGG sequence, otherwise known as the PAM. By removing this requirement, the potential targeting applications are greatly increased. Preferably, the substitution is a non-conservative substitution. In one aspect, R1333 and R1335 are substituted with glutamine. In certain aspects, the substitutions in CaCas9-PAM may be combined with the substitutions in the nuclease-inactive CaCas9-SSN6 to create a repressor which can target a much larger array of sequences. In other aspects, the substitutions in CaCas9-PAM may be combined with the substitutions in the nuclease-inactive CaCas9 fused to a transcription activator to create a gene activator which can target a much larger array of sequences. In various aspects, the substitutions in CaCas9-PAM may be combined with any one of the Cas9 nickase substitutions described herein.

In some aspects, a nucleic acid comprising a CaCas9 nucleotide sequence further comprises a nucleotide sequence encoding a heterologous peptide fused in-frame with the CaCas9 coding sequence. Examples of heterologous peptide sequences that can be fused to a Cas9 protein include nuclear localization sequences, signal peptides and protein tags. In one aspect, a nucleic acid comprising a CaCas9 nucleotide sequence further comprises a sequence encoding an NLS (e.g., SV40-NLS) fused in-frame with the CaCas9 coding sequence. In a further aspect, a nucleic acid comprising a CaCas9 nucleotide sequence further comprises a sequence encoding protein tag fused in-frame with the CaCas9 coding sequence As used herein, “tag” refers to a sequence that is useful for, e.g., purifying, expressing, solubilizing, and/or detecting a polypeptide. In certain aspects, a tag can serve multiple functions. Examples of suitable protein tags for the present invention include HA, TAP, MYC, HIS, FLAG, V5, and GST tags. In a particular aspect, the tag comprises SEQ ID NO:4.

In various aspects, a nucleic acid comprising a CaCas9 nucleotide sequence further comprises all or a portion of a plasmid (e.g., vector) sequence. For example, a nucleic acid comprising a CaCas9 nucleotide sequence can include one or more plasmid sequences selected from the group consisting of a promoter sequence (e.g., an ENO1, TEF1, MAL2, URA3, ACT1, SAP2, OP4, WH11, MET3, and HWP1 promoter sequence), an antibiotic resistance sequence (e.g., nourseothricin resistance NAT^{R}), an inducible recombination sequence (e.g., FRT sequence), and a locus-targeting sequence (e.g., ENO1, RP10, and NEUTSL) to direct integration of all or a portion of the nucleic acid into a yeast genome. As those of skill in the art would appreciate in light of the present disclosure, more than one promoter sequence can be used. For example, a TEF1 promoter sequence can be inserted downstream of, e.g., an ENO1 promoter.

In some embodiments, the locus-targeting sequence targets the CRISPR system to an intergenic space (e.g., the Neut5L locus).

In some embodiments, the plasmid comprises a Cre/Lox recombination sequence.

In one embodiment, a dominant resistance marker sequence is used. In some embodiments, the yeast strain is a prototroph. In some embodiments, the yeast strain is an auxotroph.

A variety of suitable plasmids and plasmid sequences suitable for use in the present invention are known in the art and readily available (Celik E and Calik P, Biotechnol Adv. 30(5):1108-18, 2011), including, e.g., pYES, pYC, pRS (e.g., pRS416), pD1201 (GAL1_P), pD1211 (TEF_P), pD1221 (ADH_P) and pD1231 (GPD_P). In some embodiments, the plasmid comprises an autonomously replicating sequence and yeast centromere sequence (CEN/ARS sequences) as, for example, in the pRS416 plasmid. In one embodiment, the nucleic acid comprising a CaCas9 nucleotide sequence is introduced into an autonomously replicating plasmid (e.g., pRS416), as described herein.

Particular examples of plasmids containing a CaCas9 nucleotide sequence are disclosed herein and include pV1025 (SEQ ID NO:13), pV987 (SEQ ID NO:28) and pV1201 (SEQ ID NO:29).

Other examples of plasmids containing a CaCas9 nucleotide sequence are disclosed herein and include pV1393, pV1326, pV1382, and pV1464 (FIGS. 13A-13C).

In some embodiments, as described herein, the promoter sequence is specific for the yeast system used to, e.g., enhance expression. For example, a S. cerevisiae TEF1 promoter is used if expressing in the S. cerevisiae system. Similarly, a promoter, e.g. TEF1 specific to Naumovozyma castellii is used if expressing in the Naumovozyma castellii system.

In some aspects, a nucleic acid comprising a CaCas9 nucleotide sequence also comprises a synthetic guide RNA (sgRNA) coding sequence. For example, the sgRNA coding sequence can be designed to express an sgRNA molecule targeting one or more of the sequences provided in the Supplementary Materials, Supplementary Data Files published in Vyas, V. K. et al., A Candida albicans CRISPR system permits genetic engineering of essential genes and gene families. Sci. Adv. 1, e1500248 (2015) (published online Apr. 3, 2015), the entire contents of which are incorporated herein by reference, and accessible at http://advances.sciencemag.org/cgi/content/full/1/3/e1500248/DC1. Thus, a variety of target sequences in a yeast genome can be modified using the present Candida-compatible CRISPR/Cas9 system.

As used herein, to “modify” a nucleic acid (e.g., a genome, a target gene, a target sequence) means to alter, or mutate, the nucleotide sequence of the nucleic acid, for example, by replacement (e.g., substitution), introduction, and/or deletion of one or more nucleotides in the nucleic acid.

The terms “target site” or “target sequence” are used interchangeably herein to refer to a nucleic acid sequence present in a target nucleic acid (e.g., a gene) to which a targeting segment of a sgRNA will bind, or hybridize, provided sufficient conditions for binding exist. For example, the target site (or target sequence) 5′-GAGCATATC-3′ (SEQ ID NO:97) within a target nucleic acid can be targeted by an sgRNA having the sequence 5′-GAUAUGCUC-3′ (SEQ ID NO:98). Suitable DNA/RNA binding conditions include physiological conditions normally present in a cell. Other suitable DNA/RNA binding conditions (e.g., conditions in a cell-free system) are known in the art.

In some aspects, a single sgRNA sequence can be complementary to one or more (e.g., all) of the target nucleic acid sequences that are being modified. In one aspect, a single sgRNA is complementary to a single target nucleic acid sequence. In a particular aspect in which two or more target nucleic acid sequences are to be modified, multiple sgRNA sequences (or sgRNA coding sequences) can be introduced, wherein each sgRNA sequence is complementary to (specific for) one target nucleic acid sequence. In other aspects, a single sgRNA sequence is complementary to at least two targets or more (all) of the target nucleic acid sequences.

Each sgRNA sequence can vary in length from about 8 base pairs (bp) to about 200 bp. In some aspects, the sgRNA sequence can be about 9 to about 50 bp; about 10 to about 40 bp; about 12 to about 30; about 14 to about 28; about 15 to about 25; about 16 to about 24; about 17 to about 23; about 18 to about 22; about 19 to about 21 bp in length.

The portion of each target nucleic acid sequence to which each sgRNA sequence is complementary can also vary in size. In particular aspects, the portion of each target nucleic acid sequence to which the sgRNA is complementary can be about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 39, 40, 41, 42, 43, 44, 45, 46 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 81, 82, 83, 84, 85, 86, 87 88, 89, 90, 81, 92, 93, 94, 95, 96, 97, 98, or 100 nucleotides (contiguous nucleotides) in length. In some embodiments, each sgRNA sequence can be at least about 70%, 75%, 80%, 85%, 90%, 95%, 100% etc. identical or similar to the portion of each target nucleic acid sequence. In some embodiments, each sgRNA sequence is completely or partially identical or similar to each target nucleic acid sequence. For example, each RNA sequence can differ from perfect complementarity to the portion of the target sequence by about 1, 2, 3, 4, 5, 6, 7, 8, 9, 10, 11, 12, 13, 14, 15, 16, 17, 18, 19, 20, etc., nucleotides. In some embodiments, one or more sgRNA sequences are perfectly complementary (100%) across at least about 10 to about 25 (e.g., about 20) nucleotides of the target nucleic acid. Examples of target sequences in the Candida albicans genome are provided in Table 1 below.

TABLE 1
Examples of target sequences in the Candida
albicans genome

| Gene ID | Target sequence |
|---|---|
| C1_05310W | AAAAAAAAGGTTGGGGCAAACGG |
| | (SEQ ID NO: 101) |
| CR_07070C | AAACCGATACTGTCCTTATTAGG |
| | (SEQ ID NO: 102) |
| C6_03710W | ACCATCACTAACCCACCTGATGG |
| | (SEQ ID NO: 103) |
| C1_00040W | AGAAGTTCAACGTGAAGAAGTGG |
| | (SEQ ID NO: 104) |
| C4_00600C | TCTGGACGAGGAGGTTTTGGTGG |
| | (SEQ ID NO: 105) |

In one embodiment, the sgRNA coding sequence encodes an sgRNA that targets one or more genes that encode a DNA damage checkpoint protein, including, e.g., Rad51, Rad52, Rad59, Rad9, Rad17, Rad24, Rad53, Mec3, Ddc1, Mec1, Chk1, Dun1, CDK, and Pds1. In one embodiment, the sgRNA coding sequence encodes an sgRNA that targets one or more genes of a yeast homologous repair pathway, e.g., any one or more genes of the MRX (Mre11/Rad50/Xrs2) complex. As those of skill in the art would appreciate in light of the present disclosure, any combination of modifications to such genes can be made to produce a desired result, such as, for example, to generate a yeast system capable of non-homologous end joining, or a yeast system capable of CRISPR-mediated mutagenesis in the absence of a repair template.

In one aspect, the sgRNA coding sequence is operably linked to a promoter (e.g., a different promoter than the promoter that controls expression of the CaCas9 sequence). A variety of suitable promoters for use in the present invention are known in the art. In a particular aspect, the promoter is a yeast RNA polymerase III promoter (e.g., a Candida albicans SNR52 promoter, or RDN5 promoter). In some embodiments, as described herein, the promoter sequence can be specific for the yeast system used. For example, a S. cerevisiae SNR52 promoter can be used if expressing in the S. cerevisiae system. Similarly, a promoter, e.g. SNR52 specific to Naumovozyma castellii can be used if expressing in the Naumovozyma castellii system.

As used herein, “operably linked” refers to a juxtaposition wherein the components are in a relationship permitting them to function in their intended manner. For example, a promoter is operably linked to a coding sequence if the promoter affects its transcription or expression. Thus, for example, a promoter operably linked to an sgRNA coding sequence allows for the expression of the sgRNA, which affects targeting of the CRISPR/Cas system to a gene of interest (e.g., the target gene), to enable modification of the target gene.

Particular examples of plasmids containing both a CaCas9 nucleotide sequence and a sgRNA coding sequence are disclosed herein and include pV1081 (SEQ ID NO:16), pV1086 (SEQ ID NO:17), pV1102 (SEQ ID NO:18), pV1107 (SEQ ID NO:19), pV1123 (SEQ ID NO:20), pV1126 (SEQ ID NO:21), pV1147 (SEQ ID NO:22), pV1129 (SEQ ID NO:23), pV1132 (SEQ ID NO:24), pV1138 (SEQ ID NO:25), and pV1144 (SEQ ID NO:26).

Other examples of plasmids containing both a CaCas9 nucleotide sequence and a sgRNA coding sequence are disclosed herein and include pV1393, pV1326, pV1382, and pV1464 (FIGS. 13A-13C).

In other aspects, the invention relates to a nucleic acid for delivering an sgRNA coding sequence. The nucleic acid for delivering an sgRNA coding sequence can include, for example, a promoter (e.g., an RNA polymerase III promoter), a cloning site for introducing an sgRNA coding sequence, and/or a locus-targeting sequence to direct integration of all or a portion of the nucleic acid into a yeast genome (e.g., a yeast RP10 sequence). In some aspects, the nucleic acid for delivering an sgRNA coding sequence comprises a synthetic guide RNA (sgRNA) coding sequence. For example, the sgRNA coding sequence can be designed to express an sgRNA molecule targeting one or more of the sequences provided herein using routine knowledge and skills possessed by one of ordinary skill in the art. As will be appreciated by those of skill in the art in light of the present disclosure, the sgRNA can be delivered as a DNA molecule (e.g., as nucleic acid encoding the desired sgRNA) or an RNA molecule.

In some aspects, the nucleic acid for delivering an sgRNA coding sequence includes an RNA polymerase III promoter. In a particular aspect, the RNA polymerase III promoter is a yeast (e.g., Candida albicans) SNR52 promoter.

In other aspects, the nucleic acid for delivering an sgRNA coding sequence includes a yeast (e.g., Candida albicans) RP10 sequence as a locus-targeting sequence.

In various aspects, a nucleic acid for delivering an sgRNA coding sequence further comprises all or a portion of a plasmid (e.g., vector) sequence. For example, a nucleic acid for delivering an sgRNA coding sequence can include an antibiotic resistance sequence (e.g., a sequence that confers resistance to nourseothricin (Nat)). A variety of suitable plasmids and plasmid sequences suitable for use in the present invention are known in the art (Celik E and Calik P, Biotechnol Adv. 30(5):1108-18, 2011).

Particular examples of plasmids containing a nucleic acid for delivering an sgRNA coding sequence are disclosed herein and include, e.g., pV1090 (SEQ ID NO:14).

In various aspects, the nucleic acids of the present invention comprise non-naturally occurring sequences.

In other aspects, the invention provides a kit comprising a nucleic acid comprising a Candida-compatible clustered regularly interspaced short palindromic repeat (CRISPR)-associated nuclease 9 (Cas9) variant (CaCas9) nucleotide sequence of a wild-type Cas9 coding sequence (e.g., SEQ ID NO:1). In some aspects, the kit further comprises a nucleic acid comprising a promoter (e.g., an RNA polymerase III promoter), a cloning site for introducing an sgRNA coding sequence, and a locus-targeting sequence to direct integration of all or a portion of the nucleic acid into a yeast genome (e.g., a yeast RP10 sequence).

In particular aspects, the kit comprises any one or more of pV1025 (SEQ ID NO:13), pV1090 (SEQ ID NO:14), pV1093 (SEQ ID NO:15), pV1200 (SEQ ID NO:27), and pV987 (SEQ ID NO:28).

Typically, the kits are compartmentalized for ease of use and can include one or more containers with reagents. In one embodiment, all of the kit components are packaged together. Alternatively, one or more individual components of the kit can be provided in a separate package from the other kits components. The kits can also include instructions for using the kit components.

### Genetically-Modified Yeast Cells Comprising Candida-Compatible Nucleic Acids Encoding CRISPR/Cas9 System Components

In other aspects, the present invention provides a genetically-modified yeast cell having a nucleic acid comprising a Candida-compatible clustered regularly interspaced short palindromic repeat (CRISPR)-associated nuclease 9 (Cas9) (CaCas9) nucleotide sequence. In some aspects, the CaCas9 nucleotide sequence has at least 40%, 50%, 60%, 70%, 80%, 85%, 90%, 93%, 94%, 95%, 96%, 97%, 98%, or 99% sequence identity to SEQ ID NO:1.

In some aspects, the genetically-modified yeast cell comprises a nucleic acid comprising a Candida-compatible clustered regularly interspaced short palindromic repeat (CRISPR)-associated nuclease 9 (Cas9) nucleotide sequence (CaCas9) that encodes a protein having at least 70%, 80%, 85%, 90%, 93%, 94%, 95%, 96%, 97%, 98%, or 99% sequence identity to SEQ ID NO: 5, or a fragment thereof, wherein each leucine in the protein is encoded by a codon other than CTG, e.g., CTC, TTG, CTT, CTA, and TTA. In certain aspects, the nucleic acid comprises a CaCas9 that encodes SEQ ID NO: 5.

As used herein, a yeast cell is “genetically-modified” when an exogenous source of DNA (e.g., a nucleic acid comprising a CaCas9 nucleotide sequence) has been introduced into the cell, for example, by transformation. In some aspects, the exogenous DNA is integrated into the cell's genome, either permanently or transiently. In other aspects, the exogenous DNA is not integrated into the host cell's genome (e.g., the DNA is maintained on an episomal element, such as a plasmid). The yeast cell can be further modified genetically through the activities of CRISPR/Cas9 system components.

In one aspect, the genetically-modified yeast cell contains a nucleic acid comprising a CaCas9 nucleotide sequence comprising a sequence having at least about 60%, 65%, 70%, 75%, 80%, 85%, 90%, 92%, 93%, 94%, 95%, 96%, 97%, 98%, 99% identity to SEQ ID NO:2 (e.g., operably linked to a promoter). In other aspects, the genetically-modified yeast cell contains a nucleic acid comprising a CaCas9 nucleotide sequence comprising SEQ ID NO: 2.

In other aspects, the genetically-modified yeast cell contains a nucleic acid comprising a CaCas9 nucleotide sequence that encodes a nuclease-inactive Cas9 protein, or a fragment thereof. Examples of nuclease-inactive Cas9 proteins are described hereinabove. In one aspect, the nuclease-inactive Cas9 protein comprises one or more substitutions relative to SEQ ID NO:5, wherein, e.g., the aspartate at position 10 and the histidine at position 840 in SEQ ID NO:5 have been substituted with a different amino acid (e.g., alanine) in the nuclease-inactive Cas9. In a particular aspect, the CaCas9 nucleotide sequence encoding the nuclease-inactive Cas9 comprises SEQ ID NO:3. In further aspects, the CaCas9 nucleotide sequence encoding the nuclease-inactive Cas9 further comprises all or a portion of a nucleotide sequence that encodes a repressor protein, as described herein. In one aspect, the nucleic acid comprises a CaCas9 nucleotide sequence encoding a nuclease-inactive Cas9 fused in-frame to a nucleotide sequence encoding the Candida albicans SSN6 repressor.

In some aspects, the genetically-modified yeast cell also includes a nucleotide sequence encoding an sgRNA. The nucleotide sequence encoding an sgRNA can be present in the nucleic acid (e.g., plasmid) that includes the CaCas9 nucleotide sequence, or can be in a separate nucleic acid molecule (e.g., plasmid). As will be appreciated by those of skill in the art in light of the present disclosure, the sgRNA may be designed to target a variety of sequences in a yeast genome, depending upon the desired results. For example, the sgRNA may target one or more of the sequences provided herein using routine knowledge and skills possessed by one of ordinary skill in the art. In general, the nucleic acid comprising a nucleotide sequence encoding an sgRNA will also comprise a promoter (e.g., an RNA polymerase III promoter) and a locus-targeting sequence to direct integration of all or a portion of the nucleic acid into a yeast genome (e.g., a yeast RP10 sequence).

In one embodiment, the genetically-modified yeast cell comprises an sgRNA coding sequence encoding an sgRNA that targets one or more genes of the DNA damage checkpoint protein, including, e.g., Rad51, Rad52, Rad59, Rad9, Rad17, Rad24, Rad53, Mec3, Ddc1, Mec1, Chk1, Dun1, CDK, and Pds1. In one embodiment the genetically-modified yeast cell comprises an sgRNA coding sequence encoding an sgRNA that targets one or more genes of the yeast homologous repair pathway, e.g., any one or more genes of the MRX (Mre11/Rad50/Xrs2) complex. Accordingly, as described herein, the present invention provides a yeast system wherein CRISPR-mediated mutagenesis can be obtained without a repair template. In one embodiment, the genetically-modified yeast cell is capable of non-homology end joining (NHEJ).

The genetically-modified yeast cell can be any yeast cell that is capable of being transformed with a nucleic acid that comprises a CaCas9 nucleotide sequence, and is capable of stably expressing a Cas9 protein (e.g., active Cas9, nuclease-inactive Cas9, or Cas9 nickase). In certain aspects, the yeast is a natural isolate (e.g., clinical isolate). In other aspects, the yeast is a laboratory strain. In some aspects, the yeast cell belongs to a fungal CTG clade species. Particular examples of fungal CTG clade species include, but are not limited to, Scheffersomyces (Pichia) stipitis, Candida famata, Candida tropicalis, Meyerozyma (Pichia) guilliermondii, Candida tenuis, Candida maltosa, Candida rugosa, Millerozyma (Pichia) farinosa, Candida oleophila, Candida albicans, Spathaspora passalidarum, Cylichna cylindracea, Debaryomyces hansenii, Lodderomyces elongisporus, Candida melibiosica, Candida parapsilosis, Candida lusitaniae, Candida guilliermondii, and Candida albicans SC5314.

In other aspects, the yeast cell is not a CTG clade yeast, e.g., Saccharomyces bayanus, Saccharomyces paradoxus, Saccharomyces cerevisiae RM11-1A, Saccharomyces cerevisiae 288C, Saccharomyces cerevisiae YJM789, Saccharomyces mikatae, Saccharomyces kudriavzevil, Saccharomyces castellii, Candida glabrata, Schizosaccharomyces japonicas, Schizosaccharomyces octosporus, Schizosaccharomyces pombe, Kluyveromyces lactis, Kluyveromyces waltii, Aspergillus clavatus, Aspergillus nidulans, Aspergillus fumigatus, Aspergillus niger, Aspergillus terreus, Aspergillus flavus, Aspergillus oryzae, Trichoderma reesei, Trichoderma virens, Trichoderma atroviride, Yarrowia hpolytica, Saccharomyces cerevisiae, Saccharomyces kluyveri, Coccidioides immitis RMSCC2394, Coccidioides immitis RS, Coccidioides immitis H538.4, Coccidioides immitis RMSCC3703, Coccidioides posadasii RMSCC3488, Coccidioides posadasii str. Silveira, Uncinocarpus reesii, Histoplasma capsulatum, Paracoccidioides brasiliensis Pb01, Paracoccidioides brasiliensis Pb03, Paracoccidioides brasiliensis Pb18, Mycosphaerella fijiensis, Mycosphaerella graminicola, Stagonospora nodorum, Cochliobolus heterostrophus, Pyrenophora tritici-repentis, Botrytis cinerea, Sclerotinia sclerotiorum, Chaetomium globosum, Podospera anserina, Neurospora crassa, Magnaporthe grisea, Verticillium dahliae, Nectria haematococca, Fusarium graminearum, Fusarium oxysporum, Fusarium verticillioides, Eremothecium gossypil, Puccinia graminis, Sporobolomyces roseus, Malassezia globose, Ustilago maydis, Coprinus cinereus, Laccaria bicolor, Phanerochaete chrysosporium, Postia placenta, Cryptococcus gattii R265, Cryptococcus gattii WM276, Cryptococcus neoformans H99, Cryptococcus neoformans JEC21, Batrachochytrium dendrobatidis JEL423, Batrachochytrium dendrobatidis JAM81, Phycomyces blakesleeanus, Rhizopus oryzae, and Encephalitozoon cuniculi. In a particular aspect, the yeast cell belongs to the genus Candida.

As would be apparent to those of skill in the art in light of the present disclosure, the various embodiments of the present invention can be used in a non-CTG clade yeast system, using an endonuclease (e.g., Cas9) that has been codon-optimized for that particular yeast system.

In some embodiments, the various embodiments of the present invention can be used in a yeast strain that has a natural mutation in one or more genes of, e.g., the DNA damage checkpoint proteins or genes of the homologous repair pathway, as described herein. In certain embodiments, the various embodiments of the present invention can be used in a yeast strain that is naturally capable of non-homologous end joining.

### Methods of Producing Genetically-Modified Yeast Cells Using Candida-Compatible Nucleic Acids Encoding CRISPR/Cas9 System Components

In yet another aspect, the present invention provides a method for modifying a genome of a yeast cell. The method generally comprises the steps of: a) introducing into the yeast cell a first nucleic acid comprising a Candida-compatible clustered regularly interspaced short palindromic repeat (CRISPR)-associated nuclease 9 (CaCas9) nucleotide sequence that encodes a protein sequence having at least 90% sequence identity to SEQ ID NO: 5, or a fragment thereof, wherein each leucine in the protein is encoded by a codon other than CTG or CUG; b) introducing into the yeast cell a second nucleic acid comprising an sgRNA coding sequence; and c) expressing the CaCas9 and sgRNA coding sequences in the yeast cell, thereby modifying the genome of the yeast cell. Methods of introducing nucleic acids (e.g., plasmids) into cells (e.g., yeast cells) are well known in the art and include, for example, routine methods for transforming yeast cells (e.g., by electroporation).

Suitable first nucleic acids (e.g., DNA or RNA) comprising a CaCas9 nucleotide sequence for use in the methods of the invention include, for example, the various nucleic acids comprising a CaCas9 nucleotide sequence disclosed herein. Particular examples of nucleic acids comprising a CaCas9 nucleotide sequence include pV1025 (SEQ ID NO:13), pV987 (SEQ ID NO:28), pV1201 (SEQ ID NO:29), pV1081 (SEQ ID NO:16), pV1086 (SEQ ID NO:17), pV1102 (SEQ ID NO:18), pV1107 (SEQ ID NO:19), pV1123 (SEQ ID NO:20), pV1126 (SEQ ID NO:21), pV1147 (SEQ ID NO:22), pV1129 (SEQ ID NO:23), pV1132 (SEQ ID NO:24), pV1138 (SEQ ID NO:25), and pV1144 (SEQ ID NO:26).

Suitable second nucleic acids (e.g., DNA or RNA) comprising an sgRNA coding sequence for use in the methods of the invention include, for example, the various nucleic acids comprising an sgRNA coding sequence disclosed herein. Particular examples of nucleic acids comprising an sgRNA coding sequence include pV1090 (SEQ ID NO: 14), pV1081 (SEQ ID NO:16), pV1086 (SEQ ID NO:17), pV1102 (SEQ ID NO:18), pV1107 (SEQ ID NO:19), pV1123 (SEQ ID NO:20), pV1126 (SEQ ID NO:21), pV1147 (SEQ ID NO:22), pV1129 (SEQ ID NO:23), pV1132 (SEQ ID NO:24), pV1138 (SEQ ID NO:25), and pV1144 (SEQ ID NO:26). In certain aspects, the second nucleic acid is introduced into the yeast cell bound to (e.g., in a complex with) a Cas9 protein, or fragment thereof.

In some aspects, the method further comprises introducing into the yeast cell a repair template nucleotide sequence. As used herein, a “repair template” refers to a nucleic acid sequence that is complementary to a portion of a target nucleic acid sequence that is cleaved by a Cas (e.g., Cas9) protein. A variety of nucleic acid sequences can be included in a repair template, including, e.g., a single-stranded oligonucleotide, a double-stranded oligonucleotide, a plasmid, a cDNA, a gene block (e.g., gBlocks™ Gene Fragments (IDT)), a PCR product, and the like. Thus, the size of the nucleic acid sequences can vary and will depend upon the reason for introducing the nucleic acid sequence.

For example, the one or more nucleic acid sequences can be used to replace one or more nucleotides, introduce one or more additional nucleotides, delete one or more nucleotides or a combination thereof in the target nucleic acid sequences. In a particular aspect, the repair template nucleotide sequence introduces a point mutation in the target sequences. In another aspect, the repair template replaces a mutant nucleotide with a wild-type nucleotide in the target sequences. In other aspects, the repair template may introduce a tag (e.g., a fluorescent protein such as green fluorescent protein), label and/or cleavage site. Thus, the repair template sequence can be from about 10 nucleotides to about 5000 nucleotides, about 20 to 4500 nucleotides, about 30 to 4000 nucleotides, about 50 to 3500 nucleotides, about 60 to about 3000 nucleotides, about 70 to about 2500 nucleotides, about 80 to about 2000 nucleotides, about 90 to about 1500 nucleotides, about 100 to about 1000 nucleotides, etc. In a particular aspect, the nucleic acid sequence is about 10 to about 500 nucleotides. In a particular aspect, the repair template sequence (e.g., oligonucleotide) is used to further modify (alter, edit, mutate) the cleaved target nucleic acid sequence (e.g., such oligo-mediated repair allows for precise genome editing). As will be apparent to those of skill in the art, a variety of methods for introducing nucleic acid into a yeast cell are well known and routine.

In certain aspects of the method, the first nucleic acid, and the second nucleic acids, or both, are introduced into the yeast cell on a plasmid. In one aspect, the first nucleic acid and the second nucleic acid are introduced into the yeast cell on a single plasmid. Particular examples of plasmids comprising a CaCas9 nucleotide sequence and an sgRNA coding sequence are disclosed herein and include pV1093 (SEQ ID NO:15), pV1081 (SEQ ID NO:16), pV1086 (SEQ ID NO:17), pV1102 (SEQ ID NO:18), pV1107 (SEQ ID NO:19), pV1123 (SEQ ID NO:20), pV1126 (SEQ ID NO:21), pV1147 (SEQ ID NO:22), pV1129 (SEQ ID NO:23), pV1132 (SEQ ID NO:24), pV1138 (SEQ ID NO:25), pV1144 (SEQ ID NO:26), and pV1201 (SEQ ID NO:29). Other examples of plasmids containing both a CaCas9 nucleotide sequence and a sgRNA coding sequence are disclosed herein and include pV1393, pV1326, pV1382, and pV1464 (FIGS. 13A-13C).

As described herein, however, the single plasmid may comprise an sgRNA coding sequence to express an sgRNA that targets a variety of sequences in a yeast genome, depending upon the desired results. For example, the sgRNA may target one or more of the sequences provided herein using routine knowledge and skills possessed by one of ordinary skill in the art.

In one embodiment, the sgRNA coding sequence encodes an sgRNA that targets one or more genes that encode a DNA damage checkpoint protein, including, e.g., Rad51, Rad52, Rad59, Rad9, Rad17, Rad24, Rad53, Mec3, Ddc1, Mec1, Chk1, Dun1, CDK, and Pds1. In one embodiment, the sgRNA coding sequence encodes an sgRNA that targets one or more genes of a yeast homologous repair pathway, e.g., any one or more genes of the MRX (Mre11/Rad50/Xrs2) complex.

In further aspects of the method, the first and second nucleic acids are introduced into the yeast cell on two different plasmids, in no preferred order. For example, in one aspect, the two different plasmids are pV1025 (SEQ ID NO:13) and pV1090 (SEQ ID NO:14). In another aspect, the two different plasmids are pV987 (SEQ ID NO:28) and pV1090 (SEQ ID NO:14). In a particular aspect, the pV1090 plasmid further comprises an sgRNA coding sequence to express an sgRNA that targets a variety of sequences in a yeast genome, depending upon the desired results, as described herein.

In certain aspects, the first and second nucleic acids are integrated in the genome of the yeast cell. In general, once the first and second nucleic acids are integrated into the cell's genome, the nucleic acids are expressed to produce Cas9 protein and sgRNA that can function collectively to edit the cell's genome.

### EXEMPLIFICATION

Materials and Methods

Strains and Media

Candida albicans strain SC5314 was used for all experiments unless otherwise noted. The fluconazole-resistant C. albicans strain Can90 was kindly provided by the Massachusetts General Hospital. Yeast strains were grown in YPD (1% Bacto Yeast extract, 2% Bacto Peptone, 2% Dextrose) medium supplemented with 0.27 mM uridine, and selected using Nourseothricin (Nat) at a concentration of 200 μg/ml. Transformations were performed using the lithium acetate method (27). Flipout of Nat^{R} gene from Cas9-expressing Duet vector pV1025 was done by induction of flippase by growth in Difco yeast carbon base with bovine serum albumin, and screening for isolates that had lost the Nat^{R} gene. Filamentation experiments were performed with yeast grown overnight in liquid YPD, washed twice in RPMI-1640 medium (Cat #22400-105, Life Technologies) supplemented with 10% fetal bovine serum, and incubated in RPMI+10% FBS for the indicated time at a starting OD of 0.1. Growth curves were performed in a clear-bottomed 96-well plate, incubated with shaking at 30° C. in a Tecan Saphire² plate reader, reading optical density at 600 nm every 5 minutes for the indicated time. YPD-grown overnight yeast cultures were used to inoculate these wells to an initial OD of 0.05. CRISPR-mutagenized loci were verified by sequence analysis of PCR products amplified from the target locus and by restriction digest where applicable.

Plasmids/DNA

Plasmids for CaCas9 Duet and Solo system are listed in Supplementary Table 1. The CaCas9 DNA was synthesized by BioBasic (Amherst, N.Y.), with codons optimized for expression in both C. albicans and Saccharomyces cerevisiae. All key components were verified by sequencing and restriction analysis, and vector sequences will be provided upon request. 5-10 μg of Solo and/or Duet vectors were linearized by digesting with Kpn1 and Sac1 prior to transformation for efficient targeting to the ENO1 and/or the RP10 locus. Purified repair templates (3 μg) were transformed along with the guide expression plasmids for Solo or Duet systems. Repair templates were generated with 60 bp oligonucleotide primers containing 20 bp overlap at their 3′ ends centered on the desired mutation point. Primers were extended by thermocycling with ExTaq. Most guides were either immediately adjacent to or within 15 bp of the desired mutagenesis point. Phosphorylated and annealed guide sequence containing primers were ligated into CIP-treated BsmBI digested parent vectors as depicted in FIG. 1C. Correct clones were identified by sequencing.

Computational Analysis

The diploid Candida albicans genome sequence was searched for matches to the patterns N₂₀(NGG) or (CCN)N₂₀, and selected only sequences that overlapped with features found in the most recent gff file available from the Candida Genome Database (C_albicans_SC5314_version_A22-s05-m01-r03_features.gff), excluding the chromosomes themselves. Any targets that have 6 Ts in the 20 bp before the NGG were removed, since this would result in premature termination from Pol III promoters. Since matches 13nt proximal to a PAM sequence (NGG or CCN) would also result in a cut to the genome, all sites that would be targeted by each 13 bp proximal to any PAM motif in the genome were searched. The same search was also performed with 12 bp for a stricter cutoff. The target sequences were annotated and classified based on the number of genes and intergenic regions they targeted.

### Example 1. Design of a CRISPR System for Use in Candida

To create a CRISPR system for Candida, several aspects of Candida were considered: the Cas9 gene was recoded because the leucine CUG codon is predominantly translated as serine, there are no known autonomously replicating plasmids, and there are no expression systems for small RNAs. To express a Candida-compatible Cas9 encoding DNA, a Candida/Saccharomyces-codon-optimized version of Cas9 (CaCas9) that avoids the use of the CUG codon was synthesized, ensuring compatibility with all CTG-clade species, as described herein. The CaCas9 gene (SEQ ID NO:2) was fused to sequences encoding the SV40 nuclear localization signal (NLS) and FLAG-tag (e.g., SEQ ID NO:4), for in-frame fusion to the 3′ end of the CaCas9 gene. The CaCas9 from this construct is expressed from the constitutive ENO1 promoter at the plasmid integration site. As there are no autonomously replicating plasmids in Candida, this construct was integrated by transformation into SC5314 at the ENO1 locus. The RNA polymerase III promoter, SNR52, was used to express sgRNAs necessary for Cas9 targeting.

For most genes, Candida diploids require knockout of both alleles of a gene to obtain a phenotype. To demonstrate efficacy of the Candida CRISPR system, ADE2 was chosen as the target because the ade2 mutation confers an easily visible red phenotype. The ade2-red phenotype is manifest among white ADE2/ADE2 diploids only if both alleles of the ADE2 gene are simultaneously non-functional (ade2/ade2).

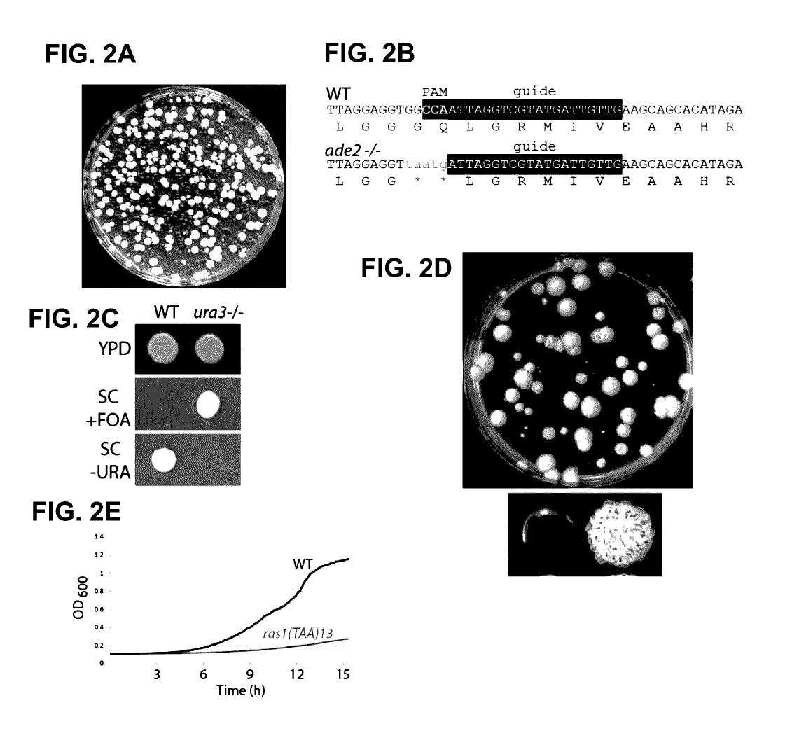

Two systems based on the design principles listed above were created. The “Duet system,” exemplified in FIG. 1A, uses the sequential integration of two plasmids. Integration of the CaCas9 expression plasmid at the ENO1 locus is first selected with Nourseothricin (Nat). By induction of the flippase gene and subsequent excision of the Nat^{R} gene, it is possible to use this marker again for selection. The second plasmid for expression of the sgRNA against ADE2 (targeted to the RP10 locus) was cotransformed with a mutagenic double-stranded oligonucleotide. This oligonucleotide is complementary to ADE2 and contains a mutation to the PAM sequence and a premature UAA stop codon (sequences shown in FIG. 2B). The second plasmid for expression of the sgRNA contains a cloning site to allow for insertion of any suitable nucleotide encoding an sgRNA of interest. No defect in the growth rate of Cas9 expressing strains was detected on YPD medium (see Materials and Methods).

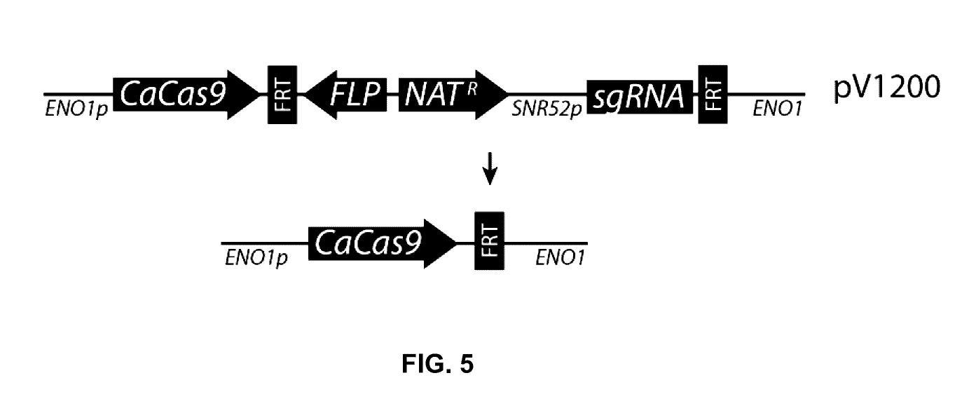

The “Solo system” (FIG. 1B) consolidates the CRISPR system with the sgRNA system by fusing them in a single plasmid construct that is then integrated at the ENO1 locus. The systems described herein permit efficient mutagenesis using a guide RNA, whose introduction is selected using the Nat resistance marker. Targeting additional genes would require the introduction of additional guides. To this end, a version of the Solo plasmid with a recyclable Nat cassette was created (FIG. 5), which permits the introduction of additional guide sequences to target other loci. Both the Duet and Solo systems feature simplified ligation of annealed oligos into the site created with BsmBI, leaving no extraneous sequences (FIG. 1C).

### Example 2. CaCas9 System Enables Highly Efficient Mutagenesis in Candida

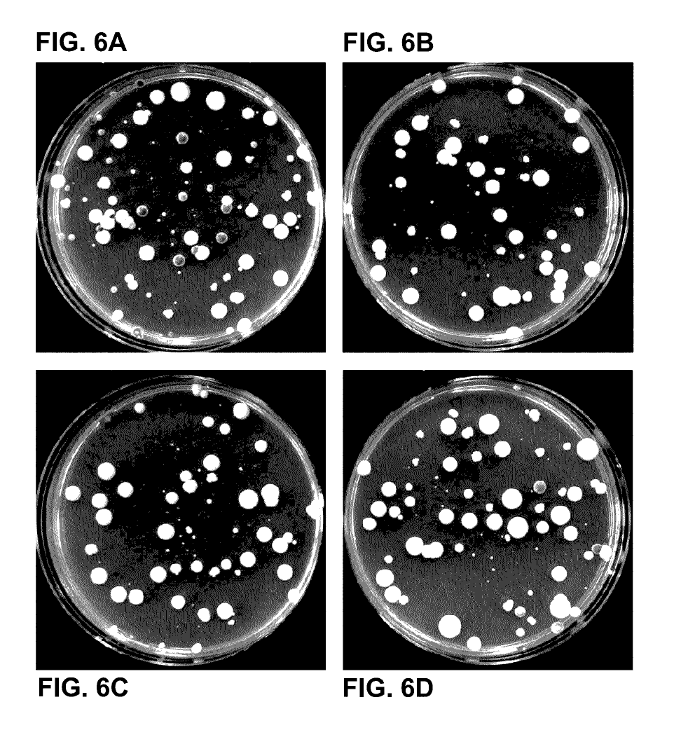

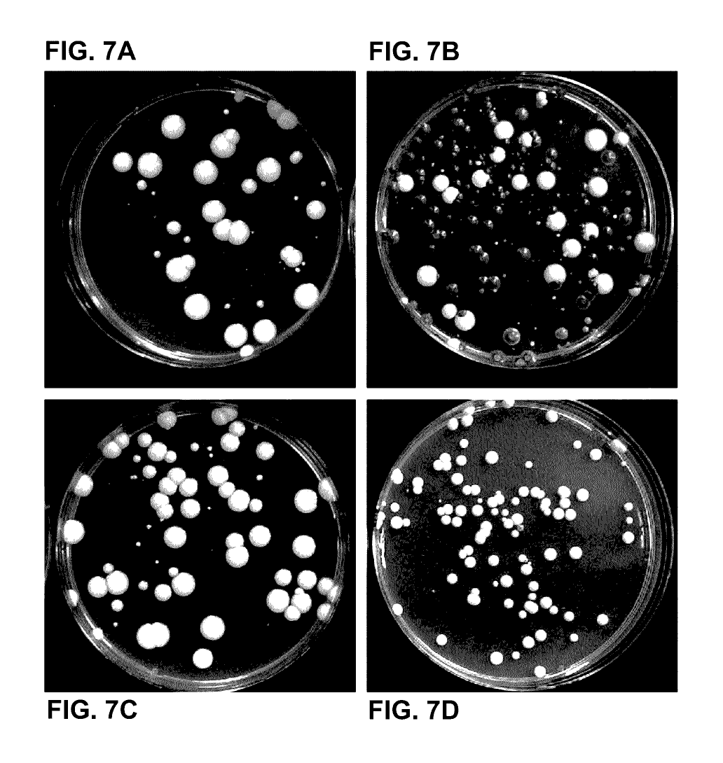

Both the Duet and Solo systems produce red ade2/ade2 transformants at high frequency (FIG. 2A, FIG. 6A, and FIG. 7B); each system uses a functional Cas9, an sgRNA against ADE2 (representing the desired target in the present example), and the complementary repair template spanning the cut site. In the absence of any one of these components only white ADE2+ colonies were obtained (FIGS. 6A-6D and FIGS. 7A-7D). The Duet system produced 20-40% red colonies among the transformants, and these were authentic CRISPR induced mutations as sequencing of the ade²/ade2 mutants revealed the UAA and the PAM mutation in the ade2 gene (FIG. 2B). The Solo system was more efficient than the Duet system; 60-80% of the transformants were red ade2/ade2 mutants (FIG. 2A and FIG. 7B). The frequency of targeting was so high that transformation with Solo plasmid and the repair template for ade2 without any selection for integration of either of the Solo Cas9 Plasmid or the repair template yielded red ade2/ade2 mutants at a rate of 2-3% (FIG. 7D).

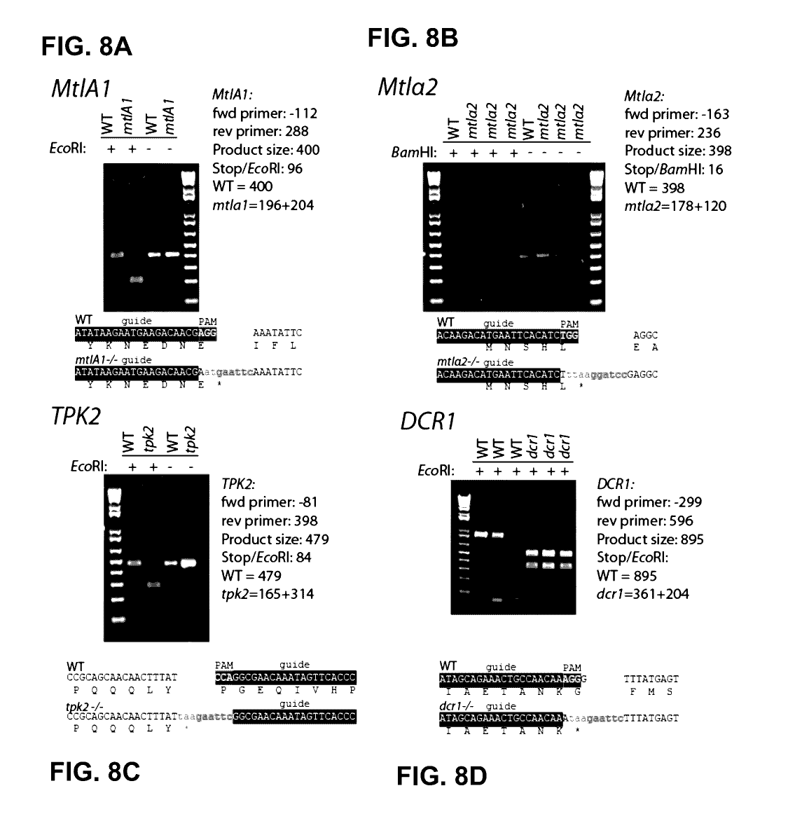

The systems described herein are generally applicable for mutagenesis of other targets. For example, mutations or truncations in URA3, RAS1, MtlA1, Mtla2, and TPK2 were readily produced using the Solo system (FIGS. 2A-2E and FIGS. 8A-8D). Transformation plates for RAS1V13 mutants provided an easy visual phenotype for identification based on colony morphology or glycogen staining with iodine (FIG. 2D). Notably, isolation of the RAS1 truncation mutants significantly reduced the growth rate (FIG. 2E) (Feng, et al., J Bacteriol 181:6339-6346 (1999)). From the transformation plates, slow growing isolates were obtained at a similar frequency to that of wrinkly colonies for RAS1V13.

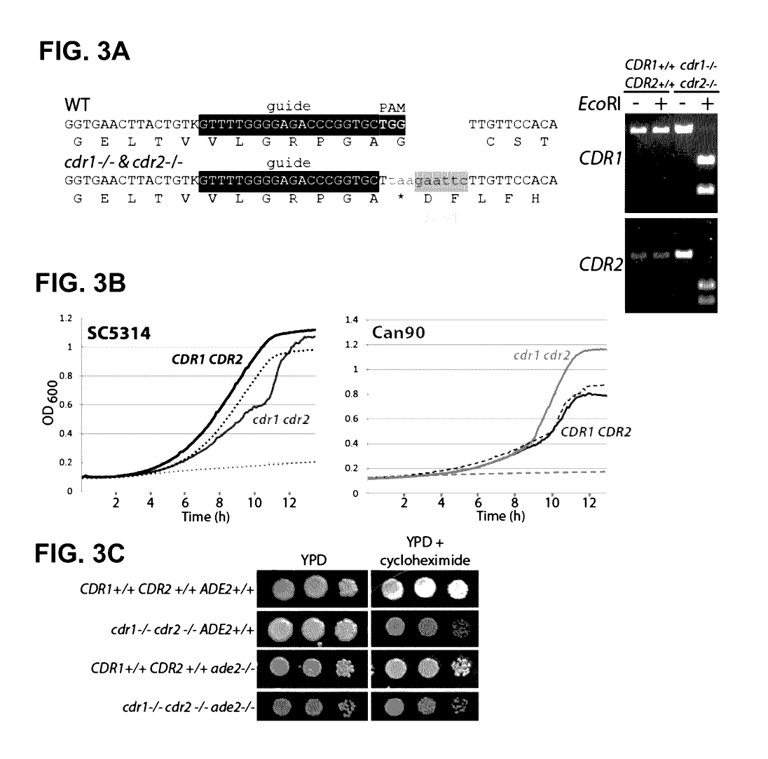

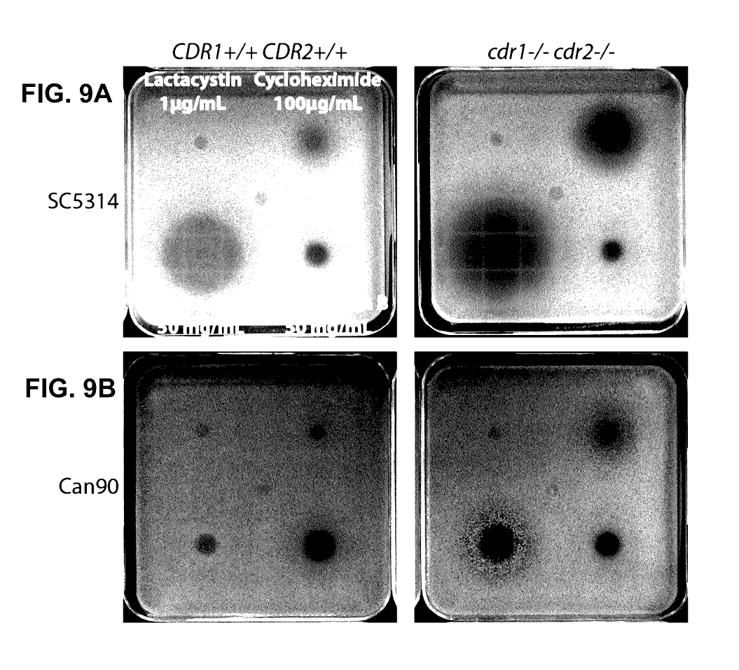

The high efficiency of the Candida CRISPR system in making homozygous knockouts enables the knock out of multiple members of a gene family with a single guide RNA. This was demonstrated by knocking out both CDR1 and CDR2, members of the multigene drug efflux pump encoding family. Loss of cdr1 or cdr2 increases sensitivity to the clinically useful azole antifungal agents (Tsao, et al., Antimicrob Agents Chemother 53:1344-1352 (2009)). To this end, an sgRNA that targeted both genes and a repair template that had homology to both CDR1 and CDR2 were designed. The repair template contained a stop codon as well as a unique restriction site, which enabled rapid genotyping of transformants (FIG. 3A). Among the transformants, drug sensitive strains that had much greater drug sensitivity than the parent were identified (FIGS. 3B and 3C; FIGS. 9A and 9B). Genotyping both by PCR and sequencing indicated these strains were double mutants of cdr1 and cdr2 (FIG. 3A).

As the present study demonstrates, four loci can be targeted with high efficiency with a single guide. Moreover, it demonstrates that a visible phenotype is not necessary to identify the intended transformants. The Candida CRISPR system was able to produce as much as ˜20% of the transformants possessing drug sensitivity. Thus, even mutants with modest phenotypic differences from wild type can now be easily identified.

A major impediment to studying Candida pathogenesis has been the paucity of antibiotic resistance markers, which coupled with diploidy and variable transformation frequency makes knockouts of a single function a considerable task. As demonstrated herein, the present system enables a single transformation experiment to mutate both copies of a gene or to delete several copies of a multigene family resulting in a discernable phenotype. Furthermore, CRISPR/Cas9 induced mutations are observed at a sufficiently high frequency such that selection is not necessary. Using a combination of guides, it has been demonstrated that both copies of three genes can be knocked out, a previously time-consuming process with no guarantee of success.

Drug resistance to azoles is a problem in the clinical treatment of Candida infections. Though several mechanisms contribute to this resistance (reviewed in Cowen, et al., Cold Spring Harb Perspect Med (2014)), upregulation of drug pumps is a common cause. To determine whether the CDR1/CDR2 CRISPR guides described herein could be used to characterize a recent fluconazole-hyper resistant clinical isolate Can90, this strain was transformed with the appropriate guides and repair templates, as done for SC5314. The cdr1/cdr1 cdr2/cdr2 homozygous double mutants (3 of 7 transformants tested) were readily identified, and no longer displayed the hyper-resistance to fluconazole or cycloheximide displayed by the parental clinical isolate, Can90 (FIG. 3B and FIG. 9B). This finding suggests a route to characterize clinical isolates of drug resistant strains of Candida. The contribution of each of the many mechanisms that render Candida resistant to antifungals—changes in ergosterol biosynthesis, upregulation of multi-drug efflux and uptake pumps, changes in cell wall composition, and the overexpression or mutation of drug target genes—can now be directly measured in clinical isolates using appropriate guides.

The ease of Saccharomyces genetics largely rests on the ability to easily produce multiple mutations in a given strain. However, without the ability to make recombinant haploids through meiosis, this is a difficult feat to achieve in Candida. To circumvent this limitation, the Solo CDR system was co-transformed alongside the sgRNA expressing Duet ADE2 vector. As the results demonstrate, strains that were simultaneously mutated at ADE2, CDR1, and CDR2 (6 loci) from a single transformation were identified using the present system (FIG. 3C).

### Example 3. Use of CaCas9 CRISPR to Target Essential Functions in Candida

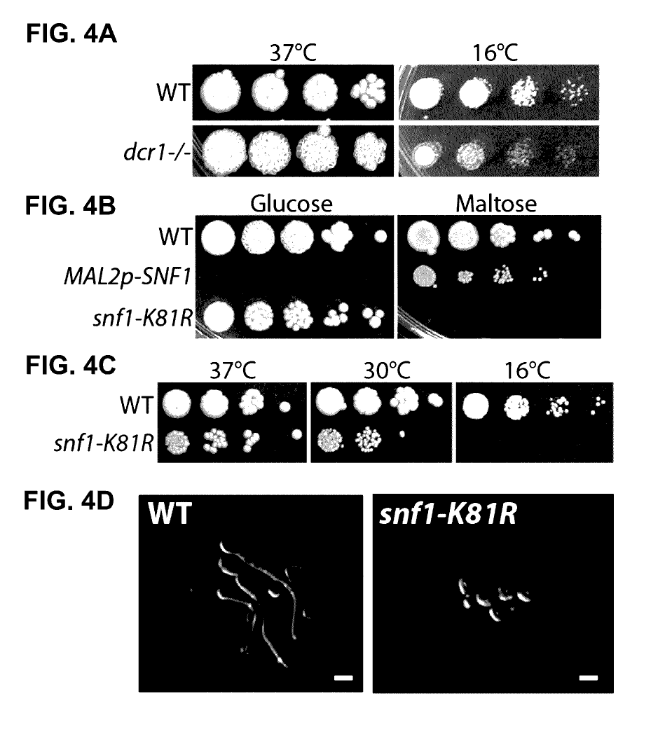

Homozygous loss of function mutations in essential genes of Candida albicans were obtained using the present CRISPR system by creating conditional alleles. Null alleles of DCR1, which is required for rRNA processing, are lethal at low temperature but viable at high temperature (Bernstein, et al., Proc Natl Acad Sci USA 109:523-528 (2012)). Transformation of SC5314 was carried out using the Solo CRISPR plasmid containing a guide directed against DCR1, and a repair template which introduced a stop codon. The transformation plates were incubated at 37° C., and transformants were screened for growth at either 37° C. or 16° C. to identify candidate dcr1/dcr1 mutants. A number of dcr1/dcr1 mutants that failed to grow at 16° C. were identified and the signature nonsense mutation confirmed (FIG. 4A and FIG. 8).

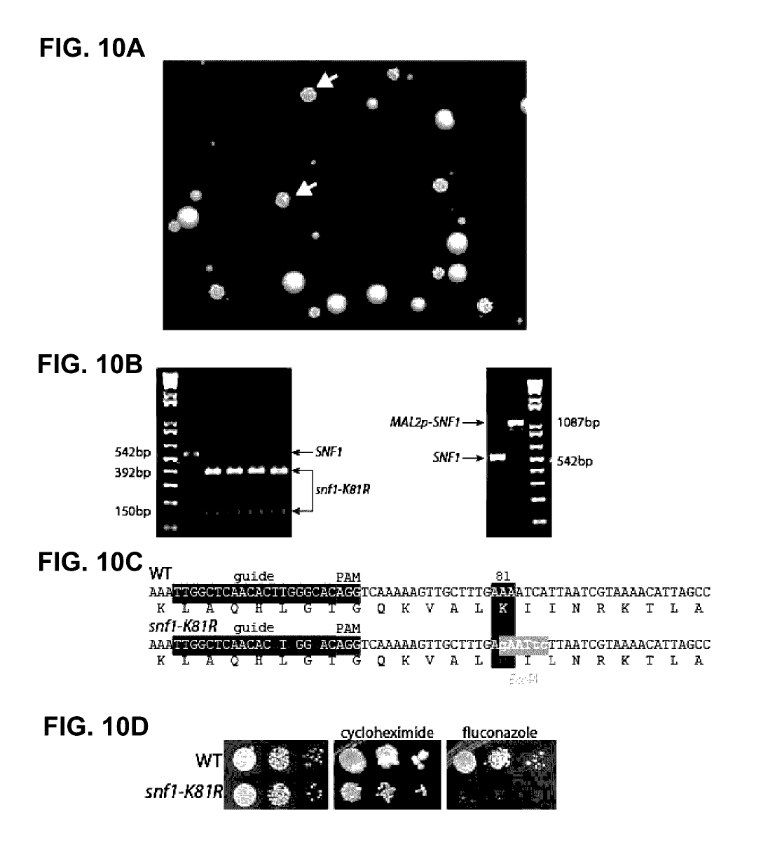

Another approach to obtaining null mutations in lethal functions is to replace the resident functional genes with the gene under the control of the inducible MAL2 promoter. To determine if a regulable promoter for SNF1, which is essential (Petter, et al., Infect Immun 65:4909-4917 (1997); Enloe, et al., J Bacteriol 182:5730-5736 (2000)), could be readily introduced, a guide was created that cut in the SNF1 promoter region and inserted a MAL2 promoter fragment with flanking homology to resident sequences, permitting SNF1 to be transcribed on maltose but not glucose. Transformation mixtures were plated onto selective maltose plates, and replica plated these onto maltose (permissive) or glucose (restrictive) media. Several transformants that only grew in maltose were identified, and confirmed that they were maltose promoter integrants (FIG. 4B and FIG. 10B), verifying the essential nature of SNF1.

Both prior attempts to knockout SNF1 function relied on the failure to obtain a homozygous gene replacement (Petter, et al., Infect Immun 65:4909-4917 (1997); Enloe, et al., J Bacteriol 182:5730-5736 (2000)) without the presence of SNF1 elsewhere in the genome. This indirect evidence suggests that the Snf1 function is essential, and implied that the kinase activity of Snf1 is required. It does not rule out the possibility that only the protein itself but not the kinase activity is required. To discriminate between these possibilities, Solo system guides were generated for SNF1, and repair templates that mutate Lysine 81 to Arginine in the ATP-binding pocket. Mutation at this conserved position either eliminates or vastly diminishes kinase activity in Saccharomyces and human Snf1/AMPK (Celenza and Carlson, Mol Cell Biol 9:5034-5044 (1989); Thornton, et al, J Biol Chem 273:12443-12450 (1998)). The K81R CRISPR transformation plates contained ˜40% wrinkled colonies (FIG. 10A), which upon further analysis was determined to be homozygous for snf1-K81R (FIGS. 10B and 10C). The snf1-K81R/snf1-K81R strains are unable to grow on maltose (FIG. 4B), consistent with the Saccharomyces snf1 mutant's failure to grow on non-glucose carbon sources (Celenza and Carlson, Mol Cell Biol 9:5034-5044 (1989); Carlson, et al., Genetics 98:25-40 (1981)). The additional phenotypes of cold sensitivity (FIG. 4C) and defective filamentous growth (FIG. 4D) are also seen in snf1 mutants in Saccharomyces (Kuchin, et al., Mol Cell Biol 22:3994-4000 (2002); Kuchin, et al., Biochem Soc Trans 31:175-177 (2003); Vyas, et al., Mol Cell Biol 23:1341-1348 (2003)). In addition, snf1-K81R was hypersensitive to fluconazole, suggesting Snf1's stress response function is required for activation of fluconazole resistance (FIGS. 10A-10D).

The high frequency of CRISPR induced mutations enables the identification of essential genes. Previously, a gene could be misconstrued as essential because low transformation frequencies and poor targeting led to the failure to obtain homozygous null mutations. The efficacy of the CRISPR technology not only overcomes this roadblock, but also permits discrimination among the functions of an essential gene. Using this technology, it was possible to determine, unexpectedly, that the kinase function of SNF1 is not required for its essential function. The prospect of uncovering all the vital functions in Candida is supported by the genomic analysis described herein, which suggests that greater than 98% of the genes are accessible to modification with the present CRISPR system. The ability to identify and analyze essential functions should facilitate the search for more effective antifungal targets.

### Example 4. Design of Nuclease-Inactive CaCas9 as Gene Repressor

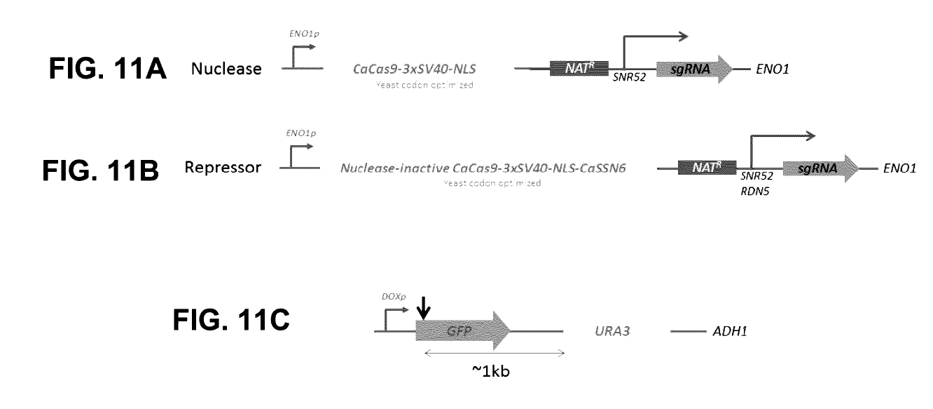

The nuclease-inactive CaCas9 contains modifications at two amino acids (D10A and H841A in SEQ ID NO:6, which is encoded by nucleotide sequence SEQ ID NO:3) resulting in a nuclease-inactive enzyme that is still capable of targeting to DNA sequences under the direction of an appropriate sgRNA. SSN6 (suppressor of Snf1 6) is a co-repressor protein that is recruited by DNA binding transcription factors to repress transcription. SSN6 does not have a DNA binding activity of its own, but will repress transcription of any promoter to which it is tethered (by fusion to a DNA binding protein). Here, Candida albicans SSN6 was fused in-frame to nuclease-inactive CaCas9 (nuclease-inactive CaCas9-SSN6) to create a chimeric repressor protein that can repress transcription in fungi (see schematic FIG. 11B). According to the present methods, the nuclease-inactive CaCas9-SSN6 gene is found in plasmids pV987 (Duet plasmid version) and pV1201 (Solo plasmid version).

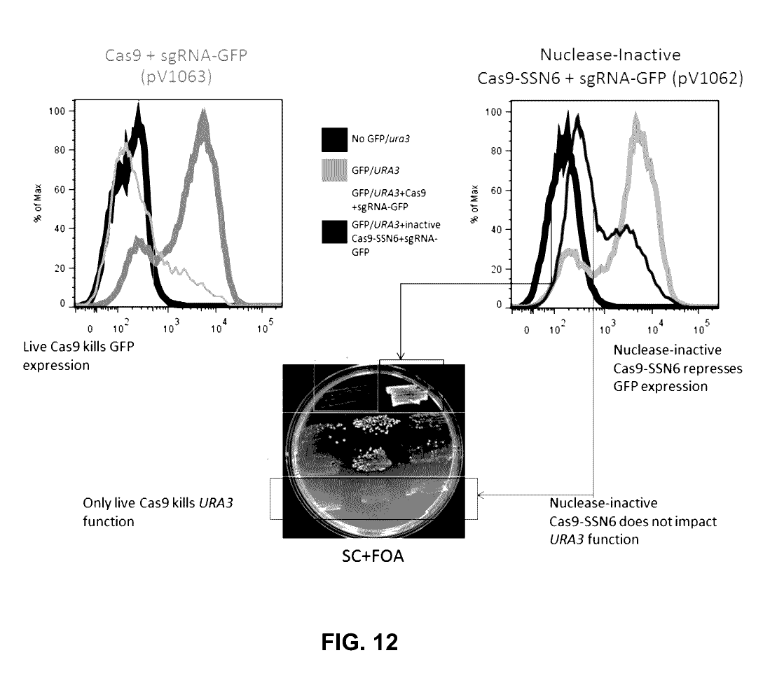

Candida albicans containing the GFP expression construct depicted in FIG. 11C was transformed with pV1062 (FIG. 11B) or pV1063 (FIG. 11A), which targets nuclease-inactive Cas9 for repression, or Cas9 cleavage of the GFP sequence, respectively. Consistent with this, reduced GFP levels were observed in pV1062 transformants (FIG. 12, right), or no GFP expression (FIG. 12, left). Consistent with cleavage of the DNA, the linked URA3 marker was lost in strains with nuclease active Cas9, likely resulting from destabilization of the cut chromosome (leading to FOA resistant colonies, as depicted in the plate in the middle of FIG. 12). FOA resistance is only possible if URA3 is inactivated; URA3+ strains are sensitive to FOA. Strains expressing nuclease-inactive Cas9-SSN6 do not lose URA3, and thus remain sensitive to FOA like the bright GFP+ strains (green histogram on left points to the position on the plate). URA-strains like the grandparent dark GFP-strain are resistant to GFP (black histogram on right points to position on FOA plate).

### Example 5. Serial Mutagenesis in C. albicans, S. Cerevisiae, and C. glabrata

As shown in FIG. 5, serial mutagenesis with the pV1200 vector requires a flippase-mediated recombination, which removes the Nat^{R} marker and guide RNA expression module at the ENO1 locus, leaving Cas9 in the genome. A similar system, pV1393 (FIG. 13A), has been generated, with some modifications. First, it targets the CRISPR system for insertion into the Neut5L locus, which is an intergenic space whose name derives from its aim to provide a neutral integration site. Second, induction of flippase completely removes CaCas9 as well as the guide expression module, leaving only an FRT insertion at Neut5L.

Vectors for serial mutagenesis in other yeast cells (e.g., Saccharomyces cerevisiae, Candida glabrata and Naumovozyma castellii—also known as Saccharomyces castellii) have also been generated. The most commonly used vectors for CRISPR mutagenesis in Saccharomyces cerevisiae have a few limitations. Most systems use auxotrophic markers for selection of Cas9 and guide plasmids, limiting their utility in prototrophs. Additionally, most separate the guide and Cas9 expression modules, which requires the use of more than one plasmid during transformation, and more than one auxotrophy in the recipient strain. The Solo system from Candida albicans could be a good template for use in Saccharomyces: it consolidates the Cas9/sgRNA modules on one plasmid, uses a dominant drug resistance marker for use in prototrophs and it contains a Cas9 whose nucleotide sequence is optimized for expression in yeast. To examine the applicability of the Solo system in Saccharomyces, the system was transferred to the pRS416 vector which provides a CEN/ARS element for episomal maintenance, and a URA3 marker, which can be used for counter-selection with FOA in ura3 auxotrophs. The promoter sequences for the sgRNA and CaCas9 were changed from one that is native to C. albicans to, e.g., Saccharomyces, to improve their expression (FIGS. 13B and 13C). The pRS416 backbone is functional in multiple yeast species, including Candida glabrata and Naumovozyma castellii, suggesting these plasmids could bring functional CRISPR mutagenesis to these species.

To demonstrate serial mutagenesis in C. albicans with pV1393, either the EFG1 and CPH1 loci or LEU2 and MET15 loci were serially targeted in SC5314. First, SC5314 was transformed with a guide targeting EFG1 or LEU2 and an appropriate repair template. After identification of nourseothricin resistant (Nat^{R}) clones with the correct mutation, they were grown in medium to induce expression of flippase (see materials and methods), and nourseothricin sensitive (Nat^{S}) clones were identified by replica plating. Nat^{S} colonies that were efg1/efg1 or leu2/leu2 were then transformed with guides and repair templates for mutagenesis of cph1/cph1 or met15/met15, respectively. Correct double mutant clones (efg1/efg1 cph1/cph1 or leu2/leu2 met15/met15) were then grown on flippase-induction medium to loop out the CRISPR system, generating Nat^{S} colonies.

Serial mutagenesis in Saccharomyces cerevisiae and Candida glabrata was also performed using the pV1382 backbone with appropriate guides, targeting ADE2, MET15, and LEU2. Strains were transformed with either pV1382 or derivatives with guides against the indicated gene with or without repair template. Mutagenesis in both Candida glabrata and Saccharomyces cerevisiae was very efficient, with over 90% of transformants displaying the red ade2 color phenotype. After overnight growth in non-selective YPD, Nat^{S} colonies were identified by replica plating. Very efficient plasmid loss in both species was observed, with rates varying from 50-90%. Mutants cured of the plasmid were successfully subjected to another round of CRISPR mutagenesis (for LEU2 and MET15) and plasmid curing.

### Example 6: CRISPR Deletion Mutants Using a Single Guide

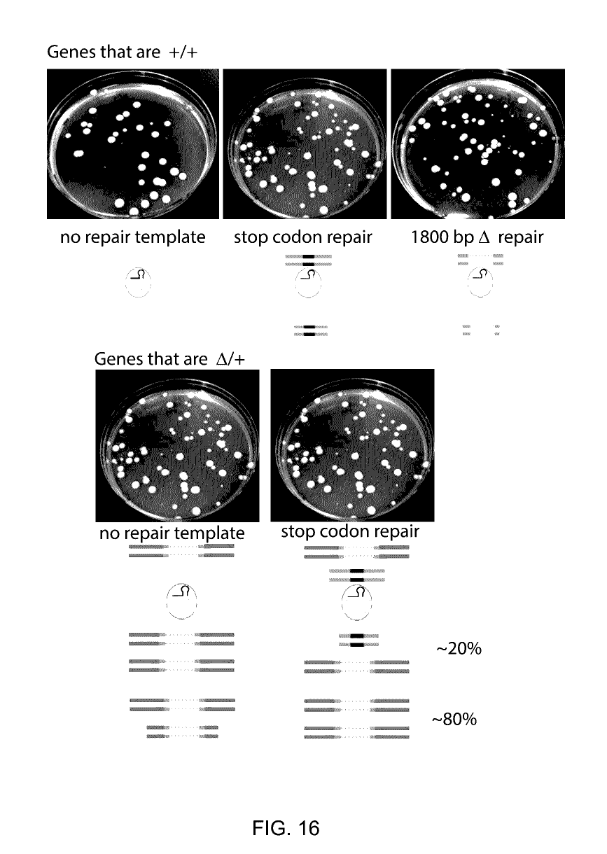

Generally, creation of deletion mutants with CRISPR utilizes two sgRNA sequences, one targeting each end of the gene, with or without a repair template. Here, it was determined whether such mutants could be generated using only a single guide sequence. As shown herein, mutagenesis at ADE2 was performed with pV1081, which contains a guide that cuts within the open reading frame alongside a repair template that introduces an early stop codon in the coding sequence. To make deletion mutants, this same guide sequence was used but changed the repair template such that it juxtaposed 50 bp upstream of the open reading frame to 50 bp downstream of the open reading frame, generating a deletion of 1652 bp. Use of this repair template with pV1081 generated ade2/ade2 mutants at a rate comparable to the stop-codon-containing repair template (FIG. 16, top). Genotyping revealed the mutants had repair template mediated repair resulting in either premature stop or deletion alleles of ade2. This same repair template design was functional in S. cerevisiae and C. glabrata.

### Example 7: Creation of Loss of Heterozygosity (LOFT) Mutants in Candida albicans

C. albicans requires a repair template in addition to Cas9/sgRNA expression for mutagenesis at a given locus possibly owing to the homologous repair machinery using the intact allele to repair the allele cleaved by Cas9/sgRNA. To test this directly, ADE2 mutagenesis was measured in a strain which contained a heterozygous deletion of ADE2. Both wild-type and ADE2 heterozygotes were transformed with plasmid pV1081 with and without repair template. In wild-type, mutagenesis of ADE2 with pV1081 required the presence of a repair template. For the ADE2 heterozygote, red ade2 colonies were obtained even in the absence of repair template (FIG. 16, bottom). When repair template was included, approximately 20% of the ade2 strains used the repair template, while the other 80% either used the other chromosome as the repair template, or homozygozed the ADE2 chromosome.

### Example 8: Repair Template Requirements in S. cerevisiae, N. castellii, and C. glabrata

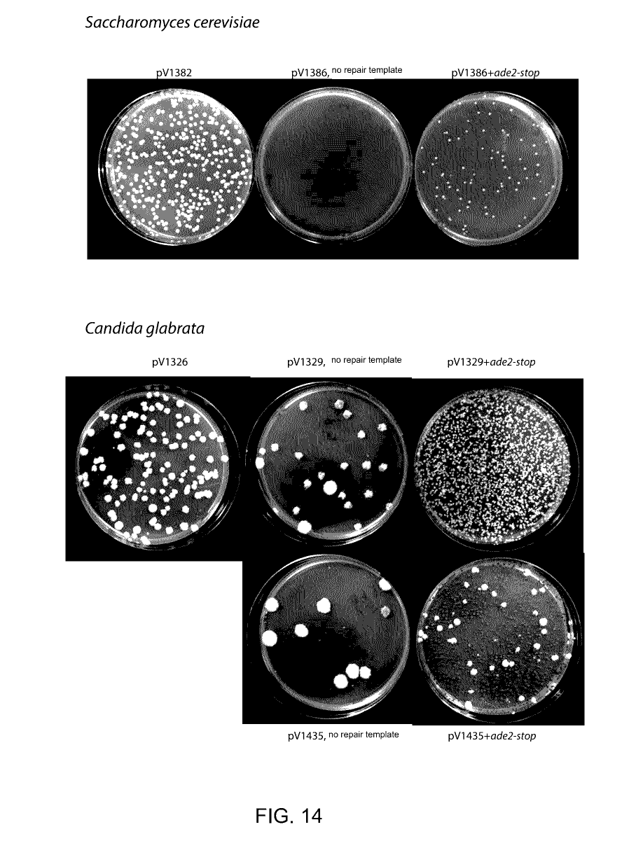

To test the repair template requirements for mutagenesis in other yeasts, S. cerevisiae, N. castellii, and C. glabrata were transformed with empty solo vectors or vectors containing guides to ADE2, both with and without repair templates, and applied selection. For Saccharomyces, ade2 mutants were obtained at a very high rate (˜100%) when a mutagenic repair template was included (FIG. 14, top). Omission of this repair template led to a failure to recover any transformants (FIG. 14, top). Transformation with an equal amount of the parent plasmid (containing a guide which does not target the genome) without repair template yielded more transformants than either ADE2 directed vector (FIG. 14, top).

In both C. glabrata and N. castellii, red ade2 mutants were obtained when the plasmid was transformed with or without a mutagenic repair template (FIG. 14 bottom, and not shown). Sequence analysis of ade2 mutants obtained without the repair template confirmed the presence of short indels, which are the hallmark of NHEJ mediated repair. When a repair template was included, the recovery rate of red ade2 improved in both species. For C. glabrata there were significant differences in the mutagenesis rate depending on the promoter used to drive CaCas9 expression. In the absence of repair template, the pV1326-based guide pV1329 (with CaENO1p driven CaCas9) had a higher rate of mutagenesis than pV1382-based guides (with CaENO1+ScTEF1 driven CaCas9—where “Sc” denotes S. cerevisiae and “Ca” denotes C. albicans). In the presence of repair template, the reverse was true, with pV1382-based vectors yielding >95% red colonies, compared to <5% with pV1326-based guides. For C. glabrata, 60-70% of ade2 mutants integrated the repair template, while the rest had similar mutations to those found in the absence of repair template. For N. castellii, the highest mutagenesis rate was obtained only after switching the expression system to the native NcTEF1 and NcSNR52 promoters (where “Nc” refers to N. castellii), and repair template-mediated and NHEJ-mediated repair was observed at rates comparable to C. glibrata (data not shown).

### Example 9: Generation of CRISPR-Derived Mutations in the Absence of Repair Template

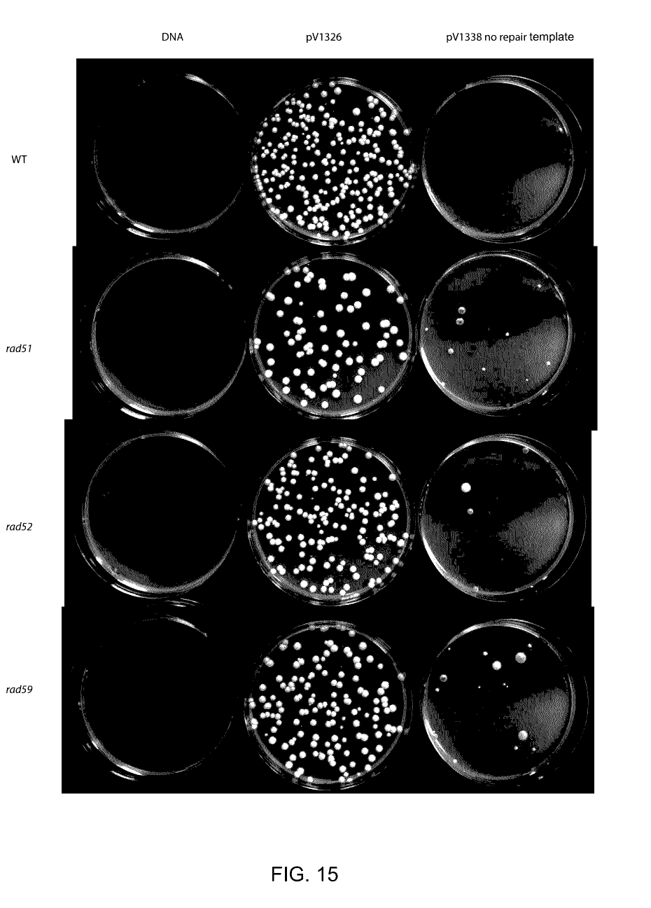

The present study examined whether mutation of the homologous repair machinery might permit the generation of CRISPR-derived mutations in the absence of repair template. To this end, WT, rad51, rad52, and rad59 strains were transformed with either an untargeted Solo plasmid pV1326, or an ADE2 directed Solo plasmid pV1338 without repair template. As shown previously, transformants were not obtained for WT with pV1338 without the addition of repair template (FIG. 15). However, in mutants of RAD51, RAD52, and RAD59, transformants were obtained, the majority of which had a red ade2 phenotype (FIG. 15). Sequence analysis of all colonies revealed they all contained indels consistent with NHEJ mediated repair. The few isolated white colonies actually contained mutations in the ADE2 locus rendering it resistant to CRISPR cleavage, while maintaining ADE2 prototrophy.

### Example 10. Identification of CRISPR Accessible Sites in the Genome

Computational analysis shows that most genes in the Candida genome can be uniquely targeted using the present invention. The most recent diploid assembly of the Candida albicans genome database (Inglis, et al., Nucleic Acids Res 40:D667-674 (2012)) for Cas9 recognition motifs—N₂₀ followed by a PAM sequence—was searched, and selected only those sequences that overlap with annotated features. Of the 6466 genes in the Candida genome, 6341 can be targeted uniquely by 601,770 guides. Of those guides, 551,175 can direct cleavage at both alleles, while 59,595 target only one of the two. A small subset of these guides target more than one location in the same gene (genes with internal repeats). The sequences of each of these guides can be found in the Supplementary Materials, Supplementary Data Files published in Vyas, V. K. et al., A Candida albicans CRISPR system permits genetic engineering of essential genes and gene families. Sci. Adv. 1, e1500248 (2015) (published online Apr. 3, 2015), the entire contents of which are incorporated herein by reference, and accessible at http://advances.sciencemag.org/cgi/content/full/1/3/e1500248/DC1. In addition, 49,195 guides that target more than one putative gene sequence, without targeting non-genic sequences, were identified. Such sequences can be found for 6023 genes. These can be used to target certain motifs or gene families for simultaneous mutagenesis using the present system, as demonstrated herein using CDR1 and CDR2.

The relevant teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

Unless defined otherwise, all technical and scientific terms used herein have the same meaning as commonly understood by one of ordinary skill in the art to which this invention pertains.

As used herein, the indefinite articles “a” and “an” should be understood to mean “at least one” unless clearly indicated to the contrary.

The phrase “and/or”, as used herein, should be understood to mean “either or both” of the elements so conjoined, i.e., elements that are conjunctively present in some cases and disjunctively present in other cases.

It should also be understood that, unless clearly indicated to the contrary, in any methods described herein that include more than one step or act, the order of the steps or acts of the method is not necessarily limited to the order in which the steps or acts of the method are recited.

TABLE 2
Plasmids used in this study

| | |
|---|---|
| pV1025 | Duet system CaCas9 expression vector, contains Nat^{R}/FLP cassette, and targeting arms for |
| | the ENO1 locus. The ENO1p is used to drive CaCas9 expression. (SEQ ID NO: 13) |
| pV1090 | Duet system sgRNA entry expression vector, contains Nat^{R} gene and the SNR52 promoter |
| | from Candida albicans driving expression of sgRNA that binds/targets Cas9, and targeting |
| | arms to direct integration to RP10. (SEQ ID NO: 14) |
| pV1093 | Solo system CaCas9/sgRNA entry expression vector, contains Nat^{R} gene, and 2kb targeting |
| | arms for the upstream and downstream of ENO1 coding region. ENO1p drives CaCas9 |
| | expression as above. (SEQ ID NO: 15) |
| pV1081 | Solo system vector to target mutagenesis of ADE2 (SEQ ID NO: 16) |
| pV1086 | Solo system vector to target mutagenesis of CDR1 and CDR2 (SEQ ID NO: 17) |
| pV1102 | Solo system vector to target mutagenesis of URA3 (SEQ ID NO: 18) |
| pV1107 | Solo system vector to target mutagenesis of RAS1 (SEQ ID NO: 19) |
| pV1123 | Solo system vector to target mutagenesis of MtlA1 (SEQ ID NO: 20) |
| pV1126 | Solo system vector to target mutagenesis of MtlAlpha2 (SEQ ID NO: 21) |
| pV1147 | Solo system vector to target mutagenesis of TPK2 (SEQ ID NO: 22) |
| pV1129 | Solo system vector to target mutagenesis of DCR1, first position (SEQ ID NO: 23) |
| pV1132 | Solo system vector to target mutagenesis of DCR1, second position (SEQ ID NO: 24) |
| pV1138 | Solo system vector to target mutagenesis of SNF1 proximal to K81 (SEQ ID NO: 25) |
| pV1144 | Solo system vector to target mutagenesis of SNF1 promoter (SEQ ID NO: 26) |
| pV1200 | Solo system CaCas9/sgRNA entry expression vector, contains Nat^{R} gene, and 2kb targeting |
| | arms for the upstream and downstream of ENO1 coding region. ENO1p drives CaCas9 |
| | expression as above. The Nat^{R} gene and SNR52p-sgRNA cassette is flanked by FRT sites, |
| | which mediate recombination when FLP expression is induced. (SEQ ID NO: 27) |
| pV987 | Duet system nuclease-inactive CaCas9 expression vector, contains Nat^{R}/FLP cassette, and |
| | targeting arms for the ENO1 locus. The nuclease-inactive CaCas9 is fused in-frame to |
| | SV40-NLS and SSN6. The ENO1p is used to drive nuclease-inactive CaCas9 expression. |
| | (SEQ ID NO: 28) |
| pV1201 | Solo system dCaCas9/sgRNA entry expression vector, contains Nat^{R} gene, and 2kb |
| | targeting arms for the upstream and downstream of ENO1 coding region. The dCaCas9 is |
| | fused in-frame to SSN6. ENO1p drives CaCas9 expression as above. (SEQ ID NO: 29) |

Oligonucleotide Sequences Used in this Study

s2RNA clonin2 Primers

| | |
|---|---|
| sgADE2 top | atttgCAACAATCATACGACCTAATg (SEQ ID NO: 30) |
| sgADE2 bottom | AAAACattaggtcgtatgattgttgc (SEQ ID NO: 31) |
| sgURA3 top | atttgAGTTTCTGCTCTCTCACTATg (SEQ ID NO: 32) |
| sgURA3 bottom | AAAACatagtgagagagcagaaactc (SEQ ID NO: 33) |
| sgRAS1 top | atttgAAATTAGTTGTTGTTGGAGGG (SEQ ID NO: 34) |
| sgRAS1 bottom | AAAACCCTCCAACAACAACTAATTTc (SEQ ID NO: 35) |
| sgMtlA1 top | atttgATATAAGAATGAAGACAACGg (SEQ ID NO: 36) |
| sgMtlA1 bottom | aaaacCGTTGTCTTCATTCTTATATc (SEQ ID NO: 37) |
| sgMt1A1pha2 top | atttgACAAGACATGAATTCACATCG (SEQ ID NO: 38) |
| sgMt1A1pha2 bottom | AAAACGATGTGAATTCATGTCTTGTc (SEQ ID NO: 39) |
| sgSnf1p top | atttgATATAATGTGTATTACTTCTG (SEQ ID NO: 40) |
| sgSnf1p bottom | AAAACAGAAGTAATACACATTATATc (SEQ ID NO: 41) |
| sgSnf1-1 top | atttgTTGGCTCAACACTTGGGCACG (SEQ ID NO: 42) |
| sgSnf1-1 bottom | AAAACGTGCCCAAGTGTTGAGCCAAc (SEQ ID NO: 43) |
| sgDcr1-1 top | atttgATAGCAGAAACTGCCAACAAg (SEQ ID NO: 44) |
| sgDcr1-1 bottom | aaaacTTGTTGGCAGTTTCTGCTATc (SEQ ID NO: 45) |
| sgDcr1-2 top | atttgTTATGAGTTACATCAACAACg (SEQ ID NO: 46) |
| sgDcr1-2 bottom | aaaacGTTGTTGATGTAACTCATAAc (SEQ ID NO: 47) |
| sgTpk2 top | atttgGGGTGAACTATTTGTTCGCCG (SEQ ID NO: 48) |
| sgTpk2 bottom | AAAACGGCGAACAAATAGTTCACCCc (SEQ ID NO: 49) |

PCR/Sequencing Primers

| | |
|---|---|
| ADE2-fwd | Aacaccccccaccaaaaagaatc (SEQ ID NO: 50) |
| ADE2-rev | Acaagtcatcgactgtgttgg (SEQ ID NO: 51) |
| CDR1-fwd | AAAACATTCAGAATTTAGCCAG (SEQ ID NO: 52) |
| CDR2-fwd | Atagaaatttaagagcttacgg (SEQ ID NO: 53) |
| CDR12-rev | Aggttgccatataaacactagcc (SEQ ID NO: 54) |
| URA3-fwd | Tttgttcttcaatgatgatttcaacc (SEQ ID NO: 55) |
| URA3-rev | Cataaattgatgtttacgtgaaagttc (SEQ ID NO: 56) |
| RAS1-fwd | TCAATTGACTAGATATAAACTCTTC (SEQ ID NO: 57) |
| RAS1-rev | TCCATCTTCATAACTAACTTGTCTT (SEQ ID NO: 58) |
| MatA1-fwd | TTCAATAGTTTTTTTCTGCGTATTGTG (SEQ ID NO: 59) |
| MtlA1-rev | TCGATCCAGCAATGGAAGATAGCTT (SEQ ID NO: 60) |
| MtlAlpha2-fwd | CTTAGTCTAACTTTATAGTTGTC (SEQ ID NO: 61) |
| Mt1A1pha2-rev | ATTCTTTCTAATAACATTTCATGCAA (SEQ ID NO: 62) |
| Snf1-fwd | TGTCATTCCGTTTCTCCTTCTA (SEQ ID NO: 63) |
| Snf1-rev | GCAAATTCAATAACCATAATG (SEQ ID NO: 64) |
| DCR1-fwd | GGTATTATTTTGACTTCATC (SEQ ID NO: 65) |
| DCR1-rev | TCACTTATTTTGACTTCATC (SEQ ID NO: 66) |
| Tpk2-fwd | TTAAAGAAACTTCACATCACCAA (SEQ ID NO: 67) |
| Tpk2-rev | ACTTTGATAGCATAATATCTAC (SEQ ID NO: 68) |

Repair Templates for mutagenesis

| | |
|---|---|
| ADE2-NT2-top | Taatggatagcaaaactgttggtattttaggaggttaatgattaggtcgtatgat |
| | tgttgaagcag (SEQ ID NO: 69) |
| ADE2-NT2- | Cggtcttgatattcaatctatgtgctgcttcaacaatcatacgacctaat (SEQ |
| bottom | ID NO: 70) |
| ADE2-NT1-top | ttgatgttgatgctttaatcaaagttcaagagaaattAACtaaagttgaaatata |
| | tccattacTACCTGAAAC (SEQ ID NO: 71) |
| ADE2-NT1- | Tatcttgaatcaatcttatggtttcaggtaatggatatatttcaacttta (SEQ |
| bottom | ID NO: 72) |
| CDR12-top | ccaggtgaacttactgtKgttttggggagacccggtgctTAAGaaTTCttgttcc |
| | acatt (SEQ ID NO: 73) |
| CDR12-bottom | tgtggaaaccataagtgttaacagcaatggtctttaacaatgtggaacaaGAAtt |
| | CTTAa (SEQ ID NO: 74) |
| URA3-top | aaatagcaaacaaaagatatgacagtcaacactTAATAATatagtgagagagcag |
| | aaact (SEQ ID NO: 75) |
| URA3-bottom | Aaataatcgttgtgctactggtgaggcatgagtttctgctctctcactat (SEQ |
| | ID NO: 76) |
| RAS1-V13-top | ATATCCACACATATACATACCATGTTGAGAGAATATAAATTAGTTGTTGTTGGAG |
| | GTGtT (SEQ ID NO: 77) |
| RAS1-V13- | AATCAATTGAATGGTTAAAGCGGATTTACCAACACCAaCACCTCCAACAACAACT |
| bottom | AATTT (SEQ ID NO: 78) |
| RAS1-TAA13- | ATATCCACACATATACATACCATGTTGAGAGAATATAAATTAGTTGTTGTTGGAG |
| top | GTtaa (SEQ ID NO: 79) |
| RAS1-TAA13- | AATCAATTGAATGGTTAAAGCGGATTTACCAACACCgaattcttaACCTCCAACA |
| bottom | ACAAC (SEQ ID NO: 80) |
| MtlA1-top | TTTAAAAAGTGTAGAGAAACTAGTTCAAGCAACATCAGTATATAAGAATGAAGAC |
| | AACGA (SEQ ID NO: 81) |
| MtlA1-bottom | TGCCTCTCACGCTTCAATTGTAAGAATATTTgaattcatTCGTTGTCTTCATTCT |
| | TATAT (SEQ ID NO: 82) |
| Mt1ALpha2-top | ACAACACTAACTCGGTACTCAAGTTATACTCACATCAATAACAAGACATGAATTC |
| | ACATC (SEQ ID NO: 83) |
| MtlAlpha2- | GCAAGCGTTGATTTATTTCAAAGAGTGCCTCggatccttaaAGATGTGAATTCAT |
| bottom | GTCTT (SEQ ID NO: 84) |
| Snf1-Mal-PCR- | TTCACAGAGTGATTATCTGAGTCGTTCATACACCCAAGAAGTTTGATATTTTTGT |
| top-fwd | CTAGT (SEQ ID NO: 85) |
| Snfl-Mal-PCR- | TGACATCTTTAACTCTATGTTATTATATAATGTGTATTACCATTGTAGTTGATTA |
| bottom-rev | TTAGT (SEQ ID NO: 86) |
| Snf1K81R-top | CTCAAGACATTAGGTGAAGGGTCATTTGGTAAAGTGAAATTGGCTCAACACcTcG |
| | GtACAGGTCAAAAAGTTGCTTTGAgAAT (SEQ ID NO: 87) |
| Snf1K81R- | TAAATATGAAATCTCTCTTTCAACACGACCCTGCATGTCgcttTTtGCTAATGTT |
| bottom | TTACGATTAATaATTcTCAAAGCAACTTT (SEQ ID NO: 88) |
| Snf1K81R- | TAAATATGAAATCTCTCTTTCAACACGACCCTGCATGTCgcttTTtGCTAATGTT |
| EcoR1-bottom | TTACGATTAAgaATTcTCAAAGCAACTTT (SEQ ID NO: 89) |
| DCR1-1-top | TTTTCTCAAAAAAATCTAGCAGCACAAAATATAGCAGAAACTGCCAACAAAtaag |
| | aattc (SEQ ID NO: 90) |
| DCR1-1-bottom | GTTGACTGGTAGATGTCCAGTTGTTGATGTAACTCATAAAgaattcttaTTTGTT |
| | GGCA (SEQ ID NO: 91) |
| DCR1-2-top | TAGCAGCACAAAATATAGCAGAAACTGCCAACAAAGGGTTTATGAGTTACATCAA |
| | CAACT (SEQ ID NO: 92) |
| DCR1-2-bottom | ACTTTATTATCTTCTTGTTGACTGGTAGATGTgaattcttAGTTGTTGATGTAAC |
| | TCATA (SEQ ID NO: 93) |
| Tpk2-top | ACAATTTCAACAACCGCAGCAACAACTTTATtaAgaattcGGCGAACAAATAGTT |
| | CACCC (SEQ ID NO: 94) |
| Tpk2-bottom | TGTTACATTTGTAGTATTTTGTCCAGTTTGGGCTGCAGCAGGGTGAACTATTTGT |
| | TCGCC (SEQ ID NO: 95) |

CDR1/2 guide sequence
GTTTTGGGGAGACCCGGTGC (SEQ ID NO: 96)
Wild-type Streptococcus pyogenes Cas9 nucleotide sequence
ATGGATAAGAAATACTCAATAGGCTTAGATATCGGCACAAATAGCGTCGGATGGGC
GGTGATCACTGATGAATATAAGGTTCCGTCTAAAAAGTTCAAGGTTCTGGGAAATAC
AGACCGCCACAGTATCAAAAAAAATCTTATAGGGGCTCTTTTATTTGACAGTGGAGA
GACAGCGGAAGCGACTCGTCTCAAACGGACAGCTCGTAGAAGGTATACACGTCGGA
AGAATCGTATTTGTTATCTACAGGAGATTTTTTCAAATGAGATGGCGAAAGTAGATG
ATAGTTTCTTTCATCGACTTGAAGAGTCTTTTTTGGTGGAAGAAGACAAGAAGCATG
AACGTCATCCTATTTTTGGAAATATAGTAGATGAAGTTGCTTATCATGAGAAATATC
CAACTATCTATCATCTGCGAAAAAAATTGGTAGATTCTACTGATAAAGCGGATTTGC
GCTTAATCTATTTGGCCTTAGCGCATATGATTAAGTTTCGTGGTCATTTTTTGATTGA
GGGAGATTTAAATCCTGATAATAGTGATGTGGACAAACTATTTATCCAGTTGGTACA
AACCTACAATCAATTATTTGAAGAAAACCCTATTAACGCAAGTGGAGTAGATGCTA
AGCGATTCTTTCTGCACGATTGAGTAAATCAAGACGATTAGAAAATCTCATTGCTCA
GCTCCCCGGTGAGAAGAAAAATGGCTTATTTGGGAATCTCATTGCTTTGTCATTGGG
TTTGACCCCTAATTTTAAATCAAATTTTGATTTGGCAGAAGATGCTAAATTACAGCTT
TCAAAAGATACTTACGATGATGATTTAGATAATTTATTGGCGCAAATTGGAGATCAA
TATGCTGATTTGTTTTTGGCAGCTAAGAATTTATCAGATGCTATTTTACTTTCAGATA
TCCTAAGAGTAAATACTGAAATAACTAAGGCTCCCCTATCAGCTTCAATGATTAAAC
GCTACGATGAACATCATCAAGACTTGACTCTTTTAAAAGCTTTAGTTCGACAACAAC
TTCCAGAAAAGTATAAAGAAATCTTTTTTGATCAATCAAAAAACGGATATGCAGGTT
ATATTGATGGGGGAGCTAGCCAAGAAGAATTTTATAAATTTATCAAACCAATTTTAG
AAAAAATGGATGGTACTGAGGAATTATTGGTGAAACTAAATCGTGAAGATTTGCTG
CGCAAGCAACGGACCTTTGACAACGGCTCTATTCCCCATCAAATTCACTTGGGTGAG
CTGCATGCTATTTTGAGAAGACAAGAAGACTTTTATCCATTTTTAAAAGACAATCGT
GAGAAGATTGAAAAAATCTTGACTTTTCGAATTCCTTATTATGTTGGTCCATTGGCG
CGTGGCAATAGTCGTTTTGCATGGATGACTCGGAAGTCTGAAGAAACAATTACCCCA
TGGAATTTTGAAGAAGTTGTCGATAAAGGTGCTTCAGCTCAATCATTTATTGAACGC
ATGACAAACTTTGATAAAAATCTTCCAAATGAAAAAGTACTACCAAAACATAGTTTG
CTTTATGAGTATTTTACGGTTTATAACGAATTGACAAAGGTCAAATATGTTACTGAA
GGAATGCGAAAACCAGCATTTCTTTCAGGTGAACAGAAGAAAGCCATTGTTGATTTA
CTCTTCAAAACAAATCGAAAAGTAACCGTTAAGCAATTAAAAGAAGATTATTTCAA
AAAAATAGAATGTTTTGATAGTGTTGAAATTTCAGGAGTTGAAGATAGATTTAATGC
TTCATTAGGTACCTACCATGATTTGCTAAAAATTATTAAAGATAAAGATTTTTTGGAT
AATGAAGAAAATGAAGATATCTTAGAGGATATTGTTTTAACATTGACCTTATTTGAA
GATAGGGAGATGATTGAGGAAAGACTTAAAACATATGCTCACCTCTTTGATGATAA
GGTGATGAAACAGCTTAAACGTCGCCGTTATACTGGTTGGGGACGTTTGTCTCGAAA
ATTGATTAATGGTATTAGGGATAAGCAATCTGGCAAAACAATATTAGATTTTTTGAA
ATCAGATGGTTTTGCCAATCGCAATTTTATGCAGCTGATCCATGATGATAGTTTGAC
ATTTAAAGAAGACATTCAAAAAGCACAAGTGTCTGGACAAGGCGATAGTTTACATG
AACATATTGCAAATTTAGCTGGTAGCCCTGCTATTAAAAAAGGTATTTTACAGACTG
TAAAAGTTGTTGATGAATTGGTCAAAGTAATGGGGCGGCATAAGCCAGAAAATATC
GTTATTGAAATGGCACGTGAAAATCAGACAACTCAAAAGGGCCAGAAAAATTCGCG
AGAGCGTATGAAACGAATCGAAGAAGGTATCAAAGAATTAGGAAGTCAGATTCTTA
AAGAGCATCCTGTTGAAAATACTCAATTGCAAAATGAAAAGCTCTATCTCTATTATC
TCCAAAATGGAAGAGACATGTATGTGGACCAAGAATTAGATATTAATCGTTTAAGT
GATTATGATGTCGATCACATTGTTCCACAAAGTTTCCTTAAAGACGATTCAATAGAC
AATAAGGTCTTAACGCGTTCTGATAAAAATCGTGGTAAATCGGATAACGTTCCAAGT
GAAGAAGTAGTCAAAAAGATGAAAAACTATTGGAGACAACTTCTAAACGCCAAGTT
AATCACTCAACGTAAGTTTGATAATTTAACGAAAGCTGAACGTGGAGGTTTGAGTGA
ACTTGATAAAGCTGGTTTTATCAAACGCCAATTGGTTGAAACTCGCCAAATCACTAA
GCATGTGGCACAAATTTTGGATAGTCGCATGAATACTAAATACGATGAAAATGATA
AACTTATTCGAGAGGTTAAAGTGATTACCTTAAAATCTAAATTAGTTTCTGACTTCC
GAAAAGATTTCCAATTCTATAAAGTACGTGAGATTAACAATTACCATCATGCCCATG
ATGCGTATCTAAATGCCGTCGTTGGAACTGCTTTGATTAAGAAATATCCAAAACTTG
AATCGGAGTTTGTCTATGGTGATTATAAAGTTTATGATGTTCGTAAAATGATTGCTA
AGTCTGAGCAAGAAATAGGCAAAGCAACCGCAAAATATTTCTTTTACTCTAATATCA
TGAACTTCTTCAAAACAGAAATTACACTTGCAAATGGAGAGATTCGCAAACGCCCTC
TAATCGAAACTAATGGGGAAACTGGAGAAATTGTCTGGGATAAAGGGCGAGATTTT
GCCACAGTGCGCAAAGTATTGTCCATGCCCCAAGTCAATATTGTCAAGAAAACAGA
AGTACAGACAGGCGGATTCTCCAAGGAGTCAATTTTACCAAAAAGAAATTCGGACA
AGCTTATTGCTCGTAAAAAAGACTGGGATCCAAAAAAATATGGTGGTTTTGATAGTC
CAACGGTAGCTTATTCAGTCCTAGTGGTTGCTAAGGTGGAAAAAGGGAAATCGAAG
AAGTTAAAATCCGTTAAAGAGTTACTAGGGATCACAATTATGGAAAGAAGTTCCTTT
GAAAAAAATCCGATTGACTTTTTAGAAGCTAAAGGATATAAGGAAGTTAAAAAAGA
CTTAATCATTAAACTACCTAAATATAGTCTTTTTGAGTTAGAAAACGGTCGTAAACG
GATGCTGGCTAGTGCCGGAGAATTACAAAAAGGAAATGAGCTGGCTCTGCCAAGCA
AATATGTGAATTTTTTATATTTAGCTAGTCATTATGAAAAGTTGAAGGGTAGTCCAG
AAGATAACGAACAAAAACAATTGTTTGTGGAGCAGCATAAGCATTATTTAGATGAG
ATTATTGAGCAAATCAGTGAATTTTCTAAGCGTGTTATTTTAGCAGATGCCAATTTA
GATAAAGTTCTTAGTGCATATAACAAACATAGAGACAAACCAATACGTGAACAAGC
AGAAAATATTATTCATTTATTTACGTTGACGAATCTTGGAGCTCCCGCTGCTTTTAAA
TATTTTGATACAACAATTGATCGTAAACGATATACGTCTACAAAAGAAGTTTTAGAT
GCCACTCTTATCCATCAATCCATCACTGGTCTTTATGAAACACGCATTGATTTGAGTC
AGCTAGGAGGTGAC (SEQ ID NO: 1)
CaCas9 encoding nucleotide sequence (codon optimized variant)
ATGGATAAAAAGTATAGTATTGGTTTAGATATTGGTACTAACTCTGTGGGTTGGGCA
GTTATCACCGACGAATATAAAGTTCCATCAAAGAAATTTAAGGTGTTAGGTAACACT
GACAGACACTCAATAAAAAAGAATCTTATCGGTGCTCTTTTGTTCGACTCCGGTGAA
ACTGCCGAGGCTACACGTTTAAAAAGAACAGCAAGAAGAAGATATACCCGTAGAAA
AAATAGAATATGTTATTTACAAGAAATCTTTTCTAATGAAATGGCTAAAGTTGATGA
TTCCTTTTTCCATAGATTGGAAGAGTCATTTTTGGTTGAAGAAGACAAAAAGCATGA
GAGACATCCAATCTTTGGGAATATAGTTGATGAAGTGGCTTACCATGAAAAATATCC
TACCATTTATCATTTAAGAAAGAAATTGGTAGATTCAACTGATAAAGCTGACCTTAG
ATTAATCTATTTAGCACTTGCCCATATGATTAAATTTAGAGGTCATTTTTTGATTGAA
GGTGATTTGAACCCAGATAATTCTGACGTGGATAAATTATTTATTCAATTAGTCCAA
ACCTACAACCAATTATTTGAGGAAAATCCAATTAATGCTAGTGGTGTCGATGCCAAA
GCTATATTATCAGCCAGATTATCAAAATCTAGACGTTTGGAAAATTTGATTGCCCAA
TTGCCAGGAGAAAAAAAGAATGGATTATTTGGAAACTTGATCGCATTATCATTGGGT
TTGACACCAAATTTTAAATCTAATTTTGATTTAGCTGAAGATGCTAAATTACAATTAT
CAAAAGACACCTATGACGACGATTTGGACAATTTACTTGCTCAAATTGGTGATCAAT
ATGCAGATTTGTTCTTAGCTGCTAAAAACTTATCTGATGCTATTTTGTTGTCTGATAT
TTTGAGAGTGAACACAGAAATAACCAAAGCTCCATTATCAGCATCTATGATCAAAC
GTTATGATGAACACCATCAGGATTTGACTTTATTGAAAGCTTTGGTGAGACAACAAT
TGCCAGAGAAGTATAAAGAAATCTTTTTCGATCAATCTAAAAACGGGTATGCAGGTT
ATATTGATGGGGGTGCCTCCCAAGAGGAATTTTACAAATTTATAAAACCTATTTTAG
AAAAGATGGATGGGACTGAGGAACTTTTGGTCAAATTGAACAGAGAAGATTTGTTA
CGTAAACAGAGAACTTTTGATAATGGTAGTATACCTCACCAAATTCATTTGGGTGAG
TTGCATGCAATTTTAAGAAGACAAGAAGATTTTTATCCATTTTTAAAAGATAATAGA
GAAAAAATCGAGAAAATTTTAACCTTTAGAATTCCATACTATGTTGGGCCTTTGGCT
AGAGGTAATTCAAGATTTGCCTGGATGACACGTAAATCAGAAGAAACTATTACCCCT
TGGAATTTTGAAGAGGTTGTTGATAAAGGAGCATCAGCACAGAGTTTTATTGAAAG
AATGACCAATTTCGATAAAAACTTACCAAATGAAAAAGTTTTACCAAAACATTCCTT
GTTATACGAATATTTTACTGTTTACAATGAACTTACAAAGGTTAAATATGTTACTGA
AGGTATGCGTAAGCCAGCCTTTTTATCTGGAGAACAGAAAAAGGCAATAGTTGATTT
ATTGTTTAAAACAAATAGAAAAGTTACTGTTAAACAATTAAAAGAAGATTACTTTAA
GAAAATTGAATGTTTTGATTCAGTTGAAATCAGTGGTGTTGAAGACAGATTTAATGC
TAGTTTAGGAACTTACCATGATTTACTTAAAATTATCAAAGATAAAGATTTCTTGGA
TAACGAAGAAAATGAAGACATTTTAGAAGACATTGTTTTAACCTTAACTTTATTCGA
AGATAGAGAGATGATTGAAGAACGTTTGAAGACTTATGCACATTTGTTTGACGATAA
AGTGATGAAACAGTTGAAAAGAAGACGTTATACTGGATGGGGTAGATTGTCTCGTA
AATTGATCAATGGAATTAGAGATAAACAAAGTGGTAAAACTATCTTGGACTTTTTGA
AATCTGACGGATTTGCTAATAGAAATTTCATGCAATTGATCCACGACGATAGTTTGA
CATTTAAAGAAGACATCCAAAAGGCCCAAGTGAGTGGGCAAGGTGATTCATTACAT
GAACATATTGCAAATTTAGCCGGATC TCCTGCTATTAAGAAAGGGATATTACAAACT
GTTAAAGTTGTGGATGAATTAGTGAAAGTAATGGGAAGACATAAACCTGAAAACAT
TGTCATTGAGATGGCAAGAGAAAATCAAACTACACAAAAAGGACAGAAAAATAGT
AGAGAACGTATGAAAAGAATAGAAGAGGGTATTAAAGAATTGGGTAGTCAAATATT
GAAAGAACACCCAGTGGAAAATACCCAGTTGCAAAATGAAAAATTATATC TTTACT
ACCTTCAAAATGGACGTGATATGTATGTTGATCAGGAATTAGATATAAATAGACTTT
CAGATTATGATGTAGATCATATAGTTCCACAATCTTTCTTGAAAGATGATTCCATAG
ACAATAAAGTATTAACTAGAAGTGATAAAAATAGAGGTAAAAGTGATAAT GTCCCA
AGTGAGGAAGTCGTCAAAAAGATGAAAAATTACTGGCGTCAACTTTTGAATGCTAA
ATTAATTACTCAAAGAAAATTTGATAATTTGACTAAAGCAGAAAGAGGTGGGCTTTC
TGAATTAGATAAAGCCGGGTTCATTAAAAGACAATTGGTCGAAACTAGACAAATTA
CTAAACATGTTGCCCAAATTTTAGATTCCCGTATGAACACTAAGTATGACGAAAATG
ATAAGTTAATACGTGAGGTTAAAGTCATTACTTTAAAATCAAAACTTGTCTCTGATT
TCAGAAAGGATTTCCAATTCTATAAAGTTAGAGAAATTAATAATTATCATCATGCTC
ATGATGCATATTTGAATGCTGTAGTTGGAACTGCTTTAATCAAGAAATACCCTAAAT
TAGAATCTGAATTTGTATATGGTGATTACAAAGTCTATGATGTTAGAAAGATGATTG
CTAAATCAGAACAAGAAATTGGTAAAGCTACAGCTAAATACTTCTTTTACTCTAACA
TTATGAATTTCTTTAAAACAGAAATTACTTTGGCAAACGGTGAAATTAGAAAAAGAC
CTCTTATTGAAACAAATGGTGAGACTGGAGAGATAGTTTGGGACAAAGGGCGTGAT
TTCGCTACTGTTAGAAAAGTTTTATCAATGCCACAAGTTAACATTGTAAAGAAAACA
GAGGTTCAAACTGGTGGTTTCTCAAAAGAAAGTATTTTGCCTAAAAGAAATAGTGAT
AAATTGATTGCCAGAAAAAAGGATTGGGATCCAAAGAAATATGGTGGTTTCGACTC
ACCAACCGTAGCCTATTCTGTTTTGGTTGTGGCAAAGGTTGAAAAGGGTAAAAGTAA
AAAGCTTAAATCAGTAAAAGAACTTTTGGGTATTACAATAATGGAAAGAAGTTCCTT
TGAAAAGAACCCTATTGATTTTTTGGAAGCTAAAGGTTATAAGGAAGTAAAGAAGG
ACTTAATAATCAAATTGCCTAAATATTCTTTATTTGAATTAGAAAATGGGAGAAAAA
GAATGTTGGCTTCTGCTGGAGAATTGCAAAAGGGTAATGAATTAGCATTGCCTTCCA
AATATGTTAACTTCTTGTATTTAGCTTCACACTATGAAAAGTTGAAAGGGTCACCAG
AAGATAACGAGCAAAAACAATTATTTGTTGAACAACACAAACACTACTTAGATGAG
ATTATAGAACAAATTAGTGAATTCAGTAAAAGAGTGATATTAGCTGATGCAAATTTA
GATAAAGTTTTGTCAGCCTATAACAAACATAGAGATAAGCCAATTAGAGAACAAGC
AGAAAACATTATTCACTTATTTACCCTTACCAATTTAGGAGCACCTGCTGCTTTCAAG
TATTTTGATACAACAATTGATCGTAAAAGATATACC TCAACAAAAGAAGTCTTAGAC
GCCACCTTAATTCATCAATCAATCACTGGATTGTATGAGACAAGAATTGATTTGTCT
CAATTGGGTGGTGATGAAGGGGCT (SEQ ID NO: 2)
Nuclease-inactive CaCas9 encoding nucleotide sequence-codon optimized CaCas9
with mutations to inactivate nuclease activity
ATGGATAAAAAGTATAGTATTGGTTTAGCTATTGGTACTAACTCTGTGGGTTGGGCA
GTTATCACCGACGAATATAAAGTTCCATCAAAGAAATTTAAGGTGTTAGGTAACACT
GACAGACACTCAATAAAAAAGAATCTTATCGGTGCTCTTTTGTTCGACTCCGGTGAA
ACTGCCGAGGCTACACGTTTAAAAAGAACAGCAAGAAGAAGATATACCCGTAGAAA
AAATAGAATATGTTATTTACAAGAAATCTTTTCTAATGAAATGGCTAAAGTTGATGA
TTCCTTTTTCCATAGATTGGAAGAGTCATTTTTGGTTGAAGAAGACAAAAAGCATGA
GAGACATCCAATCTTTGGGAATATAGTTGATGAAGTGGCTTACCATGAAAAATATCC
TACCATTTATCATTTAAGAAAGAAATTGGTAGATTCAACTGATAAAGCTGACCTTAG
ATTAATCTATTTAGCACTTGCCCATATGATTAAATTTAGAGGTCATTTTTTGATTGAA
GGTGATTTGAACCCAGATAATTCTGACGTGGATAAATTATTTATTCAATTAGTCCAA
ACCTACAACCAATTATTTGAGGAAAATCCAATTAATGCTAGTGGTGTCGATGCCAAA
GCTATATTATCAGCCAGATTATCAAAATCTAGACGTTTGGAAAATTTGATTGCCCAA
TTGCCAGGAGAAAAAAAGAATGGATTATTTGGAAACTTGATCGCATTATCATTGGGT
TTGACACCAAATTTTAAATCTAATTTTGATTTAGCTGAAGATGCTAAATTACAATTAT
CAAAAGACACCTATGACGACGATTTGGACAATTTACTTGCTCAAATTGGTGATCAAT
ATGCAGATTTGTTCTTAGCTGCTAAAAACTTATCTGATGCTATTTTGTTGTCTGATAT
TTTGAGAGTGAACACAGAAATAACCAAAGCTCCATTATCAGCATCTATGATCAAAC
GTTATGATGAACACCATCAGGATTTGACTTTATTGAAAGCTTTGGTGAGACAACAAT
TGCCAGAGAAGTATAAAGAAATCTTTTTCGATCAATCTAAAAACGGGTATGCAGGTT
ATATTGATGGGGGTGCCTCCCAAGAGGAATTTTACAAATTTATAAAACCTATTTTAG
AAAAGATGGATGGGACTGAGGAACTTTTGGTCAAATTGAACAGAGAAGATTTGTTA
CGTAAACAGAGAACTTTTGATAATGGTAGTATACCTCACCAAATTCATTTGGGTGAG
TTGCATGCAATTTTAAGAAGACAAGAAGATTTTTATCCATTTTTAAAAGATAATAGA
GAAAAAATCGAGAAAATTTTAACCTTTAGAATTCCATACTATGTTGGGCCTTTGGCT
AGAGGTAATTCAAGATTTGCCTGGATGACACGTAAATCAGAAGAAACTATTACCCCT
TGGAATTTTGAAGAGGTTGTTGATAAAGGAGCATCAGCACAGAGTTTTATTGAAAG
AATGACCAATTTCGATAAAAACTTACCAAATGAAAAAGTTTTACCAAAACATTCCTT
GTTATACGAATATTTTACTGTTTACAATGAACTTACAAAGGTTAAATATGTTACTGA
AGGTATGCGTAAGCCAGCCTTTTTATCTGGAGAACAGAAAAAGGCAATAGTTGATTT
ATTGTTTAAAACAAATAGAAAAGTTACTGTTAAACAATTAAAAGAAGATTACTTTAA
GAAAATTGAATGTTTTGATTCAGTTGAAATCAGTGGTGTTGAAGACAGATTTAATGC
TAGTTTAGGAACTTACCATGATTTACTTAAAATTATCAAAGATAAAGATTTCTTGGA
TAACGAAGAAAATGAAGACATTTTAGAAGACATTGTTTTAACCTTAACTTTATTCGA
AGATAGAGAGATGATTGAAGAACGTTTGAAGACTTATGCACATTTGTTTGACGATAA
AGTGATGAAACAGTTGAAAAGAAGACGTTATACTGGATGGGGTAGATTGTCTCGTA
AATTGATCAATGGAATTAGAGATAAACAAAGTGGTAAAACTATCTTGGACTTTTTGA
AATCTGACGGATTTGCTAATAGAAATTTCATGCAATTGATCCACGACGATAGTTTGA
CATTTAAAGAAGACATCCAAAAGGCCCAAGTGAGTGGGCAAGGTGATTCATTACAT
GAACATATTGCAAATTTAGCCGGATCTCCTGCTATTAAGAAAGGGATATTACAAACT
GTTAAAGTTGTGGATGAATTAGTGAAAGTAATGGGAAGACATAAACCTGAAAACAT
TGTCATTGAGATGGCAAGAGAAAATCAAACTACACAAAAAGGACAGAAAAATAGT
AGAGAACGTATGAAAAGAATAGAAGAGGGTATTAAAGAATTGGGTAGTCAAATATT
GAAAGAACACCCAGTGGAAAATACCCAGTTGCAAAATGAAAAATTATATCTTTACT
ACCTTCAAAATGGACGTGATATGTATGTTGATCAGGAATTAGATATAAATAGACTTT
CAGATTATGATGTAGATGCAATAGTTCCACAATCTTTCTTGAAAGATGATTCCATAG
ACAATAAAGTATTAACTAGAAGTGATAAAAATAGAGGTAAAAGTGATAATGTCCCA
AGTGAGGAAGTCGTCAAAAAGATGAAAAATTACTGGCGTCAACTTTTGAATGCTAA
ATTAATTACTCAAAGAAAATTTGATAATTTGACTAAAGCAGAAAGAGGTGGGCTTTC
TGAATTAGATAAAGCCGGGTTCATTAAAAGACAATTGGTCGAAACTAGACAAATTA
CTAAACATGTTGCCCAAATTTTAGATTCCCGTATGAACACTAAGTATGACGAAAATG
ATAAGTTAATACGTGAGGTTAAAGTCATTACTTTAAAATCAAAACTTGTCTCTGATT
TCAGAAAGGATTTCCAATTCTATAAAGTTAGAGAAATTAATAATTATCATCATGCTC
ATGATGCATATTTGAATGCTGTAGTTGGAACTGCTTTAATCAAGAAATACCCTAAAT
TAGAATCTGAATTTGTATATGGTGATTACAAAGTCTATGATGTTAGAAAGATGATTG
CTAAATCAGAACAAGAAATTGGTAAAGCTACAGCTAAATACTTCTTTTACTCTAACA
TTATGAATTTCTTTAAAACAGAAATTACTTTGGCAAACGGTGAAATTAGAAAAAGAC
CTCTTATTGAAACAAATGGTGAGACTGGAGAGATAGTTTGGGACAAAGGGCGTGAT
TTCGCTACTGTTAGAAAAGTTTTATCAATGCCACAAGTTAACATTGTAAAGAAAACA
GAGGTTCAAACTGGTGGTTTCTCAAAAGAAAGTATTTTGCCTAAAAGAAATAGTGAT
AAATTGATTGCCAGAAAAAAGGATTGGGATCCAAAGAAATATGGTGGTTTCGACTC
ACCAACCGTAGCCTATTCTGTTTTGGTTGTGGCAAAGGTTGAAAAGGGTAAAAGTAA
AAAGCTTAAATCAGTAAAAGAACTTTTGGGTATTACAATAATGGAAAGAAGTTCCTT
TGAAAAGAACCCTATTGATTTTTTGGAAGCTAAAGGTTATAAGGAAGTAAAGAAGG
ACTTAATAATCAAATTGCCTAAATATTCTTTATTTGAATTAGAAAATGGGAGAAAAA
GAATGTTGGCTTCTGCTGGAGAATTGCAAAAGGGTAATGAATTAGCATTGCCTTCCA
AATATGTTAACTTCTTGTATTTAGCTTCACACTATGAAAAGTTGAAAGGGTCACCAG
AAGATAACGAGCAAAAACAATTATTTGTTGAACAACACAAACACTACTTAGATGAG
ATTATAGAACAAATTAGTGAATTCAGTAAAAGAGTGATATTAGCTGATGCAAATTTA
GATAAAGTTTTGTCAGCCTATAACAAACATAGAGATAAGCCAATTAGAGAACAAGC
AGAAAACATTATTCACTTATTTACCCTTACCAATTTAGGAGCACCTGCTGCTTTCAAG
TATTTTGATACAACAATTGATCGTAAAAGATATACCTCAACAAAAGAAGTCTTAGAC
GCCACCTTAATTCATCAATCAATCACTGGATTGTATGAGACAAGAATTGATTTGTCT
CAATTGGGTGGTGATGAAGGGGCT (SEQ ID NO: 3)
Two point mutations to inactivate nuclease activity: D10A, H840A (double
underlined-GCT and GCA)
sV40-NLS/FLAG encoding nucleotide sequence
GATCCTAAGAAGAAAAGAAAAGTTGATCCAAAGAAAAAGCGTAAGGTGGATCCTA
AGAAAAAGAGAAAGGTTgactacaaagaccatgacggtgattataaagatcatgacatcgactacaaggatgacg
atgacaagTGATAA (SEQ ID NO: 4)
3xSV40-NLS (underlined)
3xFlag (lower case)
2xSTOP (italicized)
Wildtype Cas9 Protein Sequence
MDKKYSIGLDIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKERGHFLIEGDLNPDNS
DVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGY
AGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPA
FLSGEQKKAIVDLLEKTNRKVTVKQLKEDYFKKIECEDSVEISGVEDRFNASLGTYHDLL
KIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQG
DSLHEHIANLAGSPAIKKGILQTVKVVDELVKVMGRHKPENIVIEMARENQTTQKGQKN
SRERMKRIEEGIKELGSQILKEHPVENTQLQNEKLYLYYLQNGRDMYVDQELDINRLSD
YDVDHIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDPKK
YGGFDSPTVAYSVLVVAKVEKGKSKKLKSVKELLGITEVIERSSFEKNPIDFLEAKGYKE
VKKDLIIKLPKYSLFELENGRKRMLASAGELQKGNELALPSKYVNFLYLASHYEKLKGS
PEDNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGDEG
A (SEQ ID NO: 5)
Nuclease-inactive Cas9 Protein Sequence
MDKKYSIGLAIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKERGHFLIEGDLNPDNS
DVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGY
AGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPA
FLSGEQKKAIVDLLEKTNRKVTVKQLKEDYFKKIECEDSVEISGVEDRFNASLGTYHDLL
KIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQG
DSLHEHIANLAGSPAIKKGILQTVKVVDELVKVMGRHKPENIVIEMARENQTTQKGQKN
SRERMKRIEEGIKELGSQILKEHPVENTQLQNEKLYLYYLQNGRDMYVDQELDINRLSD
YDVDAIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYG
DYKVYDVRKMIAKSEQEIGKATAKYFFYSNIMNFEKTEITLANGEIRKRPLIETNGETGEI
VWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGS
PEDNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGDEG
A (SEQ ID NO: 6)
Two point mutations to kill nuclease: D10A, H840A (double underlined A as
shown in sequence)
SV40-NLS/FLAG peptide sequence
DPKKKRKVDPKKKRKVDPKKKRKVdykdhdgdykdhdidykddddk (SEQ ID NO: 7)
3xsV40 NLS amino acid sequence underlined
3xFLAG epitope amino acid sequence in lowercase
SNR52 promoter
GCGGCCGCaagtgattagacttagtccgttcaaatcaagcacaactctgttcattgtttcaacaagaattaattcaaaaacaggttcggt
gcataatttgcaaaaaaatattgcagcttctgtggctcgaacacagtacctccagatttcaggtttgaaatacttcagtctgacgctctcccagat
gagctaaagctgcaataagaaaacccacgccgggattcgaacccggaatcctttgattagaagtcaaaagcgataaccatttcgccacgca
ggcctacttgatgggffigtaaatggtctacttfficagacctaacagaaattttaatgaaagtcatattcttatacaataaaactgtgtcataaaag
cagatattcgactttcgtagattatataggacccaagaactaaaatttaatgccatattatgcatttttaatctgtaaaagtgttgfficcaacctatc
acaagtacgttcttgtaacttgtgtttgtagggttgcaaatgaatcataacaacatctcaacagaacatgtatagcaaagcttagtataaaatcag
tgffitgagaggcaatccaagaatgtttacatcaaagtttcaataaatatcgaccgaaactgaaaatattttaggttattgttcactffittgtaaata
tttaaacattttttggacctaaaaaaatacaaacaccaattacgtaccaagaagcatctaatcaactcccagatcaccactatacatttaaaagtc
attggtcaataactatactcgagtattgcctcatcaaagaaacaatcaaatattatagatactcactccatcacgtgataatttcactggtatggaa
aagtggaaaattttataaaaaaaaatttgatgcctttggcatagctgaaacttcggcccaataggattggagaatatgttttcgcagcgttcttac
aattaaattgtggtggaagttcgagacttgcgtaaactatttttaattt (SEQ ID NO: 8)
5′ ENo1 target
CTGCCACTACTACCACTGGGaGTtTCGTTCTTCTCGATACTATTAGCTTTACTTCCTGC
ACTAGCAGTGGTTGGATCAACAGAATCTTCATAATCATCAAAATCGTCTTTTGAAGA
CCCCCCGTTTGATGTATGGCCCTGTCTTTTCATCAAACTTTTTATATAGTTGACTGAA
CTGAGGCTAAATATGTGATCATCTTCACTATAGACAATCTTTCTCTTATTTGCACCAC
CGCCACCACTAGTCTTTGAGAAATTCTCAAAACCTTTTACGATATTACCAAGCGGGC
TCTCTTCGAAATAATCTATCTCTTTTTGATATATCGAATCCTCTAGCGTGGTTAGCTT
TCTAGTTAGTTCTTGCTTCTTAAGAATTTGCTGGATTAGTTTATTTTTCAATTCAACGT
ATTTCTCAGAGTCATCTTTAGATTTTGATGAAGATGTGCGTTCATTCGCTATATCCTT
CTTGGTCGTGTCTTTTCGATCCTCCTTGGCTGGCACTGAACTCGTCTTTTTTGGCGTTG
CTGTTCCAGACAGACTTATCTCATTAGATTTGGAACTTGTGGGTTTAACATCATTTGT
ATCTTTAGTAGACATGATTGTGCAATACCGTGATTATTTGTTTTGAAAGGTCTGTCAT
ATTTCTATCAATTTCAAAACAAAATGTTCATCAGAAAAAAGCCAAAAATGTCTCTTC
TAGTTTCTTAGTGGTGTCGCATAATACACAATGTCGCTCAACAATCCACATTCCCGG
CGCATAGCTCAAATCACATGACTACAGCTAACAATTACACAAAAAAAATTCTCTTTT
TGATGTAGCAACTATCTTCAACTAAAACATTTTCTCCTTCGGCCCATGATTGTCCTCC
GGGTCGACAGCAAGCCGTTACAATTGAGATGGAAAGCGACCTACCTTCACTCGATA
AGGTGCTTAATTGTACTTCATATAAATCTGGCCCGGATCTAAACAAATGAGTTCCAT
TAAGCCGTGGGTTCTCAATTAGGGTTTTTGTTTTTGATTTAGAAAAAAGAGATCAAG
ATTTGTTTACAGGTGATGCCTTTTTTTAGAACTTATGCGTTGCAAAAGTTGACTAACG
ATTTCTATAAGGTGATCCACACTAATTATACAAACGTACAAACAGACATACTTTTCC
TGCGTTCACCTGATGTTGGCCAGATTTCTCTCTTCATTGCATAGAACATAACCACACT
AGGGCAACAGAAAAAAAAAAAAAAAGTGCATCGGGAAGTTGTGTTCCATTCATTAT
ATGTCTACTACTGCATATGAGTAGCCCACCCACCACCACCATAGTAAGTTTTTGTGT
ATGCGCGCCGTCAGGTTATTTCATTTCTGAATTTTTCAACCACCTTACTCCCTTTATT
GTTGATTGACAATTTTGCTCACAGTAAGATCTTTTAGACTCCAATTAATATAAAATA
AGTCTGATTTTCCAATTCCTGTTTTTTCTTTTTTTTTCTGTTTCTATTTCTTTCCTTTTCT
CCC TTTTTTTTAATTCTTCATTCAATCATCAATTGATAATTCAGGAATATTACAACAA
ccc (SEQ ID NO: 9)
3′ ENo1 target
ggGTTTGCCTCTGATTAAATAAAAAAAAGCTGGTGCTTTTTTTTTCTTTTATAGGAAC
ATCTTGAATATATGAACTAATTAAATGATAATTTTTTACCCATCTTTACTCTTAATCA
CTGAGCTGCAGTCAAAGAAAAAGGGATACAGCACCTGGTGAAGAGATGAACGGAG
ACTAACTTAGACGCGTTGATTCTTTTTAATTGCACATTTTATTAATCGATGCTAACGT
CTATTTACATATATTCTTTAGAGATATTATCTAGGGCTTCAAATAATCTCTGGACAGC
AATAAAAGTCTCTTCAAAAGTATTGTATAACGGCAATGGGGCTAATCTGATTACATC
TGGTCTTCTTTCGTCACAGATTATAGCATGATCATGCAAGTACGCATTAACTCGTTCC
ATGACGTTCTTGTCCTTTTCATCGAAATGCGGTTGAAACATAATGGACAATTGACAT
CCTCTTTCAGCTGGATTCAAAGGAGTTAAAATTTTAAACCCAAATTTGGAGTTTGAT
GTACTGGATTGTGGTATGTAATACTTGGAATTCGTCAATAGATCCTGTAAAAATTGA
GTCAAAGCAACACTTTTTTCACGAAGTTTAGATACTCCACCCACTTTAGCATACACTT
CCAATGACGACTTCACAGCAACAACATCAAGAACAGAAGGATTTGACTGTCTGTAA
GAAAGAGCCGAGTTTATTGGATCAAACTCTTCTAACATTTTGAATCGTTCTTGGGAG
TTATTGCCCCACCAACCAGCTAGTCTAGGAACGAAACTGCTTTTCTTGTTCTCTATGG
TGTATTTTTCATGCACAAAAATCCCACCTATGGCTCCAGGTCCCGAGTTTAAATATTT
GTAGGAACACCAAGCAGCAAAATCTACTCCCCAATCATGTAAATTTAATGGGACATT
CCCAACTGCATGGGCAAGATCCCACCCAACTTTAATTTGTTGGCTCTTTTCCTTAGCG
TATTTAGTTATTTCCTCTATCTTGAAAAATTGACCAGTGTAGTATTGGATACCAGGAA
AACACACTAGAGCCAATTCATCCAGGTTCTCATCTATAGCCTTGATTATTCTTTCTGT
TTTAATATAAGTTTCACCAGGTTGAACTTCCAATTGAATCAAATGTTTCTCGTCGTAT
CCGAACAATTTAACAATGTTCAAAAATGCATAGTAGTCAGAAGGAAATGCTTGTTTT
TCAAATAAAATTTTGGTTCTTTTCCCCTCAGGTTTGTAAAAATGGATCAACAATGCAT
TCAAGTTTGCTGTTAAAGAACCCATAACTGCAACTTCGTTTTCCTTTGCACCAACAAT
GGGGGCTATTAATGGTAATAAGGGTAAATCGATGTCTACCCACGGTGTTAACAGTTT
GTCAGGATGATTGAAATGAGACTCAACCCCTCGTTCAACCCATGCATTTAATTCATC
ATTGATAGCTTTCTTTGTATTCTTAGGCATCAACCCAAGAGAGTTTCCACATAAATA
AATAGACTCAGTTGATGACTCATATTTATTATTTTTGATACCTAATGATCCAAAAGTT
GGTATGGCAAACTCATTTTTAAAAGTTGGGAACTTTTTGTCCAATTTCTTTGCCTCGG
CTAATGACATCTGATAATAAAATGGGGTTGGAGTAGTTGGTGGTATAACCGGAGAG
ATAGAATTGAAGAAAAAAATCGGAAACAACAAAAAAAGTTGATACCCTGTATTATG
TGGGAGATAATTGCGAATGGTGGAAAAAAAAAAGACGCCATTGAGTCTCAACAACA
ATTCTGTCAGCTGAAGAGCTTTACAATCGAGAAACTATGATTCATTCCGTTTTAATAT
GTATGTGTTTAGTAAACTCATGAATTTTATTTGTGGTCTACTTTAGTACTAACATAAT
CATTGGATAGTCAATAATGATGGTCTTCCGAGACTAATGAAATTCTATACCAAAGTC
GATATTCCAACACAGAAATTGCTCTTGCAACAAGTGCACCTGTTGATATCTAgagct
(SEQ ID NO: 10)
RP10 5′ targeting
Tggttgttaagtcagtagatgatttgttgttgtcgtttgattttgttacagcgtaaccagtgcgttttgtttgtttccacatcatacacttcactgaaac
taaataagtttgtttacattttgagacttcaggtacgacccagggttgcgacaaagtttaggtagtttgtcgtctgaatgtcgcaacaaaataggg
ctgtagccctagtcatgtgatgtgaattaacagaacaagaagaactgctggtgcgcaaaaagattatgtgtattttatgtgcgttgttatcctgca
cactaaaattgagcagtgtacacacacacatcttgggctgtatttttattcttgtttttctggtgttctctcactgttaagctctaagtgaatttgtgtgt
gctgtaatagtgtgtgtgttccaagtcccagctctcacagatactcacgcacgcccatactactgaaaatttcctgactttctgtatctaaaaattt
tttactaggaatttttttcttttacgtttttcacttgtttcatataatcaccaactcaagtacaac (SEQ ID NO: 11)
RP10 3′ targeting
Tgtttaaggataatgataactgaagagaagaattagttttttcaagtgtataatatagtttctctctattaccttttccaataatagcattttaagttttc
tattttattttgtataaaaaacataatgaaaaatacgtataagtaatataaatgagtgtgggattaagtgaatacgagatgttgtagtgataatagg
ggaaactctttggcgaaactacaagagagagtgatgtgctaataatgaacgaagaaatatgtgatttttgtatgaaatttgcaattattctgattg
aatttgggtacttgacattgaatccagaacgactatacaaatgtgctactttgtcaaaatatcctttttgagaatcggcatatttatggccctgaata
tcgactaccacattccttttacaacactacgtaaccttttgagaaagtacaagtgaaagaagtatagaattcagtgtttagtttaacgtaagtatta
ctgtggaatgctttcttcgcgacacaagcaacttgtacctgcacccttcacacaatttatttcctaaaactactccagtgcgaaaacaatagtgct
aaatatgatgatgagagaattcttaacgaacggagtaggaatgtacatactatcactagtttccaaataacaaaaataaaaaaaaaaataacat
ggaacttgtattgctaaataaattactagattttataagcaataaaaagaatttgaaaaggatgcttcatcacaactaatagtttagtttctttacttct
ccectgfttactgggttattttatttagattatgctaatataattattaatacaagaatttttatttttttaatttatgttgctgattgcccctaaaatttcaa
attectgaaattccctgagtgacttgaacccagacacacattcactcactcacacaaacaaatacacaaaattagagaacctgaatttcagatt
ctcaaattccaaaacagcaaag (SEQ ID NO: 12)
Candida albicans SSN6 nucleotide sequence
ATGTATGCGACAGCCCATACAATTAAACAACAACAACAACAACAACAACAACATCC
ACCACCACCTTTAAACGGTGGACTACATGCAAGTGGGGCTCCTCCAAATTCCCATGA
AGCAGCAGCTATTGCTCAGCAACAACAACAACAGCAGCAACACCACAATGGTCCTG
GTATGATTGTTGCCGCAGCTGCAGCTTCTGCTAACCAACAAGCTGTCCAAGCCAGAG
CCCAACAACAACAACAGCAGCAACAACAGCGATTACCTAGTTCAGCTGCTCTTAAT
GAAACTACAGTATCAACTTGGTTAGCCATTGGTTCATTAGCCGAGAGTTTAGGTGAC
ATTGAACGTGCGACAGCTTCTTACAATTCCGCTTTGAGACATTCACCAAATAACCCA
GATATTTTAGTCAAAATAGCAAATACATACCGTTCAAAAGATCAGTTTCTTAAGGCT
GCTGAATTGTATGAACAAGCTCTTAATTTCCATGTTGAGAATGGTGAAACTTGGGGA
TTATTGGGTCATTGTTACTTGATGTTGGATAATTTGCAAAGAGCTTATGCTGCTTATC
AACGTGCATTGTTTTACTTGGAAAACCCTAACGTTCCAAAATTGTGGCACGGAATTG
GTATTTTATATGACAGATATGGCTCATTAGAATATGCTGAAGAAGCCTTTGTGAGAG
TTTTGGATTTGGATCCAAATTTCGACAAGGCTAATGAAATTTATTTCCGTTTAGGGAT
CATTTATAAGCATCAAGGTAAACTACAACCAGCATTAGAATGTTTCCAATACATTTT
GAATAATCCACCACACCCATTAACTCAACCAGATGTTTGGTTTCAAATTGGTTCAGT
GTATGAACAACAAAAGGATTGGAATGGTGCTAAGGATGCTTATGAAAAAGTGTTAC
AGATTAATCCTCATCACGCTAAAGTTTTGCAACAATTGGGATGTCTTTATTCCCAAG
CAGAATCAAATCCATCAACACCAGCTAATGGTGCTGCACCACCACATAAGCCATTCC
AACAAGATTTGACCATTGCTTTAAAATATTTGAAACAATCTTTGGAAGTTGATCAAA
GTGATGCTCATTCATGGTACTATTTGGGTAGAGTAGAAATGATTAGAGGTGATTTCA
CTGCTGCTTATGAAGCTTTCCAACAAGCTGTCAATCGAGATGCAAGAAACCCAACTT
TCTGGTGTTCAATTGGTGTTTTGTACTATCAAATAAGCCAATATCGTGATGCATTGGA
TGCTTATACCAGAGCCATTAGATTAAATCCTTATATCAGTGAAGTATGGTATGATTT
GGGGACTTTGTATGAGACTTGTAATAATCAAATTAGTGATGCATTGGATGCATATAG
ACAAGCAGAAAGATTGGATCCAAATAATCCTCATATAAAGGCAAGATTAGAACAAT
TGACAAAGTATCAACAAGAAGGTAATACTCACCCACCTCAACCACCGCCAAGTTCT
CAACAACCTAGATTACCTCAAGGAATGGTTTTGGAAAGTACTCAACAACAACAGCA
ACAACAACCACCACCACCTCCACAACAACAACAACAACAACTTCAACACCAACTGC
AACTGCAACCTCAACCACAGCAACCACCTCAAACCCAATCACAACCACTGTTACTTC
AACACCAATCTTCATTGCCTCCTCAACAAATCCAACCATTACATCAACAAGCTGCAA
AGCCTTTAGTGAATCAACAACAAAGTCCACCACCACCTCACTTGATGAACTTGGGAC
AACCGGGGCAACAACCACAACAATTGCCACCACATCTTCCACCACATACCCAGCAA
CCTTCTCAAATTCAAGAAAAGCCTCCAACTCAAGAACAACCACATTATCAACCACCT
CCACCTCCACAACATCAACAGCAATCGCAATCGCAACCGCAACCTCCACACCAACC
TCAACACACTCAAAATCAACTGCCTCAATTAGCTCAATTGCCACCACACCATTCTAA
TCCTCCAGCTAAGCCACATGGTGCACCTCAACAAAGAACTGGTTTACCGGATTTATT
ACACAACTCTGCTAATATCATATCAGCTCCATCACAAGTACCTCAACCACAACAACA
ATATCAACAACCACATATTGCACCTGTTAGACAAGAACAAGTTAACCATGTTCCTTC
AATTTATCTGGCTCCTAGACCAACTGAGACAACACTTCCTCAAATCAACAACCCAAA
TGAGTCAACCACAACACAAGTTCCACAACTCAAAAAGGAGGAACCTAAACCAGAGG
CTACTGTTTCTGCTCCAGTTCCTGAGGCTATTAAAGTTCAAGATCAAGTGACAATCC
AGGAGTCAGCACCAGCAGCAGCAGCAGCAGTGTCAGCACCAGCTTCTGCTCCAGTT
GGTGATATAAAAACAGATACTGTATCTACTACTACACCTGCTACTTCAACCACTGCA
GATGCTGTGCCAGTATCTGTGTCTCAAGTTGGTGAAGCACCAAATGTTGTTCAAGAG
AAGAAAGTTCCGGACACCGAGCAGATCGTTTCACAAGTTGAAAAACCCGTGGAGTC
ACAACCAGAAGTTACACCAGCTCCAACACCAGCTCCAGCTCTTGCAACAGCACCAA
CTGAACCTGCACCTACTGATAAGGACGTTGTAATGGCTCCAAGTAAAAGTGCAACA
CCTGTTCCTCAAAGTATTGTGGAACAGAACACCAGAGTATCTGAAGCTACAAAGGC
ACCAGAATCCAATGGTAAACATGATTTAGAAGACAAGAATGATGAAGAAAAAATTT
TAAAGAGGCCAACTGTTGAAACGACTACTGAATCTGTACCAGTTAACCAACCTGTTG
AGAAAGAAAATGAAAAAGTTGAGGTtCCACCGCCACTGGAACAACCAAGTTCAGAA
AAGAGAGAAAAAGAAGTCAACGGATCAATTAAGAAACCATTGGAAAATGAAAGTA
AGGTTGATATTCCTCAATTCTCATCAAATATCACAGCTCAAAATGAAGAAGCAAAAT
CTGGAGAAGAAACTAAAAAAGATACAACCAAGACAAGTCCAGCAAAACAAGGGGA
AGTTAAGGAAGTAATACCATCATCTACAGAAACTGTATCAAAACCAGATGTTGAAA
AAGACAATAAAGAGAAAGACAAAGATGAAGATGAAGTGATGGCTGATGAAGATGA
CGTCAAAAAAGATGAAAATCCAGAACCTCCAATGAGAAAGATTGAAGAAGATGAA
AATTATGATGATGAA (SEQ ID NO: 99)
Candida albicans SSN6 protein sequence
MYATAHTIKQQQQQQQQHPPPPLNGGLHASGAPPNSHEAAAIAQQQQQQQQHHNGPG
MIVAAAAASANQQAVQARAQQQQQQQQQRLPSSAALNETTVSTWLAIGSLAESLGDIE
RATASYNSALRHSPNNPDILVKIANTYRSKDQFLKAAELYEQALNEHVENGETWGLLGH
CYLMLDNLQRAYAAYQRALFYLENPNVPKLWHGIGILYDRYGSLEYAEEAFVRVLDLD
PNEDKANEIYERLGITYKHQGKLQPALECFQYILNNPPHPLTQPDVWFQIGSVYEQQKDW
NGAKDAYEKVLQINPHHAKVLQQLGCLYSQAESNPSTPANGAAPPHKPFQQDLTIALK
YLKQSLEVDQSDAHSWYYLGRVEMIRGDFTAAYEAFQQAVNRDARNPTEWCSIGVLY
YQISQYRDALDAYTRAIRLNPYISEVWYDLGTLYETCNNQISDALDAYRQAERLDPNNP
HIKARLEQLTKYQQEGNTHPPQPPPSSQQPRLPQGMVLESTQQQQQQQPPPPPQQQQQQ
LQHQSQSQPQPQQPPQTQSQPSLLQHQSSLPPQQIQPLHQQAAKPLVNQQQSPPPPHLMN
LGQPGQQPQQLPPHLPPHTQQPSQIQEKPPTQEQPHYQPPPPPQHQQQSQSQPQPPHQPQ
HTQNQSPQLAQLPPHHSNPPAKPHGAPQQRTGLPDLLHNSANIISAPSQVPQPQQQYQQP
HIAPVRQEQVNHVPSIYSAPRPTETTLPQINNPNESTTTQVPQLKKEEPKPEATVSAPVPE
AIKVQDQVTIQESAPAAAAAVSAPASAPVGDIKTDTVSTTTPATSTTADAVPVSVSQVGE
APNVVQEKKVPDTEQIVSQVEKPVESQPEVTPAPTPAPALATAPTEPAPTDKDVVMAPS
KSATPVPQSIVEQNTRVSEATKAPESNGKHDLEDKNDEEKILKRPTVETTTESVPVNQPV
EKENEKVEVPPPSEQPSSEKREKEVNGSIKKPLENESKVDIPQFSSNITAQNEEAKSGEET
KKDTTKTSPAKQGEVKEVIPSSTETVSKPDVEKDNKEKDKDEDEVMADEDDVKKDENP
EPPMRKIEEDENYDDE (SEQ ID NO: 100)

1. A nucleic acid comprising a Candida-compatible clustered regularly interspaced short palindromic repeat (CRISPR)-associated nuclease 9 (CaCas9) nucleotide sequence that encodes a protein having at least 90% sequence identity to SEQ ID NO: 5, or a fragment thereof, wherein each leucine in the protein is encoded by a codon other than CTG or CUG.

2.-3. (canceled)

4. The nucleic acid of claim 1, wherein the CaCas9 nucleotide sequence has at least about 80% identity to SEQ ID NO: 2.

5. (canceled)

6. The nucleic acid of claim 1, wherein the CaCas9 nucleotide sequence encodes a Cas9 protein, wherein the aspartate at position 10, the glutamic acid at position 762, the histidine at position 840, the asparagine at position 863, the histidine at position 983, the aspartic acid at position 986, the arginine at position 1333, or the arginine at position 1335 in SEQ ID NO:5, or a combination thereof, has been substituted with a different amino acid in the Cas9 protein.

7.-8. (canceled)

9. The nucleic acid of claim 6, further comprising a nucleotide sequence encoding a transcription repressor or a transcription activator.

10. (canceled)

11. The nucleic acid of claim 1, further comprising a plasmid sequence.

12.-16. (canceled)

17. The nucleic acid of claim 1, wherein the nucleic acid further comprises a synthetic guide RNA (sgRNA) coding sequence.

18.-29. (canceled)

30. A genetically-modified yeast cell having a nucleic acid comprising a Candida-compatible clustered regularly interspaced short palindromic repeat (CRISPR)-associated nuclease 9 (CaCas9) nucleotide sequence that encodes a protein having at least 90% sequence identity to SEQ ID NO: 5, or fragment thereof, wherein each leucine in the protein is encoded by a codon other than CTG or CUG.

31. The genetically-modified yeast cell of claim 30, wherein the CaCas9 nucleotide sequence has at least about 80% identity to SEQ ID NO:2.

32. (canceled)

33. The genetically-modified yeast cell of claim 30, wherein the CaCas9 nucleotide sequence is integrated into the genome of the yeast cell.

34.-38. (canceled)

39. The genetically-modified yeast cell of claim 30, wherein the yeast cell belongs to a fungal CTG clade species.

40. The genetically-modified yeast cell of claim 39, wherein the fungal CTG clade species is selected from the group consisting of Scheffersomyces (Pichia) stipitis, Candida famata, Candida tropicalis, Meyerozyma (Pichia) guilliermondii, Candida tenuis, Candida maltosa, Candida rugosa, Millerozyma (Pichia) farinosa, Candida oleophila, Candida albicans, Spathaspora passalidarum, Cylichna cylindracea, Debaryomyces hansenii, Lodderomyces elongisporus, Candida melibiosica, Candida parapsilosis, Candida lusitaniae, and Candida guilliermondii.

41. A yeast cell transformed with a nucleic acid of claim 1.

42. (canceled)

43. A method for modifying a genome of a yeast cell, comprising:
a) introducing into the yeast cell a first nucleic acid comprising a Candida-compatible clustered regularly interspaced short palindromic repeat (CRISPR)-associated nuclease 9 (CaCas9) nucleotide sequence that encodes a protein sequence having at least 90% sequence identity to SEQ ID NO: 5, or a fragment thereof, wherein each leucine in the protein is encoded by a codon other than CTG or CUG;
b) introducing into the yeast cell a second nucleic acid comprising an sgRNA coding sequence; and
c) expressing the CaCas9 and sgRNA coding sequences in the yeast cell, thereby modifying the genome of the yeast cell.

44. The method of claim 43, wherein the first and second nucleic acids are introduced into the yeast cell on a single plasmid.

45. The method of claim 43, wherein the first and second nucleic acids are introduced into the yeast cell on two different plasmids.

46. The method of claim 43, further comprising integrating the CaCas9 and sgRNA coding sequences into the genome of the yeast cell.

47. (canceled)

48. The method of claim 43, wherein the sgRNA coding sequence encodes an sgRNA that targets any one or more of the sequences in Supplementary Tables 1A-1H.

49. The method of claim 43, further comprising introducing into the yeast cell a repair template.

50. The method of claim 44, wherein the single plasmid is pV1093 (SEQ ID NO:15), pV1081 (SEQ ID NO:16), pV1086 (SEQ ID NO:17), pV1102 (SEQ ID NO:18), pV1107 (SEQ ID NO:19), pV1123 (SEQ ID NO:20), pV1126 (SEQ ID NO:21), pV1147 (SEQ ID NO:22), pV1129 (SEQ ID NO:23), pV1132 (SEQ ID NO:24), pV1138 (SEQ ID NO:25), pV1144 (SEQ ID NO:26), or pV1201 (SEQ ID NO:29).

51. The method of claim 45, wherein the two different plasmids are pV1025 (SEQ ID NO:13) and pV1090 (SEQ ID NO:14).

52. (canceled)
